(19) United States
(12) Patent Application Publication
(10) Pub. No.: US 2026/0034241 A1
(43) Pub. Date: Feb. 5, 2026

(54) COMPOSITIONS AND METHODS FOR IN VIVO EXPRESSION OF CHIMERIC ANTIGEN RECEPTORS

(71) Applicant: OncoSenX, Inc., Seattle, WA (US)

(72) Inventors: Matthew Rein SCHOLZ, Seattle, WA (US); Jailal Nicholas George ABLACK, San Diego, CA (US)

(21) Appl. No.: 19/065,910

(22) Filed: Feb. 27, 2025

Related U.S. Application Data
(63) Continuation of application No. PCT/US23/73372, filed on Sep. 1, 2023.
(60) Provisional application No. 63/403,662, filed on Sep. 2, 2022.
(60) Provisional application No. 63/441,098, filed on Jan. 25, 2023.

(51) Int. Cl. A61K 48/00; A61K 40/11; A61K 40/31; A61K 40/42; A61P 37/02

(52) U.S. Cl. CPC A61K 48/0033; A61K 40/11; A61K 40/31; A61K 40/4211; A61K 48/0058; A61P 37/02

(57) ABSTRACT

Provided herein are compositions, systems, and methods suitable for in vivo delivery of CAR-encoding expression constructs. Expression constructs engineered to express a heterologous immune receptor under control of an immune cell-specific promoter are described (for example, to express a CAR under control of a T cell-specific promoter). The expression constructs can be used in combination with a lipid-based delivery vector (LDV) delivery system that facilitates effective delivery and repeat dosing.

### CROSS REFERENCE

This Application is a continuation of International Application No. PCT/US23/73372, filed Sep. 1, 2023, which claims priority to and the benefit of U.S. Provisional Patent Application No. 63/441,098, filed Jan. 25, 2023, and U.S. Provisional Patent Application No. 63/403,662, filed Sep. 2, 2022, each of which is incorporated herein by reference in its entirety.

### SEQUENCE LISTING

The instant application contains a Sequence Listing which has been submitted electronically in XML format and is hereby incorporated by reference in its entirety. Said XML copy, created on Feb. 27, 2025, is named 58477_705_301_SL.xml and is 161,064 bytes in size.

### BACKGROUND

Cell therapies that utilize heterologous immune receptors such as chimeric antigen receptors (CARs) and transgenically introduced T cell receptors can provide significant benefits to patients with cancer or other conditions. However, the number of patients able to benefit from CAR-T therapy has been limited by the complexity, labor intensity, and costs of cellular engineering methods.

### SUMMARY

Disclosed herein, in some aspects, is a system for in vivo delivery of an expression construct encoding a heterologous immune receptor, the system comprising: (a) a non-viral lipid-based delivery vector (LDV); and (b) a polynucleotide that encodes the heterologous immune receptor, wherein expression of the heterologous immune receptor is driven by an expression regulatory region that comprises an immune cell-specific promoter.

Disclosed herein, in some aspects, is a system for in vivo delivery of an expression construct encoding a heterologous immune receptor, the system comprising: (a) a non-viral delivery vector; and (b) a polynucleotide that encodes the heterologous immune receptor, wherein expression of the heterologous immune receptor is driven by an expression regulatory region comprising an immune cell-specific promoter that comprises: (i) a CD3 promoter that natively drives expression of CD3 in a mammalian cell; (ii) a CD4 promoter that natively drives expression of CD4 in a mammalian cell; (iii) a distal lymphocyte protein tyrosine kinase (dLck) promoter that natively drives expression of dLck in a mammalian cell; or (iv) an NKp46 promoter that natively drives expression of NKp46 in a mammalian cell.

Disclosed herein, in some aspects, is a system for in vivo delivery of an expression construct encoding a heterologous immune receptor, the system comprising: (a) a lipid-based delivery vector (LDV) that comprises a fusion-associated small transmembrane (FAST) protein; and (b) a polynucleotide that encodes the heterologous immune receptor.

In some embodiments, expression of the heterologous immune receptor is driven by an immune cell-specific promoter. In some embodiments, the immune cell-specific promoter comprises a mammalian promoter. In some embodiments, the immune cell-specific promoter comprises a human promoter. In some embodiments, the immune cell-specific promoter comprises a T cell-specific promoter. In some embodiments, the immune cell-specific promoter comprises a CD3 gamma promoter that natively drives expression of CD3 gamma. In some embodiments, the immune cell-specific promoter comprises a CD3 delta promoter that natively drives expression of CD3 delta. In some embodiments, the immune cell-specific promoter comprises a CD3 epsilon promoter that natively drives expression of CD3 epsilon. In some embodiments, the immune cell-specific promoter comprises a CD3 zeta promoter that natively drives expression of CD3 zeta. In some embodiments, the immune cell-specific promoter comprises a CD4 promoter that natively drives expression of CD4. In some embodiments, the immune cell-specific promoter comprises a CD8 promoter that natively drives expression of CD8. In some embodiments, the immune cell-specific promoter comprises a TRAC promoter that natively drives expression of TRAC. In some embodiments, the immune cell-specific promoter comprises a TCRB promoter that natively drives expression of TCRB. In some embodiments, the immune cell-specific promoter comprises a distal lymphocyte protein tyrosine kinase (dLck) promoter that natively drives expression of dLck. In some embodiments, the immune cell-specific promoter comprises an NKp46 promoter that natively drives expression of NKp46. In some embodiments, the expression regulatory region further comprises an enhancer. In some embodiments, the enhancer is a mammalian CD4 enhancer. In some embodiments, the enhancer is a mammalian CD8 enhancer. In some embodiments, the enhancer is a mammalian CD3 enhancer. In some embodiments, the expression regulatory region further comprises an intron. In some embodiments, the intron is a pCI intron or a CD3 intron. In some embodiments, the expression regulatory region further comprises a splice acceptor. In some embodiments, the expression regulatory region further comprises an exon or a fragment thereof. In some embodiments, the polynucleotide comprises DNA. In some embodiments, the polynucleotide comprises double stranded DNA. In some embodiments, the polynucleotide is a DNA plasmid, nanoplasmid, or minicircle. In some embodiments, the polynucleotide comprises mRNA. In some embodiments, the heterologous immune receptor is a chimeric antigen receptor (CAR). In some embodiments, the CAR is a second generation CAR. In some embodiments, the CAR is a third, fourth, or fifth generation CAR. In some embodiments, the CAR comprises an extracellular binding domain that binds to CD19. In some embodiments, the CAR comprises a CD3 zeta cytoplasmic signaling domain. In some embodiments, the CD3 zeta cytoplasmic signaling domain comprises an inactivated ITAM. In some embodiments, the CD3 zeta cytoplasmic signaling domain comprises two inactivated ITAMs. In some embodiments, the CAR comprises a T cell costimulatory cytoplasmic signaling domain. In some embodiments, the CAR comprises a CD28 cytoplasmic signaling domain. In some embodiments, the CAR comprises a 41BB zeta cytoplasmic signaling domain. In some embodiments, the CAR is a dual CAR. In some embodiments, the CAR is a universal CAR. In some embodiments, the heterologous immune receptor is a T cell receptor. In some embodiments, the polynucleotide further comprises a transgene that encodes an immunomodulatory factor. In some embodiments, the immunomodulatory factor comprises a cytokine. In some embodiments, the immunomodulatory factor comprises a cytokine receptor. In some embodiments, the immunomodulatory factor comprises a chemokine receptor. In some embodiments, the immunomodulatory factor comprises an immune co-receptor. In some embodiments, the LDV comprises a fusion-associated small transmembrane (FAST) protein. In some embodiments, the LDV is formulated for non-targeted delivery to immune cells and non-immune cells. In some embodiments, the LDV is formulated for non-targeted delivery to T cells and non-T cells. In some embodiments, the LDV is formulated for targeted delivery to T cells. In some embodiments, the LDV comprises an ionizable lipid. In some embodiments, a molar ratio of the ionizable lipid to the polynucleotide is between about 2:1 and 25:1. In some embodiments, the molar ratio is about 5:1, about 7.5:1, about 10:1, or about 15:1. In some embodiments, the ionizable lipid comprises Dlin-KC2-DMA (KC2), DODMA, DODAP, DOBAQ, DOTMA, 18:1 EPC, DOTAP, DDAB, 18:0 EPC, 18:0 DAP, or 18:0 TAP. In some embodiments, the FAST protein comprises p10, p13, p14, p15, p16, p22, or a functional domain thereof. In some embodiments, the FAST protein comprises a fusion of a first domain from a p14 FAST protein or a p10 FAST protein and a second domain from a p14 FAST protein or a p15 FAST protein. In some embodiments, the FAST protein comprises an ectodomain of p14 and an endodomain of p15.

Disclosed herein, in some aspects, is an expression construct for in vivo delivery of a heterologous immune receptor, the expression construct comprising a polynucleotide that comprises: (a) a transgene that encodes the heterologous immune receptor; and (b) an expression regulatory region, wherein the expression regulatory region comprises: (i) a T cell-specific promoter, and (ii) an enhancer or an intron.

In some embodiments, the expression construct comprises the enhancer. In some embodiments, the enhancer is a mammalian CD4 enhancer, CD3 enhancer, or CD8 enhancer. In some embodiments, the expression construct comprises the intron. In some embodiments, the intron is a pCI intron or a CD3 intron. In some embodiments, the expression regulatory region further comprises a splice acceptor. In some embodiments, the expression regulatory region further comprises an exon or a fragment thereof.

Disclosed herein, in some aspects, is a method of expressing a CAR in an immune cell, the method comprising contacting the cell with the system of or the expression construct of any one of the preceding embodiments.

Disclosed herein, in some aspects, is a method of treating a condition in a subject in need thereof, the method comprising administering to the subject an effective amount of the system or the expression construct of any one of the preceding embodiments.

In some embodiments, the administering is parenteral. In some embodiments, the administering is intravenous. In some embodiments, the administering is systemic. In some embodiments, the administering is local. In some embodiments, the administering is intratumoral. In some embodiments, the polynucleotide is not integrated into a genome of the cell. In some embodiments, the polynucleotide is not integrated into a genome of the subject. In some embodiments, the CAR is expressed transiently. In some embodiments, the method further comprises administering a second dose of the system or the expression construct to the subject. In some embodiments, the condition is cancer. In some embodiments, the condition is a B cell cancer. In some embodiments, the condition is acute lymphoblastic leukemia (ALL), multiple myeloma, acute myeloid leukemia (AML), or B cell acute lymphoblastic leukemia (B-ALL).

### BRIEF DESCRIPTION OF THE DRAWINGS

FIG. 1 provides flow cytometry GFP histograms of HEK293T (left) and HeLa (right) cells transiently transfected with EGFP reporter plasmids under the control of the indicated promoters.

FIG. 2 shows the EGFP mean fluorescence intensity (MFI) of the viable, EGFP positive populations of HEK293T (darker dots) and HeLa (lighter dots) cells transiently transfected with EGFP reporter plasmids under the control of the indicated promoters, normalized to the MFI of CMV-EGFP transfected cells.

FIG. 3 provides schematics of expression regulatory elements driving expression of a CAR comprising an anti-CD19 scFv (1D3scFv), CD28 transmembrane and cytoplasmic domains, and CD3 cytoplasmic domain with inactivating YY to FF mutations in the first and third ITAMs.

FIG. 4 is a map of a nanoplasmid with an expression regulatory region comprising a murine CD4 enhancer and a human CD4 promoter driving expression of a transgene encoding a CAR.

FIG. 5 is a map of a nanoplasmid with an expression regulatory region comprising a murine CD3 delta promoter driving expression of a transgene encoding a CAR.

FIG. 6 is a map of a nanoplasmid with an expression regulatory region comprising a human CD3 gamma promoter driving expression of a transgene encoding a CAR.

FIG. 7A provides a western blot for expression of a 1D3 CAR in HEK293T cells transiently transfected with the indicated expression constructs.

FIG. 7B provides a western blot for expression of a 1D3 CAR in Hela cells transiently transfected with the indicated expression constructs.

FIG. 8A provides flow cytometry histograms of surface CAR expression by HEK293T cells following transient transfection and staining with an anti-Rat IgG antibody to detect the 1D3 ScFv portion of the CAR.

FIG. 8B provides flow cytometry histograms of total CAR expression by HEK293T cells following transient transfection, cell fixation, permeabilization, and staining with an anti-Rat IgG antibody to detect the 1D3 ScFv portion of the CAR.

FIG. 8C shows normalized surface to total cell 1D3 CAR expression. The MFI of the populations were first normalized to the CMV-Luc control and then the ratio of surface to total cell 1D3 was determined. The ratio of surface to total cell 1D3 from three experiments is depicted, bars indicate the mean.

FIG. 9A shows a gating strategy for CD45+ hematopoietic cells (left panel), and an overlay of representative histogram of GFP expression in the viable CD45+ population from PBS (lighter histogram) and Nanoplasmid-LDV (NTC-GFP, darker histogram) treated mice. C57BL6 mice were administered 20 mg/kg of LDV encapsulating CMV-EGFP Nanoplasmid or vehicle control IV on Days 0 and 6. On day 7 (24 hours post second dose) lungs and spleen were collected and single cell suspensions were processed and analyzed by flow cytometry.

FIG. 9B quantifies the proportion of CD45+ cells in Lung (left) and Spleen (Right) that were GFP+, from mice treated as described for FIG. 9A, n=3 animals per group, *** P <0.001, ** P<0.01.

FIG. 10A provides a gating strategy of CD3+CD4+ T cells (left panel), and an overlay of representative histograms of GFP expression in the viable CD4+ T cell population from PBS and Nanoplasmid-LDV (NTC-GFP) treated animals.

FIG. 10B quantifies the proportion of CD4+ T cells from lung (left) and spleen (right) that were GFP+, from mice treated as described for FIG. 9A, n=3 animals per group, P<0.001, *P<0.05.

FIG. 11A provides a gating strategy of CD3+CD8+ T cells (left panel), and an overlay of representative histograms of GFP expression in the viable CD8+ T cell population from PBS and Nanoplasmid-LDV (NTC-GFP) treated animals.

FIG. 11B quantifies the proportion of CD8+ T cells from lung (left) and spleen (right) that were GFP+, from mice treated as described for FIG. 9A, n=3 animals per group, P<0.001, *P<0.05.

FIG. 12A shows B cell frequency among lymphocytes in PBMC on days 0, 3, and 28 for mice administered indicated nanoplasmid cargos encapsulated in FAST-LDVs at a dose of 20 mg/kg on Day 0. n=2 per group mean+SD is depicted.

FIG. 12B shows B cell viability among lymphocytes in PBMC on days 0, 3, and 28 for mice administered indicated nanoplasmid cargos encapsulated in FAST-LDVs at a dose of 20 mg/kg on Day 0. n=2 per group mean+SD is depicted.

FIG. 12C shows B cell frequency among lymphocytes in spleen on days 3, 14, and 28 for mice administered indicated nanoplasmid cargos encapsulated in FAST-LDVs at a dose of 20 mg/kg on Day 0. n=2 per group mean+SD is depicted.

FIG. 12D shows B cell viability among lymphocytes in spleen on days 3, 14, and 28 for mice administered indicated nanoplasmid cargos encapsulated in FAST-LDVs at a dose of 20 mg/kg on Day 0. n=2 per group mean+SD is depicted.

### DETAILED DESCRIPTION

Cell therapies that utilize heterologous immune receptors such as chimeric antigen receptors (CARs) and transgenically introduced T cell receptors can provide significant benefits to patients with cancer or other conditions. However, the number of patients able to benefit from CAR-T therapy has been limited by the complexity, labor intensity, and costs of cellular engineering methods. Generation of CAR-T cells can commonly involve harvesting the patient's T cells by apheresis, ex vivo lentiviral transduction, activation and expansion of transduced T cells for approximately two weeks in a cytokine-supplemented tissue culture medium, and further processing (e.g., washing and culturing the T cells, cryopreservation and revival, quality control release assays) prior to re-infusion. The entire process has to be conducted under environmentally controlled GMP-compliant conditions, which are expensive to maintain and run. This approach can be further limited by availability of suitable autologous patient cells, equipment, and facilities.

Compositions, systems, and methods that allow in vivo delivery of expression constructs that encode heterologous immune receptors have the potential to greatly streamline the treatment process and improve accessibility of these life-saving therapies. Provided herein are compositions, systems, and methods suitable for in vivo delivery of CAR-encoding expression constructs, and in some embodiments for improved ex vivo delivery.

The disclosure further provides expression constructs engineered to express a heterologous immune receptor under control of an immune cell-specific promoter (for example, to express a CAR under control of a T cell-specific promoter). The expression constructs can be used in combination with a lipid-based delivery vector (LDV) delivery system that facilitates effective delivery and repeat dosing.

### I. Expression Constructs & Polynucleotides

Compositions, systems, and methods of the disclosure can utilize an expression construct that comprises a polynucleotide to achieve in situ expression of one or more heterologous immune receptors after administering the polynucleotide to a subject using a suitable delivery vector. An expression construct or polynucleotide can comprise, for example, an expression regulatory region (e.g., a promoter, enhancer, intron, and/or exon), a transgene encoding a heterologous immune receptor, a transgene encoding an immunomodulatory factor, a polyadenylation signal, or a combination thereof.

An expression construct or polynucleotide disclosed herein can be or can comprise DNA. An expression construct or polynucleotide disclosed herein can be or can comprise double stranded DNA. For example, an expression construct or polynucleotide disclosed herein can be or comprise a plasmid, such as a nanoplasmid. In some embodiments, an expression construct or polynucleotide disclosed herein is or comprises a minicircle, a midge, a MIP, or a doggy bone. In some embodiments, an expression construct or polynucleotide comprises an R6K origin of replication. In some embodiments an expression construct or polynucleotide lacks an origin of replication. An expression construct or polynucleotide disclosed herein can be or can comprise a circular polynucleotide. An expression construct or polynucleotide disclosed herein can be or can comprise a linear polynucleotide.

In some embodiments, an expression construct or polynucleotide disclosed herein is not single stranded DNA. In some embodiments, an expression construct or polynucleotide disclosed herein lacks a component of a viral genome or lacks a viral packaging element, for example, lacks a 5′ and/or 3′ inverted terminal repeat (ITR).

In some embodiments, an expression construct or polynucleotide disclosed herein is non-integrating, e.g., does not integrate into the genome of a host cell.

In some embodiments, an expression construct or polynucleotide disclosed herein can be or can comprise single stranded DNA.

In some embodiments, a polynucleotide disclosed herein comprises RNA, for example, mRNA.

A polynucleotide can be assembled by a variety of methods, e.g., by automated solid-phase synthesis. A polynucleotide can be constructed using standard solid-phase DNA/RNA synthesis. A polynucleotide can also be constructed using a synthetic procedure. A polynucleotide can be synthesized manually or in a fully automated fashion. A polynucleotide can be a recombinant nucleic acid. In some cases, a synthetic procedure may comprise 5′-hydroxyl oligonucleotides that can be initially transformed into corresponding 5′-H-phosphonate mono esters, subsequently oxidized in the presence of imidazole to activated 5′-phosphorimidazolidates, and finally reacted with pyrophosphate on a solid support. This procedure may include a purification step after the synthesis such as PAGE, HPLC, MS, or any combination thereof. Polynucleotides can be purchased commercially.

A polynucleotide can encode a safety switch, for example, to allow deletion, killing of, or induction of apoptosis of engineered immune cells that comprise or encode a heterologous immune receptor. In some embodiments, the safety switch comprises an epitope that a therapeutic antibody can bind to induce complement-dependent cytotoxicity (CDC) and/or antibody-dependent cell-mediated cytotoxicity (ADCC). The safety switch can comprise, for example, an epitope that is recognized by rituximab. The safety switch can be combined with another domain or tag. For example, the safety switch can be part of RQR8 which further comprises a CD34 epitope that facilitates identification, isolation, sorting, or enrichment of engineered immune cells.

In some embodiments, a safety switch comprises a cytotoxic protein, such as an inducible cytotoxic protein. A safety switch can comprise, for example, a caspase or a catalytic domain thereof. A safety switch can be or comprise a caspase, for example, caspase 1, caspase 3, caspase 8, caspase 9, or a catalytic domain thereof. A safety switch can be, comprise, consist essentially of, or consist of a non-inducible caspase, such as a non-inducible caspase 1, caspase 3, caspase 8, or caspase 9, or a non-inducible protein that comprises a catalytic domain of caspase 1, caspase 3, caspase 8, or caspase 9. A safety switch can be or comprise a self-activating caspase, such as a self-activating caspase 1, self-activating caspase 3, self-activating caspase 8, or self-activating caspase 9, or a self-activating protein that comprises a catalytic domain of caspase 1, caspase 3, caspase 8, or caspase 9. A safety switch can be, comprise, consist essentially of, or consist of an inducible caspase, such as an inducible caspase 1, inducible caspase 3, inducible caspase 8, or inducible caspase 9 (iCasp9), or an inducible protein that comprises a catalytic domain of caspase 1, caspase 3, caspase 8, or caspase 9. An inducible cytotoxic protein, such as an inducible caspase disclosed herein, can be in an inactive state until contacting with a chemical or biological compound that activates the cytotoxic protein. An inducible cytotoxic protein, such as an inducible caspase disclosed herein, can be activated by contacting with a macrolide. An inducible cytotoxic protein, such as an inducible caspase disclosed herein, can be activated by contacting with rapamycin or a structural analogue thereof. An inducible cytotoxic protein, such as an inducible caspase disclosed herein, can be activated by contacting with another inducing agent, such as AP20187. An inducible cytotoxic protein can comprise caspase 9 fused to a human FK506 binding protein (FKBP) to allow conditional dimerization using the small molecule AP20187 (which can be a synthetic analog of FK506). An inducible cytotoxic protein can be a rapamycin-inducible cytotoxic protein. For example, a cytotoxic protein can comprise, consist essentially of, or consist of a rapamycin-inducible caspase, such as a rapamycin-inducible caspase 1, rapamycin-inducible caspase 3, rapamycin-inducible caspase 8, or rapamycin-inducible caspase 9, or a rapamycin-inducible protein that comprises a catalytic domain of caspase 1, caspase 3, caspase 8, or caspase 9. A rapamycin-inducible cytotoxic protein can utilize a double-rapamycin inducible system for Caspase 3 and 9 that employs RU486 and chemical inducers of dimerization (CID). A rapamycin-inducible cytotoxic protein can utilize rapamycin inducible caspase 8 system by employing the ARIAD™ homodimerization system (FKC8; ARIAD Pharmaceuticals). A rapamycin-inducible cytotoxic protein can comprise a full length rapamycin-inducible caspase 9. For example, a rapamycin-inducible cytotoxic protein can comprise a caspase recruitment domain (CARD; GenBank NM001 229) linked to two 12 kDa human FK506 binding proteins. The FK506 binding proteins can be, for example, FKBP12 (GenBank AH002 818) that optionally contain an F36V mutation. A linker (e.g., a Ser-Gly-Gly-Gly-Ser linker, or another linker disclosed herein) can connect the FK506 binding proteins and/or the FKBPs and caspase 9. A rapamycin-inducible cytotoxic protein can include a dimerization domain, such as an FKBP, FK506, and/or FRB binding protein domain, that binds to rapamycin or a structural analog thereof. Illustrative genes (e.g., human genes) encoding FKBP domains include AIP, AIPLI, FKBPIA, FKBPIB, FKBP2, FKBP3, FHBP5, FKBP6, FKBP7, FKBP8, FKBP8, FKBP9L, FKBP10, FKBP11, FKBP14, FKBP15, FKBP52, and LOC541473. Rapamycin-inducible cytotoxic proteins, including rapamycin-inducible caspases, can include (i) an FRB domain (e.g., from, based on, or derived from mTOR); (ii) an FKBP12 domain; and (iii) a caspase or functional fragment thereof. Heterodimerization between an FRB domain of a first rapaCaspase fusion protein and an FKB12 domain of a second rapaCaspase fusion protein can activate the caspase activity. In some embodiments, a first heterodimerization domain of a rapamycin-inducible cytotoxic protein disclosed herein comprises an FK506-binding protein (FKBP) and a second heterodimerization domain comprises an FRB domain (e.g., that is from, based on, or derived from mTOR). The rapamycin inducible cytotoxic protein can comprise one polypeptide chain (e.g., with domains that heterodimerize), or two polypeptide chains that dimerize. The rapamycin or structural analog thereof can bind with a high affinity to the FKBP12 protein, creating a drug-protein complex that subsequently binds to a second protein or domain, such as FKBP-rapamycin binding (FRB) domain or a derivative thereof. A rapamycin-inducible cytotoxic protein can be activated by rapamycin. A rapamycin-inducible cytotoxic protein can be activated by a structural analogue of rapamycin, such as FK506, C-20-methyllyrlrapamycin (MaRap), C16(S)-Butylsulfonamidorapamycin (C16-BS-Rap), C16-(S)-7-methylindolerapamycin (AP21976/C16-AiRap), C16-(S)-3-mehylindolerapamycin (C16-iRap), Sirolimus, Tacrolimus, Everolimus, Temsirolimus, or Deforolimus.

### A. Expression Regulatory Region

An expression construct or polynucleotide disclosed herein can comprise an expression regulatory region. An expression regulatory region can comprise, for example, a promoter (e.g., a T cell-specific promoter), an enhancer, an intron, an exon, a poly (A) sequence, a functional fragment thereof, or a combination thereof. An expression construct or polynucleotide can comprise multiple expression regulatory regions, for example two expression regulatory regions, or more.

### 1. Promoter

A promoter disclosed herein can be a mammalian promoter or derived from a mammalian promoter. A promoter disclosed herein can be a human promoter or derived from a human promoter. A promoter disclosed herein can be a murine promoter or derived from a murine promoter.

The promoter can be a promoter as found in a naturally-occurring genome. In some embodiments, a promoter is not found in a naturally-occurring genome. In some embodiments, the promoter is an engineered promoter. The promoter can be a minimal promoter.

A promoter can be an immune-cell selective promoter, for example, a promoter that results in preferential expression in immune cells as compared to non-immune cells. An immune cell-selective promoter can result in preferential expression in, for example, lymphocytes, T cells, CD4+ T cells, CD8+ T cells, alpha-beta T cells, gamma-delta T cells, T regulatory cells (Tregs), cytotoxic T lymphocytes, Th1 cells, Th2 cells, Th17 cells, Th9 cells, naïve T cells, memory T cells, effector T cells, effector-memory T cells (TEM), central memory T cells (TCM), resident memory T cells (TRM), follicular helper T cells (TFH), Natural killer T cells (NKTs), tumor-infiltrating lymphocytes (TILs), Natural killer cells (NKs), Innate Lymphoid Cells (ILCs), ILC1 cells, ILC2 cells, ILC3 cells, lymphoid tissue inducer (LTi) cells, B cells, B1 cells, B1a cells, B1b cells, B2 cells, plasma cells, B regulatory cells, memory B cells, marginal zone B cells, follicular B cells, germinal center B cells, antigen presenting cells (APCs), monocytes, macrophages, M1 macrophages, M2 macrophages, tissue-associated macrophages, dendritic cells, plasmacytoid dendritic cells, neutrophils, mast cells, basophils, eosinophils, common myeloid progenitors, common lymphoid progenitors, or any combination thereof. The expression can be preferential compared to control cells, such as fibroblasts, neurons, epithelial cells, keratinocytes, or hepatocytes, etc., or another cell type or subset disclosed herein.

A promoter can be a T-cell selective promoter, for example, a promoter that results in preferential expression in T cells as compared to non-T cells. In some embodiments, a T cell-selective promoter can limit off-target effects, e.g., limit off target effects resulting from CAR expression in non-T cells. In some embodiments, the promoter is active in peripheral blood T cells. In some embodiments, the promoter is active in tissue-localized cells (e.g., T cells), for example, tumor-infiltrating lymphocytes or tumor-infiltrating T cells.

In some embodiments, a promoter used in a composition, system, or method disclosed herein (e.g., a T cell selective promoter) is a promoter that natively drives expression of CD3 (e.g., CD3 gamma, CD3 delta, CD3 epsilon, or CD3 zeta), CD4, CD8, CD28, T Cell Receptor Beta (TCRB/TRB), T Cell Receptor Alpha Constant (TRAC), distal lymphocyte protein tyrosine kinase (dLck), proximal lymphocyte protein tyrosine kinase (pLCK), T Cell Receptor Gamma Locus (TRG), or T cell receptor delta constant (TRDC).

In some embodiments, a promoter used in a composition, system, or method disclosed herein is a promoter that natively drives expression of a cluster of differentiation (CD) protein, e.g., a promoter that natively drives expression of CD3 gamma, CD3 delta, CD3 epsilon, CD3 zeta, CD4, CD8, CD28, CD56, CD355, or another CD protein disclosed herein.

In some embodiments, a promoter used in a composition, system, or method disclosed herein is a lymphocyte protein tyrosine kinase (Lck) promoter, such as a distal lymphocyte protein tyrosine kinase (dLck) or a proximal Lck (pLck) promoter, for example, a promoter that natively drives expression of dLck or pLck. An Lck (e.g., dLck) promoter can be a promoter that is predominantly active in T cells, including peripheral blood T cells. dLck can become active after thymocyte T cell selection. In some embodiments, a pLck promoter can drive preferential or specific expression in alpha beta T cells, e.g., as compared to gamma delta T cells. Illustrative examples of Lck promoter sequences are provided in Wildin et al. “Developmental regulation of lck gene expression in T lymphocytes.” The Journal of experimental medicine 173.2 (1991): 383-393, and Wildin et al. “Functional dissection of the murine lck distal promoter.” Journal of immunology (Baltimore, Md.: 1950) 155.3 (1995): 1286-1295, which are incorporated herein by reference for such disclosure.

In some embodiments, a promoter used in a composition, system, or method disclosed herein is a CD3 promoter, such as a CD3 gamma, CD3 delta, CD3 epsilon, or CD3 zeta promoter, for example, a promoter that natively drives expression of CD3 gamma, CD3 delta, CD3 epsilon, or CD3 zeta. A CD3 (e.g., CD3 delta) promoter can become active in immature CD3+ thymocytes prior to thymocyte T cell selection, and can exhibit activity in peripheral blood T cells. In some embodiments, a CD3 (e.g., CD3 delta) promoter exhibits stronger expression in T cells than an alternative (e.g., dLck) promoter, but can also exhibit a level of expression in non-T cells, e.g., CD3 expressing granulocytes.

A promoter can be an NK cell selective promoter, for example, a promoter that results in preferential expression in NK cells as compared to non-NK cells. In some embodiments, an NK cell-selective promoter can limit off-target effects, e.g., limit off target effects resulting from CAR expression in non-NK cells. In some embodiments, the promoter is active in peripheral blood NK cells. In some embodiments, the promoter is active in tissue-localized NK cells, for example, tumor-infiltrating NK cells.

In some embodiments, a promoter used in a composition, system, or method disclosed herein (e.g., an NK cell selective promoter) is a promoter that natively drives expression of NKp46 (CD335/NRC1), NRC3, KLRB1, KLRC3, KLRD1, KLRF1 (NKp80), KLRK1 (NKG2D), NKG7, PRF1, CD160, CD244 (2B4), CTSW, FASLG, GZMA, GZMB, GZMH, IL18RAP, IL2RB, KIR2DL4, XCL1, XCL2, CD100 (SEMA4D), CD16 (FcgRIIIA), CD27, CD94, NKG2C, NKG2E, NKG2H, CD96, CRTAM, DAP12, DNAM1 (CD226), KIR2DS1, KIR2DS2, KIR2DS3, KIR2DS4, KIR2DS5, KIR3DS1, Ly49, NCR, NKp30 (NCR3), NKp44 (NCR2), NTB-A (SLAMF6), PSGLI, SLAMF7 (CRACC, CS1, CD319), CD161 (NKR-PIA, NK1.1), NKG2A, CD96, CEACAMI, KIR2DL1, KIR2DL2, KIR2DL3, KIR2DL5A, KIR2DL5B, KIR3DL1, KIR3DL2, KIR3DL3, KLRG1, LAIR1, LIR1 (ILT2, LILRB1), Ly49a, Ly49b, SIGLEC-10, SIGLEC-11, SIGLEC-14, SIGLEC-16, SIGLEC-3 (CD33), SIGLEC-5 (CD170), SIGLEC-6 (CD327), SIGLEC-7 (CD328), SIGLEC-8, SIGLEC-9 (CD329), SIGLEC-E, SIGLEC-F, SIGLEC-G, SIGLEC-H, or TIGIT.

In some embodiments, a promoter used in a composition, system, or method disclosed herein is a Natural Killer Cell P46-Related Protein (NKp46) promoter, for example, a promoter that natively drives expression of NKp46. In some embodiments, the NKp46 promoter preferentially or specifically induces expression in NK cells (e.g., human NK cells), including in the blood and lymph nodes, and irrespective of activation status. NKp46 can be detected on NK cells from the immature stage of NK cell development in the bone marrow through to NK cells isolated from different organs. An NKp46 promoter can exhibit preferential activity in NK cells compared to, for example, T cells, CD1d-restricted NKT cells (e.g., unlike CD56), and/or gamma delta T cells. In some embodiments, NKp46 also induces expression in some innate lymphoid cells (ILCs), such as ILC1 and a subset of group 3 ILCs in mucosa. An illustrative, non-limiting example of a minimal NKp46 promoter is provided by Walzer et al. “Identification, activation, and selective in vivo ablation of mouse NK cells via NKp46.” Proceedings of the National Academy of Sciences 104.9 (2007): 3384-3389, which is incorporated herein by reference for such disclosure.

A promoter can be a promoter that is responsive to an NFAT (Nuclear factor of activated T-cells) transcription factor. The NFAT family of transcription factors comprises five members (NFAT1-NFAT5), including four calcium-regulated NFAT proteins (NFAT1-4) which were first described in T lymphocytes. In some embodiments, a promoter that is responsive to an NFAT transcription factor is also a T cell selective promoter. NFAT transcription factors can regulate gene expression during T cell activation and differentiation. The conserved regions of calcium-regulated NFAT proteins can comprise two tandem domains: (1) the regulatory domain, which is also known as the NFAT-homology region (NHR) and (2) the DNA-binding domain (DBD), also known as the Rel-homology region. The NFAT proteins share a highly conserved DBD that allows NFAT members to bind to a DNA sequence in enhancers or promoter regions. The DBD comprises ˜270 amino acids and shares 64-72% sequence identity among the different NFAT members. This highly conserved domain confers the specificity to bind the DNA core sequence (A/T) GGAAA. Flanking the NHR and the DBD domains are two transcriptional activation domains (TAD) at the N- and C termini, which can be variable among different NFAT members and isoforms.

In some embodiments, a promoter used in a composition, system, or method disclosed herein (e.g., an NFAT-responsive promoter) is a promoter that natively drives expression of IFN-gamma, IL2, IL4, IL6, IL13, IL17, IL31, FOXP3, lymphotoxin beta, TNF alpha, CTLA4, CSF2, CYP3A5, p15, p21, CDK4, CDK6, c-Myc, cyclin A2, cyclin DI, cyclin D3, A1 Bcl-2, BDNF, DDIAS, c-FLIP, FasL, Nur77, TRAIL, or Trim17.

In some embodiments, a promoter disclosed herein reduces or eliminates the need for a targeted delivery vector, e.g., that targets T cells using proteins, antibodies, or other binding agents directed to T cell-specific surface molecules. For example, a system can use a selective promoter (e.g., a T-cell selective promoter) for expression only or preferentially in desired cell type(s), without selective uptake of the expression construct by T cells over other cells, (such as non-immune cells, or monocytes or macrophages).

A promoter disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to any one of SEQ ID NOs: 1-22 (e.g., SEQ ID NO: 1 or SEQ ID NO: 2).

In some embodiments, a promoter disclosed herein comprises, consists essentially of, or consists of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 50 consecutive nucleotides of any one of SEQ ID NOs: 1-22 (e.g., SEQ ID NO: 1 or SEQ ID NO: 2).

In some embodiments, a promoter disclosed herein comprises, consists essentially of, or consists of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 75 consecutive nucleotides of any one of SEQ ID NOs: 1-22 (e.g., SEQ ID NO: 1 or SEQ ID NO: 2).

In some embodiments, a promoter disclosed herein comprises, consists essentially of, or consists of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 100 consecutive nucleotides of any one of SEQ ID NOs: 1-22 (e.g., SEQ ID NO: 1 or SEQ ID NO: 2).

In some embodiments, a promoter disclosed herein comprises, consists essentially of, or consists of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 150 consecutive nucleotides of any one of SEQ ID NOs: 1-22 (e.g., SEQ ID NO: 1 or SEQ ID NO: 2).

In some embodiments, a promoter disclosed herein comprises, consists essentially of, or consists of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 200 consecutive nucleotides of any one of SEQ ID NOs: 1-22 (e.g., SEQ ID NO: 1 or SEQ ID NO: 2).

In some embodiments, a promoter disclosed herein comprises, consists essentially of, or consists of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 300 consecutive nucleotides of any one of SEQ ID NOs: 1-22 (e.g., SEQ ID NO: 1 or SEQ ID NO: 2).

In some embodiments, a promoter disclosed herein comprises, consists essentially of, or consists of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 400 consecutive nucleotides of any one of SEQ ID NOs: 1-22 (e.g., SEQ ID NO: 1 or SEQ ID NO: 2).

In some embodiments, a promoter disclosed herein comprises, consists essentially of, or consists of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 500 consecutive nucleotides of any one of SEQ ID NOs: 1-22 (e.g., SEQ ID NO: 1 or SEQ ID NO: 2).

A promoter disclosed herein can comprise, consist essentially of, or consist of a nucleotide sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity to any one of SEQ ID NOs: 1-22 (e.g., SEQ ID NO: 1 or SEQ ID NO: 2).

In some embodiments, a promoter comprises, consists essentially of, or consists of a nucleotide sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, about 99.5%, or about 100% sequence identity to any one of SEQ ID NOs: 1-22 (e.g., SEQ ID NO: 1 or SEQ ID NO: 2).

In some embodiments, the promoter comprises, consists essentially of, or consists of the nucleotide sequence of any one of SEQ ID NOs: 1-22 (e.g., SEQ ID NO: 1 or SEQ ID NO: 2).

In some embodiments, the promoter comprises a nucleotide sequence with one or more insertions, deletions, and/or substitutions relative to any one of SEQ ID NOs: 1-22 (e.g., SEQ ID NO: 1 or SEQ ID NO: 2).

For example, the promoter can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide insertions relative to any one of SEQ ID NOs: 1-22 (e.g., SEQ ID NO: 1 or SEQ ID NO: 2).

In some embodiments, the promoter comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide insertions relative to any one of SEQ ID NOs: 1-22 (e.g., SEQ ID NO: 1 or SEQ ID NO: 2).

In some embodiments, the promoter comprises 1, 2, 3, 4, 5, 6, 7, 8, 9, 10, 11, 12, 13, 14, 15, 16, 17, 18, 19, 20, 25, 30, 35, 40, 45, or 50 nucleotide insertions relative to any one of SEQ ID NOs: 1-22 (e.g., SEQ ID NO: 1 or SEQ ID NO: 2).

The one or more insertions can be at the 5′ end, the 3′ end, within the nucleotide sequence, or a combination thereof. The one or more insertions can be contiguous, non-contiguous, or a combination thereof.

In some embodiments, the promoter comprises a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide deletions relative to any one of SEQ ID NOs: 1-22 (e.g., SEQ ID NO: 1 or SEQ ID NO: 2).

In some embodiments, the promoter comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide deletions relative to any one of SEQ ID NOs: 1-22 (e.g., SEQ ID NO: 1 or SEQ ID NO: 2).

In some embodiments, the promoter comprises 1, 2, 3, 4, 5, 6, 7, 8, 9, 10, 11, 12, 13, 14, 15, 16, 17, 18, 19, 20, 25, 30, 35, 40, 45, or 50 nucleotide deletions relative to any one of SEQ ID NOs: 1-22 (e.g., SEQ ID NO: 1 or SEQ ID NO: 2).

The one or more deletions can be at the 5′ end, the 3′ end, within the nucleotide sequence, or a combination thereof. The one or more deletions can be contiguous, non-contiguous, or a combination thereof.

In some embodiments, the promoter comprises a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide substitutions relative to any one of SEQ ID NOs: 1-22 (e.g., SEQ ID NO: 1 or SEQ ID NO: 2).

In some embodiments, the promoter comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide substitutions relative to any one of SEQ ID NOs: 1-22 (e.g., SEQ ID NO: 1 or SEQ ID NO: 2).

In some embodiments, the promoter comprises 1, 2, 3, 4, 5, 6, 7, 8, 9, 10, 11, 12, 13, 14, 15, 16, 17, 18, 19, 20, 25, 30, 35, 40, 45, or 50 nucleotide substitutions relative to any one of SEQ ID NOs: 1-22 (e.g., SEQ ID NO: 1 or SEQ ID NO: 2).

The one or more substitutions can be at the 5′ end, the 3′ end, within the nucleotide sequence, or a combination thereof. The one or more substitutions can be contiguous, non-contiguous, or a combination thereof.

In some embodiments, a promoter is not an immune-cell selective promoter. In some embodiments, a promoter is not a T-cell selective promoter. In some embodiments, a promoter disclosed herein is an inducible promoter. In some embodiments, a promoter disclosed herein is a constitutive promoter. In some embodiments, a promoter disclosed herein is not a constitutive promoter. In some embodiments, a promoter disclosed herein is a cell type-selective, subset-selective, or tissue-specific promoter. In some embodiments, a promoter disclosed herein is not a cell type-selective, subset-selective, or tissue-specific promoter.

In some embodiments, a promoter disclosed herein improves selectivity of expression of an expression construct disclosed herein compared to an mRNA payload, which can be translated by any cell it is delivered to.

An expression construct can comprise or utilize a functional fragment of a promoter (e.g., promoter sequence) disclosed herein, for example, a fragment that is sufficient to drive expression of a transgene of interest in a target cell type.

An expression regulatory region disclosed herein can comprise any suitable number of promoters, e.g., operably linked to different transgenes. An expression regulatory region can comprise at least 1, at least 2, at least 3, at least 4, or at least 5 promoters. An expression regulatory region can contain at most 1, at most 2, at most 3, at most 4, or at most 5 promoters. An expression regulatory region can comprise 1, 2, 3, 4, or 5 promoters.

### 2. Enhancer

An expression regulatory region disclosed herein can comprise an enhancer, for example, a CD3 enhancer, CD4 enhancer, CD8 enhancer, or CMV enhancer. An enhancer can be an engineered enhancer. An enhancer can be a synthetic enhancer. An enhancer can be an enhancer as found in a naturally-occurring genome. In some embodiments, an enhancer is not found in a naturally-occurring genome. An enhancer can be a mammalian enhancer or derived from a mammalian enhancer. An enhancer can be a human enhancer or derived from a human enhancer. An enhancer can be a murine enhancer or derived from a murine enhancer. An enhancer can be immune cell-specific. An enhancer can be T cell-specific. In some embodiments, an enhancer is not immune cell-specific or is not T cell-specific. In some embodiments, an enhancer (e.g., a CD3 delta enhancer) disclosed herein acts in a position and/or orientation-independent manner. In some embodiments, an enhancer is an intronic enhancer. In some embodiments, an enhancer is a minimal enhancer.

In some embodiments, a composition, system, or method disclosed herein comprises or utilizes a CD8 (e.g., CD8a), CD3 (e.g., CD3 delta), CD4, or CMV enhancer.

In some embodiments, a composition, system, or method disclosed herein comprises or utilizes a CD3 enhancer, for example a CD3 delta, CD3 gamma, CD3 epsilon, or CD3 zeta enhancer. A CD3 delta enhancer can be a T cell-specific enhancer element found downstream (e.g., immediately downstream) of the 3′ UTR of mouse or human CD3 delta, that facilitates T-cell specific expression, e.g., in a position and orientation-independent manner.

In some embodiments, a composition, system, or method disclosed herein comprises or utilizes a CD8 enhancer, e.g., a CD8a enhancer, a CD8a intronic enhancer, a minimal CD8 enhancer, an E8I, E8II, E8III, E8IV, E8V, or E8VI enhancer, or a combination thereof.

In some embodiments, a composition, system, or method disclosed herein comprises or utilizes a CD4 enhancer.

An enhancer disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to any one of SEQ ID NOs: 23-30.

An enhancer disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 100 consecutive nucleotides of any one of SEQ ID NOs: 23-30.

An enhancer disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 200 consecutive nucleotides of any one of SEQ ID NOs: 23-30.

An enhancer disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 300 consecutive nucleotides of any one of SEQ ID NOs: 23-30.

An enhancer disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 400 consecutive nucleotides of any one of SEQ ID NOs: 23-30.

An enhancer disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 500 consecutive nucleotides of any one of SEQ ID NOs: 23-30.

In some embodiments, the enhancer comprises, consists essentially of, or consists of the nucleotide sequence of any one of SEQ ID NOs: 23-30.

In some embodiments, the enhancer comprises a nucleotide sequence with one or more insertions, deletions, and/or substitutions relative to any one of SEQ ID NOs: 23-30.

For example, the enhancer can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide insertions, deletions, and/or substitutions relative to any one of SEQ ID NOs: 23-30.

In some embodiments, the enhancer comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide insertions, deletions, and/or substitutions relative to any one of SEQ ID NOs: 23-30.

In some embodiments, the enhancer comprises 1, 2, 3, 4, 5, 6, 7, 8, 9, 10, 11, 12, 13, 14, 15, 16, 17, 18, 19, 20, 25, 30, 35, 40, 45, or 50 nucleotide insertions, deletions, and/or substitutions relative to any one of SEQ ID NOs: 23-30.

The one or more insertions, deletions, and/or substitutions can be at the 5′ end, the 3′ end, within the nucleotide sequence, or a combination thereof. The one or more insertions, deletions, and/or substitutions can be contiguous, non-contiguous, or a combination thereof.

An expression construct can comprise or utilize a functional fragment of an enhancer (e.g., enhancer sequence) disclosed herein, for example, a fragment that is sufficient to drive expression of a transgene of interest in a target cell type, in combination with the promoter.

An expression regulatory region disclosed herein can comprise any suitable number of enhancers, e.g., operably linked to different transgenes. An expression regulatory region can comprise at least 1, at least 2, at least 3, at least 4, or at least 5 enhancers. An expression regulatory region can contain at most 1, at most 2, at most 3, at most 4, or at most 5 enhancers. An expression regulatory region can comprise 1, 2, 3, 4, or 5 enhancers.

### 3. Intron

An expression regulatory region disclosed herein can comprise an intron or a truncated version thereof, for example, a CD3 intron or a pCI intron. An intron or a truncated version thereof can, for example, increase expression of a transgene disclosed herein, such as a transgene encoding a heterologous immune receptor (e.g., CAR) or immunomodulatory factor.

The intron can be an engineered intron. The intron can be a synthetic intron. The intron can be an intron as found in a naturally-occurring genome. In some embodiments, an intron is not found in a naturally-occurring genome. The intron can be a mammalian intron or derived from a mammalian intron. The intron can be a human intron or derived from a human intron. The intron can be a murine intron or derived from a murine intron. An intron can be, for example, a CD3 (e.g., CD3 gamma, CD3 delta, CD3 epsilon, or CD3 zeta), CD4, CD8, CD28, TCRB/TRB, TRAC, pCI, beta-globin, or minute virus of mouse (MVM) intron. An intron can be truncated relative to a wild type intron. An intron can be chimeric, for example, comprising a portion of a first intron and a portion of a second intron. The intron can be from or derived from the same source as, for example, a promoter, enhancer, exon, or transgene disclosed herein.

An intron disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to any one of SEQ ID NOs: 31-32.

In some embodiments, the intron comprises, consists essentially of, or consists of the nucleotide sequence of any one of SEQ ID NOs: 31-32.

In some embodiments, the intron comprises a nucleotide sequence with one or more insertions, deletions, and/or substitutions relative to any one of SEQ ID NOs: 31-32.

For example, the intron can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide insertions, deletions, and/or substitutions relative to any one of SEQ ID NOs: 31-32.

In some embodiments, the intron comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide insertions, deletions, and/or substitutions relative to any one of SEQ ID NOs: 31-32.

In some embodiments, the intron comprises 1, 2, 3, 4, 5, 6, 7, 8, 9, 10, 11, 12, 13, 14, 15, 16, 17, 18, 19, 20, 25, 30, 35, 40, 45, or 50 nucleotide insertions, deletions, and/or substitutions relative to any one of SEQ ID NOs: 31-32.

The one or more insertions, deletions, and/or substitutions can be at the 5′ end, the 3′ end, within the nucleotide sequence, or a combination thereof. The one or more insertions, deletions, and/or substitutions can be contiguous, non-contiguous, or a combination thereof.

An expression construct can comprise or utilize a functional fragment of an intron (e.g., intron sequence) disclosed herein, for example, a fragment that is sufficient to increase expression of a transgene of interest in a target cell type as compared to when the intron is absent.

An expression regulatory region disclosed herein can comprise any suitable number of introns, e.g., operably linked to different transgenes. An expression regulatory region can comprise at least 1, at least 2, at least 3, at least 4, or at least 5 introns. An expression regulatory region can contain at most 1, at most 2, at most 3, at most 4, or at most 5 introns. An expression regulatory region can comprise 1, 2, 3, 4, or 5 introns.

### 4. Exon

In some embodiments, an expression regulatory region disclosed herein comprises an exon or a functional fragment thereof. An exon or a truncated version thereof can, for example, increase expression of a transgene disclosed herein, such as a transgene encoding a heterologous immune receptor (e.g., CAR) or immunomodulatory factor.

The exon can be from or derived from the same source as, for example, a promoter, enhancer, intron, or transgene disclosed herein. The exon can be an engineered exon. The exon can be a synthetic exon. The exon can be an exon as found in a naturally-occurring genome. In some embodiments, an exon is not found in a naturally-occurring genome. The exon can be a mammalian exon or derived from a mammalian exon. The exon can be a human exon or derived from a human exon. The exon can be a murine exon or derived from a murine exon. An exon can be, for example, a CD3 (e.g., CD3 gamma, CD3 delta, CD3 epsilon, or CD3 zeta), CD4, CD8, CD28, TCRB, TRAC, pCI, beta-globin, or minute virus of mouse (MVM) exon. An exon can be truncated relative to a wild type exon. An exon can be chimeric, for example, comprising a portion of a first exon and a portion of a second exon.

An exon disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to any one of SEQ ID NOs: 33-35.

In some embodiments, the exon comprises, consists essentially of, or consists of the nucleotide sequence of any one of SEQ ID NOs: 33-35.

In some embodiments, the exon comprises a nucleotide sequence with one or more insertions, deletions, and/or substitutions relative to any one of SEQ ID NOs: 33-35.

For example, the exon can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide insertions, deletions, and/or substitutions relative to any one of SEQ ID NOs: 33-35.

In some embodiments, the exon comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide insertions, deletions, and/or substitutions relative to any one of SEQ ID NOs: 33-35.

In some embodiments, the exon comprises 1, 2, 3, 4, 5, 6, 7, 8, 9, 10, 11, 12, 13, 14, 15, 16, 17, 18, 19, 20, 25, 30, 35, 40, 45, or 50 nucleotide insertions, deletions, and/or substitutions relative to any one of SEQ ID NOs: 33-35.

The one or more insertions, deletions, and/or substitutions can be at the 5′ end, the 3′ end, within the nucleotide sequence, or a combination thereof. The one or more insertions, deletions, and/or substitutions can be contiguous, non-contiguous, or a combination thereof.

An expression regulatory region disclosed herein can comprise any suitable number of exons. An expression regulatory region can comprise at least 1, at least 2, at least 3, at least 4, or at least 5 exons. An expression regulatory region can contain at most 1, at most 2, at most 3, at most 4, or at most 5 exons. An expression regulatory region can comprise 1, 2, 3, 4, or 5 exons. In some embodiments, the expression regulatory region comprises a first exon or functional fragment thereof from a first source (e.g., gene) and a second exon or functional fragment thereof from a second source (e.g., second gene).

An expression regulatory region or a part thereof can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to any one of SEQ ID NOs: 1-40 and 93-94.

In some embodiments, the expression regulatory region comprises, consists essentially of, or consists of the nucleotide sequence of any one of SEQ ID NOs: 1-40 and 93-94.

In some embodiments, the expression regulatory region comprises a nucleotide sequence with one or more insertions, deletions, and/or substitutions relative to any one of SEQ ID NOs: 1-40 and 93-94.

For example, the expression regulatory region can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide insertions, deletions, and/or substitutions relative to any one of SEQ ID NOs: 1-40 and 93-94.

In some embodiments, the expression regulatory region comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide insertions, deletions, and/or substitutions relative to any one of SEQ ID NOs: 1-40 and 93-94.

In some embodiments, the expression regulatory region comprises 1, 2, 3, 4, 5, 6, 7, 8, 9, 10, 11, 12, 13, 14, 15, 16, 17, 18, 19, 20, 25, 30, 35, 40, 45, or 50 nucleotide insertions, deletions, and/or substitutions relative to any one of SEQ ID NOs: 1-40 and 93-94.

TABLE 1
illustrative expression regulatory region sequences.

| SEQ | | |
|---|---|---|
| ID | | |
| NO | Description | Sequence |
| 1 | Human CD4 | GATTTAAGCCTGATTCTGCTTAACTTTTTCCCTTGACTTTG |
| | promoter | GCATTTTCACTTTGACATGTTCCCTGAGAGCCTGGGGGGTG |
| | (homo sapiens) | GGGAACCAGCTCCAGCTGGTGACGTTTGGGGCCGGCCCAG |
| | | GCCTAGGGTGTGGAGGAGCCTTGCCATCGGGCTTCCTGTC |
| | | TCTCTTC |
| 5 | Human CD4 | CTGCAGCCTCAACTTCCTGGGCTCAAGCAATCCTCCCACCT |
| | promoter | CGGCCTCCTAAAATACTGGGATTATAGGCATGAGCCACCA |
| | (homo sapiens) | CTCCCAGCACCACTTTTTTCAGACTGGAAAAGAACACTCA |
| | | CATGTGCATCTTTAAATGACACTTGGGCTGTGGTATGGAG |
| | | AATGGCCACCAGTGAGTAGGCAGGAGCTGTTGTCCGAGCA |
| | | AGGGCTGATATTGGCATCTTGGATTGGCATGGTGGCAGTA |
| | | GTGGTAGTGCAGAGTGACTTGGGTAGATTTTGGAGCATTT |
| | | AGAAGGTACATCCACAGGAACTGGTAAATAAATACGTGG |
| | | GAGAAGTTGGGTGAAGGGGGTGTCAAAGATTACACCCAA |
| | | TTTATTTTGCTTGGGAAGTTGGTGGATGGTGAGCCCCTCAC |
| | | TGAGTGAGAAGCCTGGAGAAGCAGGTTTGGAGGGTGGTA |
| | | GTATGCAGGTGGTATGCATAGTTGGGATGTGTGTTGAGTT |
| | | TGCTATGTCCGGTGAGCTTCCCAGTGGAGATGTCCAATGG |
| | | GCAGACGGATACTCACATAGAGAGTTCATGGTAGATTCGG |
| | | GCTAGAGGAAAGCACCTGAGGCCTGGCCAGAGACGCCTA |
| | | GAGGAACAGAGCCTGGTTAACAGTCACTCCTGGTGTCTCA |
| | | GATATTCTCTGCTCAGCCCACGCCCTCTCTTCCACACTGGG |
| | | CCACCTATAAAGCCTCCACAGATACCCCTGGGGCACCCAC |
| | | TGGACACAATTGCCCTCAGGGCCCCAGAGCAAGGAGCTGT |
| | | TTGTGGGCTTACCACTGCTGTTCCCATATGCCCCCAACTGC |
| | | CTCCCACTTCTTTCCCCACAGCCTGGTCAGACATGGCACTA |
| | | CCACTAATGGAATCTTTCTTGCCATCTTTTTCTTGCCGTTTA |
| | | ACAGTGGCAGTGACACTTGACTCCTGATTAAGCCTGATTC |
| | | TGCTTAACTTTTTCCCTTGACTTTGGCATTTTCACTTTGACA |
| | | TGTTCCCTGAGAGCCTGGGGGGTGGGGAACCAGCTCCAGC |
| | | TGGTGACGTTTGGGGCCGGCCCAGGCCTAGGGTGTGGAGG |
| | | AGCCTTGCCATCGGGCTTCCTGTCTCTCTTCATTTAAGCAC |
| | | GACTCTGCAG |
| 6 | Murine CD4 | TCTAGAACAGCCGACTTAGTGGATCTGTTGGGCAACGTAA |
| | promoter | TCTAAATTTTCCATTGATTAAATTGCTAACTTTTTTTTTTAA |
| | (mus musculus) | AACATGGCATTACTGCATAGGCAGCCTAGTCTGGCCTTGA |
| | | GCTTGTGATTTTTCTGCCTAAATCACCCAGTGCTGGGGTTA |
| | | CAGTTACATGCCCGTGCCCGTGCCCGTGCCTGTAAGCCTTG |
| | | CCTCACGTTGACCTAACCAGGCTGTTTCAGCTTTTTACGGC |
| | | ACCAAACAATAGTGACATTCCAGACTCCAGCTTGATTCTG |
| | | CTCAGGCATTTTCAGTGACTTTGGCATTTTTAATTTTTCAA |
| | | CTTCCCCAACAACTGGGGGTGGGAGGGAGGGACTCCTGAG |
| | | GGCTGGCTTACGTCCGCCGTGCAGAGGAGCCTCACGACCA |
| | | GGCTTCCTGTCTTTTCATTTACGAACATCTGTGAAGGCAAA |
| | | GCAAGACTCTCTTCTTCACTAGGTACCTGTTTGCAAAGTCT |
| | | CGAG |
| 2 | Human CD3 | AACAACTGGCTACGATCCTAACAACTAATGACAGGGACAT |
| | delta promoter | TTATCTCCCTCATGAAGAAACGGTCCCAGGACCATCTCCC |
| | (homo sapiens) | ACCCAGCATCCATTGCGGTTCCCTGTGCAAGATGAGTCTCT |
| | | GAGTGGGAATCCAGCACTCTCTCCCTCTTCTTCCCCACCAC |
| | | CTTCACCCTCCTTAACGGAAAAACAAAAGGCATCTGCACC |
| | | TGCAGCCCTGCTGAGGCCCCTGCTGCTCACACTTGCAGCA |
| | | GAGGGTGGAGGCTCTGGGTTCTTGCCTTCTCTCAAAGGCC |
| | | CCAGCCCCAACAGTGATGGG |
| 7 | Human CD3 | TGCTGGGTGGGAGATGGTCCTGGGACCGTTTCTTCATGAG |
| | delta promoter | GGAGATAAATGTCCCTGTCATTAGTTGTTAGGATCGTAGC |
| | (homo sapiens) | CAGTTGTTACTTGTTGCTAGTTGTTGTTGCTGTAGGTAATG |
| | | AATTTAGGAGGGACTCTCAGATGCTTGGGGCTGGACCTCC |
| | | AAATGGAAAGCCACCTCAGTCCAGGCCTTCATTCAGGAATT |
| | | TTTTTCACTTTTTTTTTCTTTTTCTTTTTTGAGACAGAGTCT |
| | | CACTTTGTTGCCCAGGCTGGAGTGCAGTGGCGCTATCTCA |
| | | GCTCACTGCAACCTCCACCTCCGGGGTTCAAGCGATTCTCC |
| | | TGCCTCAGCCTCCTGAGTAGCTGAGATTACAGGTGCGTGC |
| | | CAGCAAGCCCGGCTAATTTTTGTATTTTTAGTAGAGACGG |
| | | GGTTTCACCTTGTTGGTCAGGCTGGTCTCGAACTCCTGACC |
| | | TTGTGATCCACCTGCCTCAGCCTCCCAAAGTGTTGGGATTA |
| | | CAGGCGTGAGACACGGCACCTGGTTTCACTATTCTTTTGA |
| | | GATCATGAAAGAGGCGCCTGGAAGGCAATAGGTGGAACA |
| | | ATTCCGAGGACAGGCCAGCACATTTTCAGGGGCCAGAGCC |
| | | CGGACTGAATGTCCAGGATCTCAGGCCCCTATCTCCCTCA |
| | | GCCAGTCAGTCAGTGAGCCAACAAAAATGTGCAGAGCTCC |
| | | CGTTATGTGCCAGGCACAAGATACAGCAGTGAGCAAGAC |
| | | AGGCATGACTCTGTCCCCAGGTAAGTGACAGTCTAACTGC |
| | | AGCAGCGTGAAGAGTTTCAGATCCTCGTAGGCCACAGGGG |
| | | AAAAAGGTCCATTTGTCTCACCGGGAGGCTCAATATTGCC |
| | | TGTCAGTCAATTATATTATTATTAATAACTACCCCTTAATG |
| | | AGCACATATGATGCGTGAGGCACCATGCAAATATCTTGTT |
| | | TGAATCCTAATAATGGTACTAATGGAATAAGACTCTGAAA |
| | | AATTTTATGGCTGGCCCAGGCCCCACAGGTAGGCAGTATT |
| | | GGACCCAGGATTCAAATCTCTGGCTGGGGTCTCTAAAGCC |
| | | CAACCTCCCACTGACAAGAAGCTGCTAGATCTGGTGTCCC |
| | | TGGCTGCCTAGTGAAGGGTCCTGAGAAAGATCAGCCTCCA |
| | | TGAGAAATCTAGCTGCTACGGCTTGCGCTATGGGGCCGAC |
| | | GGCTTCTCTCAAGGGGCTTCGAGATGTGGCAGTGTTTAGG |
| | | TTGTGTGTAAATGTGGTTGCATTGTCAATAGGGACGCTAA |
| | | AGTTCAGGCCACCTTTTCCATATTCTCTGCCAGCTCCCTGC |
| | | TCAGAGATAGAGCAATTTACACCGCTTCCTTCCTACCCTAC |
| | | CCCTAGCCCACCCCCACTCTGAAAATTTCCCACCATCAAC |
| | | GGCAGAAAGCAGAGAAGCAGACATCTTCTAGTTCCTCCCC |
| | | CACTCTCCTCTTTCCGGTACCTGTGAGTCAGCTAGGGGAG |
| | | GGCAGCTCTCACCCAGGCTGA |
| 3 | Mouse CD3 | TGATCAGAAACAAGAGGATCTGCTAATGTGGCTTGTGTAA |
| | delta promoter | TAGTTTCTCTCAGTGGATCTAAAGAGGTGGCACTTCCTTGT |
| | (mus musculus) | TTGTGAGTAAATGTAGTTGTATTGTTCATAGGAAGCCCAA |
| | | AATCTTCTTTGTTTGTTACCAGTGACTATTGCTATTTGTTTT |
| | | TTGTTACCAGGAAAGGCTATTCTAAATTCCCTTACACTTCC |
| | | TAGAATTCTGAACAAACTTTCCTCATAGTCTCTGCTTTGTA |
| | | TCAATCATCCCAAACTCTTTGCTTCAGAAAGAGCAGTTTGC |
| | | ACCTGTTTGTGTCTTACCCCACACCTAGCCCACTGTTGCTT |
| | | TGACAGTCTTACACCATCAAAGGCAGAAGGTAGAGAGGCA |
| | | GATTTCTTCTAGTTCCTCCCCCACTCTTCCTTTTTCTGAAT |
| | | ACCCATGAGTCTGTTGGCGGAGGATAGTGCTAACCCTGGC |
| | | TGATCA |
| 8 | Murine CD3 | GCCACCCAGCCACCCAGCCACCCAGACAGCCTGTGCAGCA |
| | delta promoter | GCTGGCCTGGCTCCACCCAGCATGCATTGAGCTGGGACTC |
| | (mus musculus) | TGAGTAGAGGCAATACCAGGTGTCTCCGCCCTCTCCTGTA |
| | | GAGTGTGGGCAGCAGGGGCCCCAGCAGGGGTGTTGATGCT |
| | | CTGACTTATCTCGTTTTCTCTTTTGTTGTTGTTAAGGCTTGC |
| | | AGGATAGCAGGTGGCAGGGGATGAGGTTCTAGATTCCCAC |
| | | TTGGAAACCCCTTTACCACTAGAACCTCTATGCGCAATAG |
| | | GTTGAAGACTCCACTGAGCCTTTTTCTCGATGACAGTGATG |
| | | AATGTTCCTGCCTGTTCTTCTGGCTAGGTCTGGACTTGTTT |
| | | GTCACTTGATCCCAGAACTCATGGGTTTAGGCAGAACTTTT |
| | | CCATGTATGAGACTGGAGTTCTGAATGGAAAAGCATCTTA |
| | | GAGTAGACCTTCCTTAAGAGATTTTTGACACTGTTCTTTTG |
| | | AGAACATCTAAGTGGTACCCACCTAGAAGAGAACATCCCA |
| | | ACGGGACAACTAGCTTATTTCTTGGTGTATGACCCTGGGCT |
| | | AGTTCCAGGGTCTCAGGCTATTGTCTCCTTTAGCCAGTCAA |
| | | TGCATAACAGAAATTCACAGAATTCCTATGTGATTCCAGG |
| | | CACAAAGGATAATTTTGTCCCCAGACAGATGACCTTCTGA |
| | | TTATAGCAGTGTGGAAAGTTAGATCCCCACAGCACATGGT |
| | | AGAAAAGTCTATTGTCTCCTGGGGAGGATCGATTCTACAA |
| | | ATGAGTGGATTGATTATATTGTTAAGAAGACTCTCATACT |
| | | GAGATGGCTTAATGTGGCTGGCATGATGCAAACACTCCTC |
| | | CCCCAATCCTTAGAGTGATGCTGAGGGAGACAAGCTCTGA |
| | | GACACTCCATGCATGGGTACTATGTGCTGGACCTAGGATT |
| | | CCGATGTCTGGATGAGGGCTTCTCAAGTCCATCTTCCCACT |
| | | GGCTAGAAGCAGCTTGATGTGGTGTCCCAGAGTGTCCTGT |
| | | GAAGGGTCCTGAGACTGATCAGAAACAAGAGGATCTGCT |
| | | AATGTGGCTTGTGTAATAGTTTCTCTCAGTGGATCTAAAGA |
| | | GGTGGCACTTCCTTGTTTGTGAGTAAATGTAGTTGTATTGT |
| | | TCATAGGAAGCCCAAAATCTTCTTTGTTTGTTACCAGTGAC |
| | | TATTGCTATTTGTTTTTTGTTACCAGGAAAGGCTATTCTAA |
| | | ATTCCCTTACACTTCCTAGAATTCTGAACAAACTTTCCTCA |
| | | TAGTCTCTGCTTTGTATCAATCATCCCAAAATCTTTGCTTC |
| | | AGAAAGAGCAGTTTGCACCTGTTTGTGTTTTACCCCACACC |
| | | TAGCCCACTGTTGCTTTGACAGTCTTACACCATCAAAGGC |
| | | AGAAGGTAGAGAGGCAGATTTCTTCTAGTTCCTCCCCCAC |
| | | TCTTCCTTTTTCTGAATACCCATGAGTCTGTTGGCGGAGGA |
| | | TAGTGCTAACCCTGGCT |
| 9 | Murine Lck | CTGCTCTACAGAATGAATTTCAGGTCTCAAGGTCAGCTTG |
| | promoter | GTCTACAAAGTGAGTTTCAGATCTCAAGGCCAGCCAAGAC |
| | (mus musculus) | TATGCAGTAAGACCTTAGCTAAAATAAATAAATAAATAAA |
| | | ATAAAATAAAAAGTTAGATCTTCGCTTGGCAAACCGATAG |
| | | ATTGAGGACCAGTGCTCAGGAAGGAGGCACACGGAATTC |
| | | CAGAGGCTACAGAGGGAGCCTCGCTCTGACCTGGTTAGAG |
| | | CAACTCTACTTTACTGGCTGTGTCTATGAGGTTCTGCTTGA |
| | | TTTCATTTGACAAAAAGTTTCCACAGCTAAACCAGGCAAG |
| | | GGAGCCGAAGTAGACACAGCCACCCGGGCCGCGCCAACA |
| | | GGTTTCTCTCTGCTGCTGAGAAGCAAAAGAATGTTTGAAG |
| | | AAACTCTCTGAAGGAGACTGTGGTTGAGTGGTGGGGGTAG |
| | | GGGTGCTGGGGTTGGGCTGAGGCTGAGGGTTGACTCTAAG |
| | | GAGCTGGAACCTCTCAGCTTCGGTGGCTAGGCAGGGGAGT |
| | | TGTAATGAAGAGGGACAGGGTACCCTCCTTGGTGGAGGAG |
| | | GGTGGAATGAAACTCTCGGTTTCCCCCAGAACTTGGCAAA |
| | | GTGTGTGTGATGTCTCCCAGGGTAGTCCCCCAAAGGAGGA |
| | | GGCTAGGCAGAGCTGGGAGGCAGGAAGTGGGTAACTAGA |
| | | CTAACAAAGATGCCTGCCTGTGGCGGTTTGCCCATCCCAG |
| | | GTGGGAGGGTGGGACTAGCCTTGGCCTGGGCCTCCTGTGA |
| | | ACTTGGTGCTTGAGGGCTCAGAGGGAACCCAGTCAGGAGC |
| | | TTGAATCCCACGATTCAGCGCTTCTGTCTGCGGCCAATGG |
| | | GGGCCTCTGAGCTGACGATCTCGGGTACTTTTTGTAACTTC |
| | | CAGAACAGGGCTCTAGGATGTCTGATGTTGGGGCGAGTGG |
| | | CTTAGGGCCAGCTCCTTCAGGCCTCTCTACATTCCTTCAGG |
| | | GATCATGGGCTGTGTCTGCAGCTCAAACCCTGAAGATGAC |
| | | TGGATGGAGAACATTGACGTGTGTGAAAACTGCCACTATC |
| | | CCATAGTCCCGCTGGACAGCAAGATCTCGGTAAGAG |
| 10 | Murine dLck | GAATTCAGTTGCTCGCTATCTTATTTAAAGAGCCAAATCTG |
| | promoter | GAGAAGTAGACCGTAGAGCACAGGTTCTCAATCTGTGGGT |
| | (mus musculus) | CGCAACCCCTTTGGGCATCCAAAAGCACTTTTAGACAGGT |
| | | TGCATATCAGATATCCAGTATATCAGATATTTATATTTAAA |
| | | ATTATTTAAAAGTCGTAGCAGTGGCAAGATTACGGTTACA |
| | | AAGTAGCAACGAAAATAATTTTATGCTTGGGAGTCATCAC |
| | | AGCATGAGGAAATGAGCTGTATTAACGGGTCCCAGCATTA |
| | | GGAAGGCTGAGAACCACTGCCCTAAAGAGAAGAGACAGT |
| | | CAAGTGAAAAGCAGAAAGCGACCCTGCTGTCAGTTATAAT |
| | | CTGAGGAGGGGAGAGTTGAATATTAGCTGTCAGCCCACCA |
| | | TGTGCAAACATAAGGATGTCCAGTTCAGCTCAACGGGTCA |
| | | TCACAGTTAATTTGTGATGGCGTACCAGACGTATATATTTG |
| | | CATACTAACGGTTCCTTGGCTTGGCTTGAGAATCACCCATC |
| | | CGGAAAAAGATCTGCGAGACCGAGGCGGATGAAGTTAGG |
| | | TGAGAAGGGGATTGCGAGCTGGACTATGCAAAGAGTATTC |
| | | CCAGAAATGGACCGGCGGCTGGGCTGGTGAGCCCGGAGG |
| | | CTAATCTAGCAAATTTACGCCCCCTTTTGGTCACAGCAGG |
| | | AGATGCGGGCTCAGGACCGGCAGCTGGCAGGGCAGCTGC |
| | | TCAGGCTGCGGGCCCGGCTGCACAGACTGAAAGTGGACCA |
| | | AGTCTGTCACCTGCACCAGGAGCTTCTGGATGAGGCTGAG |
| | | CTGGAGATGGAGTTAGAGTCTGGGACTGGCTTGCCTCTGG |
| | | CCCCACCGCTGCGGCATCTGGGACTCACGCGCATGAACAT |
| | | CAGTGCCAGACGCTTCACCCTCTGCTGACAGCAGACTTGG |
| | | GTGTCTCTTGCAGTATCTGGGGAGAAAGAAGGAAGGAAG |
| | | AGGGACCCCGGAGGCTCTGGCTACCTGCTGGGGAAGGTGG |
| | | GCACACTTAGGTTTCCAAAAGCTGAATTTAGAGAGCACAG |
| | | GATGGAGGGGAGGAGGAGAGGAAACTCGGGCCCCAAATG |
| | | TCTTAATAAAAAATGCATTGAATCCCATCAAGGTTTCTGTA |
| | | GACTGTCACAGAGCCTAAATAAATGTTGTTGTATATTCATC |
| | | CTGTGTCACTGGGACTTTAGGGATTCCACAACAGGAGACA |
| | | AGATGGCACCAGGTGTCCCCAACAGCTCCCGATCTATTTC |
| | | TCTAACTTTATCAAAATCAAAAGTGAGAAAAATTTAAATG |
| | | AAGATGAAGATTCATTGTGTTTTTTGATTGTGTGTGTGACC |
| | | AGAGAGGCCAGAAAAGGATTAGCTGAGCTGGAGACACAA |
| | | GCTGTTGTAAGCTGCCTGTTGTGGATGCTGCCTACGGAACT |
| | | CAGGTCCTCTGTGAGCAGCAAAGGGCTTTTTTTCTCCAGCC |
| | | CCCAAACTTAAAATCTTCTGCCATTCTCATGACAGGTGGTC |
| | | TAGGTAAATTGAAGACTTGGAGAAAGCAGAAAGTGGTAG |
| | | TTCTGTGTCATCTCTGCTATTTATTTATTTTTTCTTTTTCTGT |
| | | TTTTCTTTTTTTTTTCTTTTCTTTTTCTCTTTTTCCCTGATGC |
| | | AGGGTCTCACTAGGTAACTCCGACTGTCCTGAAGCCCATA |
| | | CTGGCCTTGAACTCACAGAGATTCACTTGCCTCCGGAGTG |
| | | CTGGGGTTAAAGGTGTGCACCACTCTGCCCGGCTTCCCCT |
| | | ATTTAACTCTATTAGACACATAGTGGGAAAATCAGGCCAC |
| | | TATGAGGCAGATCTCTATGATCTTGACGCCAGTCTGCTCTA |
| | | CAGAGCGAGTTCCAGGTCAGTAAGGACCACCTAGAAAGA |
| | | GACCCTGTCTCCAAAAATCCAAAAATAAGCAAACAAACA |
| | | AATTAAGAAAAAAACCCAGCTAGTTGCTAATTTTTGCTCC |
| | | ACGGGCAGCTAGATATGCATTACTTTAGAACACACCCGAT |
| | | AAATTCATGCACTGCTTCTTAGCACCGTGCCCCCCAGGCG |
| | | GGTGCTGAAGCTGAGGTGAGCAGCAGTAAAATGCTAAGG |
| | | TTTCTAAGTGAGGACAGCAGGGCGGTATATCAGGTCTGGA |
| | | GACACATCTGCCGGCTCTGTCACTCTCAGGGTTGGGTTGTT |
| | | TTTTTTTTTTTTCCTTCTGTGTGACTCTCAGCCTTTGTTTCTG |
| | | TGTTTCTCTCTTTGTCTGACTTTCCAACTCAGACTTCCTCCC |
| | | CCCCCCATTTCCTGTTCCCCCTTCCCTCAGCAGCTGGGGTG |
| | | CACACAGCTTCAGCTCCTACCTCACAAACCCCAACAATGC |
| | | TCTGTGGGCTCCCCTCCCCCTGCCTGCTCACTCAGCGTAGC |
| | | TCAAGAGGTGACACATTTACACTTTCTTTCCAGTGAGGTCA |
| | | GGACCCATTCTTGGCCCTGATAGGGGCCATTTGGGGTTCCT |
| | | CACATCACTGGTGGCCTGGCCCTCTGCCTGTGAGGGACAC |
| | | AATCCTCATTTTTGCCTGCTGGCAGCTTGTTGTGGAGTTCT |
| | | TAAACTCCTGCTGATGTCAGAATGTCTCTGCCTGCCACAG |
| | | ATACCTGTGCAGTATGCTTGCTCGGTCCTCTACCGGAGGC |
| | | AACTCGTGGTTATGGCTATGCGCTCCATTAGGGGTGTGGC |
| | | CTTGCCTCTACTTGAATTACCATTCTGCTGTGCTGTCCTGT |
| | | CCCCTGCCTGTTATTCTATCTATCTATCTATCTATCTATCTA |
| | | TCTATCTATCTATCTATCTATCTATCTATTATCTATCTACTA |
| | | TCTATCTATATCTACTATCTATCTATCTATCTATCTTCTATC |
| | | TGTCTCTATCATCTATGTATCTATCTGTGTATCTCTGTATGT |
| | | ATCTATGTTTGTATGTATCTATCTACCTATGTATCTATGTAT |
| | | CTATCTATGTATCTATGTATCTATCTCTATCTATCATCTATG |
| | | TATCTATGTATCTCTGTATGTATATATGTATGTATGTATGT |
| | | GTGTATGTGTGTATGTATGTATGTATGTGTGTATCTACCTA |
| | | TCTATCTATCTACCTACATCTATCTATCTATCTATCTATCTA |
| | | TCTATCGAAACAGGGTCTCTTTATGTAGCTCTGGCTGTCCT |
| | | GGAACTTACTGCATAAGTTAGTCTGGCCTGAAACTTGCAG |
| | | AGATCTGCCAGCCTCTGCCTCCGGAATGCTGGGATTAAAG |
| | | TCTTATGCCACCATGCCCCAGCCTGTCACTAGGGCCTTAAG |
| | | ACATGAGGTTTTCTTGTCTTCCTCCTGAAACAGACTGTCAA |
| | | CAGTCGATCCTTAGGCACAGGATGACATGGATACGGCTCT |
| | | GCCAGTGAATGTGGCTGCTGTTTTTACTATAGTACCTTGTC |
| | | ACCAACTATCTAACAGAGATAGATACTGTCCAGGTTCAAC |
| | | TCTGAACAAGAGATGCCTTTGGCTGTCCGGCTAAATTAAA |
| | | GGCCACCCAGTTCTTACATAGCACGGCTGGAGTTGAGTGC |
| | | AGCGCCATTTCCTTCCCTGTGAGCTAAACTGTAGCTGCTGT |
| | | GTTCTGCTCACGGGGGCAAAGGCTCTCTTTGGATCTCTTAC |
| | | CCAGGAGTATCTTGGAGGGTAGGAGGAGTGCTCCATCAAA |
| | | CAGCCTTCCCCACCATGTTTAGTGACCTTCACGGGGAGGA |
| | | AGAGGCAAGTGAATCTCTGTGAGTTCAAGGCAACATAGCA |
| | | AATTCCATAACAGTCAGGGCGACACGGTGAGTCCCTGCGT |
| | | TAAAAAAAAGATCCTCTCATTGGTTGATGACGACGACGAC |
| | | GATGATGATGATGATTGGACTTGAGGGGTCTAGGCTGGTC |
| | | TGGAACTCTGTATGTAGCCATTCCTGTCTTCACCTCCAGAA |
| | | CGCTGGGAATATGGACCTATGCCATCACACACAGCCTTCC |
| | | TCCCTAAGTGCCTCCCTCAGTATGAGTAGAAGCTGGCCTA |
| | | GAGATGTATGGGACCCAAGAGGCTGAGGGGGTCTAGGTCT |
| | | ATGGCAGCAGGAAGCCTGAGGTGCTGGAGGCTTGTGGTTG |
| | | GGTTGCTGGGGCTGGGTTGGCTGCAGAGCCCTCAGGAGAC |
| | | AGGAAGTCAGGGTGGAACGTGGGCGCGAGGAGACAGGTG |
| | | GTGACTACGACGGCGAGGGGAGCTGACACCGGGTCCAGG |
| | | CAGCCAAGCCAGGCTAGGAGCATCATGTGAATAGGCCAG |
| | | AAGACTCCCAGGCTGGGCAGGTAAGGGGTACTGGGAACA |
| | | ACAGTGTAGAGGGGGGCAATGGTGGGAACCAGGCAACAG |
| | | AACACTGCGTCTGTGGAGGCCTAGGCTCCTTAGGTGTACA |
| | | GAAACCTTTCAGAAATTCCTTCCTGGAGGTTGTCTGAGCTG |
| | | GTGGCTCTTGCCTCTGCCATCTGCTGTCCATCATTTCCCAC |
| | | CTTAGGAGGAGCACACTCCTGGGAGAGCCAGCACATGATG |
| | | TCAAGTGTTAAGAAGGCCACCAGCAGCCACCTGTCCAGGT |
| | | TCCTTGAGCTCAGAGGATAGGGTTTCTAGGTCCAAAGGTG |
| | | GCTGTGACCCTGTCTTAACCCATAGCTCCTCACGATGGTCC |
| | | GCGGCAAAGCAGGACCTTGCGTGGGGGTGTAGGCTTGACC |
| | | TGGGCTCCTTCTATGCCTGTGGCACACAGGGTGATGAGAT |
| | | GGACCGTCCTGAGGCAGGGCCCCAGGAATCAGTCCCCAGG |
| | | TCCCTAAGGCAGACCCAAGTGGCAGAAACAGATGAAAGA |
| | | GTAACCCATGGGATCC |
| 11 | Human dLck | TAGCCTGCCATTCACCTACCCTTGGCTATAATTAAAGCTTA |
| | promoter | CTGGTCCAATGCCCTGAGGTGGCATGGGAAATGGTCTCTC |
| | (homo sapiens) | GGCCTGGCCTGGCTGTAACAATGCATGGTCTGCTCACCAG |
| | | CTATTTAACAGGGATATGTCCTCTCCTCTGACTCTGACGAT |
| | | GCTCCTTTGCTGCCAAGACAGAACAAAGGTCTCTCAGCTG |
| | | CAACGTGTGCTGAGTGCTGTTGCCTCTCACCATAAGCTGA |
| | | GTGTGTGTCCGCTTGCCCCCTGCTCACTGGGCCCAAAGGCT |
| | | GCCCTTGAATCTCTTGCCCAGATGCACCCTGGAGGGCAGA |
| | | AGGGAGGGTCTATCAGACATCCTCCCCCTCAACTTTAAAC |
| | | CTCCCAGTGTCACCCTGGGACAGTAGGGGAAGATGGACCT |
| | | GGTCTGGAGATGTAGGGGACCCCCAGGGGCTGAGAGGCA |
| | | GGGGTCTATGGTGGCAGGAAGCTTGGCGTGCTAGAGGGTT |
| | | GTGGTTGGGCTGCTGGGGCCCGGTTGGCTGCGGAGCCCTC |
| | | CGGAGGAGGCAGGAAGTCAGGGTGGGACGTGGGCGCGGG |
| | | GAGACAGGTGGTGGCTACGACGGCGAAGGGAGCTGAGAC |
| | | TGTCCAGGCAGCCAGGTTAGGCCAGGAGGACCATGTGAAT |
| | | GGGGCCAGAGGGCTCCCGGGCTGGGCAGGTAAGGAGCGC |
| 12 | Human pLck | TGTGTACCCTGTGTATATGTATAGGGTGTGTGTGAGTGTGT |
| | promoter | GTGTGTGAGAGAGTGTGTGTGTGGCAGAATAGACTGCGGA |
| | (homo sapiens) | GGTGGATTTCATCTTGATATGAAAGGTCTGGAATGCATGG |
| | | TACATTAAACTTTGAGGACAGCGCTTTCCAAGCACTCTGA |
| | | GGAGCAGCCCTAGAGAAGGAGGAGCTGCAGGGACTCCGG |
| | | GGGCTTCAAAGTGAGGGCCCCACTCTGCTTCAGGCAAAAC |
| | | AGGCACACATTTATCACTTTATCTATGGAGTTCTGCTTGAT |
| | | TTCATCAGACAAAAAATTTCCACTGCTAAAACAGGCAAAT |
| | | AAACAAAAAAAAAGTTATGGCCAACAGAGTCACTGGAGG |
| | | GTTTTCTGCTGGGGAGAAGCAAGCCCGTGTTTGAAGGAAC |
| | | CCTGTGAGATGACTGTGGGCTGTGTGAGGGGAACAGCGGG |
| | | GGCTTGATGGTGGACTTCGGGAGCAGAAGCCTCTTTCTCA |
| | | GCCTCCTCAGCTAGACAGGGGAATTATAATAGGAGGTGTG |
| | | GCGTGCACACCTCTCCAGTAGGGGAGGGTCTGATAAGTCA |
| | | GGTCTCTCCCAGGCTTGGGAAAGTGTGTGTCATCTCTAGG |
| | | AGGTGGTCCTCCCAACACAGGGTACTGGCAGAGGGAGAG |
| | | GGAGGGGGCAGAGGCAGGAAGTGGGTAACTAGACTAACA |
| | | AAGGTGCCTGTGGCGGTTTGCCCATCCCAGGTGGGAGGGT |
| | | GGGGCTAGGGCTCAGGGGCCGTGTGTGAATTTACTTGTAG |
| | | CCTGAGGGCTCAGAGGGAGCACCGGTTTGGAGCTGGGACC |
| | | CCCTATTTTAGCTTTTCTGTGGCTGGTGAATGGGGATCCCA |
| | | GGATCTCACAATCTCAGGTACTTTTGGAACTTTCCAGGGC |
| | | AAGGCCCCATTATATCTGATGTTGGGGGAGCAGATCTTGG |
| | | GGAGCCCCTTCAGCCCCCTCTTCCATTCCCTCAGGGACCAT |
| | | GGGCTGTGGCTGCAGCTCACACCCGGAAGATGACTGGATG |
| | | GAAAACATCGATGTGTGTGAGAACTGCCATTATCCCATAG |
| | | TCCCACTGGATGGCAAGGGCACGGTAAGAG |
| 13 | Human pLck | TGTGGGGGTGTGTGGGGGTGCCTGTGTGTGGGTGTTTGTG |
| | promoter | TGTGTGTGAGTGTGGGTGTGTGGGGGTATCTATGGAGGGG |
| | (homo sapiens) | ATGCCTGTGTGTGTGTGTAAGGTGAGTGTGTGGATGTGTG |
| | | CATGGGTGTGGGTGAGTGTGTGGAGGTGCCTGTGTGTGTG |
| | | TATGAATGTGTGTTGGGGAGGGGAGAATGTGTGTGTGTGG |
| | | GGTGAGTGCGTGTGTGTGGGGGTGAATGCGTGTGTGTGTG |
| | | TATGTGGGTGTGACTGTGTGGGTGTGTGGACGAGTGTGTG |
| | | TGTTCCTGGGTGTGGGAGCCTGTGTGTGTAGGGGGAGTAT |
| | | GTATAGGGTGTGTGCATGAATGTGTGTGTGGGTACGTGTG |
| | | TGAGAGTGGGTGCCTGTGTGTGGGGGGTGAGTGTGTGTGT |
| | | GGGGGGGCACTTGTGGAGGGTGAGTGTATGTGTTTACTGA |
| | | GTGTGAGTGTGGGTGCCTGTGTGTGGGAGGGTGAGTCTGT |
| | | GTGTGAGTGTGTGGGGGAGTACCTGTGAGGGGTGAGTGTG |
| | | TGTGTTTATGTGAAAGTGTGTGTGTGTGGATGCCTCTGTGG |
| | | AGGTGGGATAGGGGGTGCCTCTGTGTGTGTGTGTGAGAGT |
| | | GTGTGTGTGTAGGGTGTGTATATGTATAGGGTGTGTGTGA |
| | | GTGTGTGTGTGTGAGAGAGTGTGTGTGTGGCAGAATAGAC |
| | | TGCGGAGGTGGATTTCATCTTGATATGAAAGGTCTGGAAT |
| | | GCATGGTACATTAAACTTTGAGGACAGCGCTTTCCAAGCA |
| | | CTCTGAGGAGCAGCCCTAGAGAAGGAGGAGCTGCAGGGA |
| | | CTCCGGGGGCTTCAAAGTGAGGGCCCCACTCTGCTTCAGG |
| | | CAAAACAGGCACACATTTATCACTTTATCTATGGAGTTCTG |
| | | CTTGATTTCATCAGACAAAAAATTTCCACTGCTAAAACAG |
| | | GCAAATAAACAAAAAAAAAGTTATGGCCAACAGAGTCAC |
| | | TGGAGGGTTTTCTGCTGGGGAGAAGCAAGCCCGTGTTTGA |
| | | AGGAACCCTGTGAGATGACTGTGGGCTGTGTGAGGGGAAC |
| | | AGCGGGGGCTTGATGGTGGACTTCGGGAGCAGAAGCCTCT |
| | | TTCTCAGCCTCCTCAGCTAGACAGGGGAATTATAATAGGA |
| | | GGTGTGGCGTGCACACCTCTCCAGTAGGGGAGGGTCTGAT |
| | | AAGTCAGGTCTCTCCCAGGCTTGGGAAAGTGTGTGTCATC |
| | | TCTAGGAGGTGGTCCTCCCAACACAGGGTACTGGCAGAGG |
| | | GAGAGGGAGGGGGCAGAGGCAGGAAGTGGGTAACTAGAC |
| | | TAACAAAGGTGCCTGTGGCGGTTTGCCCATCCCAGGTGGG |
| | | AGGGTGGGGCTAGGGCTCAGGGGCCGT |
| 14 | Human pLck | TGTGGGGGTGTGTGGGGGTGCCTGTGTGTGGGTGTTTGTG |
| | promoter | TGTGTGTGAGTGTGGGTGTGTGGGGGTATCTATGGAGGGG |
| | (homo sapiens) | ATGCCTGTGTGTGTGTGTAAGGTGAGTGTGTGGATGTGTG |
| | | CATGGGTGTGGGTGAGTGTGTGGAGGTGCCTGTGTGTGTG |
| | | TATGAATGTGTGTTGGGGAGGGGAGAATGTGTGTGTGTGG |
| | | GGTGAGTGCGTGTGTGTGGGGGTGAATGCGTGTGTGTGTG |
| | | TATGTGGGTGTGACTGTGTGGGTGTGTGGACGAGTGTGTG |
| | | TGTTCCTGGGTGTGGGAGCCTGTGTGTGTAGGGGGAGTAT |
| | | GTATAGGGTGTGTGCATGAATGTGTGTGTGGGTACGTGTG |
| | | TGAGAGTGGGTGCCTGTGTGTGGGGGGTGAGTGTGTGTGT |
| | | GGGGGGGCACTTGTGGAGGGTGAGTGTATGTGTTTACTGA |
| | | GTGTGAGTGTGGGTGCCTGTGTGTGGGAGGGTGAGTCTGT |
| | | GTGTGAGTGTGTGGGGGAGTACCTGTGAGGGGTGAGTGTG |
| | | TGTGTTTATGTGAAAGTGTGTGTGTGTGGATGCCTCTGTGG |
| | | AGGTGGGATAGGGGGTGCCTCTGTGTGTGTGTGTGAGAGT |
| | | GTGTGTGTGTAGGGTGTGTATATGTATAGGGTGTGTGTGA |
| | | GTGTGTGTGTGTGAGAGAGTGTGTGTGTGGCAGAATAGAC |
| | | TGCGGAGGTGGATTTCATCTTGATATGAAAGGTCTGGAAT |
| | | GCATGGTACATTAAACTTTGAGGACAGCGCTTTCCAAGCA |
| | | CTCTGAGGAGCAGCCCTAGAGAAGGAGGAGCTGCAGGGA |
| | | CTCCGGGGGCTTCAAAGTGAGGGCCCCACTCTGCTTCAGG |
| | | CAAAACAGGCACACATTTATCACTTTATCTATGGAGTTCTG |
| | | CTTGATTTCATCAGACAAAAAATTTCCACTGCTAAAACAG |
| | | GCAAATAAACAAAAAAAAAGTTATGGCCAACAGAGTCAC |
| | | TGGAGGGTTTTCTGCTGGGGAGAAGCAAGCCCGTGTTTGA |
| | | AGGAACCCTGTGAGATGACTGTGGGCTGTGTGAGGGGAAC |
| | | AGCGGGGGCTTGATGGTGGACTTCGGGAGCAGAAGCCTCT |
| | | TTCTCAGCCTCCTCAGCTAGACAGGGGAATTATAATAGGA |
| | | GGTGTGGCGTGCACACCTCTCCAGTAGGGGAGGGTCTGAT |
| | | AAGTCAGGTCTCTCCCAGGCTTGGGAAAGTGTGTGTCATC |
| | | TCTAGGAGGTGGTCCTCCCAACACAGGGTACTGGCAGAGG |
| | | GAGAGGGAGGGGGCAGAGGCAGGAAGTGGGTAACTAGAC |
| | | TAACAAAGGTGCCTGTGGCGGTTTGCCCATCCCAGGTGGG |
| | | AGGGTGGGGCTAGGGCTCAGGGGCCGTGTGTGAATTTACT |
| | | TGTAGCCTGAGGGCTCAGAGGGAGCACCGGTTTGGAGCTG |
| | | GGACCCCCTATTTTAGCTTTTCTGTGGCTGGTGAATGGGGA |
| | | TCCCAGGATCTCACAATCTCAGGTACTTTTGGAACTTTCCA |
| | | GGGCAAGGCCCCATTATATCTGATGTTGGGGGAGCAGATC |
| | | TTGGGGGAGCCCCTTCAGCCCCCTCTTCCA |
| 15 | Murine pLck | GGGTGTTGCAGGCGTCTGAGCAGGTGACACACAGTTTCAA |
| | promoter | TTCTTGTTTGTTTGTTTGTTTGTTTTTCGAGACAGGGTTTCT |
| | (mus musculus) | CTGTGTAGACTTGGCTGTCTTGGAACTCACTTTGTAGACCA |
| | | GGCTGGCCTCGAACTCAGAAATCCGCCTGCCTCTGCCTCCT |
| | | GAGTGTTGGGATTAAAGGCCTGTGCCACCACACCCAGCTTC |
| | | TTTTTTTTTTTTTTAAGATTTATTTTTATTTATTTCATGTA |
| | | TGTGACTACACTGTAGCTGTCTTCAGACACACCAGAAGAGG |
| | | GTCTCAGATCCCATTACAGATGGTTGTGAGTCACCATGTG |
| | | ATTGCTGGGAATTGAACTCAAGACCTCTGGAAGAGCACTC |
| | | AGTGCTCTTTTTGTTTGTTTGTTTGTTTGTTTGTTTGGGTTT |
| | | TTTTTTGAGACAGGGTTTCTCTCTGTAGCCGTGTGTGTGGG |
| | | GTAGCCCTGTGTGTGAGTGTGTGTGTGTGTGTTTGTGGTAT |
| | | GTTGCAAAATAGATTAAACAACTGAGAGATGGAATAGGTC |
| | | TTCTTGACATCAAAAACATGATCGTGAACCCCTTTATTAAA |
| | | TCTAACACTCAGAGACAGGAGCATCTCTGCAGGTTTGAGG |
| | | CCAGCCTGGTCTACAGAATGAATTTCAGGTCTCAAGGTCA |
| | | GCTTGGTCTACAAAGTGAGTTTCAGATCTCAAGGCCAGCC |
| | | AAGACTATGCAGTAAGACCTTAGCTAAAATAAATAAATAA |
| | | ATAAAATAAAATAAAAGTTAATCTTCGCTTGGCAAACCGA |
| | | TAATTGAGGACCAGTGCTCAGGAAGGAGACACACGGGAA |
| | | TTCCAGAGGCTACAGAGGGAGCCTCGCTCTGACCTGGTTA |
| | | GAGCAACTCTACTTTACTGGCTGTGTCTATGAGGTTCTGCT |
| | | TGATTTCATTTGACAAAAAGTTTCCACAGCTAAACCAGGC |
| | | AAGGGAGCCGAAGTAGACACAGCCACCCGGGCCGCGCCA |
| | | ACAGGTTTCTCTCTGCTGCTGAGAAGCAAAAGCCTGTTTG |
| | | AAGAAACTCTCTGAAGGAGACTGTGGTTGAGTGGTGGGGG |
| | | TAGGGGTGCTGGGGTTGGGCTGAGGCTGAGGGTTGACTCT |
| | | AAGGAGCTGGAACCTCTCAGCTTCGGTGGCTAGACAGGGG |
| | | AGTTGTAATGAAGAGGGACAGGTACCCTCCTTGGTGGAGG |
| | | AGGGTGGAATGAAACTCTCGGTTTCCCCCAGAACTTGGCA |
| | | AAGTGTGTGTGATGTCTCCCAGGTAGTCCCCCAAAGGAGG |
| | | AGGCTAGCAGAGCTGGGGCGGCAGGAAGTGGGTAACTAG |
| | | ACTAACAAAGATGCCTGTGGCGGTTTGCCCATCCCAGGTG |
| | | GGAGGGTGGGACTAGCCTTGGGCCTGGGCCTCCTGTGAAC |
| | | TTGGTGCTTGAGGGCTCAGAGGGAACCCAGTCAGGAGCTT |
| | | GAATCCCACGATTCAGCGCTTCTGTCTGCGGCCA |
| 4 | CMV promoter | CACCAAAATCAACGGGACTTTCCAAAATGTCGTAACAACT |
| | (cytomegalovirus) | CCGCCCCATTGACGCAAATGGGCGGTAGGCGTGTACGGTG |
| | | GGAGGTCTATATAAGCAGAGCTCGTTTAGTGAACCGTCAG |
| 16 | Human NKp46 | ACTAGCCACAGACAGAAAAACAGGCAGTTCATGTTTAAAG |
| | minimal | GACTCCACCTCTTTCTCTTCCTCGGGGGGAACTGGGTTTTC |
| | promoter | TTAAATACAACTGAGTTTTTGTTTACACATTCTGTAATTTC |
| | (homo sapiens) | TTTTAATTCCTGTTCCAGTATCTCACTGTGAAACTCCCTAT |
| | | GTTTTTATACGATTCTCAGGGGGTTTCCTCTGGGCATGATT |
| | | GGGCACAACTTCCCACAGTCAGCTCTGGGTACGACCTCCA |
| | | CATTGCAGAATTGAGAAGTTGACCCAGAAATGCATTTTGG |
| | | GCTGAGCAGACAATTGTCAGAGTTGCTGGCTAGACCACAG |
| | | ATGTGTCAAGGGACCACGGCCTTTCTGTAAGCTCATGGTC |
| | | AGAGGCGGAGGGGAGTTGTGAACGTTCTGATGAAAG |
| 17 | Human NKp46 | CTAGCCACAGACAGAAAAACAGGCAGTTCATGTTTAAAGG |
| | minimal | ACTCCACCTCTTTCTCTTCCTCGGGGGGAACTGGGTTTTCT |
| | promoter | TAAATACAACTGAGTTTTTGTTTACACATTCTGTAATTTCT |
| | (homo sapiens) | TTTAATTCCTGTTCCAGTATCTCACTGTGAAACTCCCTATG |
| | | TTTTTATACGATTCTCAGGGGGTTTCCTCTGGGCATGATTG |
| | | GGCACAACTTCCCACAGTCAGCTCTGGGTACGACCTCCAC |
| | | ATTGCAGAATTGAGAAGTTGACCCAGAAATGCATTTTGGG |
| | | CTGAGCAGACAATTGTCAGAGTTGCTGGCTAGACCACAGA |
| | | TGTGTCAGAGGGACCACGGCCTTTCTGTAAGCTCATGGTC |
| | | AGAGGCGGAGGGGAGTTGTGAACGTTCTGATGAAAG |
| 18 | Human NKp46 | TGGGATGACAGGCCTGAGCCACCGTGTCTGGCCAGTATTT |
| | promoter | GGTTTTCTGTTTCTGTGTTAACTCGCTTAGGATAATGGCCT |
| | (homo sapiens) | CTAGCTGCATCCATGTTGCTGCAAAGGACATAATCTTGTG |
| | | ATTTTTCAAGGCTGTATAGCGTTCTGTGGTGTATACATATC |
| | | ACATTGTCTTTATCCAGTCCACCTCTGATGGGACCTGGGTG |
| | | GATTCCATGTCTTCACTATTGTGAATCCTGCTGCAATGAAC |
| | | ATACAAGTGCATGTGTCTTTTTGGTAGAATGATTTATTTTC |
| | | CTTTGGCTATATACCCAGCGATGGGATTGCTGGGCTGAAT |
| | | GGTAACTCTGTTTGTAGTTCTCTGAAATATCTCCAAACCAA |
| | | ACTGCTTTCCACAGTGGCTGAACTAATTTACACCCACCAA |
| | | CAGTGTATAAGTGTCCCCTTTGCTCCACAATCTCACCAGCA |
| | | TCTGTTAATTTCTGGCTTTTCAGTAATGGCCATTCTGACTG |
| | | GTGTGAGATGGTATTGTTGAGGGATAATTTAGGAATCAGA |
| | | GAGACCGAGGGGTTGAGGAGGATTTATTATTATTATTATT |
| | | ATTTAGGTGCACCGGCCCCAGTCAGATTAACATCCAAAAA |
| | | GACTGAGGCTCGAACAGAGAGTCCGGTTACCTTTTAAGCA |
| | | TTTTGTGGGGTTGGGGGAGATCTGTGCAGGGGGAAGCATA |
| | | TTACAGAAGCAAGAAACAAAGGCAGTTATTCAATTGAGAC |
| | | ATGCATCACATTATTCCTTACTTTTCAAGAAAAATATGTTT |
| | | TACGACTTGAGGTTATCCTGTCTAGTGACCTTGCAGCCGCA |
| | | CGGCAAGAGAAACAGGGTCTTCACAATGCCTGGGAAAGG |
| | | GAGAGATAAGGCTCACTAGCCACAGACAGAAAAACAGGC |
| | | AGTTCATGTTTAAAGGACTCCACCTCTTTCTCTTCCTCGGG |
| | | GGGAACTGGGTTTTCTTAAATACAACTGAGTTTTTGTTTAC |
| | | ACATTCTGTAATTTCTTTTAATTCCTGTTCCAGTATCTCACT |
| | | GTGAAACTCCCTATGTTTTTATACGATTCTCAGGGGGTTTC |
| | | CTCTGGGCATGATTGGGCACAACTTCCCACAGTCAGCTCT |
| | | GGGTACGACCTCCACATTGCAGAATTGAGAAGTTGACCCA |
| | | GAAATGCATTTTGGGCTGAGCAGACAATTGTCAGAGTTGC |
| | | TGGCTAGACCACAGATGTGTCAGAGGGACCACGGCCTTTC |
| | | TGTAAGCTCATGGTCAGAGGCGGAGGGGAGTTGTGAACGT |
| | | TCTGATGAAAGCAGTCAACGTGAAAGCGCTCTGGTGATGG |
| | | GCGCTGGTGCTCACCCACCACTTCCTGTGTATCTATCTCCC |
| | | TGGCCCGCCCGGCTCAGTCCCCACTGC |
| 19 | Human NKp46 | TGGGATGACAGGCCTGAGCCACCGTGTCTGGCCAGTATTT |
| | promoter | GGTTTTCTGTTTCTGTGTTAACTCGCTTAGGATAATGGCCT |
| | (homo sapiens) | CTAGCTGCATCCATGTTGCTGCAAAGGACATAATCTTGTG |
| | | ATTTTTCAAGGCTGTATAGCGTTCTGTGGTGTATACATATC |
| | | ACATTGTCTTTATCCAGTCCACCTCTGATGGGACCTGGGTG |
| | | GATTCCATGTCTTCACTATTGTGAATCCTGCTGCAATGAAC |
| | | ATACAAGTGCATGTGTCTTTTTGGTAGAATGATTTATTTTC |
| | | CTTTGGCTATATACCCAGCGATGGGATTGCTGGGCTGAAT |
| | | GGTAACTCTGTTTGTAGTTCTCTGAAATATCTCCAAACCAA |
| | | ACTGCTTTCCACAGTGGCTGAACTAATTTACACCCACCAA |
| | | CAGTGTATAAGTGTCCCCTTTGCTCCACAATCTCACCAGCA |
| | | TCTGTTAATTTCTGGCTTTTCAGTAATGGCCATTCTGACTG |
| | | GTGTGAGATGGTATTGTTGAGGGATAATTTAGGAATCAGA |
| | | GAGACCGAGGGGTTGAGGAGGATTTATTATTATTATTATT |
| | | ATTTAGGTGCACCGGCCCCAGTCAGATTAACATCCAAAAA |
| | | GACTGAGGCTCGAACAGAGAGTCCGGTTACCTTTTAAGCA |
| | | TTTTGTGGGGTTGGGGGAGATCTGTGCAGGGGGAAGCATA |
| | | TTACAGAAGCAAGAAACAAAGGCAGTTATTCAATTGAGAC |
| | | ATGCATCACATTATTCCTTACTTTTCAAGAAAAATATGTTT |
| | | TACGACTTGAGGTTATCCTGTCTAGTGACCTTGCAGCCGCA |
| | | CGGCAAGAGAAACAGGGTCTTCACAATGCCTGGGAAAGG |
| | | GAGAGATAAGGCTCACTAGCCACAGACAGAAAAACAGGC |
| | | AGTTCATGTTTAAAGGACTCCACCTCTTTCTCTTCCTCGGG |
| | | GGGAACTGGGTTTTCTTAAATACAACTGAGTTTTTGTTTAC |
| | | ACATTCTGTAATTTCTTTTAATTCCTGTTCCAGTATCTCACT |
| | | GTGAAACTCCCTATGTTTTTATACGATTCTCAGGGGGTTTC |
| | | CTCTGGGCATGATTGGGCACAACTTCCCACAGTCAGCTCT |
| | | GGGTACGACCTCCACATTGCAGAATTGAGAAGTTGACCCA |
| | | GAAATGCATTTTGGGCTGAGCAGACAATTGTCAGAGTTGC |
| | | TGGCTAGACCACAGATGTGTCAGAGGGACCACGGCCTTTC |
| | | TGTAAGCTCATGGTCAGAGGCGGAGGGGAGTTGTGAACGT |
| | | TCTGATGAAAGCAGTCAACGTGAAAGCGCTCTGGTGATGG |
| | | GCGCTGGTGCTCACCCACCACTTCCTGTGTATCTATCTCCC |
| | | TGGCCCGCCCGGCTCAG |
| 20 | Human NKp46 | TGGGATGACAGGCCTGAGCCACCGTGTCTGGCCAGTATTT |
| | promoter | GGTTTTCTGTTTCTGTGTTAACTCGCTTAGGATAATGGCCT |
| | (homo sapiens) | CTAGCTGCATCCATGTTGCTGCAAAGGACATAATCTTGTG |
| | | ATTTTTCAAGGCTGTATAGCGTTCTGTGGTGTATACATATC |
| | | ACATTGTCTTTATCCAGTCCACCTCTGATGGGACCTGGGTG |
| | | GATTCCATGTCTTCACTATTGTGAATCCTGCTGCAATGAAC |
| | | ATACAAGTGCATGTGTCTTTTTGGTAGAATGATTTATTTTC |
| | | CTTTGGCTATATACCCAGCGATGGGATTGCTGGGCTGAAT |
| | | GGTAACTCTGTTTGTAGTTCTCTGAAATATCTCCAAACCAA |
| | | ACTGCTTTCCACAGTGGCTGAACTAATTTACACCCACCAA |
| | | CAGTGTATAAGTGTCCCCTTTGCTCCACAATCTCACCAGCA |
| | | TCTGTTAATTTCTGGCTTTTCAGTAATGGCCATTCTGACTG |
| | | GTGTGAGATGGTATTGTTGAGGGATAATTTAGGAATCAGA |
| | | GAGACCGAGGGGTTGAGGAGGATTTATTATTATTATTATT |
| | | ATTTAGGTGCACCGGCCCCAGTCAGATTAACATCCAAAAA |
| | | GACTGAGGCTCGAACAGAGAGTCCGGTTACCTTTTAAGCA |
| | | TTTTGTGGGGTTGGGGGAGATCTGTGCAGGGGGAAGCATA |
| | | TTACAGAAGCAAGAAACAAAGGCAGTTATTCAATTGAGAC |
| | | ATGCATCACATTATTCCTTACTTTTCAAGAAAAATATGTTT |
| | | TACGACTTGAGGTTATCCTGTCTAGTGACCTTGCAGCCGCA |
| | | CGGCAAGAGAAACAGGGTCTTCACAATGCCTGGGAAAGG |
| | | GAGAGATAAGGCTCACTAGCCACAGACAGAAAAACAGGC |
| | | AGTTCATGTTTAAAGGACTCCACCTCTTTCTCTTCCTCGGG |
| | | GGGAACTGGGTTTTCTTAAATACAACTGAGTTTTTGTTTAC |
| | | ACATTCTGTAATTTCTTTTAATTCCTGTTCCAGTATCTCACT |
| | | GTGAAACTCCCTATGTTTTTATACGATTCTCAGGGGGTTTC |
| | | CTCTGGGCATGATTGGGCACAACTTCCCACAGTCAGCTCT |
| | | GGGTACGACCTCCACATTGCAGAATTGAGAAGTTGACCCA |
| | | GAAATGCATTTTGGGCTGAGCAGACAATTGTCAGAGTTGC |
| | | TGGCTAGACCACAGATGTGTCAGAGGGACCACGGCCTTTC |
| | | TGTAAGCTCATGGTCAGAGGCGGAGGGGAGTTGTGAACGT |
| | | TCTGATGAAAG |
| 21 | Murine NKp46 | ACCCAAGGAGCTGAAGGGGTGGGCAACCCTATAGGTGGA |
| | promoter | ACAACAATATCAACTAACCAGTACCCCCAGAGCTCGTTTC |
| | (mus musculus) | TCTAGCTGCATATACAGCAGAAGTTGACCATCACTGGGAA |
| | | GAGAAGCCTCTTGGTCTTGCAAACTTTATATGCCCCAGTAC |
| | | AGGGGAATGCCACAGCCAAGAAGTGGGAGTGGGTGGGTA |
| | | GGGGAGCAGGGCAGGGGGATGGTATAGGGAACTTTCGGG |
| | | ATAGCATTTGAAATTTGAAATGTATATAAAGAAAGTATCT |
| | | AATAAAAAGGAAATCTTTAAGACCTAAGAGCTAAGTGTGG |
| | | CAGCACACGCTTATGATATCTGTGCTGGGAGACAGAGATA |
| | | GGTGGTTACTAGGGCTCATAGGTCAGCCAACCTTGCATAC |
| | | TTGGCAAGATCCTGGCTCTTATGGGACATTGTACCAAAAA |
| | | ATAAGTGGACATGTGTGCAAATACACATAAACCTAATAAG |
| | | GGTTGGGGAGACAGTTCAGTGATTAAAATGCTTACTATGG |
| | | TAGTGTGAAGGCCAGAGTTTATCTCCCTAGAACCCACATA |
| | | AAATGTCAGGTGGATATGGAATCCTACTTATAATTCCAGC |
| | | CTGGGAATACAGAGACAAGAGATCCCAAGAACAAGTTGG |
| | | CTAGGGAGATTCATCATATTGGTGAGCTCTGGGTTTGATT |
| | | GAGAGACCCTGCCTCAGTGAATAAGATAGAAGAGTAACCA |
| | | AGGATGAGCCAAGACATCAACTTAGGCTTCCACATGCACA |
| | | CATATATACAAGCAAATATACATACTTATGCATGCCACAC |
| | | ATGCACATGAAGATTAAAAACAGTAGAATAATAGCATTTT |
| | | ACTGAGTCAAAATAGAAAAATTATTAATATAGTATTACTC |
| | | TTGGTTTCCTGTTAAACTAATGATCAAATCCAAACACGTGC |
| | | ATGTTACACATGGGAATGCAAAGACTAAAATCCCCATATT |
| | | GGGGAGTCCTCAGACCTTTTGCTCTGTGAATCTCCCCCATG |
| | | CCTTCCCCTGAATGAATTTAAATGTTAACCCAGAAATGGG |
| | | TTGCAGACTGAGCAGTTGATGGTCAGAATAATCTTTTAGA |
| | | TCCAAAATGTGGTTAAATGTGTTATGTGAACCAAGTTTTTG |
| | | CCTGAAGTCCTAGGTCAATGGTAAAGGGAGAGCTGTGAAC |
| | | CTTTCTATTAAAAAATAAAAATGTGTGAAAGTCCCATGGT |
| | | GACTAGTGTCAGGCCTCACCCACCACATCCTGTGCATCTCC |
| | | CTCACCCACTGGACTCAATCCACACACATAGTTCAGCACT |
| | | GGTCTGGCCACTGGT |
| 22 | Murine CD8a | AAGCTTGGGAACCCAAGAACCTATCTACAAGAAAAAGAA |
| | promoter | AAGGGAGAGTGGGCACTCCTCTCCCTTCATGTCCTGTCACT |
| | (mus musculus) | CTCATATTTTTTTTATTAAGATTTAATCTTTATCCTTCTTC |
| | | TTTCAGCCACTCTTGCAGAAGAACAATTCCTAAGGGTCCCT |
| | | TCCAAGTGTTTTCTCTTCCCCAAATCTCAGTTTTCAATTCCT |
| | | CCCACTTGCTCTGCAAGGGTGCATTCTCACTCTGAGTTCCC |
| | | AGGCCCCTAAAAGGTGGTTGACACTCTTTGGTGGGGACTT |
| | | TGGGTGACATCATATCCTCACATAGGAAATCAGCTCTGTC |
| | | TGCAGCTGGCTAAAGGAGCAGTTTCCCCGACCCTACACGC |
| | | CTCCCCCACCGCACCTCCTCCGCCCT |
| 23 | Human CD8a | GCACCCCCGCCCCGCACCTTAGTTCTGCGAGCCTTTCCCCG |
| | enhancer | TCTCCAACGCGGGCAAGGCTGTCAGATCCTCAAGCCGACC |
| | (homo sapiens) | CTTCTCTGCACCGCGACCTCAGTGTCCTGCGCGGGACATC |
| | | AGAGGCCGGACGCTCCACCTGCCCCAGGGCACGCCCCGAG |
| | | CCCCCAAGCCCATCTCCTTCCTGTCGGGTGCTCACCTCTTC |
| | | CTCCCTGCGGGCCCAGGGCCTTCTTGAGGGGCTGCGGATG |
| | | CGAATGAGCCAGCGAAGGGCCGCCCGCGGCCCCTCGGCA |
| | | CCTCTGCCCAGCCGTCCCGACCAGGGGACCAGCTCAGCCC |
| | | CGGTCGGGGCCCCGGCACCCAGGACCGCTCAGGATCTCCA |
| | | TTCCAGTCCCTTCGCACTTTCTGACGGGGAAGGAGAGTTG |
| | | GGAGAACGAGACTCTCAAGTCTGGGGACTTCTACCCACCA |
| | | TTAAAGCTTTGGCATTCTGGCCATTTCTTTCTTTTTAAAAA |
| | | ATTATTATTATTATTTTTTATTTTGAGATGGGAGTCTCGCTC |
| | | TATCCCCCAGCCTGGAGTGCAGTGACTCGATCTCCGCTCG |
| | | CTGCAACTTCCGCCTCCAGGGTTCAAACGATTCTTCTGCCT |
| | | CAGCCTCCCCAGTAGCTAGGACTACAGGCTTGCGCCATCAC |
| | | GCCCGGCTAGTTTTTTTTTTGTTTTTTGTTTGTTTGTTTGTT |
| | | TGTTTTTAAAGTAGAAACAGGATTTCGCCATATTGGCCAG |
| | | GCTGGTCTCGAACTCCTGACCTCAGTTGATCCGACCGCCTC |
| | | GGCCTCCCAAAGTGTTGGAATTACAGCAGTGAGCCACCGC |
| | | GCACCAAGCCCGGCCCATTCTGGCCATATCTATAGACGGA |
| | | TGCATTGCACAGGAGGCTCAGCACTAATCGGTAGATACTG |
| | | CGAGATGCTGGGAGGTTAAGGGGCCTACCCGCAATATCTC |
| | | TGGCCAATGCCTTGGGCTAGAAATGCCATAATTAGCCGCT |
| | | CTTTTGATCCCTTGCAAAATGCGAATCCCACCGCACCTCCA |
| | | CCCCACCCGAGTGGTAATCTCCTAGTGGTAATCTAAGTGA |
| | | GCCTGTGATAAGATAAGTAGCTCCTGGTGGTGAGGGTGAG |
| | | AAATTGGGGAGCTGGAGCCCCAGCCAGGGACGAGGCTGT |
| | | AGGGGCTAGGGCGAAGATGGAGGCTGCTGGGCCCCCAGA |
| | | TGGAAGACGGTAACGTGCGCCCGCTTCGTTTTTGCTCGAG |
| | | GTCAGTCAGGTGCAGACTGAATTCGAAGTCGCTCCCTCCT |
| | | CCGCTCAACCCCGACCAGGCCAAAACTAAAGCAGCACCGC |
| | | CCCCTGCTGGGCCGACAGGGCATCAGATTTTGCTGGACGC |
| | | GGGTGACAGGCGAGATAGGGAGTGTCCCTGCTGCTAGTGC |
| | | CCCTGCTGCTAGTGCCTAGTTACCTGCAGTACTTACCCTCC |
| | | CTGGGCCTCAGTTTTTCTTCCTCTGTAAAATAAATGCGCTG |
| | | GGCCGGATCTTTTCTGAGTTCTCTTCTCCCCTACGAATTCT |
| | | AGATCCCTCCTCTGTCCTCCCTGCGCCAGGGACCTTCGGGC |
| | | GACCCTTCCCTGTACCCCCACCCCACCCTCTCTGGACCCCG |
| | | TTTCTGCCTCAGTACGGCGCGCTGAGCTCTGCCCCCTGCCC |
| | | AGGCCCTGACCCCCTCAGGAGCCGCGGT |
| 24 | Human CD8 | AAGATTTGGCTCAGAAGATGTCCCTAACCCCACGGGCTTT |
| | enhancer | CATTCTCATGACCGGGGGGACACAGGCTCAAGGCTGGCAC |
| | (homo sapiens) | TTGCGTGGCTCCAAGAGTGAGTGGCCTCAGTGCCTCACTG |
| | | GCTGCAGCCTCATGCCAGCCCCGGACAGACCAGAGAAAG |
| | | ACCCGGGTGATACCAACACAAGCAGAGGGCATGGATTGC |
| | | CTTCTCAAAAAAGTTATGTTTGGCTGGGAGTGTTGTTCACA |
| | | GCCTCTTACAGATTTTGTGGAGGTCTCTGCGGTTGGGTGAG |
| | | GGCAAAGTTACGAGGGGAAGATGTGAGGTGGTGTTGGTG |
| | | GAGGGGTGGAGGCTCCTCTTCCCCACACACCCCTCCCCAG |
| | | CACCCCTTCAGCATCCTGCAAGCCTGAAGCTGCCTGCTGG |
| | | GCTAGGCCTGCTGTCTGGGCAAGACCAGACCAGGCAAAA |
| | | GGGAGGCCATGACTCCCACCCCTCCACCCTTACCTCAGCC |
| | | TCAGCCAAGTTTGGGGGACATAGGGAGGGGAGAGCAACA |
| | | GTAAACGTGATCAGCACAAAAGGCGGGCCTGCCTTCGGAT |
| | | CACGAAGGCTCTAGCTGGGCCAGGGCAGAAGATGGAGTTT |
| | | GGGTCGTCCCTGCCTCTATCCGAGTACCTACCCTGCAGCCA |
| | | C |
| 25 | Human CD8 | AAAAATGAGCCGGGTATTGTGGTGCATGCCTATAATCCGG |
| | enhancer | GCTACTCAGGAGGCTGAGGCAGGAGAATCGCTTGAACGTG |
| | (homo sapiens) | GGAGGCGGAGGTTGCAGTGAGCTGAGATCGTACCACTGCA |
| | | CTCCAGCCTGGGTAACAGAGCAAGATTCCATCTCACAAAA |
| | | AAAAAAAAAAGTGGGGGGGGAAGTTTTTAGCTTTTCTTTT |
| | | CTTTTCTTTTCTTTTTTTTTTTTTTTTTTTTTTGAGACGGA |
| | | GTCTCGCTATCTCGCCCAGGCTGGAGTGCAGTGGTGCGATC |
| | | TCGGCTCACTGCAAGCTCCGCCTCCCGGGTTCACGCCATTCT |
| | | CCTGCCTCAACCTCCCAAGTAGCTGGGACTACAGGCACCC |
| | | GCCACAACACCCGGCTAATTTTTTGTATTTTTAGTAGAGAC |
| | | GGGGTTTCACCGTGTTAGCCAGGATGGTCTCGATCTCCTG |
| | | ACCACGTGATCCGCCCGCCTTGGCCTCCCAAAGTGCTGGG |
| | | ATTATAGGCGTGAGCTACCGCACCCGGCCAGTTTTTAGCA |
| | | TTTTTATTATTCAGGGTGCACTTTGGAACTTTTGCAGAAGG |
| | | ACCATCCCTTTTCTCGATCTTTTCTGAAAACTCTCATTGTG |
| | | AGATGAATAACTGAGAATAAAGTTCTGCCTTAAAATACAA |
| | | AGCTATACAGATTAAACACACCTGGGAAATTCCAACACCC |
| | | GAGTTAATATACTCAGTGATGATGGTGGGCCGCTCAGCTT |
| | | CAACAGAGAGGTTTTAGAGGGCTGAGAGTGCAGACATGA |
| | | CGTCATGATGAATTAGCATGTTTACAAGGTGCTAGCCTGG |
| | | TGCTGAATTAACACGTTTTTAGCGTAACATCATTTGGCTGA |
| | | TGGTGGTGATTTGCTCACATCCTCCTCTCTCCCTGTCATCC |
| | | TCCGGGGCTGAGAATCAGGTCA |
| 26 | Human CD8 | GAGTCTCGCTATCTCGCCCAGGCTGGAGTGCAGTGGTGCG |
| | minimal | ATCTCGGCTCACTGCAAGCTCCGCCTCCCGGGTTCACGCC |
| | enhancer | ATTCTCCTGCCTCAACCTCCCAAGTAGCTGGGACTACAGG |
| | (homo sapiens) | CACCCGCCACAACACCCGGCTAATTTTTTGTATTTTTAGTA |
| | | GAGACGGGGTTTCACCGTGTTAGCCAGGATGGTCTCGATC |
| | | TCCTGACCACGTGATCCGCCCGCCTTGGCCTCCCAAAGTG |
| | | CTGGGATTATAGGCGTGAGCTACCGCACCCGGCCAGTTTT |
| | | TAGCATTTTTATTATTCAGGGTGCACTTTGGAACTTTTGCA |
| | | GAAGGACCATCCCTTTTCTCGATCTTTTCTGAAAACTCTCA |
| | | TTGTGAGATGAATAACTGAGAATAAAGTTCTGCCTTAAAA |
| | | TACAAAGCTATACAGATTAAACACACCTGGGAAATTCCAA |
| | | CACCCGAGTTAATATACTCAGTGATGATGGTGGGCCGCTC |
| | | AGCTTCAACAGAGAGGTTTTAGAGGGCTGAGAGTGCAGAC |
| | | ATGACGTCATGATGAATTAGCATGTTTACAAGGTGCTAGC |
| | | CTGGTGCTGAATTAACACGTTTTTAGCGTAACATCATTTGG |
| | | CTGATGGTGGTGATTTGCTCACATCCTCCTCTCTCCCTGTC |
| | | ATCCTCCG |
| 27 | Human CD3 | TCGCCCTCCTACCTACCTGATTTCTTCCTTCATCTCCTTCCT |
| | delta enhancer | TCCTCTAGCCTCACACAGACAGTGTGTTGGGCAGTCTCTCG |
| | (homo sapiens) | CCACATCCTGGCTGTCTGGTGTTTCTAGCCCACAGGGCTCC |
| | | CTGGGTGAAGGGTCTGCGCCACCACGTTCCCACAGCTACT |
| | | TGTTGACTCCAGTCCCTATCACAACCTTTAGGGTTTTGGCT |
| | | GCCTCATCCAGCCGAGGGAGGCTGCTGTGTTCAGGAGCCC |
| | | AGCCGCACAGAAGTTTCCATGATGTCATGAATGGGAGTGG |
| | | CAGAGACAACAAGGGGCCCTGGGACTAGTTTCTCACCAAG |
| | | ACTCAGAGGGTGTGTGTAACAAAGTCCCACTGCTGCTAAG |
| | | GGGATGGCCTCAACTCTACGGCCCTGCTGACCAGGTGCCA |
| | | CTAGCTGTAAGGAGGTCTCACCTAAGCCCCTTTCGTATCCC |
| | | TTTCCCCTGTATCAAAGGCACCATTTTAGCTTCCTTTCGCC |
| | | AGGACCTGTCTCTGGCTTGCTCCTGCCTTCTGCATGATAAA |
| | | TGGGGATCAGAAACCCTTCACCAATTAAAGCACATTATCC |
| | | CGCTCCATCTACCTGGAAAGCAGGAGTTTGAGACAGGGATT |
| | | TTTTTTTTTTTGAGATGGAGTCTTGCTCTGTTGCTCAGGCT |
| | | GGAGTGCTGTGGCATGATCTTGGCTTACTGCAACCTCTGTC |
| | | TCCTGGAGTCAAGTGATTCTCCTGCCTCAGCCTCCTGAGTA |
| | | GCTGGGATTGCAAGCATGTGTCACCAAGTTGAGCTAATTT |
| | | TGTATTTTAGTAGAGACAGGGTTTCATCATGTTGGCCAGG |
| | | CTGGTCTCGAAGTGTTGNNACCTCAATGATCCACTCACTC |
| | | AGCNTCAAAGTGCTGGGATNNGANGCATGAGAGCACTGC |
| | | ACTGCTGAGACAGGATANNTTCATAGATGTCATCTTCTAN |
| | | TACNTCTAATGGTAGTGGTTCTAGGCTGGGCACAATGGCT |
| | | CATCCATGTAATCCCAGCACTTTAGGAGGCAGAGGCAAGA |
| | | GGATCACTTGAGCCTAGGAGTTCAAAACCAGCCTGGGCAA |
| | | CATAGCAACATCCCGTCTCGACCAAAAAAAAAAAAAAAA |
| | | ATCAGCTCAGTGCAGTGGTGGGCACCTGTAGTCCTAGCTA |
| | | TTTGGGAGGCTGAGGTGGGAGGATTGCATGAGGCTGGGAA |
| | | TTGGAGGCTGCAGTGAGTTATGATTGTGCCTCTGCACTCC |
| | | AGCCTGGGCAACAGAGTGAGACNNCTATCTTAAAAAAAA |
| | | AATAGCTATGGTTCAAACCTTGGAAGCCTGAGGGATTTAT |
| | | TAAAAATATAAATGCAGCCAGGCATGGTGGCTCACACCTG |
| | | TAATCCCAGCACTTGGAGTTAGCGGCAGATCACAAGATCA |
| | | GAAGATTGAGTATCACCCTCGCAACATGTTGAAACCCCGT |
| | | TCTACTAAAAATACAAAAATTAGCTGGGCATGGTGCGCAC |
| | | GCCTGTAATCCCAGCTACTCGGGAGGCTGAGGCAGGAGAA |
| | | TTGCTTGAACCCGGGAGGCGGAGGTTGCAGTGAGTCAAGA |
| | | TTGCGCCACCGCACTCCAGCCTGGTGACAGAGCGAGACTT |
| | | GGTCTCAAAATAAAATAAATAAAATAAAATAAAAGTGCT |
| | | AGAGACGCACTCTAGGCAGGGTGTTTCCAGTTGATTCTGA |
| | | AACAAAAAAAATTCAGAGTCTCTTCTTTAGAGGGAGAATA |
| | | GAGGCAATCTTTCTCTCTTATGTCTTTTATGC |
| 28 | Murine CD4 | TGTTGGGGTTCAAATTTGAGCCCCAGCTGTTAGCCCTCTG |
| | enhancer | CAAAGAAAAAAAAAAAAAAAAAAGAACAAAGGGCCTAGAT |
| | (mus musculus) | TTCCCTTCTGAGCCCCACCCTAAGATGAAGCCTCTTCTTT |
| | | CAAGGGAGTGGGGTTGGGGTGGAGGCGGATCCTGTCAGCT |
| | | TTGCTCTCTCTGTGGCTGGCAGTTTCTCCAAAGGGTAACAG |
| | | GTGTCAGCTGGCTGAGCCTAGGCTGAACCCTGAGACATGC |
| | | TACCTCTGTCTTCTCATGGCTGGAGGCAGCCTTTGTAAGTC |
| | | ACAGAAAGTAGCTGAGGGGCTCTGGAAAAAAGACAGCCA |
| | | GGGTGGAGGTAGATTGGT |
| 29 | CMV enhancer | CTAGTTATTAATAGTAATCAATTACGGGGTCATTAGTTCAT |
| | (cytomegalovirus) | AGCCCATATATGGAGTTCCGCGTTACATAACTTACGGTAA |
| | | ATGGCCCGCCTGGCTGACCGCCCAACGACCCCCGCCCATT |
| | | GACGTCAATAATGACGTATGTTCCCATAGTAACGCCAATA |
| | | GGGACTTTCCATTGACGTCAATGGGTGGAGTATTTACGGT |
| | | AAACTGCCCACTTGGCAGTACATCAAGTGTATCATATGCC |
| | | AAGTACGCCCCCTATTGACGTCAATGACGGTAAATGGCCC |
| | | GCCTGGCATTATGCCCAGTACATGACCTTATGGGACTTTCC |
| | | TACTTGGCAGTACATCTACGTATTAGTCATCGCTATTACCA |
| | | TGGTGATGCGGTTTTGGCAGTACATCAATGGGCGTGGATA |
| | | GCGGTTTGACTCACGGGGATTTCCAAGTCTCCACCCCATTG |
| | | ACGTCAATGGGAGTTTGTTTTGG |
| 30 | Murine T-cell | CGGGATTTTGCTGCCTTACCACAGCCCAAAGCAGGCTGTG |
| | specific enhancer | TGTTTAGAAGCTGAGCCACAAAGAAGTTTCCATGACATCA |
| | region of CD3 | TGAATGGGGGTGGCAGAGAAGAATATTGGGGCTCAGAGG |
| | delta | GTGTGTGTGTGTGTGTGTGTGTGTGTGTGTGTGTGTGTGAG |
| | (mus musculus) | AGAGAGAGAGAGAGAGAGAGAGAGAGAGAGACAGACAG |
| | | AGAGACAGAGAGACAGAAAGACAGAAAGACAGAAAGAC |
| | | AGAGACAGAGCTGTCAAGAATTTTCTTCTACTTCGTGGGTT |
| | | CTGGGGATTGTACTAAGGTCATGAGGCTTGTGTGGCAAGC |
| | | ACCCTTACCCACTGAGCCATCTTGCCTGCCCTCATCCTCAA |
| | | ATTAATTAAAAATAAAGAACATGATGAATT |
| 31 | pCI intron | GTAAGTATCAAGGTTACAAGACAGGTTTAAGGAGGCCAAT |
| | | AGAAACTGGGCTTGTCGAGACAGAGAAGATTCTTGCGTTT |
| | | CTGATAGGCACCTATTGGTCTTACTGACATCCACTTTGCCT |
| | | TTCTCTCCACAG |
| 32 | Human CD3 | GTAAGGGCCTACTAGGGGTCTGGAAGCCTGGGGAAGGGC |
| | gamma intron 1 | TCAAGGGAAGAGCCCATCACTAGTGAGACAGGAATATTG |
| | (truncated; homo | GTATCCCTAACCTTCAGCCTACCTCTGCTGTCACCTTAGAG |
| | sapiens) | TTCAAAGAAGGGCAAAATGGAGGCT |
| 33 | Human CD4 | ATTTAAGCACGACTCTGCAG |
| | exon 1 | |
| | (homo sapiens) | |
| 34 | Human CD4 | GGGTAGTCGAC |
| | exon 2 | |
| | (homo sapiens) | |
| 35 | Human CD3 | TGGAGCCAGTCTAGCTGCTGCACAGGCTGGCTGGCTGGCT |
| | gamma exon 1 | GGCTGCTAAGGGCTGCTCCACGCTTTTGCCGGAGGACAGA |
| | (homo sapiens) | GACTGACTAGGAACAGGGGAAGGGCCTGGCTGTCCTCATC |
| | | CTGGCTATCATTCTTCTTCAAG |
| 36 | pCI splice | TCTTACTGACATCCACTTTGCCTTTCTCTCCACAGG |
| | acceptor | |
| 37 | Murine CD4 | TGTTGGGGTTCAAATTTGAGCCCCAGCTGTTAGCCCTCTGC |
| | enhancer plus | AAAGAAAAAAAAAAAAAAAAAAGAACAAAGGGCCTAGA |
| | human CD4 | TTTCCCTTCTGAGCCCCACCCTAAGATGAAGCCTCTTCTTT |
| | promoter | CAAGGGAGTGGGGTTGGGGTGGAGGCGGATCCTGTCAGCT |
| | (synthetic | TTGCTCTCTCTGTGGCTGGCAGTTTCTCCAAAGGGTAACAG |
| | construct) | GTGTCAGCTGGCTGAGCCTAGGCTGAACCCTGAGACATGC |
| | | TACCTCTGTCTTCTCATGGCTGGAGGCAGCCTTTGTAAGTC |
| | | ACAGAAAGTAGCTGAGGGGCTCTGGAAAAAAGACAGCCA |
| | | GGGTGGAGGTAGATTGGTGCATGCAGCTTCTGCAGCCCCG |
| | | CGTGCTAGAGATTTAAGCCTGATTCTGCTTAACTTTTTCCC |
| | | TTGACTTTGGCATTTTCACTTTGACATGTTCCCTGAGAGCC |
| | | TGGGGGGTGGGGAACCAGCTCCAGCTGGTGACGTTTGGGG |
| | | CCGGCCCAGGCCTAGGGTGTGGAGGAGCCTTGCCATCGGG |
| | | CTTCCTGTCTCTCTTC |
| 38 | Human CD4 | ATTTAAGCACGACTCTGCAGTCTAGACACAGGTAAGTATC |
| | exons 1&2 + pCI | AAGGTTACAAGACAGGTTTAAGGAGGCCAATAGAAACTG |
| | intron | GGCTTGTCGAGACAGAGAAGATTCTTGCGTTTCTGATAGG |
| | (synthetic | CACCTATTGGTCTTACTGACATCCACTTTGCCTTTCTCTCC |
| | construct) | ACAGGGGTAGTCGAC |
| 39 | Mouse CD3 | TGATCAGAAACAAGAGGATCTGCTAATGTGGCTTGTGTAA |
| | promoter plus | TAGTTTCTCTCAGTGGATCTAAAGAGGTGGCACTTCCTTGT |
| | pCI intron | TTGTGAGTAAATGTAGTTGTATTGTTCATAGGAAGCCCAA |
| | (synthetic | AATCTTCTTTGTTTGTTACCAGTGACTATTGCTATTTGTTTT |
| | construct) | TTGTTACCAGGAAAGGCTATTCTAAATTCCCTTACACTTCC |
| | | TAGAATTCTGAACAAACTTTCCTCATAGTCTCTGCTTTGTA |
| | | TCAATCATCCCAAACTCTTTGCTTCAGAAAGAGCAGTTTGC |
| | | ACCTGTTTGTGTCTTACCCCACACCTAGCCCACTGTTGCTT |
| | | TGACAGTCTTACACCATCAAAGGCAGAAGGTAGAGAGGCA |
| | | GATTTCTTCTAGTTCCTCCCCCACTCTTCCTTTTTCTGAAT |
| | | ACCCATGAGTCTGTTGGCGGAGGATAGTGCTAACCCTGGC |
| | | TGATCATCTAGACACAGGTAAGTATCAAGGTTACAAGACA |
| | | GGTTTAAGGAGGCCAATAGAAACTGGGCTTGTCGAGACAG |
| | | AGAAGATTCTTGCGTTTCTGATAGGCACCTATTGGTCTTAC |
| | | TGACATCCACTTTGCCTTTCTCTCCACAGGGGTAGTCGACG |
| | | CCACC |
| 40 | Human CD3 | AACAACTGGCTACGATCCTAACAACTAATGACAGGGACAT |
| | gamma promoter | TTATCTCCCTCATGAAGAAACGGTCCCAGGACCATCTCCC |
| | plus exon 1, | ACCCAGCATCCATTGCGGTTCCCTGTGCAAGATGAGTCTCT |
| | intron 1, and | GAGTGGGAATCCAGCACTCTCTCCCTCTTCTTCCCCACCAC |
| | splice acceptor | CTTCACCCTCCTTAACGGAAAAACAAAAGGCATCTGCACC |
| | (synthetic | TGCAGCCCTGCTGAGGCCCCTGCTGCTCACACTTGCAGCA |
| | construct) | GAGGGTGGAGGCTCTGGGTTCTTGCCTTCTCTCAAAGGCC |
| | | CCAGCCCCAACAGTGATGGGTGGAGCCAGTCTAGCTGCTG |
| | | CACAGGCTGGCTGGCTGGCTGGCTGCTAAGGGCTGCTCCA |
| | | CGCTTTTGCCGGAGGACAGAGACTGACTAGGAACAGGGG |
| | | AAGGGCCTGGCTGTCCTCATCCTGGCTATCATTCTTCTTCA |
| | | AGGTAAGGGCCTACTAGGGGTCTGGAAGCCTGGGGAAGG |
| | | GCTCAAGGGAAGAGCCCATCACTAGTGAGACAGGAATATT |
| | | GGTATCCCTAACCTTCAGCCTACCTCTGCTGTCACCTTAGA |
| | | GTTCAAAGAAGGGCAAAATGGAGGCTTCTTACTGACATCC |
| | | ACTTTGCCTTTCTCTCCACAGGGGTAGTCGACGCCACC |

### 5. Nucleotides

In some embodiments, a polynucleotide or expression construct disclosed herein comprises natural, synthetic, and/or artificial nucleotide analogues or bases. In some embodiments, the synthetic or artificial nucleotide analogues or bases comprise modifications at one or more of a deoxyribose moiety, ribose moiety, phosphate moiety, nucleoside moiety, or a combination thereof.

In some embodiments, a nucleotide analogue or artificial nucleotide base comprises a nucleic acid with a modification at a 2′ hydroxyl group of the ribose moiety. In some instances, the modification includes an H, OR, R, halo, SH, SR, NH₂, NHR, NR₂, or CN, wherein R is an alkyl moiety. Illustrative alkyl moiety include, but are not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′O-methoxyethyl modification adds a methoxyethyl group to the 2′ hydroxyl group of the ribose moiety.

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate-derived overall negative charge of the oligonucleotide molecule by introducing one positive charge from the amine group per sugar and thereby improves cellular uptake properties due to its zwitterionic properties.

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer.

In some embodiments, additional modifications at the 2′ hydroxyl group include 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA).

In some embodiments, a nucleotide analogue comprises a modified base, for example, N1-methylpseudouridine, 5-propynyluridine, 5-propynylcytidine, 6-methyladenine, 6-methylguanine, N, N,-dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or are based on, mannoses, arabinoses, glucopyranoses, galactopyranoses, 4′-thioribose, and other sugars, heterocycles, or carbocycles. The term nucleotide also includes universal bases. By way of example, universal bases include but are not limited to 3-nitropyrrole, 5-nitroindole, or nebularine.

In some embodiments, one or more modifications optionally occur at the internucleotide linkage. In some instances, a modified internucleotide linkages can include, but is not limited to, phosphorothioates; phosphorodithioates; methylphosphonates; 5′-alkylenephosphonates; 5′-methylphosphonate; 3′-alkylene phosphonates; borontrifluoridates; borano phosphate esters and selenophosphates of 3′-5′linkage or 2′-5′linkage; phosphotriesters; thionoalkylphosphotriesters; hydrogen phosphonate linkages; alkyl phosphonates; alkylphosphonothioates; arylphosphonothioates; phosphoroselenoates; phosphorodiselenoates; phosphinates; phosphoramidates; 3′-alkylphosphoramidates; aminoalkylphosphoramidates; thionophosphoramidates; phosphoropiperazidates; phosphoroanilothioates; phosphoroanilidates; ketones; sulfones; sulfonamides; carbonates; carbamates; methylenehydrazos; methylenedimethylhydrazos; formacetals; thioformacetals; oximes; methyleneiminos; methylenemethyliminos; thioamidates; linkages with riboacetyl groups; aminoethyl glycine; silyl or siloxane linkages; alkyl or cycloalkyl linkages with or without heteroatoms of, for example, 1 to 10 carbons that are saturated or unsaturated and/or substituted and/or contain heteroatoms; linkages with morpholino structures, amides, or polyamides wherein the bases are attached to the aza nitrogens of the backbone directly or indirectly; and combinations thereof.

In some embodiments, one or more modifications comprise a modified phosphate backbone in which the modification generates a neutral or uncharged backbone. In some instances, the phosphate backbone is modified by alkylation to generate an uncharged or neutral phosphate backbone. As used herein, alkylation includes methylation, ethylation, and propylation. In some cases, an alkyl group, as used herein in the context of alkylation, refers to a linear or branched saturated hydrocarbon group containing from 1 to 6 carbon atoms. In some instances, exemplary alkyl groups include, but are not limited to, methyl, ethyl, n-propyl, isopropyl, n-butyl, isobutyl, sec-butyl, tert-butyl, n-pentyl, isopentyl, neopentyl, hexyl, isohexyl, 1,1-dimethylbutyl, 2,2-dimethylbutyl, 3,3-dimethylbutyl, and 2-ethylbutyl groups. In some cases, a modified phosphate is a phosphate group as described in U.S. Pat. No. 9,481,905.

In some embodiments, additional modified phosphate backbones comprise methylphosphonate, ethylphosphonate, methylthiophosphonate, or methoxyphosphonate. In some cases, the modified phosphate is methylphosphonate. In some cases, the modified phosphate is ethylphosphonate. In some cases, the modified phosphate is methylthiophosphonate. In some cases, the modified phosphate is methoxyphosphonate.

In some embodiments, one or more modifications further optionally include modifications of the deoxyribose moiety, ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

### B. Heterologous Immune Receptor

A system, method, expression construct, cell, or polynucleotide disclosed herein can comprise a transgene that encodes a heterologous immune receptor. A heterologous immune receptor disclosed herein can be a chimeric antigen receptor. In some embodiments, a heterologous immune receptor disclosed herein is a T cell receptor. A heterologous immune receptor can comprise an extracellular domain (including an extracellular binding domain), a transmembrane domain, and a cytoplasmic signaling domain.

A heterologous immune receptor can be expressed by an immune cell and configured to induce activation of and/or signaling in the immune cell upon contacting a target cell that expresses a cell surface molecule. A target cell can be a cell that is associated with a disease or condition. A target cell can be a cancer cell. A target cell can be an immune cell. A target cell can be a hematologic cancer cell. A target cell can be a solid tumor cell. A target cell can be a leukemia cell. A target cell can be a lymphoma cell. A target cell can be a myeloma cell. A target cell can be a B cell. A target cell can be a CD19+ cell. A target cell can be a T cell. A target cell can be a cell that is associated with an autoimmune or inflammatory disease. A target cell can be a fibrotic cell, e.g., a fibroblast. A target cell can be a senescent cell.

In some embodiments, a heterologous immune receptor is a chimeric antigen receptor (CAR). In some embodiments, a heterologous immune receptor is a first, second, third, fourth, or fifth generation CAR. A first generation CAR can contain a single CD3 zeta cytoplasmic signaling domain (e.g., and lack a co-stimulatory cytoplasmic signaling domain). A second generation CAR can comprise a CD3 zeta cytoplasmic signaling domain and a co-stimulatory cytoplasmic signaling domain, such as a CD28 or 41BB costimulatory domain. A third generation CAR can comprise a CD3 zeta cytoplasmic signaling domain and two co-stimulatory cytoplasmic signaling domains, for example, two of CD28, 41BB, and OX40. A fourth generation CAR can comprise a CD3 zeta cytoplasmic signaling domain and a co-stimulatory cytoplasmic signaling domain (such as a CD28 or 41BB costimulatory domain), and a protein, such as interleukin 12 (IL-12), that is constitutively or inducibly expressed upon CAR activation. A fourth generation CAR can be, for example, a T cell redirected for universal cytokine-mediated killing (TRUCK). A fifth generation CAR can be based on a second generation CAR and contain a truncated cytoplasmic IL-2 receptor β-chain domain with a binding site for the transcription factor STAT3.

In some embodiments, a heterologous immune receptor is a universal CAR, for example, an extracellular binding domain can be combined with amino acid sequence(s) from one or more components of a TCR signaling complex and/or a chimeric antigen receptor (CAR) to generate a “universal” heterologous immune receptor that can be armed and disarmed based on the presence of adapter molecule(s). An adapter molecule can direct an immune cell expressing the heterologous immune receptor to a target cell (e.g., a cancer cell), and upregulate activation of the immune cell upon encountering the target cell (e.g., leading to a cytotoxic response against the target cell). A universal CAR can be capable of binding to various adapter molecules that can confer target specificity. Adapter molecules can comprise small molecules, binding fragments of a receptor or receptor ligand, small molecules, an antibody or antigen-binding fragment thereof, or a combination thereof.

The heterologous immune receptor can comprise an N-terminal methionine. The heterologous immune receptor can lack an N-terminal methionine.

In some embodiments, a heterologous immune receptor is a dual CAR, a split CAR, or an inducible split CAR. A dual CAR can comprise two CARs with different extracellular binding domains, and thus signal induction based on two target antigens. A split CAR can comprise two CARs with different extracellular binding domains and separation of costimulatory domains (e.g., CD28 and 41BB) from CD3zeta on the distinct CAR polypeptides, thereby requiring engagement of both CARs for T cell activation.

A heterologous immune receptor can comprise a component of a TCR signaling complex, for example, an extracellular domain, transmembrane domain, and/or cytoplasmic domain of a TCR signaling complex, such as a human TCR signaling complex.

In some embodiments, a heterologous immune receptor that comprises a component of a TCR signaling complex comprises two TCR chains (e.g., an alpha chain and a beta chain, or a gamma chain and a delta chain). In some embodiments, a heterologous immune receptor that comprises a component of a TCR signaling complex comprises a single chain TCR (scTCR), e.g., comprising a TCR alpha chain variable domain and a TCR beta chain variable domain joined by a suitable linker.

In some embodiments, a heterologous immune receptor that comprises a component of a TCR signaling complex is a TCR, e.g., comprises an extracellular binding domain that comprises TCR variable regions and TCR CDRs. In some embodiments, a heterologous immune receptor that comprises a component of a TCR signaling complex is not TCR, for example, comprises a non-TCR extracellular binding domain, and comprises a component of a TCR signaling complex.

In some embodiments, incorporating a component of a TCR signaling complex into a heterologous immune receptor as disclosed herein can confer advantageous properties compared to alternate structures. In some cases, use of a component of a TCR signaling complex facilitates formation of a complex between the heterologous immune receptor and other components of a TCR signaling complex, thereby providing an activation signal that is similar to signaling induced upon native TCR activation. The component of the TCR signaling complex can be a component of the TCR signaling complex that is not CD3 zeta or lacks CD3 zeta signaling domains.

A heterologous immune receptor can comprise at least one extracellular domain of a TCR signaling complex, a transmembrane domain of a TCR signaling complex, and/or at least one cytoplasmic signaling domain of a TCR signaling complex. A heterologous immune receptor can comprise, for example, (i) an extracellular domain of TCR alpha chain constant region, TCR beta chain constant region, TCR gamma chain constant region, TCR delta chain constant region, CD3 gamma, CD3 delta, CD3 epsilon, or a functional fragment thereof; (ii) a transmembrane domain of TCR alpha chain, TCR beta chain, TCR gamma chain, TCR delta chain, CD3 gamma, CD3 delta, CD3 epsilon, CD3 zeta, or a functional fragment thereof; and/or (iii) a cytoplasmic signaling domain of CD3 gamma, CD3 delta, CD3 epsilon, or CD3 zeta.

Where a heterologous immune receptor comprises an extracellular, transmembrane, and/or cytoplasmic signaling domain of a component of a TCR signaling complex, the extracellular, transmembrane, and/or cytoplasmic signaling domains can be from the same protein or different proteins. In some embodiments, the extracellular, transmembrane, and/or cytoplasmic signaling domains are from the same protein, for example, TCR alpha chain, TCR beta chain, TCR gamma chain, TCR delta chain, CD3 gamma, CD3 delta, CD3 epsilon, or CD3 zeta. In some embodiments, the extracellular, transmembrane, and/or cytoplasmic signaling domains are from CD3 epsilon. In some embodiments, the extracellular, transmembrane, and/or cytoplasmic signaling domains are from CD3 delta. In some embodiments, the extracellular, transmembrane, and/or cytoplasmic signaling domains are from CD3 gamma. In some embodiments, the extracellular, transmembrane, and/or cytoplasmic signaling domains are from TCR alpha chain. In some embodiments, the extracellular, transmembrane, and/or cytoplasmic signaling domains are from TCR beta chain. In some embodiments, the extracellular, transmembrane, and/or cytoplasmic signaling domains are from TCR gamma chain. In some embodiments, the extracellular, transmembrane, and/or cytoplasmic signaling domains are from TCR delta chain.

A heterologous immune receptor can comprise a full length or substantially full length CD3 gamma, CD3 delta, CD3 epsilon, or CD3 zeta. A heterologous immune receptor can comprise a full length or substantially full length TCR alpha chain (e.g., constant regions, or variable plus constant regions), TCR beta chain (e.g., constant regions, or variable plus constant regions), TCR gamma chain (e.g., constant regions, or variable plus constant regions), or TCR delta chain (e.g., constant regions, or variable plus constant regions).

In some embodiments, a system disclosed herein allows use of two or more heterologous immune receptors (e.g., CARs) delivered alone or in combination, concurrently or sequentially.

### 6. Extracellular Domain

A heterologous immune receptor disclosed herein can comprise an extracellular domain. The extracellular domain can comprise an extracellular binding domain that can specifically bind to a cell surface molecule on a target cell, thereby modulating signaling by the heterologous immune receptor.

An extracellular binding domain can utilize one or more antigen-binding domains, for example, an antigen-binding domain of or derived from an antibody. In some embodiments, an extracellular binding domain disclosed herein comprises an antigen-binding domain or fragment from an antibody, such as an scFv or a nanobody.

The variable (V) region(s) of an antibody can mediate antigen binding and define the specificity of a particular antibody for an antigen. The variable region can comprise relatively invariant sequences called framework regions, and hypervariable regions, which differ considerably in sequence among antibodies of different binding specificities. The variable region can comprise four framework regions separated by three hypervariable regions. For an antibody that comprises a heavy chain and a light chain or heavy chain variable region and light chain variable region, the variable regions can fold in a manner that brings the hypervariable regions together in close proximity to create an antigen binding site. The four framework regions can largely adopt an f3-sheet configuration, while the three hypervariable regions form loops connecting, and in some cases forming part of, the f3-sheet structure.

Within hypervariable regions are amino acid residues that primarily determine the binding specificity of the antibody in most cases. Sequences comprising these residues are known as complementarity determining regions (CDRs). One antigen binding site of an antibody with heavy and light chains or variable regions therefrom can comprise six CDRs, three in the hypervariable regions of the light chain variable region, and three in the hypervariable regions of the heavy chain variable region. The CDRs in the light chain are designated L1, L2, and L3, while the CDRs in the heavy chain are designated H1, H2, and H3. CDRs can also be designated LCDR1, LCDR2, LCDR3, HCDR1, HCDR2, and HCDR3, respectively. The contribution of each CDR to antigen binding varies among antibodies. CDRs can vary in length. For example, CDRs are often 5 to 14 residues in length, but CDRs as short as 0 residues or as long as 25 residues or longer exist.

In some embodiments, HCDR3 contributes to antigen specificity more than the other CDRs. In some embodiments, an extracellular binding domain comprises an HCDR3 sequence. In some embodiments, an extracellular binding domain comprises an LCDR1, LCDR2, LCDR3, HCDR1, HCDR2, HCDR3, or a combination thereof. In some embodiments, an extracellular binding domain comprises an LCDR1, LCDR2, LCDR3, HCDR1, HCDR2, and HCDR3.

Certain antibodies or antigen-binding domains contain less than six CDRs. For example, certain antibodies lack a light chain, and can be referred to as heavy chain only antibodies (HCAbs). HCAbs have three CDRs in a variable region referred to as VHH. A single domain antibody, or nanobody, can be generated from such a VHH region of a heavy chain only antibody.

An extracellular binding domain of the disclosure can comprise complementarity determining regions (CDRs). For example, an antibody, antigen-binding fragment thereof, or antigen-binding domain can comprise CDRs. In some embodiments, the CDRs determine or substantially determine binding specificity and/or affinity for a surface molecule on a target cell. For example, the CDRs can be grafted onto a different suitable framework, or the framework region can be altered (e.g., via amino acid substitutions, deletions, and/or insertions), and the antigen-binding fragment or domain can retain binding for the target, and the extracellular binding domain remains functional despite the alterations outside of the CDRs. In some embodiments, one or more framework regions or amino acid sequences therein contribute to binding specificity and/or affinity.

CDRs in or for use in an extracellular binding domain can be identified by various methods, including but not limited to the Kabat method, the Chothia method, the IMGT method, the AHO method, and the Paratome method. Single domain antibodies can have longer CDR H1 and H3 loops compared with the respective classical CDRs, and can require different methods to identify CDRs. Single domain antibody CDRs can be identified, for example, using the single domain antibody database (SAbDab), based on common sequence elements, or based on a sequence alignment to the Chothia numbering scheme (e.g., as described by Wilton, et al. (2018). sdAb-DB: the single domain antibody database. ACS Synthetic Biology 2018 7 (11), 2480-2484 DOI: 10.1021/acssynbio.8b00407).

A subset of residues within CDRs contacts an antigen. These residues that contact antigen can be referred to as specificity-determining residues (SDRs). However, in some cases residues other than SDRs can contribute to binding activity by helping to maintain the conformation of the binding site. The number of SDRs in an antibody can vary based on the size and type of antigen that is recognized, for example, between 0-14 SDRs can be found within a CDR. SDRs can be enriched in some residues, such as tyrosine, serine, tryptophan, and asparagine.

A CDR of a sequence herein can be, for example, between 0 and 91 residues in length, between 0 and 25 residues in length, between 5 and 14 residues in length, about 0 residues in length, about 1 residue in length, about 2 residues in length, about 3 residues in length, about 4 residues in length, about 5 residues in length, about 6 residues in length, about 7 residues in length, about 8 residues in length, about 9 residues in length, about 10 residues in length, about 11 residues in length, about 12 residues in length, about 13 residues in length, about 14 residues in length, about 15 residues in length, about 16 residues in length, about 17 residues in length, about 18 residues in length, about 19 residues in length, about 20 residues in length, about 21 residues in length, about 22 residues in length, about 23 residues in length, about 24 residues in length, or about 25 residues in length.

An extracellular binding domain can comprise an antibody fragment, antigen-binding domain, or antigen-binding fragment of an antibody. Non-limiting examples of antibody fragments, antigen-binding fragments, and antigen-binding domains include Fab, Fab′, F(ab′)2, dimers and trimers of Fab conjugates, Fv, scFv, nanobodies, minibodies, dia-, tria-, and tetrabodies, and linear antibodies. Fab and Fab′ are antigen-binding fragments that can comprise the VH and CH1 domains of the heavy chain linked to the VL and CL domains of the light chain via a disulfide bond. A F(ab′) 2 can comprise two Fab or Fab′ that are joined by disulfide bonds. A Fv can comprise the VH and VL domains held together by non-covalent interactions. A scFv (single-chain variable fragment) is a fusion protein that can comprise the VH and VL domains connected by a peptide linker. Manipulation of the orientation of the VH and VL domains and the linker length can be used to create different forms of molecules that can be monomeric, dimeric (diabody), trimeric (triabody), or tetrameric (tetrabody). Minibodies can be scFv-CH3 fusion proteins that assemble into bivalent dimers.

The extracellular binding domain can be or can comprise a single domain antibody. The single domain antibody can be or can comprise a variable region of a heavy chain only antibody. Such a single domain antibody can also be known as a nanobody or VHH. The single domain antibody can be, for example, a variable region from or derived from a heavy chain only antibody from a camelid (e.g., camels: one-humped Camelus dromedaries and two-humped Camelus bactrianus; llamas: Lama glama, Lama guanicoe, and Lama vicugna; and alpacas: Vicugna pacos), a shark (e.g., a nurse shark), a wobbegong, or a spotted ratfish. Such animals have a special type of antibody called heavy chain Abs (HCAbs), that lack the entire light chain and the first heavy chain C region (CH1) compared to regular antibodies.

An extracellular binding domain can comprise an antigen-binding domain or fragment of a chimeric, humanized, or fully human antibody. An extracellular binding domain can comprise CDRs grafted onto a humanized or fully human framework sequence. An extracellular binding domain can comprise a chimeric antibody wherein a portion of the heavy and/or light chain (e.g., variable region) is identical to or homologous to a corresponding sequence in an antibody derived from a particular species or belonging to a particular antibody class or subclass, while the remainder of the chain(s) is identical or homologous to a corresponding sequence in an antibody derived from another species or belonging to another antibody class or subclass, or an antigen-binding fragment of such an antibody. In some embodiments, an extracellular binding domain comprises an antigen-binding domain or fragment that is not of a chimeric, humanized, or fully human antibody, for example, from a non-human mammalian antibody, a camelid, or another species disclosed herein.

For human administration, monoclonal antibodies or fragments thereof generated from non-human species that will be used in an extracellular binding domain can be further refined by a humanization process to reduce the likelihood of immunogenicity while preserving target specificity. Humanization processes can involve the incorporation of human DNA to the genetic sequence of the genes that produce the isolated antibodies, and/or the removal of predicted epitopes, such as T cell epitopes.

An example of a humanized antibody is a modified chimeric antibody. A chimeric antibody can be generated as described above. The chimeric antibody can be further mutated outside of the CDRs to substitute non-human sequences in the variable regions with the homologous human sequences. Another example of a humanized antibody is a CDR-grafted antibody, in which CDR sequences (e.g., from a non-human source) are introduced into the human heavy and light chain variable regions of a human antibody scaffold to replace the corresponding human CDR sequences.

A single domain antibody can be a humanized single domain antibody. The single domain antibody can comprise CDRs in a humanized framework, for example, as described by Soler et al. (2021). Effect of Humanizing Mutations on the Stability of the Llama Single-Domain Variable Region. Biomolecules, 11 (2), 163.

In some embodiments, an extracellular binding domain disclosed herein comprises an antibody scaffold domain, for example, a constant domain from an antibody.

In addition to antibodies and antigen-binding fragments or domains thereof, other compounds can also comprise antigen-binding domains that can be used in compositions, systems, and methods of the disclosure, such as in an extracellular binding domain of a heterologous immune receptor. Non-limiting examples of non-antibody antigen-binding compounds include ankyrin proteins, ankyrin repeat proteins, designed ankyrin repeat proteins (DARPins), affibodies, avimers, adnectins, anticalins, Fynomers, Kunitz domains, knottins, β-hairpin mimetics, and receptors and derivatives thereof.

Designed ankyrin repeat proteins (DARPins) can be protein scaffolds based on ankyrin repeat proteins. A DARPin can comprise one or more ankyrin repeats that comprise a shared sequence and/or structural motif. The individual ankyrin repeats can comprise a shared sequence and/or structural motif despite comprising mutations, substitutions, additions and/or deletions when compared to one other. A DARPin can comprise, for example, about 1, 2, 3, 4, 5, 6, 7, 8, 9, or 10 ankyrin repeats, or more. A DARPin can comprise an N-terminal capping repeat, one or more internal ankyrin repeats, and a C-terminal capping repeat. Each ankyrin repeat can comprise framework residues and protein-interaction residues. The framework residues can contribute to structure or folding topology, for example, the structure of an ankyrin repeat or interaction with a neighboring ankyrin repeat. Protein-interaction residues can contribute to binding of a target molecule, for example, via direct interaction with the target molecule, or by stabilizing directly-interacting residues in a conformation that allows binding.

In some embodiments, an extracellular binding domain binds to a target (e.g., antigen) expressed on or associated with a hematologic cancer cell or cell type. In some embodiments, an extracellular binding domain binds to a target (e.g., antigen) expressed on or associated with a solid tumor cell or cell type.

In some embodiments, an extracellular binding domain binds to CD19, ACE2, an Fc domain, APRIL, BAFFR, B7H6, B7H3, BCMA, CA9, CAIX, carcinoembryonic antigen, CD133, CD16, CD174, CD22, CD23, CD27, CD274, CD276, CD33, CD38, CD44, CD5, CD70, CEACAM5, CSPG4, CTLX, DNAM-1, Dsg3, E13Y IL13, E3 adnectin, EGFR, EGFRVIII, Envs, EPCAM, EPHA2, EPHB4, EPHRIN B2, ErbB, ERBB2, FAP, fibroblast activation protein, FLT3, FLT3L, FOLH1, FOLR1, FSH, FSHR, GD2, glycoprotein B, glycoprotein E2, GMCSF, GMR, gp120, gp41, GPC3, GPNMB, HBsAg, HER2, ICAM-I, IL10, IL10R, IL11, IL11Ra, IL13Ra2, IL1RAP, IL3RA, Insulin-B chain, Islet-specific glucose-6-phosphatase catalytic subunit-related protein, KDR, L1CAM, LFA-1, M2e, mesothelin, MET, MICA, MICB, MPL, MS4A1, MSLN, MUC1, myelin oligodendrocyte glycoprotein, NCAM1, Nectin-2, NKG2D, NKp30, PDCD1, PSCA, PSMA, PVR, ROR1, SARS-COV2 S protein, SDC1, SLAMF7, SSTR, T1E, TACI, TEM1, TNFRSF17, TNFRSF8, TPO, transmembrane form of IgE, TriPRIL, ULBP1, ULBP1-6, ULBP2, or VEGFR2. In some embodiments, an extracellular binding domain binds to CD19. In some embodiments, the extracellular binding domain comprises an FMC63 scFv.

In some embodiments, an extracellular binding domain binds to a surface molecule on a T cell, for example, a human T cell. The extracellular binding domain can bind to, for example, CD3, CD4, CD5, CD7, CD8, CD90, CD5, CD30, CD37, CCR4, TRB, TRAC, or TRBC1.

In some embodiments, an extracellular binding domain is or comprises a component of a receptor or a receptor ligand, for example, utilizes the naturally occurring specificity of a receptor or ligand. For example, an extracellular binding domain can comprise a receptor-binding domain or ligand-binding domain of B7H6, an Fc domain, APRIL, BCMA, CD16, CD27, CD70, CTLX, DNAM-1, E13Y IL13, E3 adnectin, EGFR, EPHB4, EPHRIN B2, ErbB1, ErbB2, ErbB3, ErbB4, FLT3, FLT3L, FSH, FSHR, GMCSF, GMR, ICAM-I, IL10, IL10R, IL11, IL11Ra, IL13Ra2, LFA-1, MICA, MICB, MPL, Nectin-2, or NKG2D.

In some embodiments, the extracellular binding domain binds to a T cell receptor, e.g., a constant domain, variable domain, CDR, LCDR3, or HCDR3 thereof. In some embodiments, the extracellular binding domain of the heterologous immune receptor binds to a particular T cell receptor clone, for example, specifically or preferentially binds to a TCR specific for a cognate autoimmune antigen.

In some embodiments, the extracellular binding domain of the heterologous immune receptor binds to a B cell receptor (BCR, e.g., IgM or IgD), e.g., a constant domain, variable domain, CDR, LCDR3, or HCDR3 thereof. In some embodiments, the extracellular binding domain of the heterologous immune receptor binds to a particular BCR clone, for example, specifically or preferentially binds to a BCR specific for a cognate autoimmune antigen.

In some embodiments, an extracellular binding domain is or comprises an autoantigen targeted by immune cells in an autoimmune disorder, or an epitope thereof. For example, a heterologous binding domain can comprise an autoantibody target, such as DSG3, factor VIII (FVIII), or an epitope thereof. The heterologous immune receptor can be, for example, a chimeric autoantibody receptor (CAAR).

In some embodiments, an extracellular binding domain binds to an autoimmunity-associated target, for example, muscle-associated receptor tyrosine kinase (MuSK), insulin peptide-major histocompatibility complex (MHC) class II complex or insulin.

In some embodiments, an extracellular binding domain binds to a senescence-associated cell surface molecule on a target cell, such as urokinase-type plasminogen activator receptor (uPAR) (e.g., human uPAR).

In some embodiments, an extracellular binding domain binds to a surface molecule on a fibrotic cell, for example, a fibroblast or an activated fibroblast. Such a heterologous immune receptor can be useful, for example, for treating or preventing fibrotic tissue, such as scar tissue that has formed or is forming. The cell surface molecule can be fibroblast activation protein (FAP). The extracellular binding domain can comprise, for example, an scFv that specifically binds to FAP (e.g., human FAP). The extracellular binding domain can be an scFv from clone 73.3 that specifically binds to FAP, a humanized version thereof, or a human FAP-specific equivalent thereof.

An extracellular binding domain can bind to an epitope of a target, e.g., cell surface molecule. Non-limiting examples of epitopes include amino acids, sugars, lipids, phosphoryl, and sulfonyl groups. An epitope can have specific three-dimensional structural characteristics, and/or specific charge characteristics. Epitopes can be conformational or linear.

A Extracellular binding domain can be selected for its affinity for one or more binding partners, such as a target cell surface molecule.

In some embodiments, an extracellular binding domain can bind to a target (e.g., surface molecule on a target cell) with a KD of, for example, less than about 100 mM, less than about 10 mM, less than about 1 mM, less than about 500 μM, less than about 100 μM, less than about 50 μM, less than about 10 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, less than about 900 pM, less than about 800 pM, less than about 700 pM, less than about 600 pM, less than about 500 pM, less than about 400 pM, less than about 300 pM, less than about 200 pM, less than about 100 pM, less than about 10 pM, or less than about 1 pM.

The heterologous immune receptor can comprise one or more additional extracellular domains as well as the extracellular binding domain.

In some embodiments, a heterologous immune receptor comprises an additional extracellular domain or amino acid sequence that is a linker or spacer. In some embodiments, a heterologous immune receptor comprises a hinge, such as an IgG hinge or a CD8 hinge.

The one or more additional extracellular domains can be an immune receptor extracellular domain. A heterologous immune receptor can comprise an extracellular domain or region that comprises (i) an extracellular binding domain, and (ii) an immune receptor extracellular domain. The immune receptor extracellular domain can contribute to the ability of the heterologous immune receptor to elicit a response upon encountering a target cell. An immune receptor extracellular domain can contribute to the structure of the heterologous immune receptor. For example, the immune receptor extracellular domain can contribute to homodimerization, heterodimerization, or multimerization that can contribute to the function of the heterologous immune receptor. The immune receptor extracellular domain can be a component of a TCR signaling complex. The component of the TCR signaling complex can be or can comprise, for example, an extracellular domain of TCR alpha chain constant region, TCR beta chain constant region, TCR gamma chain constant region, TCR delta chain constant region, CD3 gamma, CD3 delta, CD3 epsilon, CD3 zeta, a functional fragment thereof, or a combination thereof.

### 7. Transmembrane Domain

A heterologous immune receptor can comprise a transmembrane domain. Any suitable transmembrane domain can be used. In some embodiments, the heterologous immune receptor comprises a transmembrane domain of CD8. In some embodiments, the heterologous immune receptor comprises a transmembrane domain of CD28. In some embodiments, the heterologous immune receptor comprises a transmembrane domain of OX40, 41BB, or CD86.

The transmembrane domain can be a transmembrane domain of an immune receptor or TCR signaling complex component disclosed herein, for example, of a mammalian or a human TCR signaling complex. The transmembrane domain can comprise, for example, a transmembrane domain of TCR alpha chain, TCR beta chain, TCR gamma chain, TCR delta chain, CD3 gamma, CD3 delta, CD3 epsilon, or CD3 zeta. In some embodiments, a transmembrane domain is not from an immune receptor or is not from a TCR signaling complex component.

### 8. Cytoplasmic Domain and Cytoplasmic Signaling Domain

A heterologous immune receptor can comprise a cytoplasmic domain or a mutant, variant, or derivative thereof. A cytoplasmic domain can comprise a cytoplasmic signaling domain or a mutant, variant, or derivative thereof. The cytoplasmic signaling domain can contribute to the ability of the heterologous immune receptor to elicit a response. For example, the cytoplasmic signaling domain can contribute to induction of signaling and/or immune cell activation upon of binding of the heterologous immune receptor (e.g., an extracellular binding domain thereof) to a surface molecule of a target cell. In some cases, the cytoplasmic signaling domain can contribute to the induction of a pro-inflammatory response, an anti-cancer immune response, an immune tolerance-promoting response, a transcriptional response, TCR signaling, T cell activation, T cell proliferation, cytokine production, a cytotoxic response against the target cell, or a combination thereof. In some cases, the cytoplasmic signaling domain can contribute to the activation of bystander immune cells that do not comprise a heterologous immune receptor of the disclosure. A cytoplasmic signaling domain can enhance the proliferation, survival, and/or function of immune cells, and/or development of effector and/or memory immune responses (e.g., memory T cells).

A cytoplasmic signaling domain can partake in an immune cell activation pathway that involves, for example, phosphorylation, dephosphorylation, calcium release, ubiquitination, de-ubiquitination, proteolytic cleavage, protein-protein interactions, a transcriptional response, or a combination thereof. An immune cell activation pathway can comprise, for example, an innate, adaptive, STING, NFKB, inflammasome, TCR, BCR, JAK/STAT, TLR, NLR, RLR, co-stimulatory, co-inhibitory, cytokine, or chemokine signaling pathway. A cytoplasmic signaling domain can comprise one or more immunoreceptor tyrosine-based activation motifs (ITAMs). A cytoplasmic signaling domain can comprise one or more immunoreceptor tyrosine-based inhibition motifs (ITIMs).

In some embodiments, the heterologous immune receptor contains a cytoplasmic signaling domain of CD3 zeta or a functional fragment thereof. In some embodiments, the heterologous immune receptor contains a cytoplasmic signaling domain of CD3 zeta with 1, 2, or 3 functional or active ITAMs. In some embodiments, the heterologous immune receptor contains a cytoplasmic signaling domain of CD3 zeta with one inactivated ITAM or two inactivated ITAMs. In some embodiments, the heterologous immune receptor does not contain a cytoplasmic signaling domain of CD3 zeta or a functional fragment thereof.

A heterologous immune receptor can comprise a cytoplasmic signaling domain of a T cell signal two costimulatory signaling domain, or a functional fragment thereof. In some embodiments, a heterologous immune receptor does not contain a cytoplasmic signaling domain of a T cell signal two costimulatory signaling domain.

A cytoplasmic domain or cytoplasmic signaling domain can be derived from and/or interact with a kinase, (e.g., a protein kinase, a tyrosine kinase or a serine/threonine kinase, a receptor tyrosine kinase, a lipid kinase, a phosphoinositide kinase, a carbohydrate kinase, or a combination thereof), a phosphatase, a ubiquitin ligase, a caspase, an adapter protein, a transcription factor, an ion channel, or a combination thereof. A cytoplasmic domain or cytoplasmic signaling domain can contribute to interaction of the heterologous immune receptor with additional proteins or factors (e.g., members of a complex and/or signal transduction pathway).

A heterologous immune receptor can comprise a cytoplasmic signaling domain of a costimulatory immune receptor, or a functional fragment thereof. Non-limiting examples of costimulatory immune receptors include CD28, 2B4 (CD244, SLAMF4), 4-1BB (CD137), CD2 (LFA2, OX34), CD21, CD226 (DNAM1), CD27 (TNFRSF7), CD30 (TNFRSF8), CD4, CD40, CD8, CD84 (SLAMF5), CRACC (CD319, BLAME), CRTAM (CD355), DcR3, DR3 (TNFRSF25), GITR (CD357), HVEM (CD270), ICOS (CD278), LIGHT, LTBR (TNFRSF3), Ly108 (NTBA, CD352, SLAMF6), Ly9 (CD229,SLAMF3), OX40 (CD134), SLAM (CD150, SLAMF1), TIM1 (HAVCR1, KIM1), and TIM2. In some embodiments, a heterologous immune receptor does not contain a cytoplasmic signaling domain of a costimulatory immune receptor.

A heterologous immune receptor can comprise a cytoplasmic signaling domain of a co-inhibitory immune receptor, or a functional fragment thereof. Non-limiting examples of co-inhibitory immune receptors include CTLA4 (CD152), 2B4, B71 (CD80), B7H1 (CD274, PDL1), BTLA (CD272), CD160 (BY55, NK28), DR6 (CD358), Fas, LAG3 (CD223), LAIR1, Ly108, PD1 (CD279), PD1H (VISTA), TIGIT (VSIG9, VSTM3), TIM1, TIM2 (TIMD2), and TIM3 (HAVCR2, KIM3). In some embodiments, a heterologous immune receptor does not contain a cytoplasmic signaling domain of a co-inhibitory immune receptor.

A heterologous immune receptor can comprise a cytoplasmic signaling domain of an activating NK receptor, or a functional fragment thereof. Non-limiting examples of activating NK receptors include CD100 (SEMA4D), CD16 (FcgRIIIA), CD160 (BY55), CD244 (2B4, SLAMF4), CD27, CD94-NKG2C, CD94-NKG2E, CD94-NKG2H, CD96, CRTAM, DAP12, DNAM1 (CD226), KIR2DL4, KIR2DS1, KIR2DS2, KIR2DS3, KIR2DS4, KIR2DS5, KIR3DS1, Ly49, NCR, NKG2D (KLRK1, CD314), NKp30 (NCR3), NKp44 (NCR2), NKp46 (NCR1), NKp80 (KLRF1, CLEC5C), NTB-A (SLAMF6), PSGLI, and SLAMF7 (CRACC, CS1, CD319). In some embodiments, a heterologous immune receptor does not contain a cytoplasmic signaling domain of an activating NK receptor.

A heterologous immune receptor can comprise a cytoplasmic signaling domain of an inhibitory NK receptor, or a functional fragment thereof. Non-limiting examples of inhibitory NK receptors include CD161 (NKR-PIA, NK1.1), CD94-NKG2A, CD96, CEACAMI, KIR2DL1, KIR2DL2, KIR2DL3, KIR2DL4, KIR2DL5A, KIR2DL5B, KIR3DL1, KIR3DL2, KIR3DL3, KLRG1, LAIR1, LIR1 (ILT2, LILRB1), Ly49a, Ly49b, NKR-PIA (KLRB1), SIGLEC-10, SIGLEC-11, SIGLEC-14, SIGLEC-16, SIGLEC-3 (CD33), SIGLEC-5 (CD170), SIGLEC-6 (CD327), SIGLEC-7 (CD328), SIGLEC-8, SIGLEC-9 (CD329), SIGLEC-E, SIGLEC-F, SIGLEC-G, SIGLEC-H, and TIGIT. In some embodiments, a heterologous immune receptor does not contain a cytoplasmic signaling domain of an inhibitory NK receptor.

A heterologous immune receptor can comprise a cytoplasmic signaling domain of a tumor necrosis factor receptor superfamily member, or a functional fragment thereof. Non-limiting examples of tumor necrosis factor receptor superfamily members include 4-1BB (CD137), BAFFR (CD268), BCMA (CD269), CD27 (TNFRSF7), CD30 (TNFRSF8), CD40 (TNFRSF5), DcR3 (TNFRSF6B), DR3 (TNFRSF25), DR6 (CD358), EDAR, FAS (CD95), GITR (CD357), HVEM (CD270), LTBR (TNFRSF3), NGFR (CD271), OPG (TNFRSF11B), OX40 (CD134), RANK (CD265), RELT (TNFRSF19L), TACI (CD267), TNFR1 (CD120a), TNFR2 (CD120b), TRAILR1-4 (TNFRSF10A-D), TROY (TNFRSF19), TWEAKR (CD266), and XEDAR (TNFRSF27). In some embodiments, a heterologous immune receptor does not contain a cytoplasmic signaling domain of a tumor necrosis factor receptor superfamily member.

A heterologous immune receptor can comprise a cytoplasmic signaling domain of an immunoglobulin superfamily member, or a functional fragment thereof. Non-limiting examples of immunoglobulin superfamily members include CD28, ICOS (CD278), CTLA4 (CD152), PD1 (CD279), PD1H (VISTA), BTLA (CD272), B71 (CD80), B7H1 (CD274,PDL1), CD226 (DNAM1), CRTAM (CD355), TIGIT (VSIG9, VSTM3), CD96 (TACTILE), TIM1 (HAVCR1,KIM1), TIM2 (TIMD2), TIM3 (HAVCR2,KIM3), TIM4 (TIMD4), CD2 (LFA2,OX34), SLAM (CD150,SLAMF1), 2B4 (CD244,SLAMF4), Ly108 (NTBA,CD352,SLAMF6), CD84 (SLAMF5), Ly9 (CD229,SLAMF3), CRACC (CD319,BLAME), BTN1 (BTN1A1), BTN2 (BTN2A1-3), BTN3 (BTN3A1-3), LAIR1, LAG3 (CD223), and CD160 (BY55,NK28). In some embodiments, a heterologous immune receptor does not contain a cytoplasmic signaling domain of an immunoglobulin superfamily member.

A heterologous immune receptor can comprise a cytoplasmic signaling domain of an Fcγ receptor (FcγR), an Fcε receptor (FcεR), an Fcα receptor (FcαR), an Fcμ receptor (FcμR), neonatal Fc receptor (FcRn), CD4, CD5, CD8, CD21, CD22, CD27, CD28, CD32, CD40, CD45, CD66d, CD79a, CD79b, CD80, CD86, CD278 (ICOS), CD247ζ, CD247η, 41BB, DAP10, DAP12, FYN, LAT, Lck, MAPK, MHC complex, NFAT, NF-κB, PLC-γ, iC3b, C3dg, C3d, Zap70, MyD88, a functional fragment thereof, or a combination thereof. In some embodiments, the heterologous immune receptor comprises a cytoplasmic signaling domain of one or more of CD3 gamma, CD3 delta, CD3 epsilon, and CD3 zeta and a cytoplasmic signaling domain of one or more of an Fcγ receptor (FcγR), an Fcα receptor (FcεR), an Fcα receptor (FcαR), an Fcμ receptor (FcμR), neonatal Fc receptor (FcRn), CD4, CD5, CD8, CD21, CD22, CD27, CD28, CD32, CD40, CD45, CD66d, CD79a, CD79b, CD80, CD86, CD278 (ICOS), CD247ζ, CD247η, 41BB, DAP10, DAP12, FYN, LAT, Lck, MAPK, MHC complex, NFAT, NF-κB, PLC-γ, iC3b, C3dg, C3d, Zap70, and MyD88.

A heterologous immune receptor can comprise a cytoplasmic signaling domain that is derived from, interacts with, increases expression of, or activates a transcription factor, such as, for example, E2A, Pax5, EBF, PU.1, Ikaros, GATA3, Th-POK, Tbet, Bcl6, NF-κB, NFAT, AP-1, NFAT, STAT1, STAT2, STAT3, STAT4, STAT5, STATSA, STAT5B, STAT6, STAT7, IRF1, IRF2, IRF3, IRF4, IRF5, IRF6, IRF7, IRF8, IRF9, AP-1, Eomes, FoxP3, Id2, PLZF, ROR-gamma-T, TCF7, ThPOK, or any combination thereof.

A heterologous immune receptor can comprise a cytoplasmic signaling domain that is a component of a TCR signaling complex, for example, of a mammalian or a human TCR signaling complex. The cytoplasmic signaling domain can comprise, for example, a cytoplasmic signaling domain of CD3 gamma, CD3 delta, CD3 epsilon, CD3 zeta, a functional fragment thereof, or a combination thereof.

In some cases, a heterologous immune receptor of the disclosure does not contain a cytoplasmic signaling domain, but can nonetheless elicit an immune cell activation signal, for example, via a cytoplasmic signaling domain in another protein that can associate with the heterologous immune receptor. In some embodiments, a heterologous immune receptor of the disclosure that comprises constant regions from one or more TCR chains can transmit an immune cell activation signal via associated CD3 proteins that comprise cytoplasmic signaling domains (e.g., CD3 zeta, CD3 gamma, CD3 delta, CD3 epsilon, CD3 eta, or a combination thereof).

A heterologous immune receptor can comprise a component of a TCR signaling complex (e.g., extracellular, transmembrane, and/or cytoplasmic domain of TCR alpha chain constant region, TCR beta chain constant region, TCR gamma chain constant region, TCR delta chain constant region, CD3 epsilon, CD3 gamma, CD3 delta, or CD3 zeta), and further comprise one or more cytoplasmic signaling domains from a heterologous protein disclosed herein.

A heterologous immune receptor can comprise one or more cytoplasmic signaling domains or mutants, variants, or derivatives thereof. A heterologous immune receptor can comprise, for example, 0, 1, 2, 3, 4, 5, 6, 7, 8, 9, 10, or more cytoplasmic signaling domains or mutants, variants, or derivatives thereof. A heterologous immune receptor can comprise at least one, at least two, at least three, at least four, or at least five cytoplasmic signaling domains. A heterologous immune receptor can comprise at most one, at most two, at most three, at most four, at most five, or at most ten cytoplasmic signaling domains.

A cytoplasmic signaling domain can be any size. For example, in some cases, a cytoplasmic signaling domain can be at least 10, at least 15, at least 20, at least 25, at least 30, at least 40, at least 50, at least 70, or at least 100 amino acids, or more. In some embodiments, a cytoplasmic signaling domain can be at most 15, at most 20, at most 30, at most 50, at most 70, at most 100, or at most 150 amino acids, or less. A cytoplasmic signaling domain can be between about 10-200, 10-100, 10-80, 10-60, 10-40, 10-30, 10-20, 20-200, 20-100, 20-80, 20-60, 20-40, 20-30, 30-200, 30-100, 30-80, 30-60, 30-40, 40-200, 40-100, 40-80, 40-60, 50-200, 50-100, 50-80, 50-60, 80-200, 80-100, 100-200, 100-180, 100-160, 100-150, 100-140, 100-130, 100-120, 50-100, 100-120, 100-140, 100-160, 100-180, 100-150, or 150-200 amino acids.

A cytoplasmic signaling domain can be from a mammalian protein. In some cases, a cytoplasmic signaling domain is from a murine (mouse) protein. In some cases, a cytoplasmic signaling domain is from a human protein.

In some cases, a cytoplasmic signaling domain can comprise modifications compared to a wild type sequence or a sequence disclosed herein (for example, one or more insertions, deletions, and/or substitutions). Mutations can be introduced, for example, to alter (e.g., increase or decrease) the stability of an interaction between the cytoplasmic signaling domain and an interacting partner (e.g., signaling adaptor protein or enzyme). Mutations can be introduced, for example, to alter (e.g., increase or decrease) the intensity of an immune activation signal upon binding of the heterologous immune receptor to a binding partner.

### 9. Other Domains and Configuration

A heterologous immune receptor of the disclosure can comprise one or more linkers for example, between different domains of the protein. A linker can be a chemical bond, for example, a covalent bond or a non-covalent bond. A linker as described herein can include a flexible or rigid linker. A linker can be a peptide.

A linker can be selected to achieve a desired functionality of the heterologous immune receptor. For example, various linkers can be tested to identify a configuration of one or more linkers that allow a heterologous immune receptor of the disclosure to exhibit low background activity, for example, low induction of an immune activation signal in the absence of an appropriate stimulus (e.g., absence of a target cell surface molecule). Various linkers can be tested to identify a configuration of one or more linkers that allow a heterologous immune receptor of the disclosure to exhibit high inducibility, for example, strong induction of an immune activation signal in the presence of an appropriate stimulus (e.g., presence of a target cell surface molecule).

A linker can comprise a linker sequence, for example, a linker peptide sequence. The length a linker can be adjusted to allow for proper folding or to increase or decrease biological activity of the heterologous immune receptor. A linker sequence can be, for example, about 1, about 2, about 3, about 4, about 5, about 6, about 7, about 8, about 9, about 10, about 11, about 12, about 13, about 14, about 15, about 20, about 25, about 30, about 40, about 50, about 60, or about 70 amino acid residues in length. In some cases, a linker sequence can be, for example at least about 2, at least about 3, at least about 4, at least about 5, at least about 6, at least about 7, at least about 8, at least about 9, at least about 10, at least about 15, at least about 20, at least about 30, at least about 40, or at least about 50 amino acids in length. In some cases, a linker sequence can be, for example at most about 2, at most about 3, at most about 4, at most about 5, at most about 6, at most about 7, at most about 8, at most about 9, at most about 10, at most about 15, at most about 20, at most about 30, at most about 40, at most about 50, at most about 60, at most about 70, at most about 80, or at most about 100 amino acids in length. In some cases, a linker is 5-20 amino acids in length. In some cases, a linker is 10-20 amino acids in length.

A flexible linker can have a sequence containing glycine residues. The small size of the glycine residues can provide flexibility, and allow for mobility of the connected protein domains. The incorporation of serine or threonine can maintain the stability of the linker in aqueous conditions by forming hydrogen bonds with the water molecules, thereby reducing unfavorable interactions between the linker and protein moieties. In some cases, flexible linkers can also contain additional amino acids, such as threonine and alanine, to maintain flexibility, and/or polar amino acids such as lysine and glutamine, to improve solubility.

A rigid linker can have, for example, an alpha helix-structure. An alpha-helical rigid linker can act as a spacer between protein domains. A rigid linker can have a proline-rich sequence, (XP) n, with X designating alanine, lysine, glutamine, or any amino acid, and n designating a number of repeats. The presence of proline in non-helical linkers can increase stiffness, and allow for effective separation of protein domains.

A linker can comprise a hinge region, for example an amino acid sequence derived from a hinge region of an antibody or immune receptor. In some embodiments, a linker comprises a hinge region from CD8α, IgG1, or IgG4.

In some embodiments, a heterologous immune receptor comprises a linker or hinge disclosed herein or a repeat and/or variant thereof, that joins any two domains (for example, any one of the hinges or linkers of any one of SEQ ID NOs: 46, 47, and 98-109, a repeat thereof, and/or a variant thereof). The linker can comprise, consist of, or consist essentially of, e.g., one two, three, four, five, or more repeats of any of the sequences. The variant can comprise a minimum percentage sequence identity, or a number of insertions, deletions, and/or substitutions disclosed herein.

A polynucleotide encoding a heterologous immune receptor of the disclosure can be designed to encode two or more components, (e.g., components of a heterologous immune receptor, or a heterologous immune receptor and an immunomodulatory factor or safety switch) linked by one or more 2A linkers, which can be processed into separate polypeptides co-translationally or after translation. An illustrative sequence of a polynucleotide encoding a heterologous immune receptor and a safety switch is provided in SEQ ID NO: 128 (encoding a CAR with an anti-CD19 scFv, CD28, CD27, and CD3z cytoplasmic signaling domains, a 2A linker, and iCasp9), and the amino acid sequence in SEQ ID NO: 126.

Inclusion of a 2A linker can increase the likelihood that an appropriate ratio of components are produced (e.g., a 1:1, 1:2, 1:3, 1:4, or 1:5 ratio of two components). In some cases, inclusion of a 2A linker can increase the likelihood that equal or close to equal levels of two components are produced. For example, in some embodiments a heterologous immune receptor can comprise a TCR alpha chain constant region and TCR beta chain constant region, or a TCR gamma chain constant region and a TCR delta chain constant region, and inclusion of a 2A linker can increase the likelihood that equal or close to equal levels of a the paired alpha & beta or gamma and delta chain constant regions are produced.

In some cases, use of a 2A linker can allow for fewer components in a system for transgene expression and/or genome modification, e.g., inclusion of multiple components in one vector rather than separate vectors.

A recombinant nucleic acid encoding a heterologous immune receptor of the disclosure can encode a signal peptide. In some cases, a heterologous immune receptor of the disclosure comprises a signal peptide. A signal peptide can be cleaved off during processing of the protein, thus in some cases a mature heterologous immune receptor disclosed herein does not contain a signal peptide.

A signal peptide at the N-terminus of a protein can be involved in transport of the protein to or through a membrane, transport to a different membranous cellular compartment, or secretion of the protein from the cell. A recombinant nucleic acid encoding a heterologous immune receptor of the disclosure can encode a signal peptide to facilitate membrane insertion and surface localization of the heterologous immune receptor. A signal peptide can be selected for its ability to facilitate ER processing and cell surface localization of the heterologous immune receptor. Any suitable signal peptide can be used. In some embodiments, the signal peptide ca comprise a CD8α signal peptide or an IgG signal peptide.

Components of a heterologous immune receptor of the disclosure can be configured such that when the heterologous immune receptor is expressed in an engineered immune cell, contacting the immune cell with a target cell induces an immune cell activation signal (e.g., upon binding of the extracellular binding domain of the heterologous immune receptor to a surface molecule on the target cell). A heterologous immune receptor of the disclosure can comprise one or more extracellular domains, one or more transmembrane domains, and one or more cytoplasmic domains configured such that when the heterologous immune receptor is expressed in an engineered immune cell, contacting the immune cell with a target cell induces an immune cell activation signal.

In some embodiments, a heterologous immune receptor is a chimeric antigen receptor known as or within an engineered cell or construct known as Tisagenlecleucel (Kymriah®), Axicabtagene Ciloleucel (Yescarta®), Brexucabtagene Autoleucel (Tecartus™), Lisocabtagene maraleucel, Idecabtagene Vicleucel, or KTE-X19.

An illustrative CAR comprising an anti-CD19 scFv, and CD28, CD27, and CD3z cytoplasmic signaling domains is provided in SEQ ID NO: 127.

In some embodiments, a heterologous immune receptor disclosed herein or a domain thereof comprises, consists essentially of, or consists of an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to any one of SEQ ID NOs: 41-87, 96-109, and 127, or a domain thereof disclosed herein.

In some embodiments, a heterologous immune receptor disclosed herein or a domain thereof comprises, consists essentially of, or consists of an amino acid sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity or sequence similarity to any one of SEQ ID NOs: 41-87, 96-109, and 127, or a domain thereof disclosed herein.

In some embodiments, a heterologous immune receptor disclosed herein or a domain thereof comprises, consists essentially of, or consists of an amino acid sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, about 99.5%, or about 100% sequence identity or sequence similarity to any one of SEQ ID NOs: 41-87, 96-109, and 127, or a domain thereof disclosed herein.

In some embodiments, a heterologous immune receptor disclosed herein or a domain thereof comprises, consists essentially of, or consists of an amino acid sequence that is any one of SEQ ID NOs: 41-87, 96-109, and 127, or a domain thereof disclosed herein.

In some embodiments, a heterologous immune receptor disclosed herein or a domain thereof comprises, consists essentially of or consists of an amino acid sequence with one or more insertions, deletions, and/or substitutions relative to any one of SEQ ID NOs: 41-87, 96-109, and 127, or a domain thereof disclosed herein.

For example, the heterologous immune receptor or a domain thereof can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions, deletions, and/or substitutions relative to any one of SEQ ID NOs: 41-87, 96-109, and 127, or a domain thereof disclosed herein.

In some embodiments, the heterologous immune receptor or a domain thereof comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions, deletions, and/or substitutions relative to any one of SEQ ID NOs: 41-87, 96-109, and 127, or a domain thereof disclosed herein.

In some embodiments, the heterologous immune receptor or a domain thereof comprises 1, 2, 3, 4, 5, 6, 7, 8, 9, 10, 11, 12, 13, 14, 15, 16, 17, 18, 19, 20, 25, 30, 35, 40, 45, or 50 amino acid insertions, deletions, and/or substitutions relative to any one of SEQ ID NOs: 41-87, 96-109, and 127, or a domain thereof disclosed herein.

The one or more insertions, deletions, and/or substitutions can be at N-terminus, the C-terminus, within the amino acid sequence, or a combination thereof. The one or more insertions, deletions, and/or substitutions can be contiguous, non-contiguous, or a combination thereof.

TABLE 2
illustrative sequences of heterologous immune
receptors and domains or components thereof.

| SEQ | | |
|---|---|---|
| ID | | |
| NO | Description | Sequence |
| 41 | Anti-mouse | MGVPTQLLGLLLLWITDAICDIQMTQSPASLSTSLGETVTIQC |
| | CD19 CAR | QASEDIYSGLAWYQQKPGKSPQLLIYGASDLQDGVPSRFSGS |
| | (synthetic | GSGTQYSLKITSMQTEDEGVYFCQQGLTYPRTFGGGTKLELK |
| | construct) | GGGGSGGGGSGGGGSEVQLQQSGAELVRPGTSVKLSCKVSG |
| | | DTITFYYMHFVKQRPGQGLEWIGRIDPEDESTKYSEKFKNKA |
| | | TLTADTSSNTAYLKLSSLTSEDTATYFCIYGGYYFDYWGQGV |
| | | MVTVSSIEFMYPPPYLDNERSNGTIIHIKEKHLCHTQSSPKLF |
| | | WALVVVAGVLFCYGLLVTVALCVIWTNSRRNRGGQSDYMN |
| | | MTPRRPGLTRKPYQPYAPARDFAAYRPRAKFSRSAETAANL |
| | | QDPNQLFNELNLGRREEFDVLEKKRARDPEMGGKQQRRRNP |
| | | QEGVYNALQKDKMAEAYSEIGTKGERRRGKGHDGLFQGLS |
| | | TATKDTFDALHMQTLAPR |
| 42 | Anti-CD19 scFv | DIQMTQTTSSLSASLGDRVTISCRASQDISKYLNWYQQKPDG |
| | (synthetic | TVKLLIYHTSRLHSGVPSRFSGSGSGTDYSLTISNLEQEDIATY |
| | construct) | FCQQGNTLPYTFGGGTKLEITGSTSGSGKPGSGEGSTKGEVK |
| | | LQESGPGLVAPSQSLSVTCTVSGVSLPDYGVSWIRQPPRKGL |
| | | EWLGVIWGSETTYYNSALKSRLTIIKDNSKSQVFLKMNSLQT |
| | | DDTAIYYCAKHYYYGGSYAMDYWGQGTSVTVSS |
| 43 | anti-CD19 scFv | DIQMTQTTSSLSASLGDRVTISCRASQDISKYLNWYQQKPDG |
| | (synthetic | TVKLLIYHTSRLHSGVPSRFSGSGSGTDYSLTISNLEQEDIATY |
| | construct) | FCQQGNTLPYTFGGGTKLEITGGGGSGGGGSGGGGSEVKLQ |
| | | ESGPGLVAPSQSLSVTCTVSGVSLPDYGVSWIRQPPRKGLEW |
| | | LGVIWGSETTYYNSALKSRLTIIKDNSKSQVFLKMNSLQTDD |
| | | TAIYYCAKHYYYGGSYAMDYWGQGTSVTVSS |
| 44 | Anti-CD19 VL | DIQMTQTTSSLSASLGDRVTISCRASQDISKYLNWYQQKPDG |
| | (synthetic | TVKLLIYHTSRLHSGVPSRFSGSGSGTDYSLTISNLEQEDIATY |
| | construct) | FCQQGNTLPYTFGGGTKLEITG |
| 45 | Anti-CD19 VH | EVKLQESGPGLVAPSQSLSVTCTVSGVSLPDYGVSWIRQPPR |
| | (synthetic | KGLEWLGVIWGSETTYYNSALKSRLTIIKDNSKSQVFLKMNS |
| | construct) | LQTDDTAIYYCAKHYYYGGSYAMDYWGQGTSVTVSS |
| 46 | CD8 Hinge | TTTPAPRPPTPAPTIASQPLSLRPEACRPAAGGAVHTRGLDFA |
| | | CD |
| 47 | IgG1 hinge | AEPKSPDKTHTCPPCPKDPK |
| 48 | CD8 TM | IYIWAPLAGTCGVLLLSLVITLYC |
| 49 | CD28 TM | FWVLVVVGGVLACYSLLVTVAFIIFWV |
| 50 | OX40 TM | VAAILGLGLVLGLLGPLAILL |
| 51 | 41BB TM | IISFFLALTSTALLFLLFFLTLRFSVV |
| 52 | CD86 TM | WITAVLPTVIICVMVFCLILW |
| 53 | CD3 epsilon TM | VMSVATIVIVDICITGGLLLLVYYWS |
| 54 | CD3 gamma TM | GFLFAEIVSIFVLAVGVYFIA |
| 55 | CD3 delta TM | GIIVTDVIATLLLALGVFCFA |
| 56 | CD3 zeta TM | LCYLLDGILFIYGVILTALFL |
| 57 | TCR alpha TM | VIGFRILLLKVAGFNLLMTLRLW |
| 58 | TCR beta TM | ILLGKATLYAVLVSALVLMAM |
| 59 | TCR gamma 1 | YYMYLLLLLKSVVYFAIITCCLL |
| | TM | |
| 60 | TCR gamma 2 | YYTYLLLLLKSVVYFAIITCCLL |
| | TM | |
| 61 | TCR delta TM | LGLRMLFAKTVAVNFLLTAKLFF |
| 62 | CD3 zeta | RVKFSRSADAPAYKQGQNQLYNELNLGRREEYDVLDKRRG |
| | cytoplasmic | RDPEMGGKPRRKNPQEGLYNELQKDKMAEAYSEIGMKGER |
| | signaling domain | RRGKGHDGLYQGLSTATKDTYDALHMQALPPR |
| 63 | CD3 zeta | RVKFSRSADAPAYQQGQNQLYNELNLGRREEYDVLDKRRG |
| | cytoplasmic | RDPEMGGKPRRKNPQEGLYNELQKDKMAEAYSEIGMKGER |
| | signaling domain | RRGKGHDGLYQGLSTATKDTYDALHMQALPPR |
| 64 | CD28 | RSKRSRLLHSDYMNMTPRRPGPTRKHYQPYAPPRDFAAYRS |
| | cytoplasmic | |
| | signaling domain | |
| 65 | OX40 | RDQRLPPDAHKPPGGGSFRTPIQEEQADAHSTLAKI |
| | cytoplasmic | |
| | signaling domain | |
| 66 | CD27 | QRRKYRSNKGESPVEPAEPCHYSCPREEEGSTIPIQEDYRKPE |
| | cytoplasmic | PACSP |
| | signaling domain | |
| 67 | 41BB | KRGRKKLLYIFKQPFMRPVQTTQEEDGCSCRFPEEEEGGCEL |
| | cytoplasmic | |
| | signaling domain | |
| 68 | NKp80 | MQDEDGYMTLNVQSKKRSSAQTSQLTFKDYSVTLHW |
| | cytoplasmic | |
| | signaling domain | |
| 69 | CD3 epsilon | KNRKAKAKPVTRGAGAGGRQRGQNKERPPPVPNPDYEPIRK |
| | cytoplasmic | GQRDLYSGLNQRRI |
| | domain | |
| 70 | CD3 gamma | GQDGVRQSRASDKQTLLPNDQLYQPLKDREDDQYSHLQGN |
| | cytoplasmic | QLRRN |
| | domain | |
| 71 | CD3 delta | GHETGRLSGAADTQALLRNDQVYQPLRDRDDAQYSHLGGN |
| | cytoplasmic | WARNK |
| | domain | |
| 72 | CD3 zeta | RVKFSRSADAPAYQQGQNQLYNELNLGRREEYDVLDKRRG |
| | cytoplasmic | RDPEMGGKPQRRKNPQEGLYNELQKDKMAEAYSEIGMKGE |
| | domain | RRRGKGHDGLYQGLSTATKDTYDALHMQALPPR |
| 73 | CD3 epsilon | ERPPPVPNPDYEPIRKGQRDLYSGLNQR |
| | ITAM (human) | |
| 74 | CD3 gamma | DKQTLLPNDQLYQPLKDREDDQYSHLQGN |
| | ITAM (human) | |
| 75 | CD3 delta ITAM | DTQALLRNDQVYQPLRDRDDAQYSHLGGN |
| | (human) | |
| 76 | CD3 zeta | APAYQQGQNQLYNELNLGRREEYDVLDKR |
| | ITAM1 (human) | |
| 77 | CD3 zeta | PQRRKNPQEGLYNELQKDKMAEAYSEIGM |
| | ITAM2 (human) | |
| 78 | CD3 zeta | ERRRGKGHDGLYQGLSTATKDTYDALHMQ |
| | ITAM3 (human) | |
| 79 | CD3 epsilon | DGNEEMGGITQTPYKVSISGTTVILTCPQYPGSEILWQHNDK |
| | (human) | NIGGDEDDKNIGSDEDHLSLKEFSELEQSGYYVCYPRGSKPE |
| | | DANFYLYLRARVCENCMEMDVMSVATIVIVDICITGGLLLLV |
| | | YYWSKNRKAKAKPVTRGAGAGGRQRGQNKERPPPVPNPDY |
| | | EPIRKGQRDLYSGLNQRRI |
| 80 | CD3 gamma | QSIKGNHLVKVYDYQEDGSVLLTCDAEAKNITWFKDGKMIG |
| | (human) | FLTEDKKKWNLGSNAKDPRGMYQCKGSQNKSKPLQVYYR |
| | | MCQNCIELNAATISGFLFAEIVSIFVLAVGVYFIAGQDGVRQS |
| | | RASDKQTLLPNDQLYQPLKDREDDQYSHLQGNQLRRN |
| 81 | CD3 delta | FKIPIEELEDRVFVNCNTSITWVEGTVGTLLSDITRLDLGKRIL |
| | (human) | DPRGIYRCNGTDIYKDKESTVQVHYRMCQSCVELDPATVAGI |
| | | IVTDVIATLLLALGVFCFAGHETGRLSGAADTQALLRNDQVY |
| | | QPLRDRDDAQYSHLGGNWARNK |
| 82 | CD3 zeta | QSFGLLDPKLCYLLDGILFIYGVILTALFLRVKFSRSADAPAY |
| | (human) | QQGQNQLYNELNLGRREEYDVLDKRRGRDPEMGGKPQRRK |
| | | NPQEGLYNELQKDKMAEAYSEIGMKGERRRGKGHDGLYQG |
| | | LSTATKDTYDALHMQALPPR |
| 83 | TCR alpha chain | IQNPDPAVYQLRDSKSSDKSVCLFTDFDSQTNVSQSKDSDVY |
| | constant (human) | ITDKTVLDMRSMDFKSNSAVAWSNKSDFACANAFNNSIIPED |
| | | TFFPSPESSCDVKLVEKSFETDTNLNFQNLSVIGFRILLLKVAG |
| | | FNLLMTLRLWSS |
| 84 | TCR alpha beta | DLNKVFPPEVAVFEPSEAEISHTQKATLVCLATGFFPDHVELS |
| | constant (human) | WWVNGKEVHSGVSTDPQPLKEQPALNDSRYCLSSRLRVSAT |
| | | FWQNPRNHFRCQVQFYGLSENDEWTQDRAKPVTQIVSAEA |
| | | WGRADCGFTSVSYQQGVLSATILYEILLGKATLYAVLVSALV |
| | | LMAMVKRKDF |
| 85 | TCR gamma | DKQLDADVSPKPTIFLPSIAETKLQKAGTYLCLLEKFFPDVIKI |
| | constant 1 | HWQEKKSNTILGSQEGNTMKTNDTYMKFSWLTVPEKSLDKE |
| | (human) | HRCIVRHENNKNGVDQEIIFPPIKTDVITMDPKDNCSKDANDT |
| | | LLLQLTNTSAYYMYLLLLLKSVVYFAIITCCLLRRTAFCCNGE |
| | | KS |
| 86 | TCR gamma | DKQLDADVSPKPTIFLPSIAETKLQKAGTYLCLLEKFFPDIIKI |
| | constant 2 | HWQEKKSNTILGSQEGNTMKTNDTYMKFSWLTVPEESLDKE |
| | (human) | HRCIVRHENNKNGIDQEIIFPPIKTDVTTVDPKYNYSKDANDV |
| | | ITMDPKDNWSKDANDTLLLQLTNTSAYYTYLLLLLKSVVYF |
| | | AIITCCLLRRTAFCCNGEKS |
| 87 | TCR delta | SQPHTKPSVFVMKNGTNVACLVKEFYPKDIRINLVSSKKITEF |
| | constant (human) | DPAIVISPSGKYNAVKLGKYEDSNSVTCSVQHDNKTVHSTDF |
| | | EVKTDSTDHVKPKETENTKQPSKSCHKPKAIVHTEKVNMMS |
| | | LTVLGLRMLFAKTVAVNFLLTAKLFFL |

In some embodiments, provided is an engineered cell or a population thereof that comprises, encodes, and/or is capable of expressing a heterologous immune receptor disclosed herein, or a composition, method, or system comprising the engineered cell. Methods disclosed herein can comprise contacting a cell or a population of cells with a composition or system disclosed herein (e.g., a delivery vector comprising a polynucleotide), thereby expressing the heterologous immune receptor and generating an engineered immune cell. The contacting can be in vivo. For example, a delivery vector comprising an expression construct or polynucleotide can be administered to a subject (e.g., a human), the expression construct or polynucleotide can be taken up by an immune cell in the subject (e.g., a T cell), and the heterologous immune receptor can be expressed by the cell, thereby generating an engineered immune cell in vivo. A delivery vector can be used to deliver an expression construct or polynucleotide to cells in a subject without removing the cells from the subject.

An engineered immune cell that comprises or encodes the heterologous immune receptor can be an alpha beta T cell. An engineered immune cell that comprises or encodes the heterologous immune receptor can be a gamma delta T cell. Non-limiting examples of cells that can comprise or encodes the heterologous immune receptor include lymphocytes, T cells, CD4+ T cells, CD8+ T cells, alpha-beta T cells, gamma-delta T cells, T regulatory cells (Tregs), cytotoxic T lymphocytes, Th1 cells, Th2 cells, Th17 cells, Th9 cells, naïve T cells, memory T cells, effector T cells, effector-memory T cells (TEM), central memory T cells (TCM), resident memory T cells (TRM), follicular helper T cells (TFH), tumor-infiltrating lymphocytes (TILs), Innate Lymphoid Cells (ILCs), ILC1 cells, ILC2 cells, ILC3 cells, lymphoid tissue inducer (LTi) cells, B cells, B1 cells, B1a cells, B1b cells, B2 cells, plasma cells, B regulatory cells, memory B cells, marginal zone B cells, follicular B cells, germinal center B cells, antigen presenting cells (APCs), monocytes, macrophages, M1 macrophages, M2 macrophages, tissue-associated macrophages, dendritic cells, plasmacytoid dendritic cells, neutrophils, mast cells, basophils, eosinophils, and combinations thereof. In some cases, compositions, systems, and methods of the disclosure comprise T cells. In some cases, compositions, systems, and methods of the disclosure comprise primary human T cells (e.g., autologous or allogeneic primary human T cells). In some cases, compositions, systems, and methods of the disclosure comprise NK cells.

In some embodiments, an engineered immune cell comprises a disruption or deletion of one or more TCR-encoding genes, such as TRAC, TRB, TRG, and/or TRD. In some embodiments, an engineered immune cell comprises a disruption or deletion of a variable region of one or more TCR-encoding genes, such as a disruption or deletion in TRAC, TRB, TRG, and/or TRD. An engineered immune cell can be a primary cell.

### C. Immunomodulatory Factor

A system, method, expression construct, cell, or polynucleotide disclosed herein can comprise a transgene that encodes an immunomodulatory factor, for example, a cytokine, cytokine receptor, chemokine, chemokine receptor, immune co-receptor, or immune co-receptor ligand. Expression of the immunomodulatory factor can be driven by an expression regulatory region disclosed herein. In some embodiments, the heterologous immune receptor and the immunomodulatory factor are separated by an IRES, a self-cleaving linker, or a 2A linker disclosed herein.

An immunomodulatory factor can be or can comprise a cytokine or a functional fragment thereof, for example, G-CSF, GITRL, GM-CSF, IFN-α, IFN-β, IFN-γ, IL-IRA, IL-1α, IL-1B, IL-2, IL-3, IL-4, IL-5, IL-6, IL-7, IL-9, IL-10, IL-11, IL-12, IL-13, IL-14, IL-15, IL-16, IL-17, IL-18, IL-20, IL-23, LIF, LIGHT, LT-β, M-CSF, MSP, OSM, OX40L, SCF, TALL-1, TGF-β, TGF-β1, TGF-β2, TGF-β3, TNF-α, TNF-β, TRAIL, TRANCE, or TWEAK.

An immunomodulatory factor can be or can comprise a cytokine receptor or a functional fragment thereof, for example, a common gamma chain receptor, a common beta chain receptor, an interferon receptor, a TNF family receptor, a TGF-B receptor, Apo3, CD114, CD115, CD116, CD117, CD118, CD120, CD120a, CD120b, CD121, CD121a, CD121b, CD122, CD123, CD124, CD126, CD127, CD130, CD131, CD132, CD212, CD213, CD213-al, CD213a13, CD213a2, CD25, CD27, CD30, CD4, CD40, CD95 (Fas), CDw119, CDw121b, CDw125, CDw131, CDw136, CDw137 (41BB), CDw210, CDw217, GITR, HVEM, IL-11R, IL-11Ra, IL-14R, IL-15R, IL-15Ra, IL-18R, IL-18Ra, IL-18RB, IL-20R, IL-20Ra, IL-20RB, IL-9R, LIFR, LTBR, OPG, OSMR, OX40, RANK, TACI, TGF-BR1, TGF-BR2, TGF-BR3, TRAILR1, TRAILR2, TRAILR3, or TRAILR4.

An immunomodulatory factor can be or can comprise a chemokine or a functional fragment thereof, for example, ACT-2, AMAC-a, ATAC, ATAC, BLC, CCLI, CCL11, CCL13, CCL14, CCL15, CCL16, CCL17, CCL18, CCL19, CCL2, CCL20, CCL21, CCL22, CCL23, CCL24, CCL25, CCL26, CCL27, CCL3, CCL4, CCL5, CCL7, CCL8, CKb-6, CKb-8, CTACK, CX3CL1, CXCLI, CXCL10, CXCL11, CXCL12, CXCL13, CXCL14, CXCL2, CXCL3, CXCL4, CXCL5, CXCL6, CXCL7, CXCL8, CXCL9, DC-CK1, ELC, ENA-78, eotaxin, eotaxin-2, eotaxin-3, Eskine, exodus-1, exodus-2, exodus-3, fractalkine, GCP-2, GROa, GROb, GROg, HCC-1, HCC-2, HCC-4, 1-309, IL-8, ILC, IP-10, I-TAC, LAG-1, LARC, LCC-1, LD78a, LEC, Lkn-1, LMC, lymphoactin, lymphoactin b, MCAF, MCP-1, MCP-2, MCP-3, MCP-4, MDC, MDNCF, MGSA-a, MGSA-b, MGSA-g, Mig, MIP-1d, MIP-1a, MIP-1B, MIP-2a, MIP-2b, MIP-3, MIP-3a, MIP-3B, MIP-4, MIP-4a, MIP-5, MPIF-1, MPIF-2, NAF, NAP-1, NAP-2, oncostatin, PARC, PF4, PPBP, RANTES, SCM-1a, SCM-1b, SDF-1a/B, SLC, STCP-1, TARC, TECK, XCL1, or XCL2.

An immunomodulatory factor can be or can comprise a chemokine receptor or a functional fragment thereof, for example, CCR1, CCR2, CCR3, CCR4, CCR5, CCR6, CCR7, CCR8, CCR9, CCR10, CX3CR1, CXCR1, CXCR2, CXCR3, CXCR4, CXCR5, XCRI, or XCR1.

An immunomodulatory factor can be a costimulatory immune receptor, or a functional fragment thereof, for example, CD28, 2B4 (CD244, SLAMF4), 4-1BB (CD137), CD2 (LFA2, OX34), CD21, CD226 (DNAM1), CD27 (TNFRSF7), CD30 (TNFRSF8), CD4, CD40, CD8, CD84 (SLAMF5), CRACC (CD319,BLAME), CRTAM (CD355), DcR3, DR3 (TNFRSF25), GITR (CD357), HVEM (CD270), ICOS (CD278), LIGHT, LTBR (TNFRSF3), Ly108 (NTBA,CD352, SLAMF6), Ly9 (CD229,SLAMF3), OX40 (CD134), SLAM (CD150,SLAMF1), TIM1 (HAVCR1,KIM1), or TIM2.

An immunomodulatory factor can be an activating NK receptor, or a functional fragment thereof, for example, CD100 (SEMA4D), CD16 (FcgRIIIA), CD160 (BY55), CD244 (2B4, SLAMF4), CD27, CD94-NKG2C, CD94-NKG2E, CD94-NKG2H, CD96, CRTAM, DAP12, DNAM1 (CD226), KIR2DL4, KIR2DS1, KIR2DS2, KIR2DS3, KIR2DS4, KIR2DS5, KIR3DS1, Ly49, NCR, NKG2D (KLRK1, CD314), NKp30 (NCR3), NKp44 (NCR2), NKp46 (NCR1), NKp80 (KLRF1, CLEC5C), NTB-A (SLAMF6), PSGLI, or SLAMF7 (CRACC, CS1, CD319).

In some embodiments, an immunomodulatory factor comprises an Fcγ receptor (FcγR), an Fcα receptor (FcεR), an Fcα receptor (FcαR), an Fcμ receptor (FcμR), neonatal Fc receptor (FcRn), CD4, CD5, CD8, CD21, CD22, CD27, CD28, CD32, CD40, CD45, CD66d, CD79a, CD79b, CD80, CD86, CD278 (ICOS), CD247ζ, CD247η, 41BB, DAP10, DAP12, FYN, LAT, Lck, MAPK, MHC complex, NFAT, NF-κB, PLC-γ, iC3b, C3dg, C3d, Zap70, MyD88, a functional fragment thereof, or a combination thereof.

In some embodiments, an immunomodulatory factor comprises a domain that is, is derived from, interacts with, increases expression of, or activates a transcription factor, such as, for example, E2A, Pax5, EBF, PU.1, Ikaros, GATA3, Th-POK, Tbet, Bcl6, NF-κB, NFAT, AP-1, NFAT, STAT1, STAT2, STAT3, STAT4, STAT5, STATSA, STAT5B, STAT6, STAT7, IRF1, IRF2, IRF3, IRF4, IRF5, IRF6, IRF7, IRF8, IRF9, AP-1, Eomes, FoxP3, Id2, PLZF, ROR-gamma-T, TCF7, ThPOK, or any combination thereof.

### II. Delivery Vectors

Compositions, systems, and methods of the disclosure can comprise or utilize delivery vectors, e.g., for delivery of an expression construct or polynucleotide encoding a heterologous immune receptor.

A delivery vector disclosed herein can be a lipid-based delivery vector (LDV). An LDV disclosed herein can facilitate delivery of an expression construct or polynucleotide disclosed herein, and expression of a transgene of interest after in vivo administration to a subject. For example, an LDV disclosed herein can facilitate expression of a heterologous immune receptor and/or immunomodulatory factor after in vivo administration. An LDV disclosed herein can utilize an effective and re-dosable delivery platform that allows high tolerability compared to alternate formulations or approaches. An LDV can comprise a lipid membrane and/or a lipid bi-layer. An LDV can exclude an enveloped viral vector.

An LDV disclosed herein can comprise one or more (for example, two or more, three or more, four or more, five or more, one, two three, four, five, or six) lipids selected from 1,2-di-O-octadecenyl-3-trimethylammonium propane (DOTMA), 1,2-dioleoyl-3-dimethylammonium-propane (DODAP), 1,2-Dioleyloxy-3-dimethylaminopropane (DODMA), 1,2-dimyristoyl-sn-glycero-3-methoxypolyethylene glycol-2000 (DMG-PEG). 2-dioleoyl-sn-glycero-3-phosphoethanolamine (DOPE) 1,2-dioleoyl-3-trimethylammonium-propane (DOTAP), Dlin-KC2-DMA (KC2), DOBAQ, 18:1 EPC, DDAB, 18:0 EPC, 18:0 DAP, L-a-dioleoyl phosphatidyl choline (DOPC), cholesterol, DF4C11PE (rac-2,3-Di [11-(F-butyl) undecanoyl) glycero-1-phosphoethanolamine), distear-4-ynoyl L-a-phosphatidylethanolamine [DS (9-yne) PE], 18:0 TAP, dipalmitoylphosphatidylcholine (DPPC), phosphatidylcholine (PC), phosphatidic acid (PA), phosphatidylglycerol (PG), phosphatidylethanolamine (PE), phosphatidylserine (PS), distearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE), and 2,3-dioleyloxy-N-[2-(sperminecarboxamido) ethyl]-N,N-dimethyl-1-propanaminium trifluoroacetate (DOSPA).

An LDV disclosed herein can comprise one or more ionizable lipids. The charge of ionizable lipids can be dependent upon the pH of the surrounding environment. Ionizable lipids include, but are not limited to, 1,2-dioleoyl-3-dimethylammonium-propane (DODAP), 1,2-dioleoyl-3-trimethylammonium-propane (DOTAP), 1,2-dioleyloxy-3-dimethylaminopropane (DODMA), 1,2-di-O-octadecenyl-3-trimethylammonium propane (DOTMA), DLin-MC3-DMA (MC3), Dlin-KC2-DMA (KC2), DOBAQ, 18:1 EPC, DDAB, 18:0 EPC, 18:0 DAP, and 18:0 TAP.

In some embodiments, ionizable lipids in an LDV disclosed herein comprise, consist essentially of, or consist of DODAP. In some embodiments, ionizable lipids in an LDV disclosed herein comprise, consist essentially of, or consist of DODMA. In some embodiments, ionizable lipids in an LDV disclosed herein comprise, consist essentially of, or consist of DODAP and DODMA. In some embodiments, ionizable lipids (e.g., in a combination or ratio referred to herein) do not include cationic lipids, such as DOTMA and/or DOTAP.

An LDV disclosed herein can comprise one or more cationic lipids. Non-limiting examples of cationic lipids include 1,2-di-O-octadecenyl-3-trimethylammonium propane (DOTMA) and 1,2-dioleoyl-3-trimethylammonium-propane (DOTAP). In some embodiments, cationic lipids are used in a sufficiently low quantity in an LDV to reduce a pro-inflammatory response to the LDV (e.g., Th1 type cytokines or type I interferon) compared to control lipid nanoparticles. In some embodiments, an LDV does not contain or substantially lacks cationic lipids. In some embodiment, an LDV does not contain or substantially lacks DOTAP. In some embodiment, an LDV does not contain or substantially lacks DOTMA. In some embodiment, an LDV does not contain or substantially lacks cationic lipids except for DOTAP. In some embodiment, an LDV does not contain or substantially lacks cationic lipids except for DOTMA. In some embodiment, an LDV does not contain or substantially lacks cationic lipids except for DOTAP and DOTMA. In some embodiments, cationic lipids in an LDV disclosed herein comprise, consist essentially of, or consist of DOTMA. In some embodiments, cationic lipids in an LDV disclosed herein comprise, consist essentially of, or consist of DOTAP. In some embodiments, cationic lipids in an LDV disclosed herein comprise, consist essentially of, or consist of DOTAP and DOTMA.

An LDV disclosed herein can comprise one or more helper lipids. Non-limiting examples of a helper lipids include 2-dioleoyl-sn-glycero-3-phosphoethanolamine (DOPE) and L-a-dioleoyl phosphatidyl choline (DOPC). In some embodiments, helper lipids in an LDV disclosed herein comprise, consist essentially of, or consist of DOPE. In some embodiment, an LDV does not contain or substantially lacks DOPE and/or DOPC.

An LDV disclosed herein can comprise one or more PEGylated lipids. A non-limiting example of a PEGylated lipid is 1,2-dimyristoyl-sn-glycero-3-methoxypolyethylene glycol-2000 (DMG-PEG). In some embodiments, PEGylated lipids in an LDV disclosed herein comprise, consist essentially of, or consist of DMG-PEG. In some embodiment, an LDV does not contain or substantially lacks DMG-PEG.

An LDV disclosed herein can comprise cholesterol. In some embodiment, an LDV does not contain or substantially lacks cholesterol.

In some embodiments, an LDV disclosed herein comprises a combination of lipids at molar ratios appropriate to reduce toxicity, immunogenicity, or a pro-inflammatory response associated with administration of the delivery vector. For example, an LDV can comprise a combination of lipids at molar ratios appropriate to reduce production of pro-inflammatory cytokines, such as tumor necrosis factor alpha (TNF-α), interferon-gamma (IFN-γ), interleukin-6 (IL-6), type I interferon, or a combination thereof associated with administration of the delivery vector. In some embodiments, an LDV disclosed herein comprises a combination of lipids at molar ratios appropriate to reduce complement activation-related pseudoallergy (CARPA). The reduction can be in comparison to, for example, control lipid nanoparticles that comprise a higher proportion of cationic lipids. The reduction can be determined by an experiment in which empty LDV or substantially non-immunogenic cargo is administered (e.g., an expression construct or polynucleotide encoding a reporter, such as GFP). In some embodiments, the combination of lipids in the LDV make the LDV or system more suitable for high dose and/or systemic administration as compared to the control lipid nanoparticles. In some embodiments, an LDV disclosed herein exhibits broader distribution upon systemic administration compared to control lipid nanoparticles or viral vectors. In some embodiments, an LDV disclosed herein exhibits reduced accumulation in the liver upon systemic administration compared to control lipid nanoparticles or viral vectors.

An LDV disclosed herein can exhibit superior properties for delivery of a DNA expression construct or polynucleotide compared to control lipid nanoparticles. For example, in some embodiments an LDV disclosed herein requires less cationic components to neutralize the anionic charge of DNA as compared to control lipid nanoparticles.

In some embodiments, an LDV disclosed herein comprises DODAP. In some embodiments, an LDV disclosed herein comprises DODMA. In some embodiments, an LDV disclosed herein comprises DODAP and DODMA.

In some embodiments, an LDV disclosed herein comprises cationic:ionizable:helper:PEGylated lipids at a molar ratio disclosed herein. The cationic lipid(s) can comprise or consist of DOTAP. The ionizable lipid(s) can comprise or consist of DODAP. The ionizable lipid(s) can comprise or consist of DODMA. The ionizable lipid(s) can comprise or consist of DOTAP. The ionizable lipid(s) can comprise or consist of DODAP and DODMA. The ionizable lipid(s) can comprise or consist of DODAP and DOTAP. The ionizable lipid(s) can comprise or consist of DODMA and DOTAP. The ionizable lipid(s) can comprise or consist of DODAP, DODMA, and DOTAP. The helper lipid(s) can comprise or consist of DOPE. The PEGylated lipid(s) can comprise or consist of DMG-PEG.

In some embodiments, the lipids of an LDV disclosed herein comprise, consist essentially of, or consist of cationic: ionizable: helper: PEGylated lipids at a molar ratio of about 24:42:30:4.

In some embodiments, the lipids of an LDV disclosed herein comprise, consist essentially of, or consist of cationic: ionizable: helper: PEGylated lipids at a molar ratio of about 6:60:30:4.

In some embodiments, the lipids of an LDV disclosed herein comprise, consist essentially of, or consist of cationic: ionizable: helper: PEGylated lipids at a molar ratio of about 0:66:30:4.

In some embodiments, the lipids of an LDV disclosed herein comprise, consist essentially of, or consist of cationic: ionizable: helper: PEGylated lipids at a molar ratio of about 3:63:30:4.

In some embodiments, the lipids of an LDV disclosed herein comprise, consist essentially of, or consist of cationic: ionizable: helper: PEGylated lipids at a molar ratio of about 49.5:24.75:23.75:2.

In some embodiments, the lipids of an LDV disclosed herein comprise, consist essentially of, or consist of cationic: ionizable: helper: PEGylated lipids at a molar ratio of about 49.5:38.5:10:2.

In some embodiments, the lipids of an LDV disclosed herein comprise, consist essentially of, or consist of cationic: ionizable: helper: PEGylated lipids at a molar ratio of about 61.7:26.3:19:3.

In some embodiments, an LDV disclosed herein comprises ionizable, cholesterol, helper, and PEGylated lipids. In some embodiments, the lipids of an LDV disclosed herein comprise, consist essentially of, or consist of ionizable: cholesterol: helper: PEGylated lipids at a molar ratio of about 49.5:38.5:10:2. In some embodiments, the lipids of an LDV disclosed herein comprise, consist essentially of, or consist of ionizable: cholesterol: helper: PEGylated lipids at a molar ratio of about 49.5:24.75:23.75:2. In some embodiments, the lipids of an LDV disclosed herein comprise, consist essentially of, or consist of ionizable: cholesterol: helper: PEGylated lipids at a molar ratio of about 61.7:26.3:19:3.

In some embodiments, the lipids of an LDV disclosed herein comprise, consist essentially of, or consist of DOTAP, DODAP, DOPE and DMG-PEG. In some embodiments, the lipids of an LDV disclosed herein comprise, consist essentially of, or consist of DOTAP, DODAP, DOPE, and DMG-PEG, at a molar ratio or in a mole percentage of about 24:42:30:4.

In some embodiments, the lipids of an LDV disclosed herein comprise, consist essentially of, or consist of DOTAP, DODMA, DOPE and DMG-PEG. In some embodiments, the lipids of an LDV disclosed herein comprise, consist essentially of, or consist of DOTAP, DODMA, DOPE, and DMG-PEG, at a molar ratio or in a mole percentage of about 24:42:30:4.

In some embodiments, the lipids of an LDV disclosed herein comprise, consist essentially of, or consist of DOTAP, DODAP, DODMA, DOPE, and DMG-PEG. In some embodiments, the lipids of an LDV disclosed herein comprise, consist essentially of, or consist of DOTAP, DODAP, DODMA, DOPE, and DMG-PEG, at a molar ratio or in a mole percentage of about 24:21:21:30:4.

In some embodiments, the lipids of an LDV disclosed herein comprise, consist essentially of, or consist of DOTAP, DODAP, DOPE and DMG-PEG. In some embodiments, the lipids of an LDV disclosed herein comprise, consist essentially of, or consist of DOTAP, DODAP, DOPE, and DMG-PEG, at a molar ratio or in a mole percentage of about 6:60:30:4 or 3:63:30:4.

In some embodiments, the lipids of an LDV disclosed herein comprise, consist essentially of, or consist of DODAP, DOPE and DMG-PEG. In some embodiments, the lipids of an LDV disclosed herein comprise, consist essentially of, or consist of DODAP, DOPE, and DMG-PEG, at a molar ratio or in a mole percentage of about 66:30:4.

In some embodiments, the lipids of an LDV disclosed herein comprise, consist essentially of, or consist of DODAP, cholesterol, DOPE, and DMG-PEG. In some embodiments, the lipids of an LDV disclosed herein comprise, consist essentially of, or consist of DODAP, cholesterol, DOPE, and DMG-PEG, at a molar ratio or in a mole percentage of about 49.5:24.75:23.75:2, about 49.5:38.5:10:2, or about 61.7:26.3:19:3.

A delivery vector, such as an LDV, can comprise a fusogenic protein to enhance fusion of the LDV with the plasma membrane of a target cell. Non-limiting examples of fusogenic proteins include a fusion associated small transmembrane (FAST) proteins, herpes simplex virus glycoprotein H, and amphiphilic anionic peptides derived from the N-terminal segment of the HA-2 subunit of influenza virus haemagglutinin, such as the IFN7 and E5CA.

A fusogenic protein can be a fusion associated small transmembrane (FAST) protein or can comprise a functional fragment of a FAST protein, or functional fragments of two or more FAST proteins. A FAST protein can function receptor-independently, and at physiological pH. In some embodiments, use of a FAST protein in an LDV allows a minimal molar ratio of cationic and/or ionizable lipid to be used in order to neutralize the anionic charge of the nucleic acid, reducing or substantially eliminating the role of ionizable lipid in the delivery process (e.g., endosomal escape). In some embodiments, incorporation of a FAST protein in an LDV enhances intracellular delivery of an expression construct or polynucleotide disclosed herein. In some embodiments, use of a FAST protein in an LDV allows for omission or lower concentrations of cholesterol to be used, for example, compared to control lipid nanoparticles.

The FAST protein family comprises six members named according to their molecular mass in Daltons (p10, p13, p14, p15, p16, and p22).

In some embodiments, a FAST protein utilized in a compositions, system, or method disclosed herein is a native FAST protein found in the family Reoviridae, for example, found in the genus Aquareovirus or Orthoreovirus. Non-limiting examples of orthoreoviruses include BRV (Baboon orthoreovirus), MRV (Mammalian orthoreovirus), NBV (Nelson Bay orthoreovirus), BrRV (Broome orthoreovirus), RRV (Reptilian orthoreovirus), and ARV (Avian orthoreovirus). In some embodiments, a FAST protein utilized in a composition, system, or method disclosed herein comprises a FAST protein or domain thereof from ARV p10, BrRv p13, RRV p14, BRV p15, AqV p16, or AqV p22.

A FAST protein can comprise an N-terminal ectodomain on the extracellular or external side of the membrane or LDV. The ectodomain can be, for example, about 19-40 residues, with a myristoylation motif, or a myristate moiety on a glycine, such as a penultimate N-terminal glycine. A FAST protein ectodomain can comprise a hydrophobic patch.

A FAST protein can comprise a transmembrane domain that serves as a reverse signal-anchor sequence to direct a bitropic Nout/Cin type I topology in the membrane or LDV.

A FAST protein can comprise a C-terminal endodomain on the cytoplasmic or internal side of the membrane or LDV. A FAST protein endodomain can be, e.g., about 40-140 residues, with a membrane-destabilizing fusion peptide motif. A FAST protein endodomain can comprise a juxtamembrane polybasic motif. A FAST protein endodomain can comprise a membrane-proximal membrane curvature sensor (e.g., an amphipathic alpha helix, such as a helix-kink-helix membrane curvature sensor) to drive pore formation. A FAST protein endodomain can comprise a hydrophobic patch.

A FAST protein can comprise a proline-hinged loop. A FAST protein can comprise a type II polyproline helix. A FAST protein can comprise a conserved region that functions as a fusion peptide, e.g., by promoting rapid lipid bilayer destabilization and membrane merging. A FAST protein can comprise a palmitoylated cysteine residue. A FAST protein can comprise a hydrophobic patch.

Structure-function relationships between different FAST proteins have suggested that overlapping structural motifs of can be exchanged among certain FAST proteins to generate functional chimeric FAST fusion proteins. In some embodiments, a chimeric FAST protein disclosed herein exhibits superior fusion activity compared to a wild-type FAST protein.

Chimeric FAST proteins can be synthesized that combine the domains from different FAST proteins, such the p10, p14, and/or p15 peptides, to form a functional fusogenic protein.

A FAST protein used in an LDV disclosed herein can comprise the ectodomain from the p14 FAST protein or a functional portion thereof, the transmembrane domain from the p14 FAST protein, and the endodomain from the p15 FAST protein or a functional portion thereof. Such a FAST protein can be referred to as a “p14endo15” or “p14e15” FAST protein. In some embodiments, the fusion activity of p14e15 is mediated by the efficient p14 ectodomain fusion peptide and myristate moiety facilitating lipid mixing with the target cell membrane, followed by the p15 endodomain fusion-inducing lipid packing sensor (FLiPs) motif partitioning into the LDV membrane to promote pore formation and liposome-cell fusion activity.

A FAST protein used in an LDV disclosed herein can comprise the ectodomain from the p14 FAST protein or a functional portion thereof, the transmembrane domain from the p15 FAST protein, and the endodomain from the p14 FAST protein or a functional portion thereof. Such a FAST protein can be referred to herein as “p14TM15”.

A FAST protein used in an LDV disclosed herein can comprise the ectodomain from the p14 FAST protein or a functional portion thereof, the transmembrane domain from the p15 FAST protein, and the endodomain from the p15 FAST protein or a functional portion thereof. Such a FAST protein can be referred to as “p15ecto14” or “p15e14”.

A FAST protein used in an LDV disclosed herein can comprise p10, p13, p14, p15, p16, p22, or a chimeric fusion protein thereof. In some embodiments, the FAST protein is a p14/p15 chimera, p10/p14 chimera, or a p10/p15 chimera. In some embodiments, the FAST protein comprises: (i) the ectodomain and transmembrane domain of p14 and the endodomain of p15; (ii) the ectodomain of p14, and the transmembrane domain and endodomain of p15; or (iii) the ectodomain and endodomain of p14 and the transmembrane of p15.

Non-limiting examples of FAST proteins are provided in WO2012040825A1, which is incorporated herein by reference for such disclosure.

In some embodiments, a FAST protein or a domain thereof comprises an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to any one of SEQ ID NOs: 88-92.

In some embodiments, a FAST protein or a domain thereof comprises an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to at least 40 consecutive amino acids of any one of SEQ ID NOs: 88-92.

TABLE 3 provides illustrative FAST protein sequences.

TABLE 3

| SEQ | | |
|---|---|---|
| ID | | |
| NO | Description | Sequence |
| 88 | P14 | MGSGPSNFVNHAPGEAIVTGLEKGA |
| | | DKVAGTISHTIWEVIAGLVALLTFL |
| | | AFGFWLFKYLQKRRERRRQLTEFQK |
| | | RYLRNSYRLSEIQRPISQHEYEDPY |
| | | EPPSRRKPPPPPYSTYVNIDNVSAI |
| 89 | P14e15 | MGSGPSNFVNHAPGEAIVTGLEKGA |
| | (synthetic | DKVAGTISHTIWEVIAGLVALLTFL |
| | construct) | AFGFWLFKYLQWYNRKSKNKKRKEQ |
| | | IREQIELGLLSYGAGVASLPLLNVI |
| | | AHNPGSVISATPIYKGPCTGVPNSR |
| | | LLQITSGTAEENTRILNHDGRNPDG |
| | | SINV |
| 90 | P14 fusogenic | MGSGPSNFVNHAPGEAIVTGLEKGA |
| | protein | DKVAGTISHTIWE |
| | ectodomain | |
| 91 | P15 fusogenic | KLLQWYNRKSKNKKRKEQIREQIEL |
| | protein | GLLSYGAGVASLPLLNVIAHNPG |
| | endodomain | |
| 92 | P15 fusogenic | VISATPIYKGPCTGVPNSRLLQITS |
| | protein | GTAEENTRILNHDGRNPDGSINV |
| | endodomain | |

An LDV disclosed herein can comprise one or more ionizable lipids and one or more FAST proteins (e.g., a chimeric FAST protein). In some embodiments, use of a FAST protein in an LDV allows use of certain ionizable lipids and for a favorable ratio of ionizable, helper, and PEGylated lipids.

A delivery vector can comprise a cell penetrating peptide.

A molar ratio of an ionizable lipid to a polynucleotide can be between about 2.5:1 and about 20:1. In some embodiments, the molar ratio of the ionizable lipid to the polynucleotide is about 5:1, about 7.5:1, about 10:1, or about 15:1.

In some embodiments, the molar ratio of the ionizable lipid to the polynucleotide is between about 4:1 and about 7.5:1. In some embodiments, the molar ratio of the ionizable lipid to the polynucleotide is between about 2.5:1 and about 7:5:1. In some embodiments, the molar ratio of the ionizable lipid to the polynucleotide is between about 3:1 and about 7.5:1.

In some embodiments, the molar ratio of the ionizable lipid to the polynucleotide is between about 5:1 and about 10:1.

In some embodiments, the molar ratio of the ionizable lipid to the polynucleotide is between about 5:1 and about 12:1.

In some embodiments, the molar ratio of the ionizable lipid to the polynucleotide is between about 2.5:1 and about 15:1. In some embodiments, the molar ratio of the ionizable lipid to the polynucleotide is between about 5:1 and about 15:1. In some embodiments, the molar ratio of the ionizable lipid to the polynucleotide is between about 7.5:1 and about 15:1. In some embodiments, the molar ratio of the ionizable lipid to the polynucleotide is between about 2.5:1 and about 15:1.

In some embodiments, the molar ratio of the ionizable lipid to the polynucleotide is between about 5:1 and about 20:1.

In some embodiments, the molar ratio of the ionizable lipid to the polynucleotide is about 5:1.

In some embodiments, the lipids of an LDV disclosed herein comprise, consist essentially of, or consist of DOTAP: DODAP: DOPE: DMG-PEG at a molar ratio or in a mole percentage of about 24:42:30:4, and the molar ratio of ionizable lipid to polynucleotide (e.g., pDNA) is between about 5:1 and about 10:1.

In some embodiments, the lipids of an LDV disclosed herein comprise, consist essentially of, or consist of DOTAP: DODMA: DOPE: DMG-PEG at a molar ratio or in a mole percentage of about 24:42:30:4, and the molar ratio of ionizable lipid to polynucleotide (e.g., pDNA) is between about 4:1 and about 7.5:1.

In some embodiments, the lipids of an LDV disclosed herein comprise, consist essentially of, or consist of DOTAP: DODMA: DOPE: DMG-PEG at a molar ratio or in a mole percentage of about 24:42:30:4, and the molar ratio of ionizable lipid to polynucleotide (e.g., mRNA) is between about 2.5:1 to about 7.5:1.

In some embodiments, the lipids of an LDV disclosed herein comprise, consist essentially of, or consist of DOTAP: DODAP: DODMA: DOPE: DMG-PEG at a molar ratio or in a mole percentage of about 24:21:21:30:4, and the molar ratio of ionizable lipid to polynucleotide (e.g., pDNA) is between about 3:1 to about 7.5:1.

In some embodiments, the lipids of an LDV disclosed herein comprise, consist essentially of, or consist of DOTAP: DODAP: DODMA: DOPE: DMG-PEG at a molar ratio or in a mole percentage of about 24:21:21:30:4, and the molar ratio of ionizable lipid to polynucleotide (e.g., mRNA) is between about 2.5:1 to about 7.5:1.

In some embodiments, the lipids of an LDV disclosed herein comprise, consist essentially of, or consist of DOTAP: DODAP: DOPE: DMG-PEG at a molar ratio or in a mole percentage of about 3:63:30:4, and the molar ratio of ionizable lipid to polynucleotide (e.g., pDNA) is between about 7.5:1 to about 15:1.

In some embodiments, the lipids of an LDV disclosed herein comprise, consist essentially of, or consist of DOTAP: DODAP: DOPE: DMG-PEG at a molar ratio or in a mole percentage of about 3:63:30:4, and the molar ratio of ionizable lipid to polynucleotide (e.g., pDNA) is between about 5:1 to about 12:1.

In some embodiments, the lipids of an LDV disclosed herein comprise, consist essentially of, or consist of DOTAP: DODAP: DOPE: DMG-PEG at a molar ratio or in a mole percentage of about 6:60:30:4, and the molar ratio of ionizable lipid to polynucleotide (e.g., pDNA) is between about 5:1 to about 15:1.

In some embodiments, the lipids of an LDV disclosed herein comprise, consist essentially of, or consist of DOTAP: DODAP: DOPE: DMG-PEG at a molar ratio or in a mole percentage of about 6:60:30:4, and the molar ratio of ionizable lipid to polynucleotide (e.g., mRNA) is between about 5:1 to about 15:1.

In some embodiments, the lipids of an LDV disclosed herein comprise, consist essentially of, or consist of DODAP: DOPE: DMG-PEG at a molar ratio or in a mole percentage of about 66:30:4, and the molar ratio of ionizable lipid to polynucleotide (e.g., pDNA is between about 5:1 to about 20:1).

In some embodiments, the lipids of an LDV disclosed herein comprise, consist essentially of, or consist of DODAP: DOPE: DMG-PEG at a molar ratio or in a mole percentage of about 66:30:4, and the molar ratio of ionizable lipid to polynucleotide (e.g., mRNA) is between about 5:1 to about 20:1.

In some embodiments, the lipids of an LDV disclosed herein comprise, consist essentially of, or consist of DODAP: cholesterol: DOPE: DMG-PEG at a molar ratio or in a mole percentage of about 49.5:24.75:23.75:2, and the molar ratio of ionizable lipid to polynucleotide (e.g., pDNA) is between about 5:1 to about 15:1.

In some embodiments, the lipids of an LDV disclosed herein comprise, consist essentially of, or consist of DODAP: cholesterol: DOPE: DMG-PEG at a molar ratio or in a mole percentage of about 49.5:24.75:23.75:2, and the molar ratio of ionizable lipid to polynucleotide (e.g., mRNA) is between about 2.5:1 to about 15:1.

In some embodiments, the lipids of an LDV disclosed herein comprise, consist essentially of, or consist of DODAP: cholesterol: DOPE: DMG-PEG at a molar ratio or in a mole percentage of about 49.5:38.5:10:2, and the molar ratio of ionizable lipid to polynucleotide (e.g., pDNA) is between about 5:1 to about 15:1.

In some embodiments, the lipids of an LDV disclosed herein comprise, consist essentially of, or consist of DODAP: cholesterol: DOPE: DMG-PEG at a molar ratio or in a mole percentage of about 49.5:38.5:10:2, and the molar ratio of ionizable lipid to polynucleotide (e.g., mRNA) is between about 2.5:1 to about 15:1.

In some embodiments, the lipids of an LDV disclosed herein comprise, consist essentially of, or consist of DODAP: cholesterol: DOPE: DMG-PEG at a molar ratio or in a mole percentage of about 61.7:26.3:19:3, and the molar ratio of ionizable lipid to polynucleotide (e.g., pDNA) is between about 5:1 to about 15:1.

In some embodiments, the lipids of an LDV disclosed herein comprise, consist essentially of, or consist of DODAP: cholesterol: DOPE: DMG-PEG at a molar ratio or in a mole percentage of about 61.7:26.3:19:3, and the molar ratio of ionizable lipid to polynucleotide (e.g., mRNA) is between about 2.5:1 to about 15:1.

In some embodiments, an LDV comprises a vesicle size of less than about 80 nm.

In some embodiments, an LDV is untargeted, and for example, can facilitate delivery of an expression construct to a range of cell types including target cells and non-target cells (e.g., immune cells and non-immune cells, lymphocytes and non-lymphocytes, or T cells and non-T cells). Specificity of expression in target cells upon non-targeted delivery can be facilitated by an expression regulatory region, such as a cell type-specific promoter.

In some embodiments, an LDV is targeted, for example, can facilitate preferential delivery of an expression construct to a target cell type or population, such as immune cells, lymphocytes, T cells, NK cells, or NKT cells. A delivery vector can be targeted to a receptor specifically or preferentially expressed on a target cell, for example, CD3 (e.g., CD3 gamma, delta, or epsilon), CD4, or CD8. Specificity of expression in target cells upon targeted delivery can be further enhanced by an expression regulatory region, such as a cell type-specific promoter.

An illustrative method of making a lipid formulation to be used in generating an LDV can comprise heating lipids disclosed herein to 37° C., combining the lipids in ratios disclosed herein, mixing (e.g., vortex mixing), dehydrating the lipid mixture (e.g., in a rotavapor at 60 rpm for 2 hours, under vacuum), rehydrating with 100% ethanol, and sonicating at 37° C.

For generation of an LDV, a NanoAssemblr Benchtop microfluidics mixing instrument can be used to mix organic and aqueous solutions and make the LDVs. The organic solution can comprise or consist of the lipid formulation. The aqueous solution can comprise or consist of nucleic acid cargo, FAST protein (e.g., 5 nM), and acetate buffer (e.g., 10 mM, pH 4.0). The Benchtop NanoAssemblr running protocol can comprise a total flow rate of 12 mL/min and a 3:1 aqueous to organic flow rate ratio. LDVs can be dialyzed in 8000 MWCO dialysis tubing clipped at one end. The loaded tubing can be rinsed with 5 mL of double distilled water and dialyzed in 500 mL of Dialysis Buffer (ENT1844) with gentle stirring (60 rpm) at ambient temperature for 1 hour and repeated twice with fresh Dialysis Buffer. LDVs can be concentrated using a 100 kDa Ultra filter. LDVs can be filter sterilized through 0.2 μm Acrodisc Supor filters.

Non-limiting examples of LDVs are provided in WO2022067446A1, which is incorporated herein by reference for such disclosure.

In some embodiments, a lipid-based delivery vector is or comprises a lipid nanoparticle (LNP). LNPs can be formulated with cationic and/or ionizable lipids that neutralize the anionic charge of nucleic acids and facilitate the endosomal escape of encapsulated nucleic acids through charge-mediated lipid bilayer disruption. LNPs can comprise a combination of different classes of lipids such as cationic or ionizable lipids (CIL), structural lipids (e.g., phospholipid and sterol lipid) and PEG-conjugated lipid (PEG-lipid). These lipids can self-assemble into LNPs under controlled microfluidic mixing with an aqueous phase containing the nucleic acids. PEG-lipids can prevent or reduce aggregation, degradation, and opsonization of the LNPs, while the structural lipids promote the stability and integrity of the nanoparticle.

In some embodiments, an LNP comprises the ionizable lipid DLin-MC3-DMA (MC3). In some embodiments, an LNP comprises DLin-MC3-DMA/DSPC/Cholesterol/PEG-lipid with the molar ratio 50:10:38.5:1.5. In some embodiments a delivery vector is not an LNP.

In some embodiments, a lipid-based delivery vector is or comprises a liposome. A liposome can comprise a cationic lipid, such as a cationic lipid disclosed herein. Illustrative liposomes include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV).

In some instances, liposomes provided herein also comprise carrier lipids. In some embodiments the carrier lipids are phospholipids. The carrier lipids are optionally any non-phosphate polar lipids. In some instances, liposomes provided herein comprise dipalmitoylphosphatidylcholine (DPPC), phosphatidylcholine (PC; lecithin), phosphatidic acid (PA), phosphatidylglycerol (PG), phosphatidylethanolamine (PE), phosphatidylserine (PS), distearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE) and the like, or a combination thereof. In some embodiments, the liposomes further comprise a sterol (e.g., cholesterol) which modulates liposome formation. In some embodiments, a liposome comprises an electroneutral lipid.

In some embodiments, a liposome comprises a cationic lipid. Cationic lipids can have a head group with positive charge (e.g., permanent or substantially permanent positive charge). Non-limiting examples of cationic lipids for use in liposomes include 1,2-di-O-octadecenyl-3-trimethylammonium-propane (DOTMA), 1,2-dioleoyl-3-trimethylammonium-propane (DOTAP), Dimethyldioctadecylammonium bromide (DDAB), and 2,3-dioleyloxy-N-[2-(sperminecarboxamido) ethyl]-N,N-dimethyl-1-propanaminium trifluoroacetate (DOSPA). In some embodiments a delivery vector is not a liposome.

In some embodiments, a lipid-based delivery vector is or comprises a vesicle, micelle or a microsphere. In some embodiments a delivery vector is not a vesicle, a micelle, or a microsphere.

A lipid-based delivery vector can be or comprise a micelle. In some instances, the micelle is a polymeric micelle, characterized by a core shell structure, in which the hydrophobic core is surrounded by a hydrophilic shell. In some cases, the hydrophilic shell further comprises a hydrophilic polymer or copolymer and a pH sensitive component.

Illustrative hydrophilic polymers or copolymers include, but are not limited to, poly (N-substituted acrylamides), poly (N-acryloyl pyrrolidine), poly (N-acryloyl piperidine), poly (N-acryl-L-amino acid amides), poly(ethyl oxazoline), methylcellulose, hydroxypropyl acrylate, hydroxyalkyl cellulose derivatives and poly(vinyl alcohol), poly (N-isopropylacrylamide), poly (N-vinyl-2-pyrrolidone), polyethyleneglycol derivatives, and combinations thereof.

A delivery vector can be or comprise a polymeric micelle exhibiting pH-sensitive properties, e.g., formed by using pH-sensitive polymers including, but not limited to, copolymers from methacrylic acid, methacrylic acid esters and acrylic acid esters, polyvinyl acetate phthalate, hydroxypropyl methyl cellulose phthalate, cellulose acetate phthalate, or cellulose acetate trimellitate.

A delivery vector can comprise a pH-sensitive moiety, which can include, but is not limited to, an alkylacrylic acid such as methacrylic acid, ethylacrylic acid, propyl acrylic acid and butyl acrylic acid, or an amino acid such as glutamic acid.

A delivery vector disclosed herein can be a non-viral vector. In some embodiments, a non-viral vector allows for superior delivery of an expression construct or polynucleotide upon repeat dosing compared to a viral vector, for example, based on reduced immunogenicity.

A delivery vector disclosed herein can be a non-viral, lipid-based delivery vector. A non-viral, lipid-based delivery vector can be, for example, an LDV disclosed herein, a liposome, a lipoplex, a lipid nanoparticle, a vesicle, or a micelle.

In some embodiments, a delivery vector is or comprises a poloxamer, nanoparticle, polyplex, or dendrimer.

A delivery vector can be a nanoparticle, for example, an inorganic nanoparticle, such as a gold, silica, iron oxide, titanium, calcium phosphate, PLGA, poly (β-amino ester) (PBAE, e.g., PBAE-447), or hydrogel nanoparticle. In some embodiments a delivery vector is not a nanoparticle, e.g., is not an inorganic nanoparticle.

Nucleic acids can be encapsulated in particles through electrostatic association and physical entrapment. To prevent or slow the disassociation of cargo nucleic acids from nanoparticles following systemic administration, a polymerizable conjugate with a degradable, disulfide linkage can be employed. Nanoparticles can be encapsulated with a lipid coating to improve oral bioavailability, minimize enzymatic degradation and cross blood brain barrier. The nanoparticle surface can also be PEGylated to improve water solubility, circulation in vivo, and stealth properties.

A delivery vector can be a polyplex, for example, a complex of one or more polymers and nucleic acids. A polyplex can comprise cationic polymers. Fabrication of a polyplex can be based on self-assembly by ionic interactions. A polyplex can comprise polyethyleneimine, chitosan, poly (beta-amino esters), and/or polyphosphoramidate. In some embodiments a delivery vector is not a polyplex.

A delivery vector can be a dendrimer. A dendrimer can be a highly branched macromolecule with a spherical shape. The surface of dendrimer particles can be functionalized such as, for example, with positive surface charges (cationic dendrimers), which can be employed for the delivery of nucleic acids. Dendrimer-nucleic acid complexes are taken into a cell via endocytosis. In some embodiments a delivery vector is not a dendrimer.

In some embodiments, a delivery vector is or comprises a viral vector, a gamma-retroviral vector, a lentiviral vector, an adenoviral vector, or an adeno-associated viral vector. In some embodiments, a delivery vector is not a viral vector. In some embodiments, a delivery vector is not a retroviral vector. In some embodiments, a delivery vector is not a lentiviral vector. In some embodiments, a delivery vector is not an adenoviral vector. In some embodiments, a delivery vector is not an adeno-associated viral vector.

In some embodiments, a delivery vector is untargeted or is formulated for non-targeted delivery, for example, can facilitate delivery of an expression construct to a range of cell types including target cells and non-target cells (e.g., immune cells and non-immune cells, or T cells and non-T cells). Specificity of expression in target cells upon non-targeted delivery can be facilitated by an expression regulatory region, such as a cell type-specific promoter.

In some embodiments, a delivery vector is targeted or is formulated for targeted delivery, for example, can facilitate preferential delivery of an expression construct to a target cell type or population, such as immune cells, T cells, NK cells, or NKT cells. A delivery vector can be targeted to a receptor specifically or preferentially expressed on a target cell, for example, CD3 (e.g., CD3 gamma, delta, or epsilon), CD4, or CD8. Specificity of expression in target cells upon targeted delivery can be further enhanced by an expression regulatory region, such as a cell type-specific promoter.

A delivery vector disclosed herein can exclude a cell. For example, in some embodiments a delivery vector that is administered to a living subject does not include a cell, rather it comprises a polynucleotide that is delivered to the cell after administration (e.g., parenteral administration) of the delivery vector to the living subject.

A system disclosed herein (e.g., for in vivo delivery of an expression construct encoding a heterologous immune receptor) can comprise a delivery vector disclosed herein and a polynucleotide disclosed herein. The delivery vector can be, for example, a non-viral vector, a lipid-based delivery vector (LDV), or a non-viral LDV disclosed herein.

### III. Methods

In some embodiments, provided is a composition, method, or system for generating an engineered cell or a population thereof that comprises, encodes, and/or is capable of expressing a heterologous immune receptor disclosed herein. Methods disclosed herein can comprise contacting a cell or a population of cells with a composition or system disclosed herein (e.g., a delivery vector comprising a polynucleotide), thereby expressing the heterologous immune receptor and generating an engineered immune cell. The contacting can be in vivo. For example, a delivery vector comprising an expression construct or polynucleotide can be administered to a subject (e.g., a human), the expression construct or polynucleotide can be taken up by an immune cell in the subject (e.g., a T cell), and the heterologous immune receptor can be expressed by the cell, thereby generating an engineered immune cell in vivo. A delivery vector can be used to deliver an expression construct or polynucleotide to cells in a subject without removing the cells from the subject.

Delivery systems that involve genomic integration can achieve long-term stable transduction of T cells, however in some embodiments permanent transduction with a single heterologous immune receptor can lead to toxicity (e.g., related to cytokine release syndrome or permanent elimination of host cells required for homeostasis), and loss of efficacy upon mutation or loss of expression of the heterologous immune receptor target. In some embodiments, heterologous immune receptors that are only expressed for short periods (e.g., delivered as mRNA) can have limited efficacy and require frequent dosing. In some embodiments, a system disclosed herein provides a balance between transient expression and durability.

A heterologous immune receptor can be expressed in a host cell (e.g., immune cell) without genomic integration of a polynucleotide that encodes the heterologous immune receptor. For example, a heterologous immune receptor can be expressed from an episomal vector, such as a DNA, RNA, circular DNA, circular RNA, minicircle, or the like. A heterologous immune receptor can be transiently expressed. For example, expression of a heterologous immune receptor can be reduced as a nucleic acid that encodes it is degraded and/or diluted upon cellular proliferation.

In some embodiments, after administration of an expression construct, polynucleotide, delivery vector, system, or pharmaceutical composition disclosed herein, a heterologous immune receptor is expressed by immune cells (e.g., T cells) of the subject for at least about 2 days, at least about 5 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 18 weeks, at least about 20 weeks, at least about 22 weeks, at least about 24 weeks, or at least about 26 weeks. Expression of the heterologous immune receptor can be detected, for example, via flow cytometry, RT-qPCR, or RNA seq of PBMCs or tumor infiltrating lymphocytes. Expression can be above a background level or limit of detection.

In some embodiments, after administration of an expression construct, polynucleotide, delivery vector, system, or pharmaceutical composition disclosed herein, a heterologous immune receptor is expressed by immune cells (e.g., T cells) of the subject for at most about 5 days, at most about 1 week, at most about 2 weeks, at most about 3 weeks, at most about 4 weeks, at most about 5 weeks, at most about 6 weeks, at most about 7 weeks, at most about 8 weeks, at most about 9 weeks, at most about 10 weeks, at most about 11 weeks, at most about 12 weeks, at most about 13 weeks, at most about 14 weeks, at most about 15 weeks, at most about 16 weeks, at most about 18 weeks, at most about 20 weeks, at most about 22 weeks, at most about 24 weeks, at most about 26 weeks, or at most about 52 weeks. Expression of the heterologous immune receptor can be detected, for example, via flow cytometry, RT-qPCR, or RNA seq of PBMCs or tumor infiltrating lymphocytes. Expression can be above a background level or limit of detection.

In some embodiments, after administration of an expression construct, polynucleotide, delivery vector, system, or pharmaceutical composition disclosed herein, a heterologous immune receptor is expressed by immune cells (e.g., T cells) of the subject for about 5 days, about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 18 weeks, about 20 weeks, about 22 weeks, about 24 weeks, about 26 weeks, or about 52 weeks. Expression of the heterologous immune receptor can be detected, for example, via flow cytometry, RT-qPCR, or RNA seq of PBMCs or tumor infiltrating lymphocytes. Expression can be above a background level or limit of detection.

In some embodiments, after administration of an expression construct, polynucleotide, delivery vector, system, or pharmaceutical composition disclosed herein, a heterologous immune receptor is expressed by immune cells (e.g., T cells) of the subject for about 1-52 weeks, about 2-52 weeks, about 3-52 weeks, about 4-52 weeks, about 5-52 weeks, about 7-52 weeks, about 10-52 weeks, about 15-52 weeks, about 20-52 weeks, about 1-26 weeks, about 2-26 weeks, about 3-26 weeks, about 4-26 weeks, about 5-26 weeks, about 7-26 weeks, about 10-26 weeks, about 15-26 weeks, about 20-26 weeks, about 1-18 weeks, about 2-18 weeks, about 3-18 weeks, about 4-18 weeks, about 5-18 weeks, about 7-18 weeks, about 10-18 weeks, about 15-18 weeks, about 1-12 weeks, about 2-12 weeks, about 3-12 weeks, about 4-12 weeks, about 5-12 weeks, about 7-12 weeks, about 10-12 weeks, about 1-10 weeks, about 2-10 weeks, about 3-10 weeks, about 4-10 weeks, about 5-10 weeks, about 7-10 weeks, about 1-8 weeks, about 2-8 weeks, about 3-8 weeks, about 4-8 weeks, about 5-8 weeks, about 7-8 weeks, about 1-7 weeks, about 2-7 weeks, about 3-7 weeks, about 4-7 weeks, about 5-7 weeks, about 1-6 weeks, about 2-6 weeks, about 3-6 weeks, about 4-6 weeks, about 5-6 weeks, about 1-5 weeks, about 2-5 weeks, about 3-5 weeks, about 4-5 weeks, about 1-4 weeks, about 2-4 weeks, about 3-4 weeks, about 1-3 weeks, about 2-3 weeks, or about 1-2 weeks. Expression of the heterologous immune receptor can be detected, for example, via flow cytometry, RT-qPCR, or RNA seq of PBMCs or tumor infiltrating lymphocytes. Expression can be above a background level or limit of detection.

In some embodiments, after administration of an expression construct, polynucleotide, delivery vector, system, or pharmaceutical composition disclosed herein, an expression half-life of a heterologous immune receptor by immune cells (e.g., T cells) of the subject is at least about 2 days, at least about 5 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 18 weeks, at least about 20 weeks, at least about 22 weeks, at least about 24 weeks, or at least about 26 weeks. Expression of the heterologous immune receptor can be quantified, for example, via flow cytometry, RT-qPCR, or RNA seq of PBMCs or tumor infiltrating lymphocytes.

In some embodiments, after administration of an expression construct, polynucleotide, delivery vector, system, or pharmaceutical composition disclosed herein, an expression half-life of a heterologous immune receptor by immune cells (e.g., T cells) of the subject is at most about 5 days, at most about 1 week, at most about 2 weeks, at most about 3 weeks, at most about 4 weeks, at most about 5 weeks, at most about 6 weeks, at most about 7 weeks, at most about 8 weeks, at most about 9 weeks, at most about 10 weeks, at most about 11 weeks, at most about 12 weeks, at most about 13 weeks, at most about 14 weeks, at most about 15 weeks, at most about 16 weeks, at most about 18 weeks, at most about 20 weeks, at most about 22 weeks, at most about 24 weeks, at most about 26 weeks, or at most about 52 weeks. Expression of the heterologous immune receptor can be quantified, for example, via flow cytometry, RT-qPCR, or RNA seq of PBMCs or tumor infiltrating lymphocytes.

In some embodiments, after administration of an expression construct, polynucleotide, delivery vector, system, or pharmaceutical composition disclosed herein, an expression half-life of a heterologous immune receptor by immune cells (e.g., T cells) of the subject is about 5 days, about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 18 weeks, about 20 weeks, about 22 weeks, about 24 weeks, about 26 weeks, or about 52 weeks. Expression of the heterologous immune receptor can be quantified, for example, via flow cytometry, RT-qPCR, or RNA seq of PBMCs or tumor infiltrating lymphocytes.

In some embodiments, after administration of an expression construct, polynucleotide, delivery vector, system, or pharmaceutical composition disclosed herein, an expression half-life of a heterologous immune receptor by immune cells (e.g., T cells) of the subject is about 1-52 weeks, about 2-52 weeks, about 3-52 weeks, about 4-52 weeks, about 5-52 weeks, about 7-52 weeks, about 10-52 weeks, about 15-52 weeks, about 20-52 weeks, about 1-26 weeks, about 2-26 weeks, about 3-26 weeks, about 4-26 weeks, about 5-26 weeks, about 7-26 weeks, about 10-26 weeks, about 15-26 weeks, about 20-26 weeks, about 1-18 weeks, about 2-18 weeks, about 3-18 weeks, about 4-18 weeks, about 5-18 weeks, about 7-18 weeks, about 10-18 weeks, about 15-18 weeks, about 1-12 weeks, about 2-12 weeks, about 3-12 weeks, about 4-12 weeks, about 5-12 weeks, about 7-12 weeks, about 10-12 weeks, about 1-10 weeks, about 2-10 weeks, about 3-10 weeks, about 4-10 weeks, about 5-10 weeks, about 7-10 weeks, about 1-8 weeks, about 2-8 weeks, about 3-8 weeks, about 4-8 weeks, about 5-8 weeks, about 7-8 weeks, about 1-7 weeks, about 2-7 weeks, about 3-7 weeks, about 4-7 weeks, about 5-7 weeks, about 1-6 weeks, about 2-6 weeks, about 3-6 weeks, about 4-6 weeks, about 5-6 weeks, about 1-5 weeks, about 2-5 weeks, about 3-5 weeks, about 4-5 weeks, about 1-4 weeks, about 2-4 weeks, about 3-4 weeks, about 1-3 weeks, about 2-3 weeks, or about 1-2 weeks. Expression of the heterologous immune receptor can be quantified, for example, via flow cytometry, RT-qPCR, or RNA seq of PBMCs or tumor infiltrating lymphocytes.

In some embodiments, an expression construct or polynucleotide encoding a heterologous immune receptor disclosed herein is integrated into the genome of a host cell. An expression construct or polynucleotide disclosed herein (e.g., encoding one or more components of a heterologous immune receptor) can be inserted into the genome of a cell in a targeted manner (e.g., at one or more specific sites), or an untargeted manner (e.g., at one or more non-specific sites). For targeted integration, a polynucleotide sequence to be inserted can be flanked by homology arms comprising sequences that are complementary to a genomic DNA sequence to be targeted for insertion (e.g., via homologous recombination and/or homology-directed repair). A double stranded break can be introduced at a target site in the genome, and the homology arms can promote insertion of the polynucleotide. In some cases, a polynucleotide can be excised from a vector (e.g., via a nuclease), and inserted into the genome of the cell.

An expression construct or polynucleotide disclosed herein can be inserted in a safe harbor locus. A safe harbor can comprise a genomic location where an expression construct or polynucleotide can integrate and function without substantially perturbing endogenous activity, for example, with a relatively low impact on local or global gene expression. For example, one or more expression constructs or polynucleotides, disclosed herein can be inserted into any one of HPRT, an AAVS site (e.g., AAVS1, AAVS2, etc.), CCR5, hROSA26, and/or any combination thereof. An expression construct or polynucleotide disclosed herein can be inserted in an intergenic region. An expression construct or polynucleotide disclosed herein can be inserted in a non-coding region. An expression construct or polynucleotide disclosed herein can be inserted within a gene. In some cases, an expression construct or polynucleotide disclosed herein can disrupt a gene it is inserted into (e.g., reduce or eliminate expression of the disrupted gene). A disrupted gene can be for example, an endogenous TCR gene (e.g., TRAC, TCRB/TRB, TRG, TRD), or an immune checkpoint gene (e.g., PD-1, CTLA-4). An expression construct or polynucleotide disclosed herein can be inserted adjacent to or near to a promoter.

A variety of enzymes can catalyze insertion of foreign DNA into a host genome. Non-limiting examples of gene editing tools and techniques include CRISPR, TALEN, zinc finger nuclease (ZFN), meganuclease, Mega-TAL, and transposon-based systems.

A CRISPR system can be utilized to facilitate insertion of an expression construct or polynucleotide disclosed herein encoding a heterologous immune receptor or a component thereof into a cell genome. For example, a CRISPR system can introduce a double stranded break at a target site in a genome or a random site of a genome, and the expression construct or polynucleotide can be inserted, e.g., via homology-directed repair or homologous recombination.

Non-limiting examples of Cas proteins that can be used in the CRISPR systems include Cas1, Cas1B, Cas2, Cas3, Cas4, Cas5, Cas6, Cas7, Cas8, Cas9 (also known as Csn1 or Csx12), Cas10, Csy1, Csy2, Csy3, Cse1, Cse2, Cscl, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, and modified versions thereof. An unmodified CRISPR enzyme can have DNA cleavage activity, such as Cas9. A CRISPR enzyme can direct cleavage of one or both strands at a target sequence, such as within a target sequence and/or within a complement of a target sequence. For example, a CRISPR enzyme can direct cleavage of one or both strands within or within about 1, 2, 3, 4, 5, 6, 7, 8, 9, 10, 15, 20, 25, 50, 100, 200, 500, or more base pairs from the first or last nucleotide of a target sequence. A Cas protein can be a high fidelity Cas protein. Alternatives to S. pyogenes Cas9 may include RNA-guided endonucleases from the Cpf1 family that display cleavage activity in mammalian cells.

A transposon-based system can be utilized for insertion of an expression construct or polynucleotide disclosed herein encoding a heterologous immune receptor of the disclosure or a component thereof into a genome. A transposon can comprise a polynucleotide that can be inserted into a DNA sequence. A class I transposon can be transcribed into an RNA intermediate, then reverse transcribed and inserted into a DNA sequence. A class II transposon can comprise a DNA sequence that is excised from one DNA sequence and/or inserted into another DNA sequence. A class II transposon system can comprise (i) a transposon vector that contains a sequence (e.g., comprising a transgene) flanked by inverted terminal repeats, and (ii) a source for the transposase enzyme.

A transposon system (e.g., class II transposon system) can direct the integration of an expression construct or polynucleotide disclosed herein encoding a heterologous immune receptor or a component thereof, while leaving behind the rest of the vector. A transposon and a transposase can be introduced into a cell. In some cases, a vector that encodes a transposase and comprises an expression construct or polynucleotide disclosed herein is introduced into a cell, and the transposase is expressed and mediates insertion of the transposon, expression construct, and/or polynucleotide into the genome. In some cases, one vector that encodes a transposase is introduced into a cell, and a second vector that comprises a transposon is introduced into the cell, and the transposase is expressed and mediates insertion of the transposon into the genome. In some cases, a vector that comprises a transposon is introduced into a cell, and transposase mediates insertion of a transposon into the genome (e.g., a transposase expressed from a different vector, a transposase introduced as a protein, or a transposase introduced as an RNA that is translated in the cell). In some cases, a vector that encodes a transposase is introduced into a cell, and the transposase is expressed and mediates insertion of a transposon into the genome (e.g., a transposon introduced on a separate vector).

A transposon system can be used for insertion of an expression construct or polynucleotide disclosed herein in an untargeted manner (e.g., at one or more non-specific sites in a genome). A transposon system can be used for insertion of an expression construct or polynucleotide disclosed herein in a targeted manner (e.g., at one or more specific sites in a genome).

Examples of transposon based systems that can be used include, but are not limited to, TcBuster (e.g., derived from the red flour beetle Tribolium castaneum); sleeping beauty (e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol2 (e.g., derived from medaka fish); and spinON.

In some cases, one or more expression constructs or polynucleotides disclosed herein can be inserted randomly into the genome of a cell. For instance, an expression construct or polynucleotide disclosed herein can encode its own promoter or can be inserted into a position where it is under the control of an endogenous promoter. Alternatively or additionally, an expression construct or polynucleotide disclosed herein can be inserted into a gene, such as an intron of a gene, an exon of a gene, a promoter, or a non-coding region.

One or more expression constructs, polynucleotides, systems, or pharmaceutical compositions disclosed herein and/or gene editing components can be delivered to a cell by any suitable method, for example, using any suitable delivery vector disclosed herein.

In some cases, an expression construct, polynucleotide, system, or pharmaceutical composition disclosed herein and/or gene editing components of the disclosure can be delivered to cells without the use of a delivery vector. In some cases, one or more expression constructs, polynucleotides, systems, or pharmaceutical compositions disclosed herein and/or gene editing components of the disclosure can be delivered to cells via delivery vector(s), and one or more expression constructs, polynucleotides, systems, or pharmaceutical compositions disclosed herein and/or gene editing components can be delivered without the use of delivery vector(s).

In some embodiments, cells are genetically engineered to comprise an expression construct, polynucleotide, delivery vector, or system disclosed herein that encodes a heterologous immune receptor of the disclosure ex vivo. For example, cells can be taken from a subject in one or more blood draws and/or apheresis procedures, modified ex vivo to comprise an expression construct or polynucleotide (e.g., not integrated into the genome, or in some embodiments integrated into the genome), optionally selected and/or expanded before and/or after modification, and re-introduced into the subject or a different subject by infusion or injection.

In some cases, cells are genetically engineered to comprise an expression construct, polynucleotide, delivery vector, system, or pharmaceutical composition disclosed herein of the disclosure in vivo. For example, a delivery vector can be used to deliver gene editing components to cells in a subject without removing the cells from the subject. Delivery vectors can be delivered in vivo by administration to an individual subject, for example, by systemic, local, and/or parenteral administration (e.g., intravenous, intraperitoneal, intramuscular, subdermal, or intracranial infusion) or topical application.

Methods to introduce expression constructs, polynucleotides, and/or gene editing components into a cell include, but are not limited to, electroporation, sonoporation, use of a gene gun, lipofection, calcium phosphate transfection, use of dendrimers, microinjection, and use of viral vectors including adenoviral, AAV, and retroviral vectors.

Cells (e.g., ex vivo modified cells) can be selected or enriched for having or not having one or more given factors (e.g., cells may be separated based on the presence or absence of one or more factors). Selection techniques include positive selection and negative selection techniques, (e.g., fluorescent activated cell sorting (FACS) or magnetic activated cell sorting (MACS)). In some cases, cells can be selected before modification, for example, to enrich for a population of cells disclosed herein (e.g., immune cells, such as T cells or a T cell subset disclosed herein, such as gamma delta T cells or alpha beta T cells). Cells can be selected after modification, for example, to enrich for a population of cells disclosed herein (e.g., engineered immune cells that express a heterologous immune receptor of the disclosure). Engineered immune cells that comprise a heterologous immune receptor of the disclosure can be selected or enriched based on a tag or marker, such as an epitope tag. The tag or marker can be appended to the heterologous immune receptor. In some embodiments, the tag or marker is not appended to the heterologous immune receptor. The tag or marker can be co-expressed with the heterologous immune receptor as disclosed herein. The tag or marker can comprise a reporter gene, such as a fluorescent protein.

Cells can be selected, enriched, or expanded on the basis of being positive or negative for a given factor. In some embodiments, cells are selected, enriched, or expanded on the basis of being positive for two or more factors. In some embodiments, cells can be selected, enriched, or expanded on the basis of being positive for one or more factors, and negative for one or more factors. In some cases, a cell can be selected for being CD3+. In some cases, a cell can be selected for being CD8+. In some cases, a cell can be selected for being negative for CD19, CD20, or a combination thereof.

In some cases, a selectable marker is introduced to a cell (e.g., together with or as part of an expression construct, polynucleotide, delivery vector, system, or pharmaceutical composition disclosed herein encoding a heterologous immune receptor), so that cells that comprise the heterologous immune receptor express the selectable marker and can be selected, enriched, or expanded. In some cases, a selectable marker is an antibiotic resistance gene, and cells that do not express the antibiotic resistance gene can be killed by treatment with the antibiotic (e.g., to select or enrich for cells that comprise a heterologous immune receptor). In some embodiments, the selectable marker is an epitope tag.

Expression of a heterologous immune receptor of the disclosure can be quantified, for example, by qPCR, RNA sequencing, western blot, or flow cytometry.

Cells, such as engineered immune cells, can be expanded ex vivo and/or in vitro (e.g., in embodiments where the cells are modified ex vivo). T cells can be expanded, for example, by treatment with agents that elicit CD3 and CD28 signaling (e.g., CD3/CD28 dynabeads). In some cases, cells can be expanded by treatment with an anti-CD2 antibody, an anti-CD3 antibody, an anti-CD27 antibody, an anti-CD28 antibody, an anti-4-1BB antibody, an anti-ICOS antibody, a protein kinase C activator (e.g., PMA, bryostatin, optionally in the presence of ionomycin). One or more of the agents can be coated onto beads. In some cases, cells that express a heterologous immune receptor of the disclosure can be selectively expanded. For example, a population of cells comprising engineered immune cells and non-engineered immune cells can be treated with an agent that elicits signaling through the heterologous immune receptor of the disclosure. In some cases, engineered immune cells that express a heterologous immune receptor of the disclosure can be selectively expanded by treatment with a ligand for the extracellular binding domain and a co-stimulatory agent (e.g., an anti-CD28 antibody). The ligand for the extracellular domain and the co-stimulatory agent can be coupled. Expansion can comprise incubation with one or more growth factors and/or cytokines. For example, cells can be expanded in the presence of serum (e.g., fetal bovine or human serum), IL-2, IFN-g, IL-4, IL-7, GM-CSF, IL-10, IL-21, IL-15, TGF beta, TNF alpha, or a combination thereof.

Cells can be expanded for or for about several hours (e.g., about 3 hours) to or to about 14 days or any hourly integer value in between. In another embodiment, the mixture may be cultured for or for about 21 days or for up to or for up to about 21 days.

In some embodiments, selected cells can be expanded ex vivo and/or in vitro before gene editing or delivery of an expression construct, polynucleotide, delivery vector, system, or pharmaceutical composition disclosed herein, after gene editing or delivery of an expression construct, polynucleotide, delivery vector, system, or pharmaceutical composition disclosed herein, before selection, after selection, before expansion, after expansion, or a combination thereof. In some embodiments, selected cells can be expanded ex vivo and/or in vitro before gene editing or delivery of an expression construct, polynucleotide, delivery vector, system, or pharmaceutical composition disclosed herein. In some embodiments, selected cells can be expanded ex vivo and/or in vitro after gene editing or delivery of an expression construct, polynucleotide, delivery vector, system, or pharmaceutical composition disclosed herein. In some embodiments, selected cells can be expanded ex vivo and/or in vitro before selection and/or enrichment. In some embodiments, selected cells can be expanded ex vivo and/or in vitro after selection and/or enrichment. In some embodiments, selected cells can be expanded ex vivo and/or in vitro before expansion. In some embodiments, selected cells can be expanded ex vivo and/or in vitro after expansion.

Cells of the disclosure can be cryopreserved, e.g., frozen in the presence of a cryopreservative such as DMSO, and stored at a low temperature (e.g., at −80° C. or in liquid nitrogen). Cells of the disclosure can be cryopreserved before gene editing, after gene editing, before selection, after selection, before expansion, after expansion, or a combination thereof.

Disclosed herein are methods that comprise administering to a subject an expression construct, polynucleotide, system, delivery vector, and/or pharmaceutical composition disclosed herein. The methods can comprise treating a subject in need thereof. The subject can have a disease or condition, such as a cancer. The cancer can be a solid tumor. The cancer can be a liquid tumor. The cancer can be a hematologic tumor. The cancer can be an immune cell cancer. The cancer can be a B cell cancer. The cancer can be a T cell cancer. The cancer can be a myeloid cell cancer. The cancer ca be a leukemia. The cancer ca be a lymphoma. The cancer ca be a myeloma. The cancer can be a carcinoma. The cancer can be a sarcoma. The cancer can be an adenoma. The cancer can be, for example, B-cell lymphoma, mantle cell lymphoma, multiple myeloma, acute lymphoblastic leukemia (ALL), non-Hodgkin lymphoma, T cell lymphoblastic leukemia, follicular lymphoma, anaplastic large cell lymphoma (ALCL), peripheral T cell lymphoma-not otherwise specified (PTCL-NOS), angioimmunoblastic T-cell lymphoma (AITL), cutaneous T-cell lymphoma (CTCL), adult T-cell lymphoma and leukemia (ATLL), or T-cell acute lymphoblastic leukemia or lymphoma (T-ALL/LBL).

In some embodiments, a method disclosed herein can be advantageous for treating a T cell malignancy (for example, using a heterologous immune receptor that targets a surface molecule on a T cell). In some embodiments, a method disclosed herein can be advantageous for treating an autoimmune condition associated with T cell dysfunction or T cell-mediated autoimmune responses (for example, using a heterologous immune receptor that targets a surface molecule on a T cell). For example, a system disclosed herein can utilize a heterologous immune receptor that targets a surface molecule on T cells (e.g., CD3, CD4, CD5, CD7, CD8, CD90, CD5, CD30, CD37, CCR4, TRB, TRAC, or TRBC1), and can circumvent the need to transfect and expand a population of T cells ex vivo. Ex vivo expansion of T cells with a heterologous immune receptor (e.g., CAR) that targets T cells can be impractical due to killing of the transformed cells via fratricide. A system or method disclosed herein can be used to transfect a limited population of immune cells (e.g., T cells) in vivo, and can be used for targeting T cells in vivo for treatment of a T cell malignancy or a T cell-mediated autoimmune disorder.

In some embodiments, the extracellular binding domain of the heterologous immune receptor binds to a T cell receptor, e.g., a constant domain, variable domain, CDR, LCDR3, or HCDR3 thereof. In some embodiments, the extracellular binding domain of the heterologous immune receptor binds to a particular T cell receptor clone, for example, specifically or preferentially binds to a TCR specific for a cognate autoimmune antigen.

In some embodiments, the extracellular binding domain of the heterologous immune receptor binds to a B cell receptor (BCR, e.g., IgM or IgD), e.g., a constant domain, variable domain, CDR, LCDR3, or HCDR3 thereof. In some embodiments, the extracellular binding domain of the heterologous immune receptor binds to a particular BCR clone, for example, specifically or preferentially binds to a BCR specific for a cognate autoimmune antigen.

In some embodiments, a method disclosed herein can be used for treating an autoimmune disorder. In some embodiments, a method disclosed herein can be used for treating, reducing, or preventing fibrosis, such as cardiac fibrosis (e.g., with a CAR targeting FAP).

In some embodiments, a method disclosed herein can be used for treating a condition associated with cellular senescence (e.g., with a CAR targeting a senescence-associated cell surface molecule on a target cell, such as urokinase-type plasminogen activator receptor (uPAR)).

Illustrative methods of treatment can include administration of an expression construct, polynucleotide, system, and/or delivery vector disclosed herein, including as part of a pharmaceutical composition. The expression construct, polynucleotide, system, and/or delivery vector can be administered in an amount effective to treat or prevent a disease or condition. “Treatment” (and grammatical variations thereof such as “treat” or “treating”) can refer to clinical intervention in an attempt to alter the natural course of the individual (subject) being treated, and can be performed either for prophylaxis or during the course of clinical pathology. Desirable effects of treatment can include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

An expression construct, polynucleotide, system, delivery vector, and/or pharmaceutical composition can be administered to an individual subject, for example, by parenteral administration or topical application. An expression construct, polynucleotide, system, delivery vector, and/or pharmaceutical composition can be administered to an individual subject, for example, by intravenous, intraperitoneal, intramuscular, subdermal, intracerebral, intracerebroventricular, intra-articular, intraarterial, intrathecal, intracapsular, subcapsular, intraorbital, intracardiac, intradermal, subcutaneous, subarachnoid, or intracranial injection or infusion. The administration can be via localized injection or infusion. The administration can be via systemic injection or infusion. The administration can be via intravenous injection or infusion. The administration can be via intratumoral injection or infusion. The administering can be local. The administering can be systemic.

Various dosing schedules can be used. In some embodiments, an expression construct, polynucleotide, system, and/or delivery vector is administered to a subject once. In some embodiments, an expression construct, polynucleotide, system, and/or delivery vector is administered to a subject two or more times. In some embodiments, a system disclosed herein comprising a non-integrating polynucleotide can allow for repeat administration with reduced likelihood of toxicity (e.g., cytokine release syndrome), based on a limited number of copies of a polynucleotide that are degraded over time and/or become diluted as cells expressing the heterologous immune receptor are activated and divide.

Immune signaling and/or activation of a cell expressing a heterologous immune receptor disclosed herein can be induced upon binding of the extracellular binding domain of the heterologous immune receptor to a target cell surface molecule. Activation of the cell can comprise, for example, proliferation, receptor clustering, secretion of cytokines, production of chemokines, killing of target cells, communicating to other immune cells, or a combination thereof.

Administering an expression construct, polynucleotide, system, delivery vector, or a pharmaceutical composition comprising the same to a subject in need thereof can prolong cancer survival, for example, as demonstrated in a mouse model of cancer. In some embodiments, administering an expression construct, polynucleotide, system, or pharmaceutical composition to a subject with cancer prolongs cancer survival by at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, or at least 250 fold, for example, in a mouse model of cancer. The increase in cancer survival can be, for example, compared to control subjects that are not administered the expression construct, polynucleotide, system, or pharmaceutical composition, or compared to control subjects that are administered a control expression construct, polynucleotide, system, or pharmaceutical composition.

Administering an expression construct, polynucleotide, system, delivery vector, or a pharmaceutical composition comprising the same to a subject in need thereof can reduce tumor volume or tumor burden, for example, as demonstrated in a mouse model of cancer. In some embodiments, administering an expression construct, polynucleotide, system, or pharmaceutical composition to a subject with cancer reduces tumor volume or tumor burden by at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, or at least 250 fold, for example, in a mouse model of cancer. The reduction in tumor volume or tumor burden can be, for example, compared to control subjects that are not administered the expression construct, polynucleotide, system, or pharmaceutical composition, or compared to control subjects that are administered a control expression construct, polynucleotide, system, or pharmaceutical composition.

In some embodiments, after administration of an expression construct, polynucleotide, delivery vector, system, or pharmaceutical composition disclosed herein, a heterologous immune receptor is functional in immune cells (e.g., T cells) of the subject for at least about 2 days, at least about 5 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 18 weeks, at least about 20 weeks, at least about 22 weeks, at least about 24 weeks, or at least about 26 weeks. Functionality of the heterologous immune receptor can be determined, for example, by assaying activation, cytotoxicity, or cytokine production of the immune cells in response to cells expressing a cell surface molecule targeted by the heterologous immune receptor, e.g., ex vivo after isolating cells from the subject.

In some embodiments, after administration of an expression construct, polynucleotide, delivery vector, system, or pharmaceutical composition disclosed herein, a heterologous immune receptor is functional in immune cells (e.g., T cells) of the subject for at most about 5 days, at most about 1 week, at most about 2 weeks, at most about 3 weeks, at most about 4 weeks, at most about 5 weeks, at most about 6 weeks, at most about 7 weeks, at most about 8 weeks, at most about 9 weeks, at most about 10 weeks, at most about 11 weeks, at most about 12 weeks, at most about 13 weeks, at most about 14 weeks, at most about 15 weeks, at most about 16 weeks, at most about 18 weeks, at most about 20 weeks, at most about 22 weeks, at most about 24 weeks, at most about 26 weeks, or at most about 52 weeks. Functionality of the heterologous immune receptor can be determined, for example, by assaying activation, cytotoxicity, or cytokine production of the immune cells in response to cells expressing a cell surface molecule targeted by the heterologous immune receptor, e.g., ex vivo after isolating cells from the subject.

In some embodiments, after administration of an expression construct, polynucleotide, delivery vector, system, or pharmaceutical composition disclosed herein, a heterologous immune receptor is functional in immune cells (e.g., T cells) of the subject for about 5 days, about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 18 weeks, about 20 weeks, about 22 weeks, about 24 weeks, about 26 weeks, or about 52 weeks. Functionality of the heterologous immune receptor can be determined, for example, by assaying activation, cytotoxicity, or cytokine production of the immune cells in response to cells expressing a cell surface molecule targeted by the heterologous immune receptor, e.g., ex vivo after isolating cells from the subject.

In some embodiments, after administration of an expression construct, polynucleotide, delivery vector, system, or pharmaceutical composition disclosed herein, a heterologous immune receptor is functional in immune cells (e.g., T cells) of the subject for about 1-52 weeks, about 2-52 weeks, about 3-52 weeks, about 4-52 weeks, about 5-52 weeks, about 7-52 weeks, about 10-52 weeks, about 15-52 weeks, about 20-52 weeks, about 1-26 weeks, about 2-26 weeks, about 3-26 weeks, about 4-26 weeks, about 5-26 weeks, about 7-26 weeks, about 10-26 weeks, about 15-26 weeks, about 20-26 weeks, about 1-18 weeks, about 2-18 weeks, about 3-18 weeks, about 4-18 weeks, about 5-18 weeks, about 7-18 weeks, about 10-18 weeks, about 15-18 weeks, about 1-12 weeks, about 2-12 weeks, about 3-12 weeks, about 4-12 weeks, about 5-12 weeks, about 7-12 weeks, about 10-12 weeks, about 1-10 weeks, about 2-10 weeks, about 3-10 weeks, about 4-10 weeks, about 5-10 weeks, about 7-10 weeks, about 1-8 weeks, about 2-8 weeks, about 3-8 weeks, about 4-8 weeks, about 5-8 weeks, about 7-8 weeks, about 1-7 weeks, about 2-7 weeks, about 3-7 weeks, about 4-7 weeks, about 5-7 weeks, about 1-6 weeks, about 2-6 weeks, about 3-6 weeks, about 4-6 weeks, about 5-6 weeks, about 1-5 weeks, about 2-5 weeks, about 3-5 weeks, about 4-5 weeks, about 1-4 weeks, about 2-4 weeks, about 3-4 weeks, about 1-3 weeks, about 2-3 weeks, or about 1-2 weeks. Functionality of the heterologous immune receptor can be determined, for example, by assaying activation, cytotoxicity, or cytokine production of the immune cells in response to cells expressing a cell surface molecule targeted by the heterologous immune receptor, e.g., ex vivo after isolating cells from the subject.

Pharmaceutical compositions of the present disclosure can comprise a composition disclosed herein and a pharmaceutically acceptable excipient, vehicle, carrier, or diluent. A pharmaceutical composition can comprise, for example, an expression construct or polynucleotide disclosed herein, a delivery vector disclosed herein, and a pharmaceutically acceptable excipient, vehicle, carrier, or diluent. A pharmaceutical composition can be formulated, for example, for systemic, local, parenteral, intratumoral, intravenous, intraperitoneal, subcutaneous, transdermal, or intramuscular administration. These compositions can take the form of solutions, suspensions, emulsion, tablets, pills, capsules, powders, sustained-release formulations, and the like.

### IV. Embodiments

Embodiment 1. A system for in vivo delivery of an expression construct encoding a heterologous immune receptor, the system comprising: (a) a non-viral lipid-based delivery vector (LDV); and (b) a polynucleotide that encodes the heterologous immune receptor, wherein expression of the heterologous immune receptor is driven by an expression regulatory region that comprises an immune cell-specific promoter.
Embodiment 2. A system for in vivo delivery of an expression construct encoding a heterologous immune receptor, the system comprising: (a) a non-viral delivery vector; and (b) a polynucleotide that encodes the heterologous immune receptor, wherein expression of the heterologous immune receptor is driven by an expression regulatory region comprising an immune cell-specific promoter that comprises: (i) a CD3 promoter that natively drives expression of CD3 in a mammalian cell; (ii) a CD4 promoter that natively drives expression of CD4 in a mammalian cell; (iii) a distal lymphocyte protein tyrosine kinase (dLck) promoter that natively drives expression of dLck in a mammalian cell; or (iv) an NKp46 promoter that natively drives expression of NKp46 in a mammalian cell.
Embodiment 3. A system for in vivo delivery of an expression construct encoding a heterologous immune receptor, the system comprising: (a) a lipid-based delivery vector (LDV) that comprises a fusion-associated small transmembrane (FAST) protein; and (b) a polynucleotide that encodes the heterologous immune receptor.
Embodiment 4. The system of embodiment 3, wherein expression of the heterologous immune receptor is driven by an immune cell-specific promoter.
Embodiment 5. The system of embodiment 1, 2 or 4, wherein the immune cell-specific promoter comprises a mammalian promoter.
Embodiment 6. The system of embodiment 1, 2, or 4, wherein the immune cell-specific promoter comprises a human promoter.
Embodiment 7. The system of any one of embodiments 1, 2, and 4-6, wherein the immune cell-specific promoter comprises a T cell-specific promoter.
Embodiment 8. The system of any one of embodiments 1, 2, and 4-6, wherein the immune cell-specific promoter comprises a CD3 gamma promoter that natively drives expression of CD3 gamma.
Embodiment 9. The system of any one of embodiments 1, 2, and 4-6, wherein the immune cell-specific promoter comprises a CD3 delta promoter that natively drives expression of CD3 delta.
Embodiment 10. The system of any one of embodiments 1, 2, and 4-6, wherein the immune cell-specific promoter comprises a CD3 epsilon promoter that natively drives expression of CD3 epsilon.
Embodiment 11. The system of any one of embodiments 1, 2, and 4-6, wherein the immune cell-specific promoter comprises a CD3 zeta promoter that natively drives expression of CD3 zeta.
Embodiment 12. The system of any one of embodiments 1, 2, and 4-6, wherein the immune cell-specific promoter comprises a CD4 promoter that natively drives expression of CD4.
Embodiment 13. The system of any one of embodiments 1 and 4-6, wherein the immune cell-specific promoter comprises a CD8 promoter that natively drives expression of CD8.
Embodiment 14. The system of any one of embodiments 1 and 4-6, wherein the immune cell-specific promoter comprises a TRAC promoter that natively drives expression of TRAC.
Embodiment 15. The system of any one of embodiments 1 and 4-6, wherein the immune cell-specific promoter comprises a TCRB promoter that natively drives expression of TCRB.
Embodiment 16. The system of any one of embodiments 1, 2, and 4-6, wherein the immune cell-specific promoter comprises a distal lymphocyte protein tyrosine kinase (dLck) promoter that natively drives expression of dLck.
Embodiment 17. The system of any one of embodiments 1, 2, and 4-6, wherein the immune cell-specific promoter comprises an NKp46 promoter that natively drives expression of NKp46.
Embodiment 18. The system of embodiment 1 or embodiment 2, wherein the expression regulatory region further comprises an enhancer.
Embodiment 19. The system of embodiment 18, wherein the enhancer is a mammalian CD4 enhancer.
Embodiment 20. The system of embodiment 18, wherein the enhancer is a mammalian CD8 enhancer.
Embodiment 21. The system of embodiment 18, wherein the enhancer is a mammalian CD3 enhancer.
Embodiment 22. The system of any one of embodiments 1, 2, and 18-21, wherein the expression regulatory region further comprises an intron.
Embodiment 23. The system of embodiment 22, wherein the intron is a pCI intron or a CD3 intron.
Embodiment 24. The system of any one of embodiments 1-2 and 18-23, wherein the expression regulatory region further comprises a splice acceptor.
Embodiment 25. The system of any one of embodiments 1-2 and 18-24, wherein the expression regulatory region further comprises an exon or a fragment thereof.
Embodiment 26. The system of any one of embodiments 1-25, wherein the polynucleotide comprises DNA.
Embodiment 27. The system of any one of embodiments 1-25, wherein the polynucleotide comprises double stranded DNA.
Embodiment 28. The system of any one of embodiments 1-25, wherein the polynucleotide is a DNA plasmid, nanoplasmid, or minicircle.
Embodiment 29. The system of embodiment 3, wherein the polynucleotide comprises mRNA.
Embodiment 30. The system of any one of embodiments 1-29, wherein the heterologous immune receptor is a chimeric antigen receptor (CAR).
Embodiment 31. The system of embodiment 30, wherein the CAR is a second generation CAR.
Embodiment 32. The system of embodiment 30, wherein the CAR is a third, fourth, or fifth generation CAR.
Embodiment 33. The system of any one of embodiments 30-32, wherein the CAR comprises an extracellular binding domain that binds to CD19.
Embodiment 34. The system of any one of embodiments 30-33, wherein the CAR comprises a CD3 zeta cytoplasmic signaling domain.
Embodiment 35. The system of embodiment 34, wherein the CD3 zeta cytoplasmic signaling domain comprises an inactivated ITAM.
Embodiment 36. The system of embodiment 34, wherein the CD3 zeta cytoplasmic signaling domain comprises two inactivated ITAMs.
Embodiment 37. The system of any one of embodiments 30-36, wherein the CAR comprises a T cell costimulatory cytoplasmic signaling domain.
Embodiment 38. The system of any one of embodiments 30-37, wherein the CAR comprises a CD28 cytoplasmic signaling domain.
Embodiment 39. The system of any one of embodiments 30-38, wherein the CAR comprises a 41BB zeta cytoplasmic signaling domain.
Embodiment 40. The system of any one of embodiments 30-39, wherein the CAR is a dual CAR.
Embodiment 41. The system of any one of embodiments 30-32 and 34-40, wherein the CAR is a universal CAR.
Embodiment 42. The system of any one of embodiments 1-29, wherein the heterologous immune receptor is a T cell receptor.
Embodiment 43. The system of any one of embodiments 1-42, wherein the polynucleotide further comprises a transgene that encodes an immunomodulatory factor.
Embodiment 44. The system of embodiment 43, wherein the immunomodulatory factor comprises a cytokine.
Embodiment 45. The system of embodiment 43, wherein the immunomodulatory factor comprises a cytokine receptor.
Embodiment 46. The system of embodiment 43, wherein the immunomodulatory factor comprises a chemokine receptor.
Embodiment 47. The system of embodiment 43, wherein the immunomodulatory factor comprises an immune co-receptor.
Embodiment 48. The system of embodiment 1, wherein the LDV comprises a fusion-associated small transmembrane (FAST) protein.
Embodiment 49. The system of any one of embodiments 1 and 3-48, wherein the LDV is formulated for non-targeted delivery to immune cells and non-immune cells.
Embodiment 50. The system of any one of embodiments 1 and 3-49, wherein the LDV is formulated for non-targeted delivery to T cells and non-T cells.
Embodiment 51. The system of any one of embodiments 1 and 3-48, wherein the LDV is formulated for targeted delivery to T cells.
Embodiment 52. The system of any one of embodiments 1 and 3-51, wherein the LDV comprises an ionizable lipid.
Embodiment 53. The system of embodiment 52, wherein a molar ratio of the ionizable lipid to the polynucleotide is between about 2:1 and 25:1.
Embodiment 54. The system of embodiment 53, wherein the molar ratio is about 5:1, about 7.5:1, about 10:1, or about 15:1.
Embodiment 55. The system of any one of embodiments 52-54, wherein the ionizable lipid comprises Dlin-KC2-DMA (KC2), DODMA, DODAP, DOBAQ, DOTMA, 18:1 EPC, DOTAP, DDAB, 18:0 EPC, 18:0 DAP, or 18:0 TAP.
Embodiment 56. The system of any one of embodiments 3 and 26-55, wherein the FAST protein comprises p10, p13, p14, p15, p16, p22, or a functional domain thereof.
Embodiment 57. The system of any one of embodiments 3 and 26-55, wherein the FAST protein comprises a fusion of a first domain from a p14 FAST protein or a p10 FAST protein and a second domain from a p14 FAST protein or a p15 FAST protein.
Embodiment 58. The system of any one of embodiments 3 and 26-55, wherein the FAST protein comprises an ectodomain of p14 and an endodomain of p15.
Embodiment 59. An expression construct for in vivo delivery of a heterologous immune receptor, the expression construct comprising a polynucleotide that comprises: (a) a transgene that encodes the heterologous immune receptor; and (b) an expression regulatory region, wherein the expression regulatory region comprises: (i) a T cell-specific promoter, and (ii) an enhancer or an intron.
Embodiment 60. The expression construct of embodiment 59, wherein the expression construct comprises the enhancer.
Embodiment 61. The expression construct of embodiment 60, wherein the enhancer is a mammalian CD4 enhancer, CD3 enhancer, or CD8 enhancer.
Embodiment 62. The expression construct of any one of embodiments 59-61, wherein the expression construct comprises the intron.
Embodiment 63. The expression construct of embodiment 62, wherein the intron is a pCI intron or a CD3 intron.
Embodiment 64. The expression construct of any one of embodiments 59-63, wherein the expression regulatory region further comprises a splice acceptor.
Embodiment 65. The expression construct of any one of embodiments 59-64, wherein the expression regulatory region further comprises an exon or a fragment thereof.
Embodiment 66. A method of expressing a CAR in an immune cell, the method comprising contacting the cell with the system of any one of embodiments 1-58 or the expression construct of any one of embodiments 59-65.
Embodiment 67. A method of treating a condition in a subject in need thereof, the method comprising administering to the subject an effective amount of the system of any one of embodiments 1-58 or the expression construct of any one of embodiments 59-65.
Embodiment 68. The method of embodiment 67, wherein the administering is parenteral.
Embodiment 69. The method of embodiment 67, wherein the administering is intravenous.
Embodiment 70. The method of embodiment 67, wherein the administering is systemic.
Embodiment 71. The method of embodiment 67, wherein the administering is local.
Embodiment 72. The method of embodiment 67, wherein the administering is intratumoral.
Embodiment 73. The method of embodiment 66, wherein the polynucleotide is not integrated into a genome of the cell.
Embodiment 74. The method of embodiment 67, wherein the polynucleotide is not integrated into a genome of the subject.
Embodiment 75. The method of any one of embodiments 66-74, wherein the CAR is expressed transiently.
Embodiment 76. The method of any one of embodiments 67-75, further comprising administering a second dose of the system or the expression construct to the subject.
Embodiment 77. The method of any one of embodiments 67-76, wherein the condition is cancer.
Embodiment 78. The method of embodiment 77, wherein the condition is a B cell cancer.
Embodiment 79. The method of embodiment 77, wherein the condition is acute lymphoblastic leukemia (ALL), multiple myeloma, acute myeloid leukemia (AML), or B cell acute lymphoblastic leukemia (B-ALL).
Embodiment 80. The method of embodiment 77, wherein the condition is a T cell cancer.

### V. Additional Sequences

TABLE 4
additional illustrative sequences

| SEQ | | |
|---|---|---|
| ID | | |
| NO | Description | Sequence |
| 93 | BGH pA (CpG | CTACTGTGCCTTCTAGTTGCCAGCCATCTGTTGTTTGCCC |
| | free-long) | CTCCCCCTTGCCTTCCTTGACCCTGGAAGGTGCCACTCCC |
| | | ACTGTCCTTTCCTAATAAAATGAGGAAATTGCATCACATT |
| | | GTCTGAGTAGGTGTCATTCTATTCTGGGGGGTGGGGTGG |
| | | GGCAGGACAGCAAGGGGGAGGATTGGGAAGACAATAGC |
| | | AGGCATGCTGGGGATGCAGTGGGCTCTATGG |
| 94 | Beta globin | AATAAAGGAAATTTATTTTCATTGCAATAGTGTGTTGGAA |
| | poly(A) signal | TTTTTTGTGTCTCTCA |
| 95 | Anti-mouse | ATGGGTGTCCCTACCCAGCTCCTGGGACTGCTCCTGCTGT |
| | CD19 CAR | GGATCACCGACGCCATCTGCGACATCCAGATGACCCAGA |
| | (nucleotide; | GCCCTGCCAGCCTGTCTACCAGCCTGGGCGAGACAGTGA |
| | synthetic | CCATCCAGTGTCAGGCCAGCGAGGACATCTACTCTGGCC |
| | construct) | TGGCTTGGTATCAGCAGAAGCCCGGCAAGAGCCCTCAGC |
| | | TGCTGATCTACGGCGCCAGCGACCTGCAGGACGGCGTGC |
| | | CTAGCAGATTCAGCGGCAGCGGCTCCGGAACCCAGTACA |
| | | GCCTGAAGATCACCAGCATGCAGACCGAGGACGAGGGC |
| | | GTGTACTTCTGCCAGCAAGGCCTGACCTACCCTAGAACCT |
| | | TCGGAGGAGGCACCAAGCTGGAACTGAAGGGCGGAGGC |
| | | GGAAGTGGAGGCGGAGGATCTGGCGGCGGAGGCTCTGA |
| | | AGTGCAGCTGCAGCAGTCTGGCGCTGAACTGGTCCGGCC |
| | | TGGCACTAGCGTGAAGCTGTCCTGCAAGGTGTCCGGCGA |
| | | CACCATCACCTTCTACTACATGCACTTCGTGAAGCAGAG |
| | | GCCAGGACAGGGCCTGGAATGGATCGGCAGAATCGACCC |
| | | TGAGGACGAGAGCACCAAGTACAGCGAGAAGTTCAAGA |
| | | ACAAGGCCACCCTGACCGCCGACACCAGCAGCAACACCG |
| | | CCTACCTGAAGCTGTCTAGCCTGACCTCCGAGGACACCG |
| | | CCACCTACTTTTGCATCTACGGCGGCTACTACTTCGACTA |
| | | CTGGGGCCAGGGCGTGATGGTCACCGTGTCCAGCATCGA |
| | | GTTCATGTACCCCCCTCCCTACCTGGACAACGAGAGAAG |
| | | CAACGGCACCATCATCCACATCAAAGAAAAGCACCTGTG |
| | | CCACACCCAGAGCAGCCCCAAGCTGTTCTGGGCCCTGGT |
| | | GGTGGTGGCCGGCGTGCTGTTCTGTTACGGCCTGCTGGTC |
| | | ACAGTGGCCCTGTGCGTGATCTGGACCAACAGCAGAAGA |
| | | AACAGAGGCGGCCAGAGCGACTACATGAACATGACCCCC |
| | | AGAAGGCCAGGCCTGACCAGAAAGCCCTACCAGCCCTAC |
| | | GCCCCTGCCAGAGACTTCGCCGCCTACAGACCCAGAGCC |
| | | AAGTTCAGCAGATCCGCCGAGACAGCCGCCAACCTGCAG |
| | | GATCCCAACCAGCTGTTCAACGAGCTGAACCTGGGCAGA |
| | | CGGGAGGAGTTCGACGTGCTGGAAAAGAAGAGAGCCAG |
| | | GGACCCCGAGATGGGCGGCAAGCAGCAGAGAAGAAGAA |
| | | ACCCTCAGGAAGGCGTCTACAACGCCCTGCAGAAAGACA |
| | | AGATGGCCGAGGCCTACAGCGAGATCGGCACCAAGGGC |
| | | GAGAGAAGAAGGGGCAAGGGCCACGATGGCCTGTTCCA |
| | | GGGCCTGTCCACCGCCACCAAGGACACCTTCGACGCCCT |
| | | GCACATGCAGACCCTGGCCCCCAGATGA |
| 96 | CD8α signal | MLLLVTSLLLCELPHPAFLLIP |
| | peptide | |
| 97 | CD4 signal | MNRGVPFRHLLLVLQLALLPAATQG |
| | peptide | |
| 98 | Flexible linker/ | GGGGSGGGGSGGGGS |
| | linker subunit | |
| 99 | Flexible linker/ | GGGGS |
| | linker subunit | |
| 100 | Flexible linker/ | GGGS |
| | linker subunit | |
| 101 | Flexible linker/ | GG |
| | linker subunit | |
| 102 | Flexible linker/ | KESGSVSSEQLAQFRSLD |
| | linker subunit | |
| 103 | Flexible linker/ | EGKSSGSGSESKST |
| | linker subunit | |
| 104 | Flexible linker/ | GSAGSAAGSGEF |
| | linker subunit | |
| 105 | Rigid | EAAAK |
| | linker/linker | |
| | subunit | |
| 106 | Rigid | EAAAR |
| | linker/linker | |
| | subunit | |
| 107 | Rigid | PAPAP |
| | linker/linker | |
| | subunit | |
| 108 | Rigid | AEAAAKEAAAKA |
| | linker/linker | |
| | subunit | |
| 109 | Spacer | SGGGSDP |
| 110 | F2A | APVKQTLNFDLLKLAGDVESNPGP |
| 111 | P2A | ATNFSLLKQAGDVEENPGP |
| 112 | E2A | QCTNYALLKLAGDVESNPGP |
| 113 | T2A | EGRGSLLTCGDVEENPGP |
| 114 | GSG-F2A | GSGAPVKQTLNFDLLKLAGDVESNPGP |
| 115 | GSG-P2A | GSGATNFSLLKQAGDVEENPGP |
| 116 | GSG-E2A | GSGQCTNYALLKLAGDVESNPGP |
| 117 | GSG-T2A | GSGEGRGSLLTCGDVEENPGP |
| 118 | Furin-F2A | RRKRAPVKQTLNFDLLKLAGDVESNPGP |
| 119 | Furin-P2A | RRKRATNFSLLKQAGDVEENPGP |
| 120 | Furin-E2A | RRKRQCTNYALLKLAGDVESNPGP |
| 121 | Furin-T2A | RRKREGRGSLLTCGDVEENPGP |
| 122 | Furin-GSG-F2A | RRKRGSGAPVKQTLNFDLLKLAGDVESNPGP |
| 123 | Furin-GSG-P2A | RRKRGSGATNFSLLKQAGDVEENPGP |
| 124 | Furin-GSG-E2A | RRKRGSGQCTNYALLKLAGDVESNPGP |
| 125 | Furin-GSG-T2A | RRKRGSGEGRGSLLTCGDVEENPGP |
| 126 | aCD19-CD28- | MLLLVTSLLLCELPHPAFLLIPDIQMTQTTSSLSASLGDRVTI |
| | CD27-CD3-2A- | SCRASQDISKYLNWYQQKPDGTVKLLIYHTSRLHSGVPSRF |
| | iCasp9 | SGSGSGTDYSLTISNLEQEDIATYFCQQGNTLPYTFGGGTKL |
| | (synthetic | EITGSTSGSGKPGSGEGSTKGEVKLQESGPGLVAPSQSLSVT |
| | construct) | CTVSGVSLPDYGVSWIRQPPRKGLEWLGVIWGSETTYYNSA |
| | | LKSRLTIIKDNSKSQVFLKMNSLQTDDTAIYYCAKHYYYGG |
| | | SYAMDYWGQGTSVTVSSAAAIEVMYPPPYLDNEKSNGTIIH |
| | | VKGKHLCPSPLFPGPSKPFWVLVVVGGVLACYSLLVTVAFII |
| | | FWVRSKRSRLLHSDYMNMTPRRPGPTRKHYQPYAPPRDFA |
| | | AYRSASGGGGSGGGGSQRRKYRSNKGESPVEPAEPCHYSCP |
| | | REEEGSTIPIQEDYRKPEPACSPGGGGSGGGGSRVKFSRSAD |
| | | APAYQQGQNQLYNELNLGRREEYDVLDKRRGRDPEMGGK |
| | | PRRKNPQEGLYNELQKDKMAEAYSEIGMKGERRRGKGHD |
| | | GLYQGLSTATKDTYDALHMQALPPRTSGSGATNFSLLKQA |
| | | GDVEENPGPMGVQVETISPGDGRTFPKRGQTCVVHYTGML |
| | | EDGKKVDSSRDRNKPFKFMLGKQEVIRGWEEGVAQMSVG |
| | | QRAKLTISPDYAYGATGHPGIIPPHATLVFDVELLKLEGGGG |
| | | SGGGGSGAMVGALESLRGNADLAYILSMEPCGHCLIINNVN |
| | | FCRESGLRTRTGSNIDCEKLRRRFSSLHFMVEVKGDLTAKK |
| | | MVLALLELARQDHGALDCCVVVILSHGCQASHLQFPGAVY |
| | | GTDGCPVSVEKIVNIFNGTSCPSLGGKPKLFFIQACGGEQKD |
| | | HGFEVASTSPEDESPGSNPEPDATPFQEGLRTFDQLDAISSLP |
| | | TPSDIFVSYSTFPGFVSWRDPKSGSWYVETLDDIFEQWAHSE |
| | | DLQSLLLRVANAVSVKGIYKQMPGCFNFLRKKLFFKTSAS |
| 127 | aCD19-CD28- | MLLLVTSLLLCELPHPAFLLIPDIQMTQTTSSLSASLGDRVTI |
| | CD27-CD3 CAR | SCRASQDISKYLNWYQQKPDGTVKLLIYHTSRLHSGVPSRF |
| | (synthetic | SGSGSGTDYSLTISNLEQEDIATYFCQQGNTLPYTFGGGTKL |
| | construct) | EITGSTSGSGKPGSGEGSTKGEVKLQESGPGLVAPSQSLSVT |
| | | CTVSGVSLPDYGVSWIRQPPRKGLEWLGVIWGSETTYYNSA |
| | | LKSRLTIIKDNSKSQVFLKMNSLQTDDTAIYYCAKHYYYGG |
| | | SYAMDYWGQGTSVTVSSAAAIEVMYPPPYLDNEKSNGTIIH |
| | | VKGKHLCPSPLFPGPSKPFWVLVVVGGVLACYSLLVTVAFII |
| | | FWVRSKRSRLLHSDYMNMTPRRPGPTRKHYQPYAPPRDFA |
| | | AYRSASGGGGSGGGGSQRRKYRSNKGESPVEPAEPCHYSCP |
| | | REEEGSTIPIQEDYRKPEPACSPGGGGSGGGGSRVKFSRSAD |
| | | APAYQQGQNQLYNELNLGRREEYDVLDKRRGRDPEMGGK |
| | | PRRKNPQEGLYNELQKDKMAEAYSEIGMKGERRRGKGHD |
| | | GLYQGLSTATKDTYDALHMQALPPR |
| 128 | aCD19-CD28- | ATGCTGCTGCTGGTCACAAGCCTGCTGCTGTGCGAGCTGC |
| | CD27-CD3-2A- | CCCACCCCGCCTTTCTGCTGATCCCCGACATCCAGATGAC |
| | iCasp9 | CCAGACCACCAGCAGCCTGAGCGCCAGCCTGGGCGACAG |
| | | AGTGACCATCAGCTGCCGGGCCAGCCAGGACATCAGCAA |
| | | GTACCTGAACTGGTATCAGCAGAAACCCGACGGCACCGT |
| | | GAAGCTGCTGATCTACCACACCAGCCGGCTGCACAGCGG |
| | | CGTGCCCAGCAGATTTTCTGGCAGCGGATCTGGCACCGA |
| | | CTACAGCCTGACCATCTCCAACCTGGAACAGGAAGATAT |
| | | CGCTACCTACTTCTGTCAGCAGGGCAACACCCTGCCCTAC |
| | | ACCTTCGGCGGAGGCACCAAGCTGGAAATCACCGGCAGC |
| | | ACCAGCGGCTCCGGCAAGCCTGGATCTGGCGAGGGCAGC |
| | | ACCAAGGGCGAAGTGAAGCTGCAGGAAAGCGGCCCTGG |
| | | CCTGGTCGCCCCTAGCCAGAGCCTGTCCGTGACCTGTACC |
| | | GTGTCCGGCGTGTCCCTGCCCGACTACGGCGTGTCCTGGA |
| | | TCAGACAGCCCCCCAGAAAGGGCCTGGAATGGCTGGGCG |
| | | TGATCTGGGGCAGCGAGACAACCTACTACAACAGCGCCC |
| | | TGAAGTCCCGGCTGACCATCATCAAGGACAACAGCAAGA |
| | | GCCAGGTGTTCCTGAAGATGAACAGCCTGCAGACCGACG |
| | | ACACCGCCATCTACTACTGCGCCAAGCACTACTACTACG |
| | | GCGGCAGCTACGCCATGGACTACTGGGGCCAGGGCACCA |
| | | GCGTGACAGTCTCTTCTGCGGCCGCAATTGAAGTTATGTA |
| | | TCCTCCTCCTTACCTAGACAATGAGAAGAGCAATGGAAC |
| | | CATTATCCATGTGAAAGGGAAACACCTTTGTCCAAGTCC |
| | | CCTATTTCCCGGACCTTCTAAGCCCTTTTGGGTGCTGGTG |
| | | GTGGTTGGGGGAGTCCTGGCTTGCTATAGCTTGCTAGTAA |
| | | CAGTGGCCTTTATTATTTTCTGGGTGAGGAGTAAGAGGA |
| | | GCAGGCTCCTGCACAGTGACTACATGAACATGACTCCCC |
| | | GCCGCCCTGGGCCCACCCGCAAGCATTACCAGCCCTATG |
| | | CCCCACCACGCGACTTCGCAGCCTATCGCTCCGCTAGCG |
| | | GAGGTGGAGGTTCTGGAGGTGGTGGAAGTCAAAGAAGG |
| | | AAGTACCGCAGCAACAAAGGAGAATCTCCCGTCGAGCCA |
| | | GCCGAGCCCTGTCATTATTCATGCCCAAGGGAGGAGGAG |
| | | GGAAGTACAATCCCAATTCAAGAAGACTACAGGAAGCCC |
| | | GAACCTGCATGCAGTCCAGGTGGAGGCGGTTCTGGAGGC |
| | | GGTGGCTCCCGGGTGAAATTCTCACGGTCTGCAGACGCA |
| | | CCCGCTTACCAGCAAGGCCAGAACCAACTCTATAACGAG |
| | | CTCAATCTAGGACGAAGAGAGGAGTACGATGTTTTGGAC |
| | | AAGAGACGTGGCCGGGACCCTGAGATGGGGGGAAAGCC |
| | | GAGAAGGAAGAACCCTCAGGAAGGCCTGTACAATGAAC |
| | | TGCAGAAAGATAAGATGGCGGAGGCCTACAGTGAGATTG |
| | | GGATGAAAGGCGAGCGCCGGAGGGGCAAGGGGCACGAT |
| | | GGCCTTTACCAGGGTCTCAGTACAGCCACCAAGGACACC |
| | | TACGACGCCCTTCACATGCAGGCCCTGCCCCCTCGCACTA |
| | | GTGGCTCCGGAGCCACGAACTTCTCTCTGTTAAAGCAAG |
| | | CAGGAGACGTGGAAGAAAACCCCGGTCCCATGGGAGTG |
| | | CAGGTGGAAACCATCTCCCCAGGAGACGGGCGCACCTTC |
| | | CCCAAGCGCGGCCAGACCTGCGTGGTGCACTACACCGGG |
| | | ATGCTTGAAGATGGAAAGAAAGTGGACTCCTCCCGGGAC |
| | | AGAAACAAGCCCTTTAAGTTTATGCTAGGCAAGCAGGAG |
| | | GTGATCCGAGGCTGGGAAGAAGGGGTTGCCCAGATGAGT |
| | | GTGGGTCAGAGAGCCAAACTGACTATATCTCCAGATTAT |
| | | GCCTATGGTGCCACTGGGCACCCAGGCATCATCCCACCA |
| | | CATGCCACTCTCGTCTTCGATGTGGAGCTTCTAAAACTGG |
| | | AAGGTGGAGGCGGTTCAGGCGGCGGCGGCAGCGGCGCC |
| | | ATGGTCGGTGCTCTTGAGAGTTTGAGGGGAAATGCAGAT |
| | | TTGGCTTACATCCTGAGCATGGAGCCCTGTGGCCACTGCC |
| | | TCATTATCAACAATGTGAACTTCTGCCGTGAGTCCGGGCT |
| | | CCGCACCCGCACTGGCTCCAACATCGACTGTGAGAAGTT |
| | | GCGGCGTCGCTTCTCCTCGCTGCATTTCATGGTGGAGGTG |
| | | AAGGGCGACCTGACTGCCAAGAAAATGGTGCTGGCTTTG |
| | | CTGGAGCTGGCGCGGCAGGACCACGGTGCTCTGGACTGC |
| | | TGCGTGGTGGTCATTCTCTCTCACGGCTGTCAGGCCAGCC |
| | | ACCTGCAGTTCCCAGGGGCTGTCTACGGCACAGATGGAT |
| | | GCCCTGTGTCGGTCGAGAAGATTGTGAACATCTTCAATG |
| | | GGACCAGCTGCCCCAGCCTGGGAGGGAAGCCCAAGCTCT |
| | | TTTTCATCCAGGCCTGTGGTGGGGAGCAGAAAGACCATG |
| | | GGTTTGAGGTGGCCTCCACTTCCCCTGAAGACGAGTCCCC |
| | | TGGCAGTAACCCCGAGCCAGATGCCACCCCGTTCCAGGA |
| | | AGGTTTGAGGACCTTCGACCAGCTGGACGCCATATCTAG |
| | | TTTGCCCACACCCAGTGACATCTTTGTGTCCTACTCTACT |
| | | TTCCCAGGTTTTGTTTCCTGGAGGGACCCCAAGAGTGGCT |
| | | CCTGGTACGTTGAGACCCTGGACGACATCTTTGAGCAGT |
| | | GGGCTCACTCTGAAGACCTGCAGTCCCTCCTGCTTAGGGT |
| | | CGCTAATGCTGTTTCGGTGAAAGGGATTTATAAACAGAT |
| | | GCCTGGTTGCTTTAATTTCCTCCGGAAAAAACTTTTCTTT |
| | | AAAACATCAGCTAGT |
| 129 | iCasp9 | GSGATNFSLLKQAGDVEENPGPMGVQVETISPGDGRTFPKR |
| | (synthetic | GQTCVVHYTGMLEDGKKVDSSRDRNKPFKFMLGKQEVIRG |
| | construct) | WEEGVAQMSVGQRAKLTISPDYAYGATGHPGIIPPHATLVF |
| | | DVELLKLEGGGGSGGGGSGAMVGALESLRGNADLAYILSM |
| | | EPCGHCLIINNVNFCRESGLRTRTGSNIDCEKLRRRFSSLHF |
| | | MVEVKGDLTAKKMVLALLELARQDHGALDCCVVVILSHG |
| | | CQASHLQFPGAVYGTDGCPVSVEKIVNIFNGTSCPSLGGKP |
| | | KLFFIQACGGEQKDHGFEVASTSPEDESPGSNPEPDATPFQE |
| | | GLRTFDQLDAISSLPTPSDIFVSYSTFPGFVSWRDPKSGSWY |
| | | VETLDDIFEQWAHSEDLQSLLLRVANAVSVKGIYKQMPGCF |
| | | NFLRKKLFFKTSAS |

The degree of sequence identity between two sequences can be determined, for example, by comparing the two sequences using computer programs designed for this purpose, such as global or local alignment algorithms. Non-limiting examples include BLASTp, BLASTn, Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, SSEARCH2SEQ, or another suitable method or algorithm. A global alignment algorithm, such as a Needleman and Wunsch algorithm, can be used to align two sequences over their entire length, maximizing the number of matches and minimizes the number of gaps. Default settings can be used.

To generate similarity scores for two amino acid sequences, scoring matrices can be used that assign positive scores for some non-identical amino acids (e.g., amino acids with similar physio-chemical properties and/or amino acids that exhibit frequent substitutions in orthologs, homologs, or paralogs), Non-limiting examples of scoring matrices include PAM30, PAM70, PAM250, BLOSUM45, BLOSUM50, BLOUM62, BLOSUM80, and BLOSUM90.

Amino acids can include genetically encoded and non-genetically encoded occurring amino acids. Amino acids can include naturally occurring and non-naturally occurring amino acids. Amino acids can be L forms or D forms. Substitutions disclosed herein can include conservative and/or non-conservative amino acid substitutions. A conservative amino acid substitution can be a substitution of one amino acid for another amino acid of similar biochemical properties (e.g., charge, size, and/or hydrophobicity). A non-conservative amino acid substitution can be a substitution of one amino acid for another amino acid with different biochemical properties (e.g., charge, size, and/or hydrophobicity). A conservative amino acid change can be, for example, a substitution that has minimal effect on the secondary or tertiary structure of a polypeptide. A conservative amino acid change can be an amino acid change from one hydrophilic amino acid to another hydrophilic amino acid. Hydrophilic amino acids can include Thr (T), Ser(S), His (H), Glu (E), Asn (N), Gln (Q), Asp (D), Lys (K) and Arg (R). A conservative amino acid change can be an amino acid change from one hydrophobic amino acid to another hydrophilic amino acid. Hydrophobic amino acids can include Ile (I), Phe (F), Val (V), Leu (L), Trp (W), Met (M), Ala (A), Gly (G), Tyr (Y), and Pro (P). A conservative amino acid change can be an amino acid change from one acidic amino acid to another acidic amino acid. Acidic amino acids can include Glu (E) and Asp (D). A conservative amino acid change can be an amino acid change from one basic amino acid to another basic amino acid. Basic amino acids can include His (H), Arg (R) and Lys (K). A conservative amino acid change can be an amino acid change from one polar amino acid to another polar amino acid. Polar amino acids can include Asn (N), Gln (Q), Ser(S) and Thr (T). A conservative amino acid change can be an amino acid change from one nonpolar amino acid to another nonpolar amino acid. Nonpolar amino acids can include Leu (L), Val (V), Ile (I), Met (M), Gly (G) and Ala (A). A conservative amino acid change can be an amino acid change from one aromatic amino acid to another aromatic amino acid. Aromatic amino acids can include Phe (F), Tyr (Y) and Trp (W). A conservative amino acid change can be an amino acid change from one aliphatic amino acid to another aliphatic amino acid. Aliphatic amino acids can include Ala (A), Val (V), Leu (L) and

Ile (I). In some embodiments, a conservative amino acid substitution is an amino acid change from one amino acid to another amino acid within one of the following groups: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu.

A protein or polypeptide disclosed herein can comprise an N-terminal methionine. A protein or polypeptide disclosed herein can lack an N-terminal methionine.

### VI. Examples

### D. Example 1: Illustrative Expression Regulatory Regions

Expression regulatory regions were designed using elements related to human and/or mouse CD3 or CD4 for expression in human and mouse T cells.

A first expression regulatory region, denoted CD4pmE, utilized a human CD4 (IMD79, OKT4D, CD4mut) promoter fused a mouse CD4 enhancer. A second expression regulatory region, denoted mCD34, utilized the murine promoter for Cd3d/T3d. A third expression regulatory region, denoted hCD3Y, utilized a human CD3 gamma (CD3G, T3G, IMD17, CD3GAMMA; CD3-GAMMA) promoter fragment. Plasmids were constructed with each of the expression regulatory regions driving expression of an EGFP reporter.

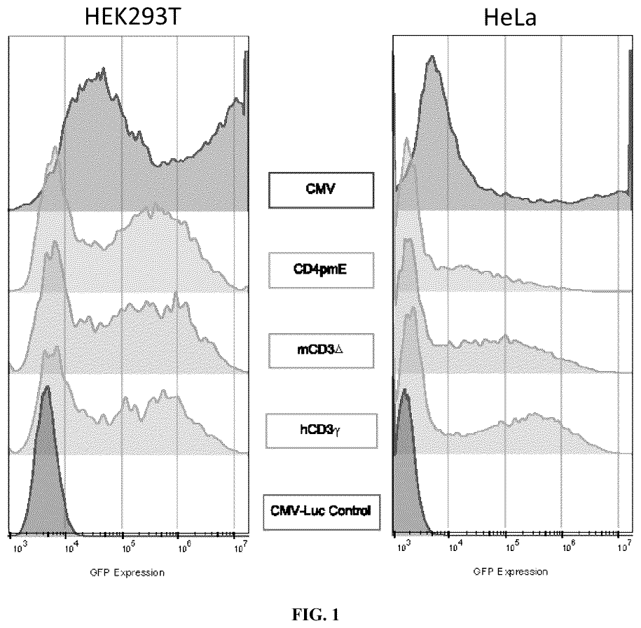

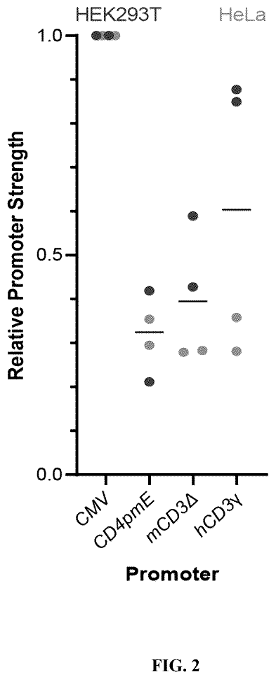

Each of the promoters was shown to be active in both HEK293T cells and HeLa cells compared to control when coupled to an EGFP reporter (FIG. 1 and FIG. 2). The cells were transiently transfected with EGFP reporter plasmids under the control of the indicated promoters. 48 hours post transfection, the cells were stained with E660 viability stain and evaluated by flow cytometry. EGFP fluorescence of viable cells is depicted as off-set histograms (FIG. 1). CMV-Luciferase was used to establish the EGFP negative gate among viable cells. FIG. 2 shows fluorescence intensity (MFI) of the viable, EGFP positive populations for each promoter normalized to the MFI of CMV-EGFP transfected cells.

In some embodiments, certain cell lines including HEK293T and HeLa cells exhibit altered promoter activity as compared to primary cells, for example, can exhibit expression driven from promoters that would not normally be active in a cell type of origin. Expression in these cells can demonstrate functionality of an expression construct but may not reflect specificity in primary cells. For example, in some embodiments, a CD3 (e.g., CD3 gamma or CD3 delta) or CD4 promoter disclosed herein does not elicit or substantially does not drive expression in a primary cell that is substantially CD3 negative or substantially CD4 negative, respectively.

### E. Example 2: 1D3 CAR Expression

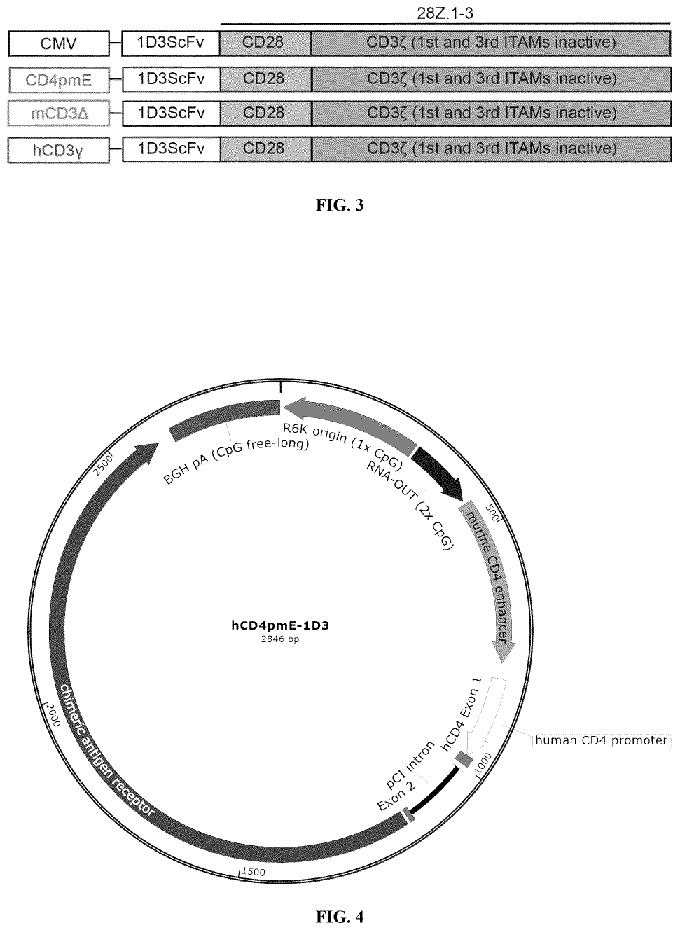

The expression regulatory regions described in EXAMPLE 1 were used to drive expression of a murine anti-CD19 chimeric antigen receptor (CAR). The CD19 CAR was composed of the scFv of clone 1D3 (specific for mouse CD19), fused to the transmembrane and cytoplasmic domain of murine CD28 and the cytoplasmic domain of murine CD3 zeta, where the 1st and 3rd ITAMs are inactivated by double YY to FF mutations (FIG. 3).

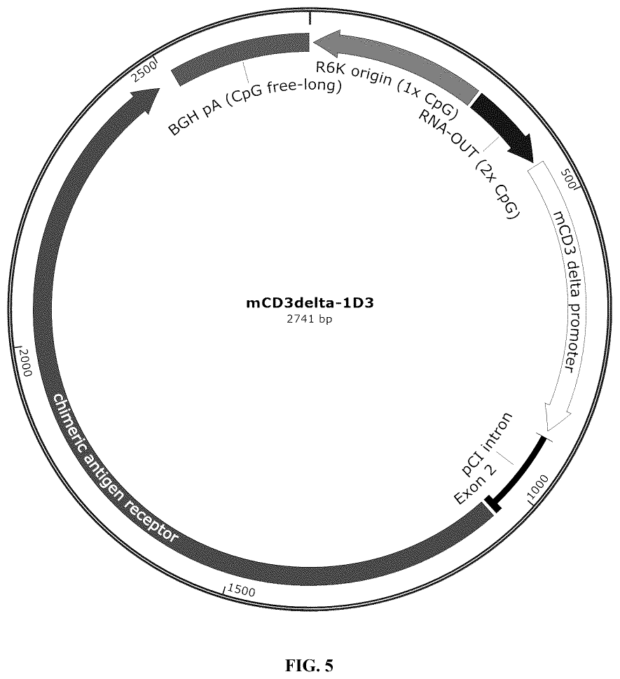

The CAR sequence was cloned downstream of the chosen expression regulatory region into a Nanoplasmid backbone. The expression regulatory regions further comprised exon and intron sequences as shown in FIG. 4 (CD4pmE expression regulatory region), FIG. 5 (mCD3delta) and FIG. 6 (hCD3gamma).

HEK293T or HeLa cells were transfected with the indicated plasmids using Lipofectamine 3000 according to a standard protocol. 48 hours later 1D3 expression was detected by Western blot with anti-CD247 antibody.

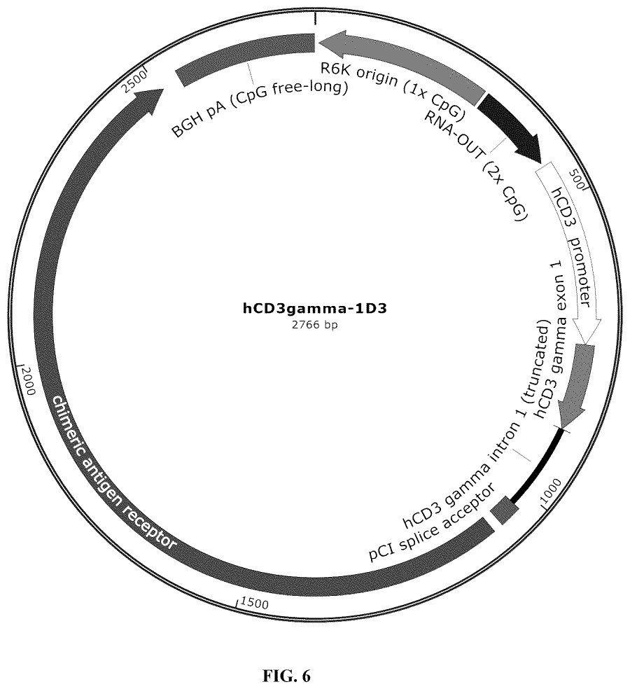

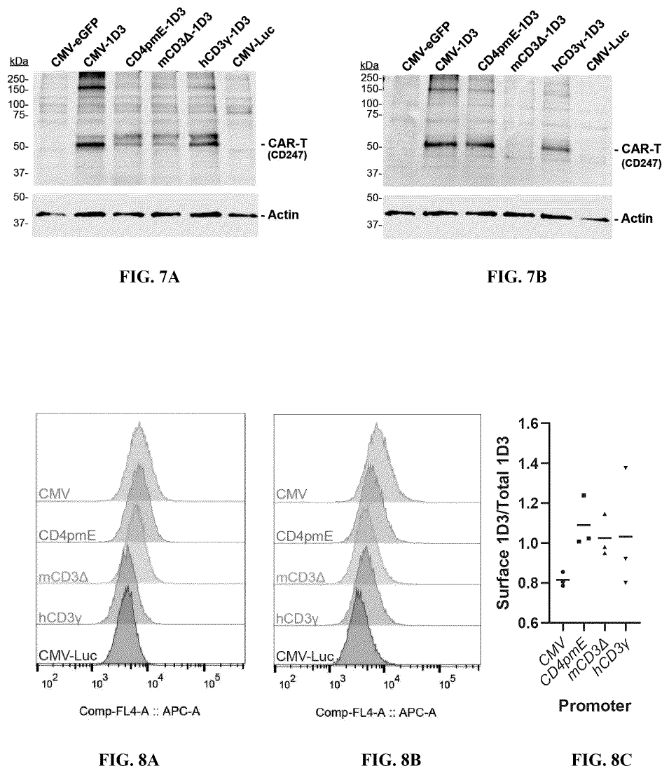

Expression of the ˜50 kDa CAR was detected by western blot using an antibody for the cytoplasmic domain of CD3C/CD247 (FIG. 7A (HEK293T) and FIG. 7B (HeLa)). Some higher molecular weight bands were detected with the strongest promoter (CMV) and with hCD3γ. CAR expression levels generally followed the pattern observed for EGFP.

Surface and total cell expression of the CAR was evaluated by flow cytometry with staining for the 1D3 ScFv domain using an anti-Rat IgG antibody. For total cell staining, cells were fixed and permeabilized prior to staining.

Surprisingly, similar cell surface levels of CAR expression were observed for the four promoters (FIG. 8A). Total cell staining showed an expression pattern more similar to western blot and reporter assays, with higher CAR expression from the CMV promoter (FIG. 8B). Quantification of relative surface vs total CAR expression suggested that a higher proportion of CAR was surface expressed when driven by the CD4pmE, mCD34, or hCD3Y promoter (FIG. 8C).

These results demonstrate expression and surface localization of a CAR in human cells when expression is driven by the CD4pmE, mCD34, or hCD3γ promoter.

### F. Example 3: In Vivo Transduction of T Cells Using LDV Encapsulated Nanoplasmids

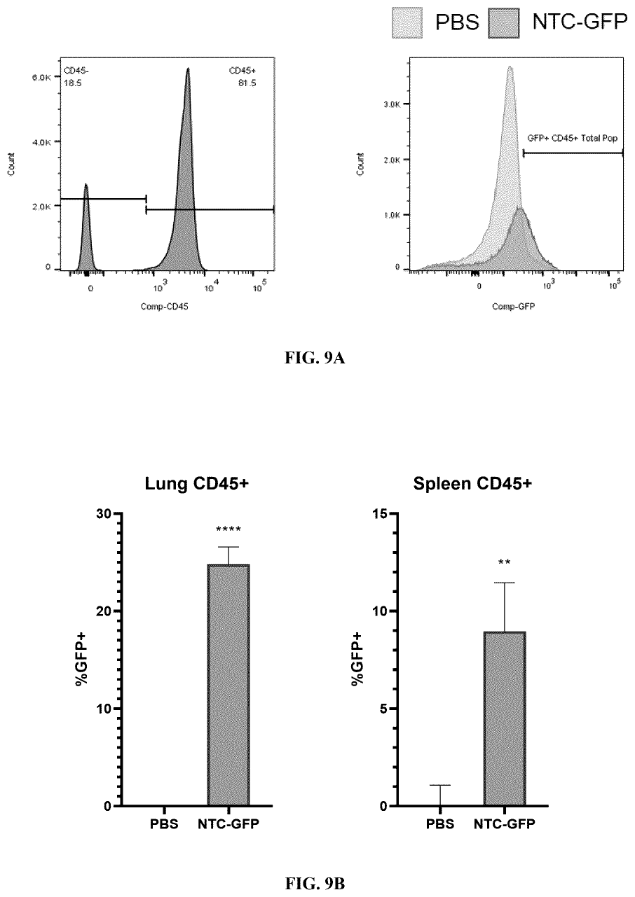

To test delivery of plasmid DNA cargoes to T cells, mice were administered lipid-based delivery vectors (LDVs) encapsulating a nanoplasmid reporter construct expressing GFP from a CMV promoter. Groups of three animals were administered either vehicle or 20 mg/kg CMV-GFP-LDVs on days 0 and 6. On Day 7 (24 hours post second dose), lungs and spleen were collected from each animal and the frequencies of GFP positive T cells were determined by flow cytometry. LDV-treated animals demonstrated a sub-population of GFP positive, CD45 positive (nucleated hematopoietic) cells compared to PBS control (FIG. 9A). CD45+ cells in the lungs and spleen demonstrated mean GFP positive frequencies of 25% and 9%, respectively (FIG. 9B).

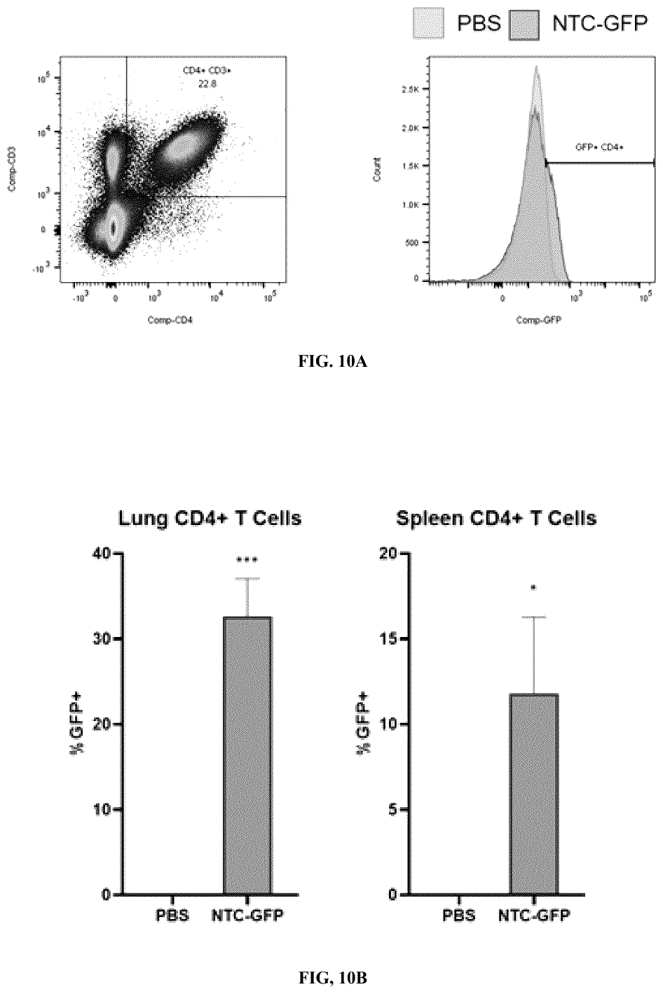

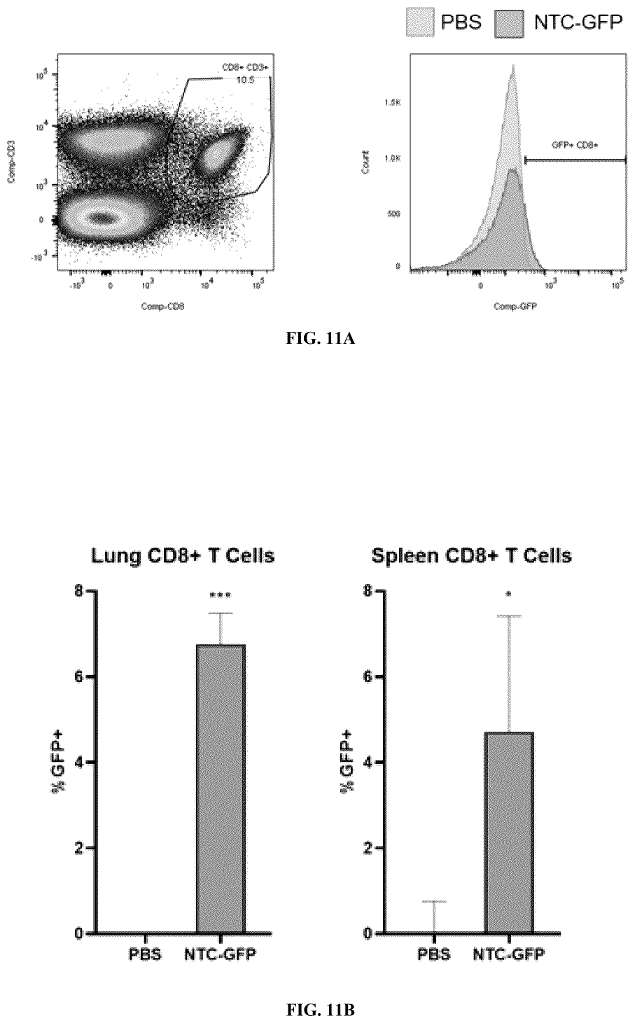

Further analysis of T cells revealed delivery of the GFP reporter cargo and GFP expression in CD4+ T cells of the lung and spleen (FIG. 10A) at mean frequencies of 33% and 12%, respectively (FIG. 10B). Similarly, the frequencies of GFP expression in CD8+ T cells of the lung and spleen were 6.5% and 4.5%, respectively (FIG. 11A, FIG. 11B). These data demonstrate that LDVs can effectively deliver nanoplasmid DNA cargoes to T cells in vivo by IV administration.

### G. Example 4: Reduction of B Cells Following In Vivo Administration of LDV Encapsulated Nanoplasmids Encoding Anti-CD19 CAR

The 1D3.28Z.1-3 CAR receptor nanoplasmid constructs driven by either CMV promoter or one of three T cell promoters described in EXAMPLE 1 were encapsulated in FAST-LDVs. The FAST-LDVs were not targeted to any particular cell type. Groups of six mice were administered a single intravenous 20 mg/kg dose of one encapsulated expression construct encoding 1D3 CAR on day 0. Blood and spleen were collected on days 3, 14, and 28 and analyzed by flow cytometry for the frequency of viable B cells among PBMCs and splenocytes.

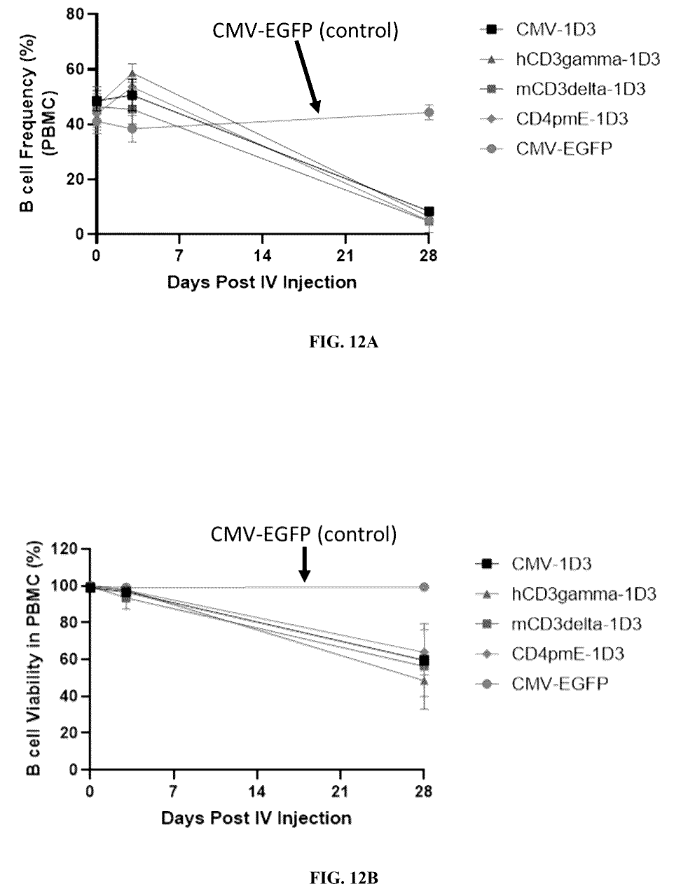

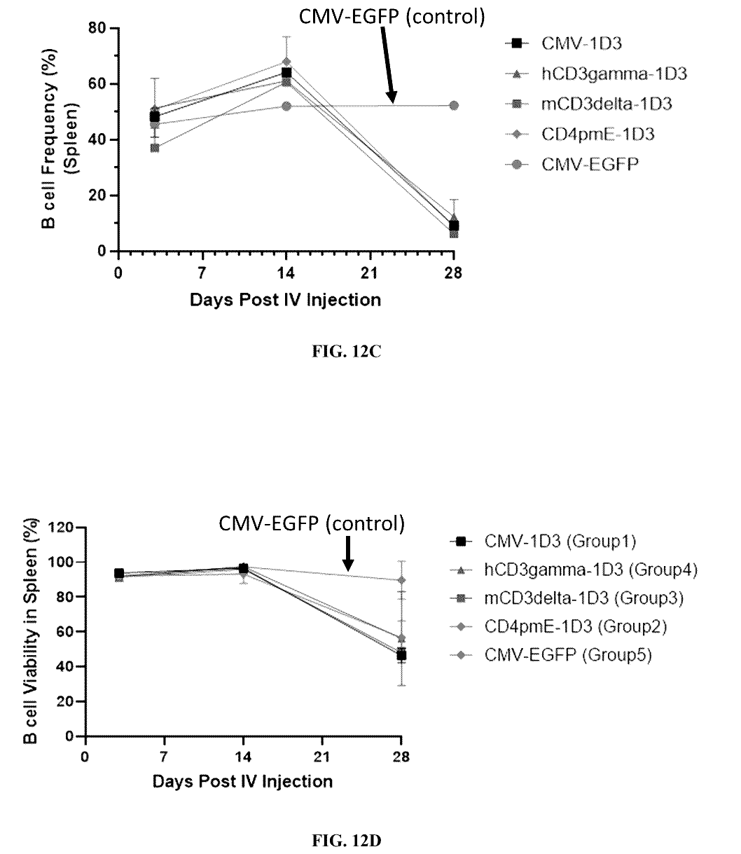

Animals receiving a FAST-LDV encapsulated 1D3 CAR showed normal B cell frequencies in PBMC at day 3; however, by day 28 they showed a 4 to 10-fold decline in the frequency of viable B cells (FIG. 12A). While the viability of detected B220+B cells was normal (>98%) at baseline and at day 3, by day 28 the viability of remaining B cells in the PBMC dropped to approximately 60% in all groups (FIG. 12B). Control animals receiving CMV-GFP LDVs, had no change in the circulating frequency of B cells or their viability at baseline or days 3 and 28.

In the spleen, B cell frequency increased on day 14 but declined 4 to 5-fold at day 28 (FIG. 12C). B cell viability in the spleen remained normal up to day 14 and by day 28 decreased to nearly 50% in CAR-treated animals (FIG. 12D). Again, the control CMV-GFP FAST-LDV treated mice showed normal B cell frequency and viability in the spleen up to day 28.

These results show that in vivo administration of FAST-LDVs encapsulating CAR-encoding nanoplasmids can result in expression of functional CARs in vivo, as demonstrated by reduced frequency and viability of B cells targeted by the CAR. Effects were observed for CARS driven by all four promoters, supporting sufficient T cell CAR expression for in vivo CAR-T functionality after intravenous administration of a single dose of the expression construct.

### H. Example 5: Additional Materials and Methods

Cloning and plasmid constructs: P10-CMV-EGFP was developed in house using gene synthesis and traditional cloning techniques. CD4pmE, mCD34 and hCD3γ promoters were generated by a commercial vendor. The promoters were cloned into p10-CMV-EGFP vector (replacing the CMV promoter) using BamHI and Sall. In a control plasmid, the CMV promoter was left in the expression vector. All reporter constructs were verified by direct sequencing. The 1D3-28Z.1-3 clones were synthesized from the published sequence and cloned directly downstream of the chosen promoters by into a Nanoplasmid backbone.

Cell lines, culture conditions, and transfection: HeLa and HEK293T cells were procured from ATCC and maintained in complete high glucose DMEM supplemented with 10% FBS, 2 mM L-Glutamine and 1×Penicillin/Streptomycin. Cells were maintained at 37° C. and 5% CO2. Cells were transfected with plasmid DNA using Lipofectamine 3000 according to a standard protocol.

Western Blot: Cell lysates were prepared in RIPA buffer. 12-15 ug of total cell lysate was resolved on precast 8-16% Tris-Glycine gels and transferred to nitrocellulose membrane. Membranes were blocked in undiluted Intercept Blocking Buffer and probed with rabbit anti-CD247 (ProteinTech, 1:500 dilution) and mouse anti-β-Actin (LICOR 1:1000) diluted in Intercept T20 Diluent overnight at 4° C. with rotating. Membranes were washed 3 times in PBS-T (0.2% Tween-20). Goat anti-Rabbit IgG (H+L) IRDye680 and goat anti-mouse IgG (H+L) IRDye800 were diluted 1:10,000 in interceptor T20 diluent and incubated with membranes for 45 minutes at room temperature and constant rotating. Membranes were washed three times in PBS-T and once in PBS before image collection using the LICOR Odyssey DLx and Empiria acquisition software.

Flow cytometry: HEK293T and HeLa cells were trypsinized to single cell suspensions prior to staining. Spleens were manually homogenized to single cell suspensions using syringe plungers and sterile petri plates. For EGFP reporter analysis single cell suspension were stained for viability with E660 fixable viability stain and data acquired live on an AccuriC6+ flow cytometer.

For 1D3 surface and total cell staining, single cell suspensions were stained with Zombie Green™ Fixable Viability Kit per manufacturer's dilution on ice for 5 minutes and quenched by adding 1× MojoSort™ Buffer. When needed cells were fixed cells with 150 μl Cyto-Fast™ Fix/Perm Buffer, washed with 1×Mojo Buffer and spun down at 2000 rpm for 5 minutes at 4° C. Once washed, fixed cells were stored overnight at 4° C. The cells were then split into two conditions-surface stain and total stain. The total stain samples were washed with 1× Cyto-Fast™ perm-wash solution and resuspended in 0.05 mL of perm-wash solution. Cells were stained with an antibody cocktail made with perm buffer and either Jackson Alexa Fluor® 647 Mouse Anti-Rat IgG (H+L) or Biolegend APC Goat anti-rat IgG (minimal x-reactivity), and 0.05 mL of the cocktail was added to cells. The surface stain cells were resuspended in 0.05 mL of 1×Mojo Buffer and 0.05 mL of the antibody cocktail made with 1×Mojo buffer. Buffer was added to the cells. Both were incubated for 20 minutes on ice in the dark. The IC stained samples were washed with 1×Perm Wash three times while the surface stained samples were washed with 1×Mojo Buffer three times. Samples were then filtered and run on an AccuriC6+ flow cytometer.

B cell phenotyping was performed using cells stained with E660 for viability and rat anti-mouse B220 FitC (biolegend). Surface staining was performed as described for 1D3.

Mice: 8-12 week old female C57BL6 mice were procured from a commercial vendor. Animals were housed in an ALAAC certified animal facility. All animal procedures were performed with IACUC oversight. A pre-treatment blood sample was collected 3 days prior to dosing via sub mandibular vein. Mice were given a single 5 mL/kg dose IV (tail vein) of 4 mg/mL LDVs (20 mg/kg final dose). On days 3, 14, and 28, two mice from each group were sacrificed and terminal blood (for PBMC isolation) and spleen were collected for analysis. PBMC were isolated by 2 rounds of Red Blood Cell Lysis buffer according to the manufacturer's directions.

1.-80. (canceled)

81. A system for delivery of an expression construct encoding a heterologous immune receptor, the system comprising:
(a) a non-viral lipid-based delivery vector (LDV); and
(b) a polynucleotide that encodes the heterologous immune receptor, wherein expression of the heterologous immune receptor is driven by an expression regulatory region that comprises an immune cell-specific promoter.

82. The system of claim 81, wherein the immune cell-specific promoter comprises a T cell-specific promoter.

83. The system of claim 81, wherein the immune cell-specific promoter comprises:
(i) a CD3 promoter that natively drives expression of CD3 in a mammalian cell;
(ii) a CD4 promoter that natively drives expression of CD4 in a mammalian cell;
(iii) a distal lymphocyte protein tyrosine kinase (dLck) promoter that natively drives expression of dLck in a mammalian cell; or
(iv) an NKp46 promoter that natively drives expression of NKp46 in a mammalian cell.

84. The system of claim 81, wherein the expression regulatory region further comprises an enhancer.

85. The system of claim 81, wherein the expression regulatory region further comprises an intron.

86. The system of claim 81, wherein the polynucleotide comprises DNA.

87. The system of claim 81, wherein the polynucleotide is a DNA plasmid, nanoplasmid, or minicircle.

88. The system of claim 81, wherein the heterologous immune receptor is a chimeric antigen receptor (CAR).

89. The system of claim 88, wherein the CAR comprises an extracellular binding domain that binds to CD19.

90. The system of claim 81, wherein the heterologous immune receptor is a T cell receptor.

91. The system of claim 81, wherein the polynucleotide further comprises a transgene that encodes an immunomodulatory factor.

92. The system of claim 81, wherein the LDV comprises a fusion-associated small transmembrane (FAST) protein.

93. The system of claim 92, wherein the FAST protein comprises an ectodomain of p14 and an endodomain of p15.

94. The system of claim 81, wherein the LDV is formulated for non-targeted delivery to T cells and non-T cells.

95. The system of claim 81, wherein the LDV is formulated for targeted delivery to T cells.

96. A method of expressing a CAR in a cell, the method comprising contacting the cell with the system of claim 81.

97. The method of claim 96, wherein the contacting is in vivo.

98. The method of claim 96, wherein the CAR is expressed transiently by the cell.

99. A system for delivery of an expression construct encoding a heterologous immune receptor, the system comprising:
(a) a lipid-based delivery vector (LDV) that comprises a fusion-associated small transmembrane (FAST) protein; and
(b) a polynucleotide that encodes the heterologous immune receptor.

100. An expression construct for delivery of a heterologous immune receptor, the expression construct comprising a polynucleotide that comprises:
(a) a transgene that encodes the heterologous immune receptor; and
(b) an expression regulatory region, wherein the expression regulatory region comprises:
(i) a T cell-specific promoter, and
(ii) an enhancer or an intron.


SEQUENCE LISTING (130 sequences)

SEQ ID NO: 1 - DNA, 169 bp, Homo sapiens
  source 1..169: mol_type = genomic DNA
gatttaagcctgattctgcttaactttttcccttgactttggcattttcactttgacatgttccctgagagcctggggggtggggaaccagctccagctggtgacgtttggggccggcccaggcctagggtgtggaggagccttgccatcgggcttcctgtctctcttc

SEQ ID NO: 2 - DNA, 302 bp, Homo sapiens
  source 1..302: mol_type = genomic DNA
aacaactggctacgatcctaacaactaatgacagggacatttatctccctcatgaagaaacggtcccaggaccatctcccacccagcatccattgcggttccctgtgcaagatgagtctctgagtgggaatccagcactctctccctcttcttccccaccaccttcaccctccttaacggaaaaacaaaaggcatctgcacctgcagccctgctgaggcccctgctgctcacacttgcagcagagggtggaggctctgggttcttgccttctctcaaaggccccagccccaacagtgatggg

SEQ ID NO: 3 - DNA, 454 bp, Mus musculus
  source 1..454: mol_type = genomic DNA
tgatcagaaacaagaggatctgctaatgtggcttgtgtaatagtttctctcagtggatctaaagaggtggcacttccttgtttgtgagtaaatgtagttgtattgttcataggaagcccaaaatcttctttgtttgttaccagtgactattgctatttgttttttgttaccaggaaaggctattctaaattcccttacacttcctagaattctgaacaaactttcctcatagtctctgctttgtatcaatcatcccaaactctttgcttcagaaagagcagtttgcacctgtttgtgtcttaccccacacctagcccactgttgctttgacagtcttacaccatcaaaggcagaaggtagagaggcagatttcttctagttcctcccccactcttcctttttctgaatacccatgagtctgttggcggaggatagtgctaaccctggctgatca

SEQ ID NO: 4 - DNA, 120 bp, Cytomegalovirus
  source 1..120: mol_type = genomic DNA
caccaaaatcaacgggactttccaaaatgtcgtaacaactccgccccattgacgcaaatgggcggtaggcgtgtacggtgggaggtctatataagcagagctcgtttagtgaaccgtcag

SEQ ID NO: 5 - DNA, 1096 bp, Homo sapiens
  source 1..1096: mol_type = genomic DNA
ctgcagcctcaacttcctgggctcaagcaatcctcccacctcggcctcctaaaatactgggattataggcatgagccaccactcccagcaccacttttttcagactggaaaagaacactcacatgtgcatctttaaatgacacttgggctgtggtatggagaatggccaccagtgagtaggcaggagctgttgtccgagcaagggctgatattggcatcttggattggcatggtggcagtagtggtagtgcagagtgacttgggtagattttggagcatttagaaggtacatccacaggaactggtaaataaatacgtgggagaagttgggtgaagggggtgtcaaagattacacccaatttattttgcttgggaagttggtggatggtgagcccctcactgagtgagaagcctggagaagcaggtttggagggtggtagtatgcaggtggtatgcatagttgggatgtgtgttgagtttgctatgtccggtgagcttcccagtggagatgtccaatgggcagacggatactcacatagagagttcatggtagattcgggctagaggaaagcacctgaggcctggccagagacgcctagaggaacagagcctggttaacagtcactcctggtgtctcagatattctctgctcagcccacgccctctcttccacactgggccacctataaagcctccacagatacccctggggcacccactggacacaattgccctcagggccccagagcaaggagctgtttgtgggcttaccactgctgttcccatatgcccccaactgcctcccacttctttccccacagcctggtcagacatggcactaccactaatggaatctttcttgccatctttttcttgccgtttaacagtggcagtgacacttgactcctgattaagcctgattctgcttaactttttcccttgactttggcattttcactttgacatgttccctgagagcctggggggtggggaaccagctccagctggtgacgtttggggccggcccaggcctagggtgtggaggagccttgccatcgggcttcctgtctctcttcatttaagcacgactctgcag

SEQ ID NO: 6 - DNA, 492 bp, Mus musculus
  source 1..492: mol_type = genomic DNA
tctagaacagccgacttagtggatctgttgggcaacgtaatctaaattttccattgattaaattgctaacttttttttttaaaacatggcattactgcataggcagcctagtctggccttgagcttgtgatttttctgcctaaatcacccagtgctggggttacagttacatgcccgtgcccgtgcccgtgcctgtaagccttgcctcacgttgacctaaccaggctgtttcagctttttacggcaccaaacaatagtgacattccagactccagcttgattctgctcaggcattttcagtgactttggcatttttaatttttcaacttccccaacaactgggggtgggagggagggactcctgagggctggcttacgtccgccgtgcagaggagcctcacgaccaggcttcctgtcttttcatttacgaacatctgtgaaggcaaagcaagactctcttcttcactaggtacctgtttgcaaagtctcgag

SEQ ID NO: 7 - DNA, 1469 bp, Homo sapiens
  source 1..1469: mol_type = genomic DNA
tgctgggtgggagatggtcctgggaccgtttcttcatgagggagataaatgtccctgtcattagttgttaggatcgtagccagttgttacttgttgctagttgttgttgctgtaggtaatgaatttaggagggactctcagatgcttggggctggacctccaaatggaaagccacctcagtccaggccttcattcaggaatttttttcactttttttttctttttcttttttgagacagagtctcactttgttgcccaggctggagtgcagtggcgctatctcagctcactgcaacctccacctccggggttcaagcgattctcctgcctcagcctcctgagtagctgagattacaggtgcgtgccagcaagcccggctaatttttgtatttttagtagagacggggtttcaccttgttggtcaggctggtctcgaactcctgaccttgtgatccacctgcctcagcctcccaaagtgttgggattacaggcgtgagacacggcacctggtttcactattcttttgagatcatgaaagaggcgcctggaaggcaataggtggaacaattccgaggacaggccagcacattttcaggggccagagcccggactgaatgtccaggatctcaggcccctatctccctcagccagtcagtcagtgagccaacaaaaatgtgcagagctcccgttatgtgccaggcacaagatacagcagtgagcaagacaggcatgactctgtccccaggtaagtgacagtctaactgcagcagcgtgaagagtttcagatcctcgtaggccacaggggaaaaaggtccatttgtctcaccgggaggctcaatattgcctgtcagtcaattatattattattaataactaccccttaatgagcacatatgatgcgtgaggcaccatgcaaatatcttgtttgaatcctaataatggtactaatggaataagactctgaaaaattttatggctggcccaggccccacaggtaggcagtattggacccaggattcaaatctctggctggggtctctaaagcccaacctcccactgacaagaagctgctagatctggtgtccctggctgcctagtgaagggtcctgagaaagatcagcctccatgagaaatctagctgctacggcttgcgctatggggccgacggcttctctcaaggggcttcgagatgtggcagtgtttaggttgtgtgtaaatgtggttgcattgtcaatagggacgctaaagttcaggccaccttttccatattctctgccagctccctgctcagagatagagcaatttacaccgcttccttcctaccctacccctagcccacccccactctgaaaatttcccaccatcaacggcagaaagcagagaagcagacatcttctagttcctcccccactctcctctttccggtacctgtgagtcagctaggggagggcagctctcacccaggctga

SEQ ID NO: 8 - DNA, 1473 bp, Mus musculus
  source 1..1473: mol_type = genomic DNA
gccacccagccacccagccacccagacagcctgtgcagcagctggcctggctccacccagcatgcattgagctgggactctgagtagaggcaataccaggtgtctccgccctctcctgtagagtgtgggcagcaggggccccagcaggggtgttgatgctctgacttatctcgttttctcttttgttgttgttaaggcttgcaggatagcaggtggcaggggatgaggttctagattcccacttggaaacccctttaccactagaacctctatgcgcaataggttgaagactccactgagcctttttctcgatgacagtgatgaatgttcctgcctgttcttctggctaggtctggacttgtttgtcacttgatcccagaactcatgggtttaggcagaacttttccatgtatgagactggagttctgaatggaaaagcatcttagagtagaccttccttaagagatttttgacactgttcttttgagaacatctaagtggtacccacctagaagagaacatcccaacgggacaactagcttatttcttggtgtatgaccctgggctagttccagggtctcaggctattgtctcctttagccagtcaatgcataacagaaattcacagaattcctatgtgattccaggcacaaaggataattttgtccccagacagatgaccttctgattatagcagtgtggaaagttagatccccacagcacatggtagaaaagtctattgtctcctggggaggatcgattctacaaatgagtggattgattatattgttaagaagactctcatactgagatggcttaatgtggctggcatgatgcaaacactcctcccccaatccttagagtgatgctgagggagacaagctctgagacactccatgcatgggtactatgtgctggacctaggattccgatgtctggatgagggcttctcaagtccatcttcccactggctagaagcagcttgatgtggtgtcccagagtgtcctgtgaagggtcctgagactgatcagaaacaagaggatctgctaatgtggcttgtgtaatagtttctctcagtggatctaaagaggtggcacttccttgtttgtgagtaaatgtagttgtattgttcataggaagcccaaaatcttctttgtttgttaccagtgactattgctatttgttttttgttaccaggaaaggctattctaaattcccttacacttcctagaattctgaacaaactttcctcatagtctctgctttgtatcaatcatcccaaaatctttgcttcagaaagagcagtttgcacctgtttgtgttttaccccacacctagcccactgttgctttgacagtcttacaccatcaaaggcagaaggtagagaggcagatttcttctagttcctcccccactcttcctttttctgaatacccatgagtctgttggcggaggatagtgctaaccctggct

SEQ ID NO: 9 - DNA, 1076 bp, Mus musculus
  source 1..1076: mol_type = genomic DNA
ctgctctacagaatgaatttcaggtctcaaggtcagcttggtctacaaagtgagtttcagatctcaaggccagccaagactatgcagtaagaccttagctaaaataaataaataaataaaataaaataaaaagttagatcttcgcttggcaaaccgatagattgaggaccagtgctcaggaaggaggcacacggaattccagaggctacagagggagcctcgctctgacctggttagagcaactctactttactggctgtgtctatgaggttctgcttgatttcatttgacaaaaagtttccacagctaaaccaggcaagggagccgaagtagacacagccacccgggccgcgccaacaggtttctctctgctgctgagaagcaaaagaatgtttgaagaaactctctgaaggagactgtggttgagtggtgggggtaggggtgctggggttgggctgaggctgagggttgactctaaggagctggaacctctcagcttcggtggctaggcaggggagttgtaatgaagagggacagggtaccctccttggtggaggagggtggaatgaaactctcggtttcccccagaacttggcaaagtgtgtgtgatgtctcccagggtagtcccccaaaggaggaggctaggcagagctgggaggcaggaagtgggtaactagactaacaaagatgcctgcctgtggcggtttgcccatcccaggtgggagggtgggactagccttggcctgggcctcctgtgaacttggtgcttgagggctcagagggaacccagtcaggagcttgaatcccacgattcagcgcttctgtctgcggccaatgggggcctctgagctgacgatctcgggtactttttgtaacttccagaacagggctctaggatgtctgatgttggggcgagtggcttagggccagctccttcaggcctctctacattccttcagggatcatgggctgtgtctgcagctcaaaccctgaagatgactggatggagaacattgacgtgtgtgaaaactgccactatcccatagtcccgctggacagcaagatctcggtaagag

SEQ ID NO: 10 - DNA, 4654 bp, Mus musculus
  source 1..4654: mol_type = genomic DNA
gaattcagttgctcgctatcttatttaaagagccaaatctggagaagtagaccgtagagcacaggttctcaatctgtgggtcgcaacccctttgggcatccaaaagcacttttagacaggttgcatatcagatatccagtatatcagatatttatatttaaaattatttaaaagtcgtagcagtggcaagattacggttacaaagtagcaacgaaaataattttatgcttgggagtcatcacagcatgaggaaatgagctgtattaacgggtcccagcattaggaaggctgagaaccactgccctaaagagaagagacagtcaagtgaaaagcagaaagcgaccctgctgtcagttataatctgaggaggggagagttgaatattagctgtcagcccaccatgtgcaaacataaggatgtccagttcagctcaacgggtcatcacagttaatttgtgatggcgtaccagacgtatatatttgcatactaacggttccttggcttggcttgagaatcacccatccggaaaaagatctgcgagaccgaggcggatgaagttaggtgagaaggggattgcgagctggactatgcaaagagtattcccagaaatggaccggcggctgggctggtgagcccggaggctaatctagcaaatttacgcccccttttggtcacagcaggagatgcgggctcaggaccggcagctggcagggcagctgctcaggctgcgggcccggctgcacagactgaaagtggaccaagtctgtcacctgcaccaggagcttctggatgaggctgagctggagatggagttagagtctgggactggcttgcctctggccccaccgctgcggcatctgggactcacgcgcatgaacatcagtgccagacgcttcaccctctgctgacagcagacttgggtgtctcttgcagtatctggggagaaagaaggaaggaagagggaccccggaggctctggctacctgctggggaaggtgggcacacttaggtttccaaaagctgaatttagagagcacaggatggaggggaggaggagaggaaactcgggccccaaatgtcttaataaaaaatgcattgaatcccatcaaggtttctgtagactgtcacagagcctaaataaatgttgttgtatattcatcctgtgtcactgggactttagggattccacaacaggagacaagatggcaccaggtgtccccaacagctcccgatctatttctctaactttatcaaaatcaaaagtgagaaaaatttaaatgaagatgaagattcattgtgttttttgattgtgtgtgtgaccagagaggccagaaaaggattagctgagctggagacacaagctgttgtaagctgcctgttgtggatgctgcctacggaactcaggtcctctgtgagcagcaaagggctttttttctccagcccccaaacttaaaatcttctgccattctcatgacaggtggtctaggtaaattgaagacttggagaaagcagaaagtggtagttctgtgtcatctctgctatttatttattttttctttttctgtttttcttttttttttcttttctttttctctttttccctgatgcagggtctcactaggtaactccgactgtcctgaagcccatactggccttgaactcacagagattcacttgcctccggagtgctggggttaaaggtgtgcaccactctgcccggcttcccctatttaactctattagacacatagtgggaaaatcaggccactatgaggcagatctctatgatcttgacgccagtctgctctacagagcgagttccaggtcagtaaggaccacctagaaagagaccctgtctccaaaaatccaaaaataagcaaacaaacaaattaagaaaaaaacccagctagttgctaatttttgctccacgggcagctagatatgcattactttagaacacacccgataaattcatgcactgcttcttagcaccgtgccccccaggcgggtgctgaagctgaggtgagcagcagtaaaatgctaaggtttctaagtgaggacagcagggcggtatatcaggtctggagacacatctgccggctctgtcactctcagggttgggttgttttttttttttttccttctgtgtgactctcagcctttgtttctgtgtttctctctttgtctgactttccaactcagacttcctcccccccccatttcctgttcccccttccctcagcagctggggtgcacacagcttcagctcctacctcacaaaccccaacaatgctctgtgggctcccctccccctgcctgctcactcagcgtagctcaagaggtgacacatttacactttctttccagtgaggtcaggacccattcttggccctgataggggccatttggggttcctcacatcactggtggcctggccctctgcctgtgagggacacaatcctcatttttgcctgctggcagcttgttgtggagttcttaaactcctgctgatgtcagaatgtctctgcctgccacagatacctgtgcagtatgcttgctcggtcctctaccggaggcaactcgtggttatggctatgcgctccattaggggtgtggccttgcctctacttgaattaccattctgctgtgctgtcctgtcccctgcctgttattctatctatctatctatctatctatctatctatctatctatctatctatctatctattatctatctactatctatctatatctactatctatctatctatctatcttctatctgtctctatcatctatgtatctatctgtgtatctctgtatgtatctatgtttgtatgtatctatctacctatgtatctatgtatctatctatgtatctatgtatctatctctatctatcatctatgtatctatgtatctctgtatgtatatatgtatgtatgtatgtgtgtatgtgtgtatgtatgtatgtatgtgtgtatctacctatctatctatctacctacatctatctatctatctatctatctatctatcgaaacagggtctctttatgtagctctggctgtcctggaacttactgcataagttagtctggcctgaaacttgcagagatctgccagcctctgcctccggaatgctgggattaaagtcttatgccaccatgccccagcctgtcactagggccttaagacatgaggttttcttgtcttcctcctgaaacagactgtcaacagtcgatccttaggcacaggatgacatggatacggctctgccagtgaatgtggctgctgtttttactatagtaccttgtcaccaactatctaacagagatagatactgtccaggttcaactctgaacaagagatgcctttggctgtccggctaaattaaaggccacccagttcttacatagcacggctggagttgagtgcagcgccatttccttccctgtgagctaaactgtagctgctgtgttctgctcacgggggcaaaggctctctttggatctcttacccaggagtatcttggagggtaggaggagtgctccatcaaacagccttccccaccatgtttagtgaccttcacggggaggaagaggcaagtgaatctctgtgagttcaaggcaacatagcaaattccataacagtcagggcgacacggtgagtccctgcgttaaaaaaaagatcctctcattggttgatgacgacgacgacgatgatgatgatgattggacttgaggggtctaggctggtctggaactctgtatgtagccattcctgtcttcacctccagaacgctgggaatatggacctatgccatcacacacagccttcctccctaagtgcctccctcagtatgagtagaagctggcctagagatgtatgggacccaagaggctgagggggtctaggtctatggcagcaggaagcctgaggtgctggaggcttgtggttgggttgctggggctgggttggctgcagagccctcaggagacaggaagtcagggtggaacgtgggcgcgaggagacaggtggtgactacgacggcgaggggagctgacaccgggtccaggcagccaagccaggctaggagcatcatgtgaataggccagaagactcccaggctgggcaggtaaggggtactgggaacaacagtgtagaggggggcaatggtgggaaccaggcaacagaacactgcgtctgtggaggcctaggctccttaggtgtacagaaacctttcagaaattccttcctggaggttgtctgagctggtggctcttgcctctgccatctgctgtccatcatttcccaccttaggaggagcacactcctgggagagccagcacatgatgtcaagtgttaagaaggccaccagcagccacctgtccaggttccttgagctcagaggatagggtttctaggtccaaaggtggctgtgaccctgtcttaacccatagctcctcacgatggtccgcggcaaagcaggaccttgcgtgggggtgtaggcttgacctgggctccttctatgcctgtggcacacagggtgatgagatggaccgtcctgaggcagggccccaggaatcagtccccaggtccctaaggcagacccaagtggcagaaacagatgaaagagtaacccatgggatcc

SEQ ID NO: 11 - DNA, 679 bp, Homo sapiens
  source 1..679: mol_type = genomic DNA
tagcctgccattcacctacccttggctataattaaagcttactggtccaatgccctgaggtggcatgggaaatggtctctcggcctggcctggctgtaacaatgcatggtctgctcaccagctatttaacagggatatgtcctctcctctgactctgacgatgctcctttgctgccaagacagaacaaaggtctctcagctgcaacgtgtgctgagtgctgttgcctctcaccataagctgagtgtgtgtccgcttgccccctgctcactgggcccaaaggctgcccttgaatctcttgcccagatgcaccctggagggcagaagggagggtctatcagacatcctccccctcaactttaaacctcccagtgtcaccctgggacagtaggggaagatggacctggtctggagatgtaggggacccccaggggctgagaggcaggggtctatggtggcaggaagcttggcgtgctagagggttgtggttgggctgctggggcccggttggctgcggagccctccggaggaggcaggaagtcagggtgggacgtgggcgcggggagacaggtggtggctacgacggcgaagggagctgagactgtccaggcagccaggttaggccaggaggaccatgtgaatggggccagagggctcccgggctgggcaggtaaggagcgc

SEQ ID NO: 12 - DNA, 1070 bp, Homo sapiens
  source 1..1070: mol_type = genomic DNA
tgtgtaccctgtgtatatgtatagggtgtgtgtgagtgtgtgtgtgtgagagagtgtgtgtgtggcagaatagactgcggaggtggatttcatcttgatatgaaaggtctggaatgcatggtacattaaactttgaggacagcgctttccaagcactctgaggagcagccctagagaaggaggagctgcagggactccgggggcttcaaagtgagggccccactctgcttcaggcaaaacaggcacacatttatcactttatctatggagttctgcttgatttcatcagacaaaaaatttccactgctaaaacaggcaaataaacaaaaaaaaagttatggccaacagagtcactggagggttttctgctggggagaagcaagcccgtgtttgaaggaaccctgtgagatgactgtgggctgtgtgaggggaacagcgggggcttgatggtggacttcgggagcagaagcctctttctcagcctcctcagctagacaggggaattataataggaggtgtggcgtgcacacctctccagtaggggagggtctgataagtcaggtctctcccaggcttgggaaagtgtgtgtcatctctaggaggtggtcctcccaacacagggtactggcagagggagagggagggggcagaggcaggaagtgggtaactagactaacaaaggtgcctgtggcggtttgcccatcccaggtgggagggtggggctagggctcaggggccgtgtgtgaatttacttgtagcctgagggctcagagggagcaccggtttggagctgggaccccctattttagcttttctgtggctggtgaatggggatcccaggatctcacaatctcaggtacttttggaactttccagggcaaggccccattatatctgatgttgggggagcagatcttggggagccccttcagccccctcttccattccctcagggaccatgggctgtggctgcagctcacacccggaagatgactggatggaaaacatcgatgtgtgtgagaactgccattatcccatagtcccactggatggcaagggcacggtaagag

SEQ ID NO: 13 - DNA, 1346 bp, Homo sapiens
  source 1..1346: mol_type = genomic DNA
tgtgggggtgtgtgggggtgcctgtgtgtgggtgtttgtgtgtgtgtgagtgtgggtgtgtgggggtatctatggaggggatgcctgtgtgtgtgtgtaaggtgagtgtgtggatgtgtgcatgggtgtgggtgagtgtgtggaggtgcctgtgtgtgtgtatgaatgtgtgttggggaggggagaatgtgtgtgtgtggggtgagtgcgtgtgtgtgggggtgaatgcgtgtgtgtgtgtatgtgggtgtgactgtgtgggtgtgtggacgagtgtgtgtgttcctgggtgtgggagcctgtgtgtgtagggggagtatgtatagggtgtgtgcatgaatgtgtgtgtgggtacgtgtgtgagagtgggtgcctgtgtgtggggggtgagtgtgtgtgtgggggggcacttgtggagggtgagtgtatgtgtttactgagtgtgagtgtgggtgcctgtgtgtgggagggtgagtctgtgtgtgagtgtgtgggggagtacctgtgaggggtgagtgtgtgtgtttatgtgaaagtgtgtgtgtgtggatgcctctgtggaggtgggatagggggtgcctctgtgtgtgtgtgtgagagtgtgtgtgtgtagggtgtgtatatgtatagggtgtgtgtgagtgtgtgtgtgtgagagagtgtgtgtgtggcagaatagactgcggaggtggatttcatcttgatatgaaaggtctggaatgcatggtacattaaactttgaggacagcgctttccaagcactctgaggagcagccctagagaaggaggagctgcagggactccgggggcttcaaagtgagggccccactctgcttcaggcaaaacaggcacacatttatcactttatctatggagttctgcttgatttcatcagacaaaaaatttccactgctaaaacaggcaaataaacaaaaaaaaagttatggccaacagagtcactggagggttttctgctggggagaagcaagcccgtgtttgaaggaaccctgtgagatgactgtgggctgtgtgaggggaacagcgggggcttgatggtggacttcgggagcagaagcctctttctcagcctcctcagctagacaggggaattataataggaggtgtggcgtgcacacctctccagtaggggagggtctgataagtcaggtctctcccaggcttgggaaagtgtgtgtcatctctaggaggtggtcctcccaacacagggtactggcagagggagagggagggggcagaggcaggaagtgggtaactagactaacaaaggtgcctgtggcggtttgcccatcccaggtgggagggtggggctagggctcaggggccgt

SEQ ID NO: 14 - DNA, 1551 bp, Homo sapiens
  source 1..1551: mol_type = genomic DNA
tgtgggggtgtgtgggggtgcctgtgtgtgggtgtttgtgtgtgtgtgagtgtgggtgtgtgggggtatctatggaggggatgcctgtgtgtgtgtgtaaggtgagtgtgtggatgtgtgcatgggtgtgggtgagtgtgtggaggtgcctgtgtgtgtgtatgaatgtgtgttggggaggggagaatgtgtgtgtgtggggtgagtgcgtgtgtgtgggggtgaatgcgtgtgtgtgtgtatgtgggtgtgactgtgtgggtgtgtggacgagtgtgtgtgttcctgggtgtgggagcctgtgtgtgtagggggagtatgtatagggtgtgtgcatgaatgtgtgtgtgggtacgtgtgtgagagtgggtgcctgtgtgtggggggtgagtgtgtgtgtgggggggcacttgtggagggtgagtgtatgtgtttactgagtgtgagtgtgggtgcctgtgtgtgggagggtgagtctgtgtgtgagtgtgtgggggagtacctgtgaggggtgagtgtgtgtgtttatgtgaaagtgtgtgtgtgtggatgcctctgtggaggtgggatagggggtgcctctgtgtgtgtgtgtgagagtgtgtgtgtgtagggtgtgtatatgtatagggtgtgtgtgagtgtgtgtgtgtgagagagtgtgtgtgtggcagaatagactgcggaggtggatttcatcttgatatgaaaggtctggaatgcatggtacattaaactttgaggacagcgctttccaagcactctgaggagcagccctagagaaggaggagctgcagggactccgggggcttcaaagtgagggccccactctgcttcaggcaaaacaggcacacatttatcactttatctatggagttctgcttgatttcatcagacaaaaaatttccactgctaaaacaggcaaataaacaaaaaaaaagttatggccaacagagtcactggagggttttctgctggggagaagcaagcccgtgtttgaaggaaccctgtgagatgactgtgggctgtgtgaggggaacagcgggggcttgatggtggacttcgggagcagaagcctctttctcagcctcctcagctagacaggggaattataataggaggtgtggcgtgcacacctctccagtaggggagggtctgataagtcaggtctctcccaggcttgggaaagtgtgtgtcatctctaggaggtggtcctcccaacacagggtactggcagagggagagggagggggcagaggcaggaagtgggtaactagactaacaaaggtgcctgtggcggtttgcccatcccaggtgggagggtggggctagggctcaggggccgtgtgtgaatttacttgtagcctgagggctcagagggagcaccggtttggagctgggaccccctattttagcttttctgtggctggtgaatggggatcccaggatctcacaatctcaggtacttttggaactttccagggcaaggccccattatatctgatgttgggggagcagatcttgggggagccccttcagccccctcttcca

SEQ ID NO: 15 - DNA, 1444 bp, Mus musculus
  source 1..1444: mol_type = genomic DNA
gggtgttgcaggcgtctgagcaggtgacacacagtttcaattcttgtttgtttgtttgtttgtttttcgagacagggtttctctgtgtagacttggctgtcttggaactcactttgtagaccaggctggcctcgaactcagaaatccgcctgcctctgcctcctgagtgttgggattaaaggcctgtgccaccacacccagcttcttttttttttttttaagatttatttttatttatttcatgtatgtgactacactgtagctgtcttcagacacaccagaagagggtctcagatcccattacagatggttgtgagtcaccatgtgattgctgggaattgaactcaagacctctggaagagcactcagtgctctttttgtttgtttgtttgtttgtttgtttgggtttttttttgagacagggtttctctctgtagccgtgtgtgtggggtagccctgtgtgtgagtgtgtgtgtgtgtgtttgtggtatgttgcaaaatagattaaacaactgagagatggaataggtcttcttgacatcaaaaacatgatcgtgaacccctttattaaatctaacactcagagacaggagcatctctgcaggtttgaggccagcctggtctacagaatgaatttcaggtctcaaggtcagcttggtctacaaagtgagtttcagatctcaaggccagccaagactatgcagtaagaccttagctaaaataaataaataaataaaataaaataaaagttaatcttcgcttggcaaaccgataattgaggaccagtgctcaggaaggagacacacgggaattccagaggctacagagggagcctcgctctgacctggttagagcaactctactttactggctgtgtctatgaggttctgcttgatttcatttgacaaaaagtttccacagctaaaccaggcaagggagccgaagtagacacagccacccgggccgcgccaacaggtttctctctgctgctgagaagcaaaagcctgtttgaagaaactctctgaaggagactgtggttgagtggtgggggtaggggtgctggggttgggctgaggctgagggttgactctaaggagctggaacctctcagcttcggtggctagacaggggagttgtaatgaagagggacaggtaccctccttggtggaggagggtggaatgaaactctcggtttcccccagaacttggcaaagtgtgtgtgatgtctcccaggtagtcccccaaaggaggaggctagcagagctggggcggcaggaagtgggtaactagactaacaaagatgcctgtggcggtttgcccatcccaggtgggagggtgggactagccttgggcctgggcctcctgtgaacttggtgcttgagggctcagagggaacccagtcaggagcttgaatcccacgattcagcgcttctgtctgcggcca

SEQ ID NO: 16 - DNA, 400 bp, Homo sapiens
  source 1..400: mol_type = genomic DNA
actagccacagacagaaaaacaggcagttcatgtttaaaggactccacctctttctcttcctcggggggaactgggttttcttaaatacaactgagtttttgtttacacattctgtaatttcttttaattcctgttccagtatctcactgtgaaactccctatgtttttatacgattctcagggggtttcctctgggcatgattgggcacaacttcccacagtcagctctgggtacgacctccacattgcagaattgagaagttgacccagaaatgcattttgggctgagcagacaattgtcagagttgctggctagaccacagatgtgtcaagggaccacggcctttctgtaagctcatggtcagaggcggaggggagttgtgaacgttctgatgaaag

SEQ ID NO: 17 - DNA, 400 bp, Homo sapiens
  source 1..400: mol_type = genomic DNA
ctagccacagacagaaaaacaggcagttcatgtttaaaggactccacctctttctcttcctcggggggaactgggttttcttaaatacaactgagtttttgtttacacattctgtaatttcttttaattcctgttccagtatctcactgtgaaactccctatgtttttatacgattctcagggggtttcctctgggcatgattgggcacaacttcccacagtcagctctgggtacgacctccacattgcagaattgagaagttgacccagaaatgcattttgggctgagcagacaattgtcagagttgctggctagaccacagatgtgtcagagggaccacggcctttctgtaagctcatggtcagaggcggaggggagttgtgaacgttctgatgaaag

SEQ ID NO: 18 - DNA, 1361 bp, Homo sapiens
  source 1..1361: mol_type = genomic DNA
tgggatgacaggcctgagccaccgtgtctggccagtatttggttttctgtttctgtgttaactcgcttaggataatggcctctagctgcatccatgttgctgcaaaggacataatcttgtgatttttcaaggctgtatagcgttctgtggtgtatacatatcacattgtctttatccagtccacctctgatgggacctgggtggattccatgtcttcactattgtgaatcctgctgcaatgaacatacaagtgcatgtgtctttttggtagaatgatttattttcctttggctatatacccagcgatgggattgctgggctgaatggtaactctgtttgtagttctctgaaatatctccaaaccaaactgctttccacagtggctgaactaatttacacccaccaacagtgtataagtgtcccctttgctccacaatctcaccagcatctgttaatttctggcttttcagtaatggccattctgactggtgtgagatggtattgttgagggataatttaggaatcagagagaccgaggggttgaggaggatttattattattattattatttaggtgcaccggccccagtcagattaacatccaaaaagactgaggctcgaacagagagtccggttaccttttaagcattttgtggggttgggggagatctgtgcagggggaagcatattacagaagcaagaaacaaaggcagttattcaattgagacatgcatcacattattccttacttttcaagaaaaatatgttttacgacttgaggttatcctgtctagtgaccttgcagccgcacggcaagagaaacagggtcttcacaatgcctgggaaagggagagataaggctcactagccacagacagaaaaacaggcagttcatgtttaaaggactccacctctttctcttcctcggggggaactgggttttcttaaatacaactgagtttttgtttacacattctgtaatttcttttaattcctgttccagtatctcactgtgaaactccctatgtttttatacgattctcagggggtttcctctgggcatgattgggcacaacttcccacagtcagctctgggtacgacctccacattgcagaattgagaagttgacccagaaatgcattttgggctgagcagacaattgtcagagttgctggctagaccacagatgtgtcagagggaccacggcctttctgtaagctcatggtcagaggcggaggggagttgtgaacgttctgatgaaagcagtcaacgtgaaagcgctctggtgatgggcgctggtgctcacccaccacttcctgtgtatctatctccctggcccgcccggctcagtccccactgc

SEQ ID NO: 19 - DNA, 1351 bp, Homo sapiens
  source 1..1351: mol_type = genomic DNA
tgggatgacaggcctgagccaccgtgtctggccagtatttggttttctgtttctgtgttaactcgcttaggataatggcctctagctgcatccatgttgctgcaaaggacataatcttgtgatttttcaaggctgtatagcgttctgtggtgtatacatatcacattgtctttatccagtccacctctgatgggacctgggtggattccatgtcttcactattgtgaatcctgctgcaatgaacatacaagtgcatgtgtctttttggtagaatgatttattttcctttggctatatacccagcgatgggattgctgggctgaatggtaactctgtttgtagttctctgaaatatctccaaaccaaactgctttccacagtggctgaactaatttacacccaccaacagtgtataagtgtcccctttgctccacaatctcaccagcatctgttaatttctggcttttcagtaatggccattctgactggtgtgagatggtattgttgagggataatttaggaatcagagagaccgaggggttgaggaggatttattattattattattatttaggtgcaccggccccagtcagattaacatccaaaaagactgaggctcgaacagagagtccggttaccttttaagcattttgtggggttgggggagatctgtgcagggggaagcatattacagaagcaagaaacaaaggcagttattcaattgagacatgcatcacattattccttacttttcaagaaaaatatgttttacgacttgaggttatcctgtctagtgaccttgcagccgcacggcaagagaaacagggtcttcacaatgcctgggaaagggagagataaggctcactagccacagacagaaaaacaggcagttcatgtttaaaggactccacctctttctcttcctcggggggaactgggttttcttaaatacaactgagtttttgtttacacattctgtaatttcttttaattcctgttccagtatctcactgtgaaactccctatgtttttatacgattctcagggggtttcctctgggcatgattgggcacaacttcccacagtcagctctgggtacgacctccacattgcagaattgagaagttgacccagaaatgcattttgggctgagcagacaattgtcagagttgctggctagaccacagatgtgtcagagggaccacggcctttctgtaagctcatggtcagaggcggaggggagttgtgaacgttctgatgaaagcagtcaacgtgaaagcgctctggtgatgggcgctggtgctcacccaccacttcctgtgtatctatctccctggcccgcccggctcag

SEQ ID NO: 20 - DNA, 1264 bp, Homo sapiens
  source 1..1264: mol_type = genomic DNA
tgggatgacaggcctgagccaccgtgtctggccagtatttggttttctgtttctgtgttaactcgcttaggataatggcctctagctgcatccatgttgctgcaaaggacataatcttgtgatttttcaaggctgtatagcgttctgtggtgtatacatatcacattgtctttatccagtccacctctgatgggacctgggtggattccatgtcttcactattgtgaatcctgctgcaatgaacatacaagtgcatgtgtctttttggtagaatgatttattttcctttggctatatacccagcgatgggattgctgggctgaatggtaactctgtttgtagttctctgaaatatctccaaaccaaactgctttccacagtggctgaactaatttacacccaccaacagtgtataagtgtcccctttgctccacaatctcaccagcatctgttaatttctggcttttcagtaatggccattctgactggtgtgagatggtattgttgagggataatttaggaatcagagagaccgaggggttgaggaggatttattattattattattatttaggtgcaccggccccagtcagattaacatccaaaaagactgaggctcgaacagagagtccggttaccttttaagcattttgtggggttgggggagatctgtgcagggggaagcatattacagaagcaagaaacaaaggcagttattcaattgagacatgcatcacattattccttacttttcaagaaaaatatgttttacgacttgaggttatcctgtctagtgaccttgcagccgcacggcaagagaaacagggtcttcacaatgcctgggaaagggagagataaggctcactagccacagacagaaaaacaggcagttcatgtttaaaggactccacctctttctcttcctcggggggaactgggttttcttaaatacaactgagtttttgtttacacattctgtaatttcttttaattcctgttccagtatctcactgtgaaactccctatgtttttatacgattctcagggggtttcctctgggcatgattgggcacaacttcccacagtcagctctgggtacgacctccacattgcagaattgagaagttgacccagaaatgcattttgggctgagcagacaattgtcagagttgctggctagaccacagatgtgtcagagggaccacggcctttctgtaagctcatggtcagaggcggaggggagttgtgaacgttctgatgaaag

SEQ ID NO: 21 - DNA, 1296 bp, Mus musculus
  source 1..1296: mol_type = genomic DNA
acccaaggagctgaaggggtgggcaaccctataggtggaacaacaatatcaactaaccagtacccccagagctcgtttctctagctgcatatacagcagaagttgaccatcactgggaagagaagcctcttggtcttgcaaactttatatgccccagtacaggggaatgccacagccaagaagtgggagtgggtgggtaggggagcagggcagggggatggtatagggaactttcgggatagcatttgaaatttgaaatgtatataaagaaagtatctaataaaaaggaaatctttaagacctaagagctaagtgtggcagcacacgcttatgatatctgtgctgggagacagagataggtggttactagggctcataggtcagccaaccttgcatacttggcaagatcctggctcttatgggacattgtaccaaaaaataagtggacatgtgtgcaaatacacataaacctaataagggttggggagacagttcagtgattaaaatgcttactatggtagtgtgaaggccagagtttatctccctagaacccacataaaatgtcaggtggatatggaatcctacttataattccagcctgggaatacagagacaagagatcccaagaacaagttggctagggagattcatcatattggtgagctctgggtttgattgagagaccctgcctcagtgaataagatagaagagtaaccaaggatgagccaagacatcaacttaggcttccacatgcacacatatatacaagcaaatatacatacttatgcatgccacacatgcacatgaagattaaaaacagtagaataatagcattttactgagtcaaaatagaaaaattattaatatagtattactcttggtttcctgttaaactaatgatcaaatccaaacacgtgcatgttacacatgggaatgcaaagactaaaatccccatattggggagtcctcagaccttttgctctgtgaatctcccccatgccttcccctgaatgaatttaaatgttaacccagaaatgggttgcagactgagcagttgatggtcagaataatcttttagatccaaaatgtggttaaatgtgttatgtgaaccaagtttttgcctgaagtcctaggtcaatggtaaagggagagctgtgaacctttctattaaaaaataaaaatgtgtgaaagtcccatggtgactagtgtcaggcctcacccaccacatcctgtgcatctccctcacccactggactcaatccacacacatagttcagcactggtctggccactggt

SEQ ID NO: 22 - DNA, 391 bp, Mus musculus
  source 1..391: mol_type = genomic DNA
aagcttgggaacccaagaacctatctacaagaaaaagaaaagggagagtgggcactcctctcccttcatgtcctgtcactctcatattttttttattaagatttaatctttatccttcttctttcagccactcttgcagaagaacaattcctaagggtcccttccaagtgttttctcttccccaaatctcagttttcaattcctcccacttgctctgcaagggtgcattctcactctgagttcccaggcccctaaaaggtggttgacactctttggtggggactttgggtgacatcatatcctcacataggaaatcagctctgtctgcagctggctaaaggagcagtttccccgaccctacacgcctcccccaccgcacctcctccgccct

SEQ ID NO: 23 - DNA, 1642 bp, Homo sapiens
  source 1..1642: mol_type = genomic DNA
gcacccccgccccgcaccttagttctgcgagcctttccccgtctccaacgcgggcaaggctgtcagatcctcaagccgacccttctctgcaccgcgacctcagtgtcctgcgcgggacatcagaggccggacgctccacctgccccagggcacgccccgagcccccaagcccatctccttcctgtcgggtgctcacctcttcctccctgcgggcccagggccttcttgaggggctgcggatgcgaatgagccagcgaagggccgcccgcggcccctcggcacctctgcccagccgtcccgaccaggggaccagctcagccccggtcggggccccggcacccaggaccgctcaggatctccattccagtcccttcgcactttctgacggggaaggagagttgggagaacgagactctcaagtctggggacttctacccaccattaaagctttggcattctggccatttctttctttttaaaaaattattattattattttttattttgagatgggagtctcgctctatcccccagcctggagtgcagtgactcgatctccgctcgctgcaacttccgcctccagggttcaaacgattcttctgcctcagcctccccagtagctaggactacaggcttgcgccatcacgcccggctagttttttttttgttttttgtttgtttgtttgtttgtttttaaagtagaaacaggatttcgccatattggccaggctggtctcgaactcctgacctcagttgatccgaccgcctcggcctcccaaagtgttggaattacagcagtgagccaccgcgcaccaagcccggcccattctggccatatctatagacggatgcattgcacaggaggctcagcactaatcggtagatactgcgagatgctgggaggttaaggggcctacccgcaatatctctggccaatgccttgggctagaaatgccataattagccgctcttttgatcccttgcaaaatgcgaatcccaccgcacctccaccccacccgagtggtaatctcctagtggtaatctaagtgagcctgtgataagataagtagctcctggtggtgagggtgagaaattggggagctggagccccagccagggacgaggctgtaggggctagggcgaagatggaggctgctgggcccccagatggaagacggtaacgtgcgcccgcttcgtttttgctcgaggtcagtcaggtgcagactgaattcgaagtcgctccctcctccgctcaaccccgaccaggccaaaactaaagcagcaccgccccctgctgggccgacagggcatcagattttgctggacgcgggtgacaggcgagatagggagtgtccctgctgctagtgcccctgctgctagtgcctagttacctgcagtacttaccctccctgggcctcagtttttcttcctctgtaaaataaatgcgctgggccggatcttttctgagttctcttctcccctacgaattctagatccctcctctgtcctccctgcgccagggaccttcgggcgacccttccctgtacccccaccccaccctctctggaccccgtttctgcctcagtacggcgcgctgagctctgccccctgcccaggccctgaccccctcaggagccgcggt

SEQ ID NO: 24 - DNA, 639 bp, Homo sapiens
  source 1..639: mol_type = genomic DNA
aagatttggctcagaagatgtccctaaccccacgggctttcattctcatgaccggggggacacaggctcaaggctggcacttgcgtggctccaagagtgagtggcctcagtgcctcactggctgcagcctcatgccagccccggacagaccagagaaagacccgggtgataccaacacaagcagagggcatggattgccttctcaaaaaagttatgtttggctgggagtgttgttcacagcctcttacagattttgtggaggtctctgcggttgggtgagggcaaagttacgaggggaagatgtgaggtggtgttggtggaggggtggaggctcctcttccccacacacccctccccagcaccccttcagcatcctgcaagcctgaagctgcctgctgggctaggcctgctgtctgggcaagaccagaccaggcaaaagggaggccatgactcccacccctccacccttacctcagcctcagccaagtttgggggacatagggaggggagagcaacagtaaacgtgatcagcacaaaaggcgggcctgccttcggatcacgaaggctctagctgggccagggcagaagatggagtttgggtcgtccctgcctctatccgagtacctaccctgcagccac

SEQ ID NO: 25 - DNA, 910 bp, Homo sapiens
  source 1..910: mol_type = genomic DNA
aaaaatgagccgggtattgtggtgcatgcctataatccgggctactcaggaggctgaggcaggagaatcgcttgaacgtgggaggcggaggttgcagtgagctgagatcgtaccactgcactccagcctgggtaacagagcaagattccatctcacaaaaaaaaaaaaaagtggggggggaagtttttagcttttcttttcttttcttttcttttttttttttttttttttttgagacggagtctcgctatctcgcccaggctggagtgcagtggtgcgatctcggctcactgcaagctccgcctcccgggttcacgccattctcctgcctcaacctcccaagtagctgggactacaggcacccgccacaacacccggctaattttttgtatttttagtagagacggggtttcaccgtgttagccaggatggtctcgatctcctgaccacgtgatccgcccgccttggcctcccaaagtgctgggattataggcgtgagctaccgcacccggccagtttttagcatttttattattcagggtgcactttggaacttttgcagaaggaccatcccttttctcgatcttttctgaaaactctcattgtgagatgaataactgagaataaagttctgccttaaaatacaaagctatacagattaaacacacctgggaaattccaacacccgagttaatatactcagtgatgatggtgggccgctcagcttcaacagagaggttttagagggctgagagtgcagacatgacgtcatgatgaattagcatgtttacaaggtgctagcctggtgctgaattaacacgtttttagcgtaacatcatttggctgatggtggtgatttgctcacatcctcctctctccctgtcatcctccggggctgagaatcaggtca

SEQ ID NO: 26 - DNA, 653 bp, Homo sapiens
  source 1..653: mol_type = genomic DNA
gagtctcgctatctcgcccaggctggagtgcagtggtgcgatctcggctcactgcaagctccgcctcccgggttcacgccattctcctgcctcaacctcccaagtagctgggactacaggcacccgccacaacacccggctaattttttgtatttttagtagagacggggtttcaccgtgttagccaggatggtctcgatctcctgaccacgtgatccgcccgccttggcctcccaaagtgctgggattataggcgtgagctaccgcacccggccagtttttagcatttttattattcagggtgcactttggaacttttgcagaaggaccatcccttttctcgatcttttctgaaaactctcattgtgagatgaataactgagaataaagttctgccttaaaatacaaagctatacagattaaacacacctgggaaattccaacacccgagttaatatactcagtgatgatggtgggccgctcagcttcaacagagaggttttagagggctgagagtgcagacatgacgtcatgatgaattagcatgtttacaaggtgctagcctggtgctgaattaacacgtttttagcgtaacatcatttggctgatggtggtgatttgctcacatcctcctctctccctgtcatcctccg

SEQ ID NO: 27 - DNA, 1720 bp, Homo sapiens
  source 1..1720: mol_type = genomic DNA
  misc_difference 830: note = a, c, t, g, unknown or other
  misc_difference 831: note = a, c, t, g, unknown or other
  misc_difference 856: note = a, c, t, g, unknown or other
  misc_difference 872: note = a, c, t, g, unknown or other
  misc_difference 873: note = a, c, t, g, unknown or other
  misc_difference 876: note = a, c, t, g, unknown or other
  misc_difference 909: note = a, c, t, g, unknown or other
  misc_difference 910: note = a, c, t, g, unknown or other
  misc_difference 931: note = a, c, t, g, unknown or other
  misc_difference 935: note = a, c, t, g, unknown or other
  misc_difference 1233: note = a, c, t, g, unknown or other
  misc_difference 1234: note = a, c, t, g, unknown or other
tcgccctcctacctacctgatttcttccttcatctccttccttcctctagcctcacacagacagtgtgttgggcagtctctcgccacatcctggctgtctggtgtttctagcccacagggctccctgggtgaagggtctgcgccaccacgttcccacagctacttgttgactccagtccctatcacaacctttagggttttggctgcctcatccagccgagggaggctgctgtgttcaggagcccagccgcacagaagtttccatgatgtcatgaatgggagtggcagagacaacaaggggccctgggactagtttctcaccaagactcagagggtgtgtgtaacaaagtcccactgctgctaaggggatggcctcaactctacggccctgctgaccaggtgccactagctgtaaggaggtctcacctaagcccctttcgtatccctttcccctgtatcaaaggcaccattttagcttcctttcgccaggacctgtctctggcttgctcctgccttctgcatgataaatggggatcagaaacccttcaccaattaaagcacattatcccgctccatctacctggaaagcaggagtttgagacagggatttttttttttttgagatggagtcttgctctgttgctcaggctggagtgctgtggcatgatcttggcttactgcaacctctgtctcctggagtcaagtgattctcctgcctcagcctcctgagtagctgggattgcaagcatgtgtcaccaagttgagctaattttgtattttagtagagacagggtttcatcatgttggccaggctggtctcgaagtgttgnnacctcaatgatccactcactcagcntcaaagtgctgggatnngangcatgagagcactgcactgctgagacaggatannttcatagatgtcatcttctantacntctaatggtagtggttctaggctgggcacaatggctcatccatgtaatcccagcactttaggaggcagaggcaagaggatcacttgagcctaggagttcaaaaccagcctgggcaacatagcaacatcccgtctcgaccaaaaaaaaaaaaaaaaatcagctcagtgcagtggtgggcacctgtagtcctagctatttgggaggctgaggtgggaggattgcatgaggctgggaattggaggctgcagtgagttatgattgtgcctctgcactccagcctgggcaacagagtgagacnnctatcttaaaaaaaaaatagctatggttcaaaccttggaagcctgagggatttattaaaaatataaatgcagccaggcatggtggctcacacctgtaatcccagcacttggagttagcggcagatcacaagatcagaagattgagtatcaccctcgcaacatgttgaaaccccgttctactaaaaatacaaaaattagctgggcatggtgcgcacgcctgtaatcccagctactcgggaggctgaggcaggagaattgcttgaacccgggaggcggaggttgcagtgagtcaagattgcgccaccgcactccagcctggtgacagagcgagacttggtctcaaaataaaataaataaaataaaataaaagtgctagagacgcactctaggcagggtgtttccagttgattctgaaacaaaaaaaattcagagtctcttctttagagggagaatagaggcaatctttctctcttatgtcttttatgc

SEQ ID NO: 28 - DNA, 339 bp, Mus musculus
  source 1..339: mol_type = genomic DNA
tgttggggttcaaatttgagccccagctgttagccctctgcaaagaaaaaaaaaaaaaaaaaagaacaaagggcctagatttcccttctgagccccaccctaagatgaagcctcttctttcaagggagtggggttggggtggaggcggatcctgtcagctttgctctctctgtggctggcagtttctccaaagggtaacaggtgtcagctggctgagcctaggctgaaccctgagacatgctacctctgtcttctcatggctggaggcagcctttgtaagtcacagaaagtagctgaggggctctggaaaaaagacagccagggtggaggtagattggt

SEQ ID NO: 29 - DNA, 467 bp, Cytomegalovirus
  source 1..467: mol_type = genomic DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgg

SEQ ID NO: 30 - DNA, 388 bp, Mus musculus
  source 1..388: mol_type = genomic DNA
cgggattttgctgccttaccacagcccaaagcaggctgtgtgtttagaagctgagccacaaagaagtttccatgacatcatgaatgggggtggcagagaagaatattggggctcagagggtgtgtgtgtgtgtgtgtgtgtgtgtgtgtgtgtgtgtgagagagagagagagagagagagagagagagagacagacagagagacagagagacagaaagacagaaagacagaaagacagagacagagctgtcaagaattttcttctacttcgtgggttctggggattgtactaaggtcatgaggcttgtgtggcaagcacccttacccactgagccatcttgcctgccctcatcctcaaattaattaaaaataaagaacatgatgaatt

SEQ ID NO: 31 - DNA, 133 bp, unidentified
  source 1..133: mol_type = unassigned DNA
gtaagtatcaaggttacaagacaggtttaaggaggccaatagaaactgggcttgtcgagacagagaagattcttgcgtttctgataggcacctattggtcttactgacatccactttgcctttctctccacag

SEQ ID NO: 32 - DNA, 144 bp, synthetic construct
  source 1..144: mol_type = other DNA
gtaagggcctactaggggtctggaagcctggggaagggctcaagggaagagcccatcactagtgagacaggaatattggtatccctaaccttcagcctacctctgctgtcaccttagagttcaaagaagggcaaaatggaggct

SEQ ID NO: 33 - DNA, 20 bp, Homo sapiens
  source 1..20: mol_type = genomic DNA
atttaagcacgactctgcag

SEQ ID NO: 34 - DNA, 11 bp, Homo sapiens
  source 1..11: mol_type = genomic DNA
gggtagtcgac

SEQ ID NO: 35 - DNA, 142 bp, Homo sapiens
  source 1..142: mol_type = genomic DNA
tggagccagtctagctgctgcacaggctggctggctggctggctgctaagggctgctccacgcttttgccggaggacagagactgactaggaacaggggaagggcctggctgtcctcatcctggctatcattcttcttcaag

SEQ ID NO: 36 - DNA, 36 bp, synthetic construct
  source 1..36: mol_type = other DNA
tcttactgacatccactttgcctttctctccacagg

SEQ ID NO: 37 - DNA, 539 bp, synthetic construct
  source 1..539: mol_type = other DNA
tgttggggttcaaatttgagccccagctgttagccctctgcaaagaaaaaaaaaaaaaaaaaagaacaaagggcctagatttcccttctgagccccaccctaagatgaagcctcttctttcaagggagtggggttggggtggaggcggatcctgtcagctttgctctctctgtggctggcagtttctccaaagggtaacaggtgtcagctggctgagcctaggctgaaccctgagacatgctacctctgtcttctcatggctggaggcagcctttgtaagtcacagaaagtagctgaggggctctggaaaaaagacagccagggtggaggtagattggtgcatgcagcttctgcagccccgcgtgctagagatttaagcctgattctgcttaactttttcccttgactttggcattttcactttgacatgttccctgagagcctggggggtggggaaccagctccagctggtgacgtttggggccggcccaggcctagggtgtggaggagccttgccatcgggcttcctgtctctcttc

SEQ ID NO: 38 - DNA, 175 bp, synthetic construct
  source 1..175: mol_type = other DNA
atttaagcacgactctgcagtctagacacaggtaagtatcaaggttacaagacaggtttaaggaggccaatagaaactgggcttgtcgagacagagaagattcttgcgtttctgataggcacctattggtcttactgacatccactttgcctttctctccacaggggtagtcgac

SEQ ID NO: 39 - DNA, 615 bp, synthetic construct
  source 1..615: mol_type = other DNA
tgatcagaaacaagaggatctgctaatgtggcttgtgtaatagtttctctcagtggatctaaagaggtggcacttccttgtttgtgagtaaatgtagttgtattgttcataggaagcccaaaatcttctttgtttgttaccagtgactattgctatttgttttttgttaccaggaaaggctattctaaattcccttacacttcctagaattctgaacaaactttcctcatagtctctgctttgtatcaatcatcccaaactctttgcttcagaaagagcagtttgcacctgtttgtgtcttaccccacacctagcccactgttgctttgacagtcttacaccatcaaaggcagaaggtagagaggcagatttcttctagttcctcccccactcttcctttttctgaatacccatgagtctgttggcggaggatagtgctaaccctggctgatcatctagacacaggtaagtatcaaggttacaagacaggtttaaggaggccaatagaaactgggcttgtcgagacagagaagattcttgcgtttctgataggcacctattggtcttactgacatccactttgcctttctctccacaggggtagtcgacgccacc

SEQ ID NO: 40 - DNA, 640 bp, synthetic construct
  source 1..640: mol_type = other DNA
aacaactggctacgatcctaacaactaatgacagggacatttatctccctcatgaagaaacggtcccaggaccatctcccacccagcatccattgcggttccctgtgcaagatgagtctctgagtgggaatccagcactctctccctcttcttccccaccaccttcaccctccttaacggaaaaacaaaaggcatctgcacctgcagccctgctgaggcccctgctgctcacacttgcagcagagggtggaggctctgggttcttgccttctctcaaaggccccagccccaacagtgatgggtggagccagtctagctgctgcacaggctggctggctggctggctgctaagggctgctccacgcttttgccggaggacagagactgactaggaacaggggaagggcctggctgtcctcatcctggctatcattcttcttcaaggtaagggcctactaggggtctggaagcctggggaagggctcaagggaagagcccatcactagtgagacaggaatattggtatccctaaccttcagcctacctctgctgtcaccttagagttcaaagaagggcaaaatggaggcttcttactgacatccactttgcctttctctccacaggggtagtcgacgccacc

SEQ ID NO: 41 - protein, 475 aa, synthetic construct
MGVPTQLLGLLLLWITDAICDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 42 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 43 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 108 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITG

SEQ ID NO: 45 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 46 - protein, 45 aa, synthetic construct
TTTPAPRPPTPAPTIASQPLSLRPEACRPAAGGAVHTRGLDFACD

SEQ ID NO: 47 - protein, 20 aa, synthetic construct
AEPKSPDKTHTCPPCPKDPK

SEQ ID NO: 48 - protein, 24 aa, synthetic construct
IYIWAPLAGTCGVLLLSLVITLYC

SEQ ID NO: 49 - protein, 27 aa, synthetic construct
FWVLVVVGGVLACYSLLVTVAFIIFWV

SEQ ID NO: 50 - protein, 21 aa, synthetic construct
VAAILGLGLVLGLLGPLAILL

SEQ ID NO: 51 - protein, 27 aa, synthetic construct
IISFFLALTSTALLFLLFFLTLRFSVV

SEQ ID NO: 52 - protein, 21 aa, synthetic construct
WITAVLPTVIICVMVFCLILW

SEQ ID NO: 53 - protein, 26 aa, synthetic construct
VMSVATIVIVDICITGGLLLLVYYWS

SEQ ID NO: 54 - protein, 21 aa, synthetic construct
GFLFAEIVSIFVLAVGVYFIA

SEQ ID NO: 55 - protein, 21 aa, synthetic construct
GIIVTDVIATLLLALGVFCFA

SEQ ID NO: 56 - protein, 21 aa, synthetic construct
LCYLLDGILFIYGVILTALFL

SEQ ID NO: 57 - protein, 23 aa, synthetic construct
VIGFRILLLKVAGFNLLMTLRLW

SEQ ID NO: 58 - protein, 21 aa, synthetic construct
ILLGKATLYAVLVSALVLMAM

SEQ ID NO: 59 - protein, 23 aa, synthetic construct
YYMYLLLLLKSVVYFAIITCCLL

SEQ ID NO: 60 - protein, 23 aa, synthetic construct
YYTYLLLLLKSVVYFAIITCCLL

SEQ ID NO: 61 - protein, 23 aa, synthetic construct
LGLRMLFAKTVAVNFLLTAKLFF

SEQ ID NO: 62 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 41 aa, synthetic construct
RSKRSRLLHSDYMNMTPRRPGPTRKHYQPYAPPRDFAAYRS

SEQ ID NO: 65 - protein, 36 aa, synthetic construct
RDQRLPPDAHKPPGGGSFRTPIQEEQADAHSTLAKI

SEQ ID NO: 66 - protein, 48 aa, synthetic construct
QRRKYRSNKGESPVEPAEPCHYSCPREEEGSTIPIQEDYRKPEPACSP

SEQ ID NO: 67 - protein, 42 aa, synthetic construct
KRGRKKLLYIFKQPFMRPVQTTQEEDGCSCRFPEEEEGGCEL

SEQ ID NO: 68 - protein, 36 aa, synthetic construct
MQDEDGYMTLNVQSKKRSSAQTSQLTFKDYSVTLHW

SEQ ID NO: 69 - protein, 55 aa, synthetic construct
KNRKAKAKPVTRGAGAGGRQRGQNKERPPPVPNPDYEPIRKGQRDLYSGLNQRRI

SEQ ID NO: 70 - protein, 45 aa, synthetic construct
GQDGVRQSRASDKQTLLPNDQLYQPLKDREDDQYSHLQGNQLRRN

SEQ ID NO: 71 - protein, 45 aa, synthetic construct
GHETGRLSGAADTQALLRNDQVYQPLRDRDDAQYSHLGGNWARNK

SEQ ID NO: 72 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 73 - protein, 28 aa, Homo sapiens
ERPPPVPNPDYEPIRKGQRDLYSGLNQR

SEQ ID NO: 74 - protein, 29 aa, Homo sapiens
DKQTLLPNDQLYQPLKDREDDQYSHLQGN

SEQ ID NO: 75 - protein, 29 aa, Homo sapiens
DTQALLRNDQVYQPLRDRDDAQYSHLGGN

SEQ ID NO: 76 - protein, 29 aa, Homo sapiens
APAYQQGQNQLYNELNLGRREEYDVLDKR

SEQ ID NO: 77 - protein, 29 aa, Homo sapiens
PQRRKNPQEGLYNELQKDKMAEAYSEIGM

SEQ ID NO: 78 - protein, 29 aa, Homo sapiens
ERRRGKGHDGLYQGLSTATKDTYDALHMQ

SEQ ID NO: 79 - protein, 185 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 80 - protein, 160 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 81 - protein, 150 aa, Homo sapiens
FKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 82 - protein, 143 aa, Homo sapiens
QSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 83 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 84 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 85 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 86 - protein, 189 aa, Homo sapiens
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 87 - protein, 153 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 88 - protein, 125 aa, synthetic construct
MGSGPSNFVNHAPGEAIVTGLEKGADKVAGTISHTIWEVIAGLVALLTFLAFGFWLFKYLQKRRERRRQLTEFQKRYLRNSYRLSEIQRPISQHEYEDPYEPPSRRKPPPPPYSTYVNIDNVSAI

SEQ ID NO: 89 - protein, 154 aa, synthetic construct
MGSGPSNFVNHAPGEAIVTGLEKGADKVAGTISHTIWEVIAGLVALLTFLAFGFWLFKYLQWYNRKSKNKKRKEQIREQIELGLLSYGAGVASLPLLNVIAHNPGSVISATPIYKGPCTGVPNSRLLQITSGTAEENTRILNHDGRNPDGSINV

SEQ ID NO: 90 - protein, 38 aa, synthetic construct
MGSGPSNFVNHAPGEAIVTGLEKGADKVAGTISHTIWE

SEQ ID NO: 91 - protein, 48 aa, synthetic construct
KLLQWYNRKSKNKKRKEQIREQIELGLLSYGAGVASLPLLNVIAHNPG

SEQ ID NO: 92 - protein, 48 aa, synthetic construct
VISATPIYKGPCTGVPNSRLLQITSGTAEENTRILNHDGRNPDGSINV

SEQ ID NO: 93 - DNA, 228 bp, synthetic construct
  source 1..228: mol_type = other DNA
ctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatgg

SEQ ID NO: 94 - DNA, 56 bp, synthetic construct
  source 1..56: mol_type = other DNA
aataaaggaaatttattttcattgcaatagtgtgttggaattttttgtgtctctca

SEQ ID NO: 95 - DNA, 1428 bp, synthetic construct
  source 1..1428: mol_type = other DNA
atgggtgtccctacccagctcctgggactgctcctgctgtggatcaccgacgccatctgcgacatccagatgacccagagccctgccagcctgtctaccagcctgggcgagacagtgaccatccagtgtcaggccagcgaggacatctactctggcctggcttggtatcagcagaagcccggcaagagccctcagctgctgatctacggcgccagcgacctgcaggacggcgtgcctagcagattcagcggcagcggctccggaacccagtacagcctgaagatcaccagcatgcagaccgaggacgagggcgtgtacttctgccagcaaggcctgacctaccctagaaccttcggaggaggcaccaagctggaactgaagggcggaggcggaagtggaggcggaggatctggcggcggaggctctgaagtgcagctgcagcagtctggcgctgaactggtccggcctggcactagcgtgaagctgtcctgcaaggtgtccggcgacaccatcaccttctactacatgcacttcgtgaagcagaggccaggacagggcctggaatggatcggcagaatcgaccctgaggacgagagcaccaagtacagcgagaagttcaagaacaaggccaccctgaccgccgacaccagcagcaacaccgcctacctgaagctgtctagcctgacctccgaggacaccgccacctacttttgcatctacggcggctactacttcgactactggggccagggcgtgatggtcaccgtgtccagcatcgagttcatgtacccccctccctacctggacaacgagagaagcaacggcaccatcatccacatcaaagaaaagcacctgtgccacacccagagcagccccaagctgttctgggccctggtggtggtggccggcgtgctgttctgttacggcctgctggtcacagtggccctgtgcgtgatctggaccaacagcagaagaaacagaggcggccagagcgactacatgaacatgacccccagaaggccaggcctgaccagaaagccctaccagccctacgcccctgccagagacttcgccgcctacagacccagagccaagttcagcagatccgccgagacagccgccaacctgcaggatcccaaccagctgttcaacgagctgaacctgggcagacgggaggagttcgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgttccagggcctgtccaccgccaccaaggacaccttcgacgccctgcacatgcagaccctggcccccagatga

SEQ ID NO: 96 - protein, 22 aa, synthetic construct
MLLLVTSLLLCELPHPAFLLIP

SEQ ID NO: 97 - protein, 25 aa, synthetic construct
MNRGVPFRHLLLVLQLALLPAATQG

SEQ ID NO: 98 - protein, 15 aa, synthetic construct
GGGGSGGGGSGGGGS

SEQ ID NO: 99 - protein, 5 aa, synthetic construct
GGGGS

SEQ ID NO: 100 - protein, 4 aa, synthetic construct
GGGS

SEQ ID NO: 101 - skipped

SEQ ID NO: 102 - protein, 18 aa, synthetic construct
KESGSVSSEQLAQFRSLD

SEQ ID NO: 103 - protein, 14 aa, synthetic construct
EGKSSGSGSESKST

SEQ ID NO: 104 - protein, 12 aa, synthetic construct
GSAGSAAGSGEF

SEQ ID NO: 105 - protein, 5 aa, synthetic construct
EAAAK

SEQ ID NO: 106 - protein, 5 aa, synthetic construct
EAAAR

SEQ ID NO: 107 - protein, 5 aa, synthetic construct
PAPAP

SEQ ID NO: 108 - protein, 12 aa, synthetic construct
AEAAAKEAAAKA

SEQ ID NO: 109 - protein, 7 aa, synthetic construct
SGGGSDP

SEQ ID NO: 110 - protein, 24 aa, synthetic construct
APVKQTLNFDLLKLAGDVESNPGP

SEQ ID NO: 111 - protein, 19 aa, synthetic construct
ATNFSLLKQAGDVEENPGP

SEQ ID NO: 112 - protein, 20 aa, synthetic construct
QCTNYALLKLAGDVESNPGP

SEQ ID NO: 113 - protein, 18 aa, synthetic construct
EGRGSLLTCGDVEENPGP

SEQ ID NO: 114 - protein, 27 aa, synthetic construct
GSGAPVKQTLNFDLLKLAGDVESNPGP

SEQ ID NO: 115 - protein, 22 aa, synthetic construct
GSGATNFSLLKQAGDVEENPGP

SEQ ID NO: 116 - protein, 23 aa, synthetic construct
GSGQCTNYALLKLAGDVESNPGP

SEQ ID NO: 117 - protein, 21 aa, synthetic construct
GSGEGRGSLLTCGDVEENPGP

SEQ ID NO: 118 - protein, 28 aa, synthetic construct
RRKRAPVKQTLNFDLLKLAGDVESNPGP

SEQ ID NO: 119 - protein, 23 aa, synthetic construct
RRKRATNFSLLKQAGDVEENPGP

SEQ ID NO: 120 - protein, 24 aa, synthetic construct
RRKRQCTNYALLKLAGDVESNPGP

SEQ ID NO: 121 - protein, 22 aa, synthetic construct
RRKREGRGSLLTCGDVEENPGP

SEQ ID NO: 122 - protein, 31 aa, synthetic construct
RRKRGSGAPVKQTLNFDLLKLAGDVESNPGP

SEQ ID NO: 123 - protein, 26 aa, synthetic construct
RRKRGSGATNFSLLKQAGDVEENPGP

SEQ ID NO: 124 - protein, 27 aa, synthetic construct
RRKRGSGQCTNYALLKLAGDVESNPGP

SEQ ID NO: 125 - protein, 25 aa, synthetic construct
RRKRGSGEGRGSLLTCGDVEENPGP

SEQ ID NO: 126 - protein, 984 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSASGGGGSGGGGSQRRKYRSNKGESPVEPAEPCHYSCPREEEGSTIPIQEDYRKPEPACSPGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGGGGSGAMVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 127 - protein, 559 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSASGGGGSGGGGSQRRKYRSNKGESPVEPAEPCHYSCPREEEGSTIPIQEDYRKPEPACSPGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - DNA, 2952 bp, synthetic construct
  source 1..2952: mol_type = other DNA
atgctgctgctggtcacaagcctgctgctgtgcgagctgccccaccccgcctttctgctgatccccgacatccagatgacccagaccaccagcagcctgagcgccagcctgggcgacagagtgaccatcagctgccgggccagccaggacatcagcaagtacctgaactggtatcagcagaaacccgacggcaccgtgaagctgctgatctaccacaccagccggctgcacagcggcgtgcccagcagattttctggcagcggatctggcaccgactacagcctgaccatctccaacctggaacaggaagatatcgctacctacttctgtcagcagggcaacaccctgccctacaccttcggcggaggcaccaagctggaaatcaccggcagcaccagcggctccggcaagcctggatctggcgagggcagcaccaagggcgaagtgaagctgcaggaaagcggccctggcctggtcgcccctagccagagcctgtccgtgacctgtaccgtgtccggcgtgtccctgcccgactacggcgtgtcctggatcagacagccccccagaaagggcctggaatggctgggcgtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgacagtctcttctgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccctgggcccacccgcaagcattaccagccctatgccccaccacgcgacttcgcagcctatcgctccgctagcggaggtggaggttctggaggtggtggaagtcaaagaaggaagtaccgcagcaacaaaggagaatctcccgtcgagccagccgagccctgtcattattcatgcccaagggaggaggagggaagtacaatcccaattcaagaagactacaggaagcccgaacctgcatgcagtccaggtggaggcggttctggaggcggtggctcccgggtgaaattctcacggtctgcagacgcacccgcttaccagcaaggccagaaccaactctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggctccggagccacgaacttctctctgttaaagcaagcaggagacgtggaagaaaaccccggtcccatgggagtgcaggtggaaaccatctccccaggagacgggcgcaccttccccaagcgcggccagacctgcgtggtgcactacaccgggatgcttgaagatggaaagaaagtggactcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggtggaggcggttcaggcggcggcggcagcggcgccatggtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagt

SEQ ID NO: 129 - protein, 423 aa, synthetic construct
GSGATNFSLLKQAGDVEENPGPMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGGGGSGAMVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 130 - protein, 5 aa, synthetic construct
SGGGS